(19) United States
(12) Patent Application Publication
(10) Pub. No.: US 2024/0417393 A1
(43) Pub. Date: Dec. 19, 2024

(54) CERTAIN 2,5-DIAZABICYCLO[4.2.0]OCTANES AS GLP-1 RECEPTOR MODULATORS

(71) Applicant: ASTRAZENECA AB, Sodertalje (SE)

(72) Inventors: Magnus POLLA, Sodertalje (SE); Joakim BERGMAN, Sodertalje (SE); Johan SUNDELL, Sodertalje (SE); Jonas BRÅNALT, Sodertalje (SE); Ekaterina RATKOVA, Sodertalje (SE); Johan KAJANUS, Sodertalje (SE); Magnus JOHANSSON, Sodertalje (SE)

(21) Appl. No.: 18/698,404

(22) PCT Filed: Oct. 4, 2022
(86) PCT No.: PCT/EP2022/077530

§ 371 Date: Apr. 4, 2024

Related U.S. Application Data
(60) Provisional application No. 63/262,105, filed on Oct. 5, 2021.
(60) Provisional application No. 63/264,441, filed on Nov. 23, 2021.

(51) Int. Cl. C07D 405/14; A61K 31/498

(52) U.S. Cl. CPC C07D 405/14; A61K 31/498

(57) ABSTRACT

There are disclosed certain 2,5-diazabicyclo[4.2.0]octanes of formula (I), and pharmaceutically acceptable salts thereof, together with compositions containing them and their use in therapy. The compounds are GLP-1 receptor modulators and are thereby particularly useful in the treatment or prophylaxis of cardiovascular disease and metabolic conditions, for example Type 2 diabetes.

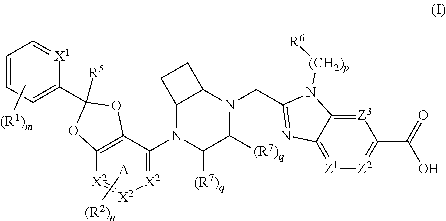

### TECHNICAL FIELD

The technical field relates to certain 2,5-diazabicyclo[4.2.0]octanes, to their use in the treatment of cardiovascular disease and metabolic conditions, for example type 2 diabetes, and to pharmaceutical compositions containing them.

### BACKGROUND

Obesity and type 2 diabetes (T2D) are major and growing health problems worldwide (Lancet, 2014, 9922, 1068-1083). The two diseases are strongly associated with each other, with obesity proceeding development of insulin resistance and T2D. T2D is associated with several comorbidities including cardiovascular disease, renal disease, hypertension, stroke, non-alcoholic fatty liver disease (NAFLD) and non-alcoholic steatohepatitis (NASH) (Lancet, 2005, 9468, 1415-1428).

Incretin hormones including GLP-1 (glucagon-like peptide-1) and GIP (glucose-dependent insulinotropic polypeptide) are gut peptides that are secreted after nutrient intake and stimulate insulin secretion (Diabetes ObesMetab., 2018, 20(Suppl.1), 5-21). GLP-1 secretion from the gut is impaired in obese subjects which may indicate a role in the pathophysiology of obesity (Regulatory Peptides, 2004, 122, 209-217).

GLP-1 is secreted from the L-cells in the lower gut in response to food intake. GLP-1 stimulates insulin secretion from the pancreatic P-cells, in a glucose dependent manner (Diabetologia, 1993, 36, 741-744). GLP-1 also inhibits glucagon secretion, reduces appetite and slows down gastric emptying. The GLP-1 receptor is also present in the heart, kidneys and immune system and activation has been shown to reduce blood pressure, increase natriuresis and decrease inflammation.

GLP-1 is a 37-amino acid peptide, post-translationally processed from pro-glucagon, a 158 amino acid precursor polypeptide (www.uniprot.org, pro-glucagon entry P01275). Several other peptides are also derived from proglucagon and processed in a tissue specific manor, including glucagon and oxyntomodulin. GLP-1 has very short half-life in vivo as it is rapidly degraded by dipeptidyl peptidase-4 (DPP-IV) (Front. Endocrinol. 2019, 10, Article 260, 1-10).

Incretin-based glucose- and body weight-lowering medications include GLP-1 receptor agonists, DPP-IV inhibitors and more recently also combinations of GLP-1 agonists and glucose-dependent insulinotropic polypeptide (GIP) agonists (Peptides, 2020, 125, Article 170202). Traditionally GLP-1 analogues are peptide hormones which have been modified to minimize DPP-IV cleavage and are administered as injectables. The first oral GLP-1 peptide was recently approved but bioavailability is low and the drug needs to be administered in the fasting state, 30 min before nutrient intake which may limit patient compliance (JAMA, 2017, 318(15), 1460-1470). The injectable peptides show increased efficacy over the oral peptides but are limited by the route of administration. Small molecule GLP-1 receptor agonists are in development from several companies and are expected to provide a therapeutic benefit versus peptide based therapies due to early use in the treatment paradigm.

Pharmacological stimulation of GLP-1 receptors has been shown to significantly reduce HbAlc levels, provide long term weight loss and reduce blood pressure. GLP-1 receptor agonists have also been shown to reduce cardiovascular events and prolong life in high-risk patients with T2D and are therefore recommended by the European Association for the Study of Diabetes (EASD) and American Diabetes Association (ADA) in patients with multiple risk factors of cardiovascular disease (CVD) independent of the patients glycemic control (Diabetes Care, 2020, 43, 487-493).

There remains a need for an easily-administered prevention and/or treatment for cardiometabolic and associated diseases.

WO2018/109607 discloses 6-carboxylic acids of benzimidazoles and 4-aza-, 5-aza-, 7-aza- and 4,7-diazabenzimidazoles as GLP-1 receptor agonists, processes to make said compounds, and methods comprising administering said compounds to a mammal in need thereof.

WO2019/239319 and WO2019/239371 disclose 6-carboxylic acids of benzimidazoles and 4-aza-, 5-aza- and 7-aza-benzimidazoles as GLP-1 receptor agonists, processes to make said compounds, and methods comprising administering said compounds to a mammal in need thereof.

WO2020/103815 disclose GLP-1 receptor agonist compounds and pharmaceutical compositions thereof, for use in e.g. treating type 2 diabetes mellitus, pre-diabetes, obesity, non-alcoholic fatty liver disease, non-alcoholic steatohepatitis and cardiovascular disease.

WO2020/207474 disclose GLP-1 receptor agonist compounds and pharmaceutical compositions thereof, for use in e.g. treating type 2 diabetes mellitus, pre-diabetes, obesity, non-alcoholic fatty liver disease, non-alcoholic steatohepatitis and cardiovascular disease.

WO2020/234726 disclose combinations of GLP-1 receptor agonist compounds and pharmaceutical compositions thereof and an acetyl-CoA carboxylase (ACC) inhibitor or a diacylglycerol acyltransferase (DGAT2) inhibitor, or a ketohexokinase (KHK) inhibitor or farnesoid X receptor (FXR) agonist, for use in e.g. treating type 2 diabetes mellitus, pre-diabetes, obesity, non-alcoholic fatty liver disease, non-alcoholic steatohepatitis and related diseases.

WO2020/263695 discloses glucagon-like peptide-1 receptor agonists and therapeutic uses of the compounds to treat type II diabetes mellitus.

WO2021/081207 discloses compounds that bind to and act as agonists or modulators of the glucagon-like peptide-1 receptor (GLP-1R) and act as agonists or modulators of GLP-1R. The disclosure further relates to the use of the compounds for the treatment and/or prevention of diseases and/or conditions by said compounds.

WO2021/018023 discloses compounds for modulating a Glucagon-like peptide-1 (GLP-1) receptor, and a pharmaceutical use thereof.

WO2021/096284 and WO2021/096304 discloses compounds that act as GLP-1 receptor agonists, for use as therapeutic agents for metabolic diseases.

WO2021/112538 discloses compounds which serves as a GLP-1 receptor agonist and may be useful in the prevention or treatment of a disease associated with GLP-1 activity.

WO2021/154796 discloses GLP-1R agonists, and compositions, methods, and kits thereof. Such compounds are generally useful for treating a GLP-1R mediated disease or condition.

WO2021/160127 discloses GLP-1 agonists, pharmaceutical compositions, and methods of use thereof.

WO2021116874 discloses of solid forms of 2-[[4-[(S)-2-(5-chloropyridin-2-yl)-2-methylbenzo[d][1,3]dioxol-4-yl]piperidin-1-yl]methyl]-1-[[(S)-oxetan-2-yl]methyl]-1H-benzo[d]imidazole-6-carboxylic acid, 1,3-dihydroxy-2-(hydroxymethyl)propan-2-amine salt for pharmaceutical use.

CN113493447A discloses a compound that can be used as a GLP-1 receptor agonist. WO2021197464 discloses fused imidazole derivatives, preparation methods and medical use as a therapeutic agent, especially as GLP-1 receptor agonists.

CN113480534A discloses benzimidazole or azabenzimidazole-6-carboxylate compound that can activate GLP-1R downstream signaling pathway.

WO2021154796 discloses compounds as GLP-1R agonists, and compositions, methods, and kits thereof.

WO2021219019 discloses GLP-1 agonists of formula I, including pharmaceutically acceptable salts and solvates thereof, pharmaceutical compositions, and methods of using the same.

WO2021244645 discloses five-membered heteroaromatic imidazole compounds I and their medical use.

WO2021249492 discloses methyl-substituted benzobisoxazole compound and the use thereof in the preparation of drugs for treating related diseases.

CN113816948A discloses fused imidazole derivatives as GLP-1 receptor agonist in the treatment of diabetes.

WO2021254470 discloses preparation of 6-oxo-3,6-dihydropyridine derivative and a pharmaceutical composition containing the derivative, are used as therapeutic agents, in particular as GLP-1 receptors agonist and in the preparation of drugs for the treatment and/or prevention of diabetes.

WO2022007979 discloses a fused imidazole derivative, a preparation method therefor, a pharmaceutical composition containing the derivative, and the use of same as a therapeutic agent, in particular the use thereof as a GLP-1 receptor agonist.

CN113831337A discloses heterocyclic nitrogen compounds as GLP-1 receptor agonist.

WO2022068772 discloses a kind of benzimidazole derivative, its preparation method and application as GLP-1R agonists.

WO2022042691 discloses GLP-1 agonists, including pharmaceutically acceptable salts and solvates thereof, and pharmaceutical compositions including the same.

WO2022040600 discloses compounds that may be used as a glucagon-like peptide-1 receptors (GLP-1R) agonist.

WO2022028572 discloses GLP-1 agonists, including pharmaceutically acceptable salts and solvates thereof, and pharmaceutical compositions including the same.

WO2022031994 discloses compounds and pharmaceutical compositions thereof, for use in, e.g. treating type 2 diabetes mellitus, pre-diabetes, obesity, non-alc. fatty liver disease, non-alc. steatohepatitis, and cardiovascular disease.

CN114591308A discloses piperazine-imidazole containing GLP-1R receptor agonist compounds and application thereof.

WO2022111624 discloses benzimidazole derivatives that are agonists of a glucagon-like peptide-1 receptor (GLP-1R).

WO2022109182 discloses polyheterocyclic benzimidazole compounds and their preparation and use in the treatment of GLP-1R mediated diseases.

CN114478497A discloses a kind of aryl alkyl acid GLP-1 receptor agonist, its preparation method and application in treatment or prevention of GLP-1-mediated diseases and related diseases.

WO2022078380 discloses compounds that are GLP-1 agonists.

WO2022078407 discloses compounds that are GLP-1 agonists.

WO2022078152 discloses a kind of benzimidazolone compounds, their preparation method and application as GLP-1 receptor agonist.

CN114716423A discloses 5,6-dihydro-1,2,4-triazine compounds as GLP-1 receptor agonist.

CN114634510A discloses imidazolopyridine derivatives, which can be used to prepare drugs for treating GLP-1 receptor agonist mediated diseases.

CN114591296A discloses aromatic heterocyclic derivatives as GLP-1R agonists.

WO2022192430 discloses GLP-1R agonists and compositions, methods, and kits thereof.

WO2022192428 discloses GLP-1R agonists and compositions, methods, and kits thereof.

WO2022184849 discloses GLP-1R agonists, uses and pharmaceutical compositions thereof.

CN114907351A discloses tricyclic GLP-1 receptor agonists.

WO2022165076 discloses substituted benzimidazolecarboxylic acids which are GLP-1 receptor modulator compounds.

CN114805336A discloses fused imidazole compounds that are GLP-1 receptor agonists.

CN114763352A discloses benzimidazole derivatives and its application as GLP-1 receptor agonist.

J. Med. Chem. 2022, 65, 12, 8208-8226 discloses A Small-Molecule Oral Agonist of the Human Glucagon-like Peptide-1 Receptor.

Cell Research 2020, (39), 1140-1142 discloses structural insights into the activation of GLP-1R by a small molecule agonist.

An object is to provide novel GLP-1 receptor modulators useful in therapy. A further object is to provide novel compounds having improved safety profile, e.g with regards to selectivity for the GLP-1 receptor over e.g. phosphodiesterase 3 (PDE3) and/or having is improved metabolic stability in the body.

### SUMMARY

There is provided compounds that are modulators of the glucagon-like peptide-1 (GLP-1) receptor, their use as medicaments, pharmaceutical compositions containing them and synthetic routes to their production.

In one embodiment, there is provided a compound of Formula (I),

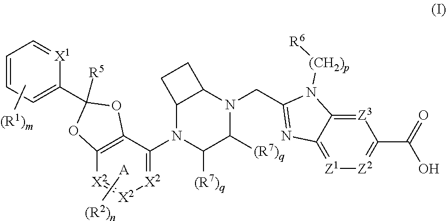

wherein
X¹ is N or C;
X² is independently N or C₁₋₂, provided that no more than two atoms in the aromatic ring A are N;
Z¹ is N or CR³;
Z² and Z³ are each independently N or CR⁴, provided that when Z¹ or Z³ is N, Z² is CR⁴;
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
R⁷ is independently selected from F, C₁₋₂alkyl and OC₁₋₂alkyl, wherein said C₁₋₂alkyl is substituted by 0, 1, 2 or 3 substituents independently selected from F;
m is 0, 1, 2 or 3;
n is 0 or 1;
p is 1, 2 or 3;
q is 0, 1 or 2;
or a pharmaceutically acceptable salt thereof.

The compounds of Formula (I) are modulators of the GLP-1 receptor. Thus, the compounds of Formula (I) can be used as a medicament, in particular for disorders, disease or conditions responsive to modulation of the GLP-1 receptor, and more specifically cardiovascular disease and metabolic conditions.

In another embodiment there is provided a compound of Formula (I), or a pharmaceutically acceptable salt of a compound of Formula (I), wherein the stereochemistry is undefined, e.g. a racemate or a mixture of diastereomers.

In another embodiment there is provided a compound of Formula (I), or a pharmaceutically acceptable salt of a compound of Formula (I), wherein the stereochemistry is defined.

In another embodiment there is provided a pharmaceutical formulation comprising a therapeutically effective amount of a compound of Formula (I), or a pharmaceutically acceptable salt of a compound of Formula (I), and a pharmaceutically acceptable diluent, excipient and/or inert carrier.

In a further embodiment there is provided a pharmaceutical formulation comprising a compound of Formula (I), or a pharmaceutically acceptable salt of a compound of Formula is (I), for use in the treatment of a condition where modulation of the GLP-1 receptor would be beneficial.

In a further embodiment there is provided a compound of Formula (I), or a pharmaceutically acceptable salt of a compound of Formula (I), for use in therapy, especially in the treatment of cancer in a mammal, particularly a human.

In a further embodiment there is provided the use of a compound of Formula (I), or a pharmaceutically acceptable salt of a compound of Formula (I), for the manufacture of a medicament for the treatment of cardiovascular disease and metabolic conditions.

According to another aspect there is provided a process for the preparation of compounds of Formula (I), or pharmaceutically acceptable salts of compounds of Formula (I), and the intermediates used in the preparation thereof.

The compounds of Formula (I) described herein have the advantage that they may be more efficacious, be less toxic, be more selective, be more potent, produce fewer side effects, be more easily absorbed, and/or have a better pharmacokinetic profile (e.g. higher oral bioavailability and/or lower clearance), than compounds known in the prior art.

### BRIEF DESCRIPTION OF THE DRAWINGS

FIG. 1 shows the molecular structure of Intermediate 169, 4-chlorobenzyl (1R,6R)-2,5-diazabicyclo[4.2.0]octane-2-carboxylate.

FIG. 2 shows the molecular structure of Example 3b, 4-chloro-2-(((1R,6R)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid.

### DETAILED DESCRIPTION

This detailed description and its specific examples, while indicating embodiments, are intended for purposes of illustration only. Therefore, there is no limitation to the illustrative embodiments described in this specification. In addition, it is to be appreciated that various features that are, for clarity reasons, described in the context of separate embodiments, also may be combined to form a single embodiment. Conversely, various features that are, for brevity reasons, described in the context of a single embodiment, also may be combined to form sub-combinations thereof. is Listed below are definitions of various terms used in the specification and claims.

It is to be understood that where in this specification a group is qualified by “defined above” the said group encompasses the first occurring and broadest definition as well as each and all of the other definitions for that group.

In this specification, the term “modulator” is used to describe a compound that exhibit varying receptor agonism, either full agonism, or partial agonism.

It is to be understood that in this specification “C₁₋₄” means a carbon group having 1, 2, 3 or 4 carbon atoms.

It is to be understood that in this specification “C₁₋₂” means a carbon group having 1 or 2 carbon atoms.

In this specification, unless stated otherwise, the term “alkyl” includes both straight and branched chain alkyl groups and may be, but is not limited to, methyl, ethyl, n-propyl, i-propyl, n-butyl, sec-butyl, iso-butyl or tert-butyl.

It is to be understood that in this specification “(5- to 6-membered)heteroaryl” means an aromatic ring with 5 to 6 atoms and containing one or more heteroatoms independently selected from nitrogen, oxygen or sulphur.

It is to be understood that in this specification “(6-membered)heteroaryl” means an aromatic ring with 6 atoms and containing one or more heteroatoms independently selected from nitrogen, oxygen or sulphur.

It is to be understood that in this specification “(6-membered)heteroaryl” means for example pyridine.

It is to be understood that in this specification “(5-membered)heteroaryl” means an aromatic ring with 5 atoms and containing one or more heteroatoms independently selected from nitrogen, oxygen or sulphur.

It is to be understood that in this specification “(4- to 6-membered)heterocycloalkyl” means a partially or completely saturated ring system with 4 to 6 atoms and wherein at least one of the ring carbon atoms is replaced with a heteroatom independently selected from nitrogen, oxygen or sulphur.

It is to be understood that in this specification a “heterocycloalkyl” substituent may be attached via a nitrogen atom having the appropriate valences, or via any is ring carbon atom.

It is to be understood that in this specification a “heterocycloalkyl” or “heteroaryl” substituent may be further substituted, for example by a substituent selected from C₁₋₂alkyl.

In this specification, unless stated otherwise, the term “pharmaceutically acceptable” is used to characterize a moiety (e.g. a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

There is provided compounds of Formula (I) wherein X¹, X², Z¹, Z², Z³, R¹-R⁷, m, n, p and q are as defined in Formula (I).

In one embodiment X¹ is N or C₁₋₂.

In a further embodiment X¹ is N.

In still a further embodiment X¹ is C₁₋₂.

R¹ is 0, 1, 2 or 3 substituents independently selected from F, Cl, Br, CN, OCH₃, OCFH₂, OCF₂H, OCF₃, CH₃, CFH₂, CF₂H and CF₃.

In one embodiment X² is independently N or C, provided that no more than two atoms in the aromatic ring A are N.

In a further embodiment X² is C.

In one embodiment Z¹ is N or CR³.

In a further embodiment Z¹ is N.

In still a further embodiment Z¹ is CR³.

R³ is selected from H, F, Cl, N(CH₃)₂, C₁₋₂alkyl and OC₁₋₂alkyl, wherein said C₁₋₂alkyl is substituted by 0, 1, 2 or 3 F.

In one embodiment Z² and Z³ are each independently N or CR⁴, provided that when Z¹ or Z³ is N, Z² is CR⁴.

In a further embodiment Z¹ and Z² are N.

In still a further embodiment Z¹ and Z³ are N.

In still a further embodiment Z² and Z³ are N.

In still a further embodiment Z¹ is N, Z² and Z³ are CR⁴.

In still a further embodiment Z² is N, Z¹ and Z³ are CR⁴.

In still a further embodiment Z³ is N, Z¹ and Z² are CR⁴.

In still a further embodiment Z¹, Z² and Z³ are CR⁴.

R⁴ is independently selected from H, F, Cl, OH, CH₃, CFH₂, CF₂H, CF₃, OCH₃, OCFH₂, OCF₂H and OCF₃.

In one embodiment R¹ is 0, 1, 2 or 3 substituents independently selected from F, Cl, Br, CN, OCH₃, OCFH₂, OCF₂H, OCF₃, CH₃, CFH₂, CF₂H and CF₃.

In a further embodiment R¹ is 0, 1 or 2 substituents independently selected from F, Cl, Br, CN, OCH₃, OCFH₂, OCF₂H, OCF₃, CH₃, CFH₂, CF₂H and CF₃.

In still a further embodiment R¹ is 0, 1, 2 or 3 substituents independently selected from F, Cl, Br, CN, OCH₃

In still a further embodiment R¹ is 0, 1 or 2 substituents independently selected from F, Cl, Br, CN, OCH₃.

In still a further embodiment R¹ is 0, 1 or 2 substituents independently selected from F, Cl and CN.

In still a further embodiment R¹ is 0 or 1 substituents selected from F, Cl and CN.

In one embodiment R² is selected from 0 or 1 F, Cl or CN.

In one embodiment R³ is selected from H, F, Cl, N(CH₃)₂, C₁₋₂alkyl and OC₁₋₂alkyl, wherein said C₁₋₂alkyl is substituted by 0, 1, 2 or 3 F.

In a further embodiment R³ is selected from H, F, Cl, C₁₋₂alkyl and OC₁₋₂alkyl, wherein said C₁₋₂alkyl is substituted by 0, 1, 2 or 3 F.

In still a further embodiment R³ is selected from H, F, Cl, CH₃, CFH₂, CF₂H, CF₃, OCH₃, OCFH₂, OCF₂H and OCF₃.

In still a further embodiment R³ is selected from H, F, Cl, CH₃ and OCH₃.

In one embodiment R⁴ is independently selected from H, F, Cl, OH, CH₃, CFH₂, CF₂H, is CF₃, OCH₃, OCFH₂, OCF₂H and OCF₃.

In a further embodiment R⁴ is independently selected from H, F, Cl, OH, CH₃ and OCH₃-In still a further embodiment R⁴ is independently selected from H, F, Cl, CH₃ and OCH₃.

In still a further embodiment R⁴ is independently selected from H, F and Cl.

In one embodiment R⁵ is selected from H, CH₃, CFH₂, CF₂H and CF₃.

In a further embodiment R⁵ is selected from H, and CH₃.

In still a further embodiment R⁵ is CH₃.

In one embodiment R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5 to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F.

In a further embodiment R⁶ is selected from C₁₋₄alkyl, O(C₁₋₄alkyl) and S(C₁₋₄alkyl), wherein said C₁₋₄alkyl is substituted by 0 or 1 substituent selected from CN or OCH₃, and 0, 1,2 or 3F.

In still a further embodiment R⁶ is selected from cyclopropyl, cyclobutyl, O(cyclopropyl) or S(cyclopropyl), said cyclopropyl and cyclobutyl is substituted by 0 or 1 substituent selected from CN, OCH₃, OCFH₂, OCF₂H, OCF₃ and CH₂CN and 0, 1, 2 or 3 F.

In still a further embodiment R⁶ is selected from (4- to 6-membered)heterocycloalkyl and (5 to 6-membered)heteroaryl, wherein said (4- to 6-membered)heterocycloalkyl and (5- to 6-membered)heteroaryl is substituted by 0 or 1 substituent selected from C₁₋₂alkyl.

In still a further embodiment R⁶ is selected from (5- to 6-membered)heteroaryl, wherein said (5- to 6-membered)heteroaryl is substituted by 0 or 1 substituent selected from C₁₋₂alkyl.

In still a further embodiment R⁶ is selected from (4- to 6-membered)heterocycloalkyl, is wherein said (4- to 6-membered)heterocycloalkyl is substituted by 0 or 1 substituent selected from C₁₋₂alkyl.

In still a further embodiment R⁶ is oxetan-2-yl.

In one embodiment R⁷ is independently selected from F, C₁₋₂alkyl and OC₁₋₂alkyl, wherein said C₁₋₂alkyl is substituted by 0, 1, 2 or 3 substituents independently selected from F.

In a further embodiment R⁷ is independently selected from F, C₁₋₂alkyl and OC₁₋₂alkyl.

In still a further embodiment R⁷ is independently selected from F, CH₃ and OCH₃.

In one embodiment m is 0, 1, 2 or 3.

In a further embodiment m is 0, 1, or 2.

In still a further embodiment m is 1 or 2

In still a further embodiment m is 0 or 1.

In still a further embodiment m is 1.

In still a further embodiment m is 0.

In one embodiment n is 0 or 1.

In a further embodiment n is 1.

In still a further embodiment n is 0.

In one embodiment p is 1, 2 or 3.

In a further embodiment p is 1 or 2.

In still a further embodiment p is 1.

In one embodiment q is 0, 1 or 2.

In a further embodiment q is 0 or 1.

In still a further embodiment q is 0.

In one embodiment, there is provided a compound of Formula (Ia),

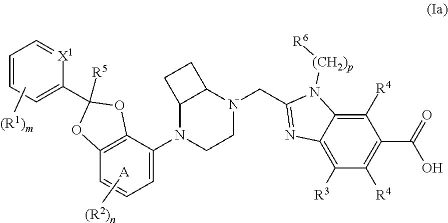

wherein
X¹ is N or C;
R¹ is independently selected from F, Cl, Br, CN, OCH₃, OCFH₂, OCF₂H, OCF₃, CH₃, CFH₂, CF₂H and CF₃;
R² is selected from F, Cl or CN;
is R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
m is 0, 1, 2 or 3;
n is 0 or 1;
p is 1, 2 or 3;
or a pharmaceutically acceptable salt thereof.

In a further embodiment, there is provided a compound of Formula (Ia), wherein
X¹ is N;
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
m is 0, 1 or 2;
n is 0 or 1;
p is 1, 2 or 3;
or a pharmaceutically acceptable salt thereof.

In still a further embodiment, there is provided a compound of Formula (Ia),
wherein
X¹ is N;
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl wherein said (4- to 6-membered)heterocycloalkyl is substituted by 0 or 1 substituent selected from C₁₋₂alkyl and;
m is 0, 1 or 2;
n is 0 or 1;
p is 1;
or a pharmaceutically acceptable salt thereof.

In one embodiment, there is provided a compound of Formula (Ib),

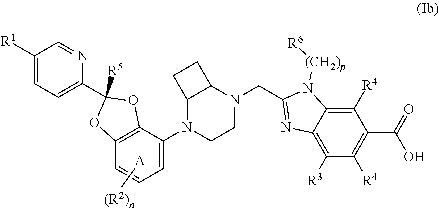

wherein
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

In a further embodiment, there is provided a compound of Formula (Ib), is wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

In still a further embodiment, there is provided a compound of Formula (Ib),
wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl wherein said (4- to 6-membered)heterocycloalkyl is substituted by 0 or 1 substituent selected from C₁₋₂alkyl and;
n is 0 or 1;
p is 1;
is or a pharmaceutically acceptable salt thereof.

In one embodiment, there is provided a compound of Formula (Ic),

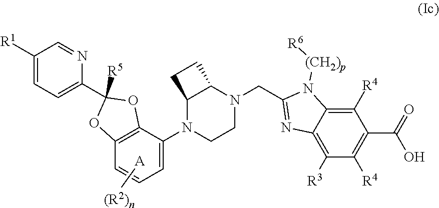

wherein
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
is or a pharmaceutically acceptable salt thereof.

In a further embodiment, there is provided a compound of Formula (Ic), wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

In still a further embodiment, there is provided a compound of Formula (Ic), wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl wherein said (4- to 6-membered)heterocycloalkyl is substituted by 0 or 1 substituent selected from C₁₋₂alkyl and;
n is 0 or 1;
p is 1;
or a pharmaceutically acceptable salt thereof.

In one embodiment, there is provided a compound of Formula (Id),

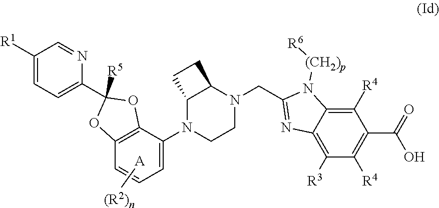

wherein
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

In a further embodiment, there is provided a compound of Formula (Id), wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

In still a further embodiment, there is provided a compound of Formula (Id), wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
is R⁶ is selected from (4- to 6-membered)heterocycloalkyl wherein said (4- to 6-membered)heterocycloalkyl is substituted by 0 or 1 substituent selected from C₁₋₂alkyl and;
n is 0 or 1;
p is 1;
or a pharmaceutically acceptable salt thereof.

In one embodiment, there is provided a compound of Formula (Ie),

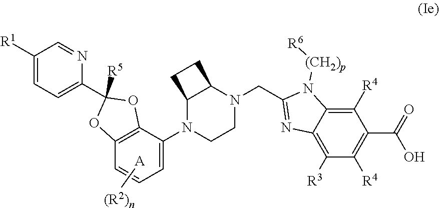

wherein
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected is from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

In a further embodiment, there is provided a compound of Formula (Ie), wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

In still a further embodiment, there is provided a compound of Formula (Ie), wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
is R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl wherein said (4- to 6-membered)heterocycloalkyl is substituted by 0 or 1 substituent selected from C₁₋₂alkyl and;
n is 0 or 1;
p is 1;
or a pharmaceutically acceptable salt thereof.

In one embodiment, there is provided a compound of Formula (If),

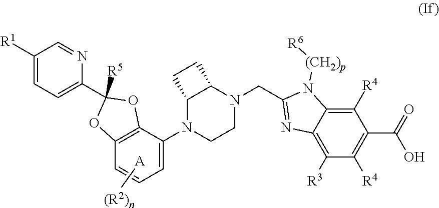

wherein
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 is F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

In a further embodiment, there is provided a compound of Formula (If), wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

In still a further embodiment, there is provided a compound of Formula (If), wherein
R¹ is independently selected from F, Cl and CN;
R² is selected from F, Cl or CN;
is R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl wherein said (4- to 6-membered)heterocycloalkyl is substituted by 0 or 1 substituent selected from C₁₋₂alkyl and;
n is 0 or 1;
p is 1;
or a pharmaceutically acceptable salt thereof.

In one embodiment the compounds of Formula (I) are selected from:
2-(((1R*,6S*)-5-((R*)-2-(5-Chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-((((2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
4-Chloro-2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1R,6R)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-((((R 2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R*,6S*)-5-((R*)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-((((2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-((((2-(4-Chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
rel-4-Chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylic acid,
2-(((1RS,6RS)-5-(2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1RS,6RS)-5-(2-(5-Chloropyridin-2-yl)-2-methylbenzo[d][1,3]dioxol-4-yl)-2,5-diazabicyclo[4.2.0]octan-2-yl)methyl)-5-methoxy-1-(((S)-oxetan-2-yl)methyl)-1H-benzo[d]imidazole-6-carboxylic acid,
rac-2-(((1R,6R)-5-(2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-methoxy-1H-benzo[d]imidazole-6-carboxylic acid,
rac-2-(((1R,6R)-5-(2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-fluoro-1H-benzo[d]imidazole-6-carboxylic acid,
2-(((1R*,6S*)-5-((R*)-2-(4-Cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
4-Chloro-2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-((((R 2-(4-Cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-((((2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-((((2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
4-Chloro-2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-((((R 2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylic acid,
rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylic acid,
rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylic acid,
rel-4-Chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylic acid,
rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-(2-cyclopropoxyethyl)-4-methoxy-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
4-Chloro-2-(((1R,6S)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6 is carboxylic acid,
4-Chloro-2-(((1S,6S)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1S,6R)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6 is carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1R,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1R,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1S,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1S,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
4-Chloro-2-(((1R,6S)-5-((S)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylic acid,
4-Chloro-2-(((1R,6R)-5-((S)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylic acid,
4-Chloro-2-(((1S,6S)-5-((S)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylic acid,
4-Chloro-2-(((1S,6R)-5-((S)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylic acid,
2-(((1R,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-5-methoxy-1-(((S)-oxetan-2-yl)methyl)-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-5-methoxy-1-(((S)-oxetan-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-5-methoxy-1-(((S)-oxetan-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-5-methoxy-1-(((S)-oxetan-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-methoxy-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-methoxy-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-methoxy-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-methoxy-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-fluoro-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-fluoro-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-fluoro-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-fluoro-1H-benzo[d]imidazole-6-carboxylic acid,
2-(((1R,6S)-5-((S)-2-(4-Cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6R)-5-((S)-2-(4-Cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6S)-5-((S)-2-(4-Cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6R)-5-((S)-2-(4-Cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
4-Chloro-2-(((1R,6S)-5-((S)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1R,6R)-5-((S)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1S,6S)-5-((S)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1S,6R)-5-((S)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6S)-5-((S)-2-(4-Cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1R,6R)-5-((S)-2-(4-Cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1S,6S)-5-((S)-2-(4-Cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1S,6R)-5-((S)-2-(4-Cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1R,6S)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6R)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6 is carboxylic acid,
2-(((1S,6S)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1S,6R)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
4-Chloro-2-(((1R,6S)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1R,6R)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1S,6S)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1S,6R)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6 is carboxylic acid,
2-(((1R,6S)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6R)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1S,6S)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1S,6R)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6r)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylic acid,
4-Chloro-2-(((1R,6S)-5-((S)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylic acid,
4-Chloro-2-(((1R,6R)-5-((S)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylic acid,
4-Chloro-2-(((1S,6S)-5-((S)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylic acid,
4-Chloro-2-(((1S,6R)-5-((S)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(2-cyclopropoxyethyl)-4-methoxy-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(2-cyclopropoxyethyl)-4-methoxy-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(2-cyclopropoxyethyl)-4-methoxy-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(2-cyclopropoxyethyl)-4-methoxy-1H-benzo[d]imidazole-6-carboxylic acid,
and pharmaceutically acceptable salts thereof.

It shall be noted that any one of these specific compounds may be disclaimed from any of the herein mentioned embodiments.

In one embodiment there is provided a process for the preparation of compounds of Formula is (I), or pharmaceutically acceptable salts of compounds of Formula (I), and the intermediates used in the preparation thereof.

Another embodiment is a product obtainable by any of the processes or examples disclosed herein.

### Medical and Pharmaceutical Use

The compounds of Formula (I) and their pharmaceutically acceptable salts are believed to be useful in the prevention or treatment of cardiovascular disease and metabolic conditions, including but not limited to type 2 diabetes (T2D), obesity, non-alcoholic fatty liver disease (NAFLD) and non-alcoholic steatohepatitis (NASH), in a mammal, particularly a human.

For the avoidance of doubt, as used herein, the term “treatment” includes therapeutic and/or prophylactic treatment.

When a compound or salt described herein is administered as therapy for treating a disorder, a “therapeutically effective amount” is an amount sufficient to reduce or completely alleviate symptoms or other detrimental effects of the disorder, cure the disorder, reverse, completely stop, or slow the progress of the disorder or reduce the risk of the disorder getting worse.

The compounds described herein are thus indicated both in the therapeutic and/or prophylactic treatment of these conditions.

The compounds described herein have the advantage that they may be more efficacious, be less toxic, be more selective, be more potent, produce fewer side effects, be more easily absorbed, and/or have a better pharmacokinetic profile (e.g. higher oral bioavailability and/or lower clearance), than compounds known in the prior art.

For the above-mentioned therapeutic indications, the dosage administered will vary with the compound employed, the mode of administration and the treatment desired. However, in general, satisfactory results are obtained when the compounds are administered at a dosage of the solid form of between 1 mg and 2000 mg per day.

The compounds of Formula (I), and pharmaceutically acceptable derivatives thereof, may be used on their own, or in the form of appropriate pharmaceutical compositions in which the compound or derivative is in admixture with a pharmaceutically acceptable is adjuvant, diluent or carrier. Thus, another aspect concerns a pharmaceutical composition comprising a novel compound of Formula (I), or a pharmaceutically acceptable salt thereof, in admixture with a pharmaceutically acceptable adjuvant, diluent or carrier. Administration may be by, but is not limited to, enteral (including oral, sublingual or rectal), intranasal, inhalation, intravenous, topical or other parenteral routes. Conventional procedures for the selection and preparation of suitable pharmaceutical Formulations are described in, for example, Pharmaceuticals—The Science of Dosage Form Designs, M. E. Aulton, Churchill Livingstone, 2^{nd} Ed. 2002. In one embodiment the pharmaceutical composition comprises less than 80% and in another embodiment less than 50% of a compound of Formula (I), or a pharmaceutically acceptable salt thereof.

In one embodiment there is provided a compound selected from any one of the compounds of Formula (I), or a pharmaceutically acceptable salt of a compound of Formula (I), for use in therapy, especially in the prevention or treatment of cardiovascular disease and metabolic conditions, including but not limited to type 2 diabetes (T2D), obesity, non-alcoholic fatty liver disease (NAFLD) and non-alcoholic steatohepatitis (NASH).

These and other embodiments are described in greater detail herein below, where further aspects will be apparent to one skilled in the art from reading this specification.

### Combination Therapy

The compounds of Formula (I), or pharmaceutically acceptable salts thereof, may also be administered in conjunction with other compounds used for the treatment of the above conditions.

In another embodiment, there is a combination therapy wherein a compound selected from any one of the compounds of Formula (I), or a pharmaceutically acceptable salt thereof, and a second active ingredient are administered concurrently, sequentially or in admixture, for the treatment of one or more of the conditions listed above. Such a combination may be used in combination with one or more further active ingredients.

When used in a combination therapy, it is contemplated that the a compound selected from any one of the compounds of Formula (I), or pharmaceutically acceptable salts thereof, is and the other active ingredients may be administered in a single composition, completely separate compositions, or a combination thereof. It also is contemplated that the active ingredients may be administered concurrently, simultaneously, sequentially, or separately. The particular composition(s) and dosing frequency(ies) of the combination therapy will depend on a variety of factors, including, for example, the route of administration, the condition being treated, the species of the patient, any potential interactions between the active ingredients when combined into a single composition, any interactions between the active ingredients when they are administered to the animal patient, and various other factors known to physicians (in the context of human patients), veterinarians (in the context of non-human patients), and others skilled in the art.

### Pharmaceutical Compositions

There is provided a method of treatment of a condition where modulation of GLP-1 receptor is required, which method comprises administration of a therapeutically effective amount of a compound selected from any one of the compounds of Formula (I) to a person suffering from, or susceptible to, such a condition.

The compounds of Formula (I) will normally be administered via the oral, topical, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable salt thereof, in a pharmaceutically acceptable dosage form. Depending upon the disorder and patient to be treated and the route of administration, the compositions may be administered at varying doses. Conventional procedures for the selection and preparation of suitable pharmaceutical Formulations are described in, for example, Pharmaceuticals—The Science of Dosage Form Designs, M. E. Aulton, Churchill Livingstone, 2^{nd} Ed. 2002.

In one embodiment suitable daily doses of the compounds of Formula (I) in therapeutical treatment of humans are about 0.0001-100 mg/kg body weight, in another embodiment about 0.01-10 mg/kg body weight.

The optimum dosage and frequency of administration will depend on the particular condition being treated and its severity; the species of the patient; the age, sex, size and weight, diet, and general physical condition of the particular patient; brain/body weight ratio; other medication the patient may be taking; the route of administration; the Formulation; and is various other factors known to physicians and others skilled in the art.

According to a further aspect there is thus provided a pharmaceutical Formulation comprising a compound selected from any one of the compounds of Formula (I), or pharmaceutically acceptable derivatives thereof, in admixture with a pharmaceutically acceptable adjuvant, diluent and/or carrier.

The compound of Formula (I) may be present in the pharmaceutical Formulation in a concentration from 0.1 to 99.5%, such as from 0.5 to 95%, by weight of the total Formulation.

### Preparation of the Compounds

The protection and deprotection of functional groups is described in Protective Groups in Organic Synthesis, 4^{th} Ed, T. W. Greene and P. G. M. Wuts, Wiley-Interscience (2006) and Protecting Groups, 3^{rd} Ed, P. J. Kocienski, Georg Thieme Verlag (2005).

A further embodiment encompasses pharmaceutically acceptable salts of the compounds of Formula (I).

A salt of a compound selected from any one of Formula (I) may be advantageous due to one or more of its chemical or physical properties, such as stability in differing temperatures and humidities, or a desirable solubility in H₂O, oil, or other solvent. In some instances, a salt may be used to aid in the isolation or purification of the compound. In some embodiments (particularly where the salt is intended for administration to an animal, e.g. a human, or is a reagent for use in making a compound or salt intended for administration to an animal), the salt is pharmaceutically acceptable.

The term “pharmaceutically acceptable” is used to characterize a moiety (e.g. a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

Where the compound is sufficiently basic, pharmaceutically acceptable salts include, but are not limited to, inorganic or organic acid addition salts.

For reviews on suitable salts, see Berge et al., J. Pharm. Sci., 1977, 66, 1-19 or Handbook of Pharmaceutical Salts: Properties, selection and use, P. H. Stahl, P. G. Vermuth, is IUPAC, Wiley-VCH, 2002.

Where an acid co-former is a solid at r.t. and there is no or only partial proton transfer between the compound of Formula (I) and such an acid co-former, a co-crystal of the co-former and compound of Formula (I) may result rather than a salt. All such co-crystal forms of the compound of Formula (I) are encompassed herein.

It is also to be understood that certain compounds of Formula (I) may exist in solvated form, e.g. hydrates, including solvates of a pharmaceutically acceptable salt of a compound of Formula (I).

In a further embodiment, certain compounds of Formula (I) may exist as racemates and racemic mixtures, single enantiomers, individual diastereomers and diastereomeric mixtures. Certain compounds of Formula (I) may also contain linkages (e.g. carbon-carbon bonds, carbon-nitrogen bonds such as amide bonds) wherein bond rotation is restricted about that particular linkage, e.g. restriction resulting from the presence of a ring bond or double bond. Stereoisomers may be separated using conventional techniques, e.g. chromatography or fractional crystallization, or the stereoisomers may be made by stereoselective synthesis.

In a further embodiment, the compounds of Formula (I) encompass any isotopically-labelled (or “radio-labelled”) derivatives of a compound of Formula (I). Such a derivative is a derivative of a compound of Formula (I) wherein one or more atoms are replaced by an atom having an atomic mass or mass number different from the atomic mass or mass number typically found in nature. Examples of isotopes that may be incorporated include ²H (also written as “D” for deuterium).

In a further embodiment, the compounds of Formula (I) may be administered in the form of a prodrug which is broken down in the human or animal body to give a compound of the Formula (I).

Various forms of prodrugs are known in the art. For examples of prodrug derivatives, see: Nature Reviews Drug Discovery 2008, 7, 255 and references cited therein. Intermediate compounds may also exist in enantiomeric forms and may be used as purified enantiomers, diastereomers, racemates or mixtures.

### EXAMPLES

The following examples are non-limiting examples.

### General Conditions

(i) operations were carried out at room temperature (rt), i.e. in the range 17 to 25° C. and under an atmosphere of an inert gas such as N₂ unless otherwise stated;
(ii) where reactions refer to being degassed or purged, this can be performed for example by purging the reaction solvent with a constant flow of nitrogen for a suitable period of time (for example 5 to 10 min) or by repeatedly evacuating the vessel and backfill with appropriate inert atmosphere (for example nitrogen (g) or argon (g));
(iii) where reactions refer to the use of a microwave reactor, one of the following microwave reactors were used: Biotage Initiator, Personal Chemistry Emrys Optimizer, Personal Chemistry Smith Creator or CEM Explorer;
(iv) in general, the course of reactions was followed by thin layer chromatography (TLC) and/or analytical high performance liquid chromatography (HPLC or UPLC) which was usually coupled to a mass spectrometer (LCMS);
(v) when necessary, organic solutions were dried over anhydrous MgSO₄ or Na₂SO₄, or by using ISOLUTE® Phase Separator, and work-up procedures were carried out using traditional phase separating techniques;
(vi) It is understood that washing solutions used in the work-up procedures or reagent used for acidifying such as e.g. Brine, NaHCO₃, NH₄Cl, HCl, NaH₂PO₄ are presumed to be aqueous solutions unless otherwise stated;
(vii), evaporations were carried out either by rotary evaporation in vacuo or in a Genevac HT-4/EZ-2 or Biotage V10;
(viii) unless otherwise stated, flash column chromatography was performed on straight phase silica, using either Merck Silica Gel (Art. 9385) or prep-packed cartridges such as Biotage® SNAP cartridges (40-63 m silica, 4-330 g), Biotage® Sfar Silica HC D cartridges (20 μm, 10-100 g), Interchim puriFlash™ cartridges (25 μm, 4-120 g), Interchim puriFlash™ cartridges (50 μm, 25-330 g), Grace™ GraceResolv™ Silica Flash Cartridges (4-120 g) or Agela Flash Colum Silica-CS cartridges (80-330 g), or on reversed phase silica using Agela Technologies C-18, spherical cartridges (20-35 μm, 100A, 80-330 g), manually or automated using a Grace Reveleris®×2 Flash system or similar system;
(ix) preparative reverse phase HPLC and preparative reverse phase SFC were is performed using standard HPLC and SFC instruments, respectively, equipped with either a MS and/or UV triggered fraction collecting instrument, using either isocratic or a gradient of the mobile phase as described in the experimental section, and one of the following methods as described below;

### HPLC Prep Methods:

PrepMethod A: The compound was purified by preparative HPLC on a Kromasil C8 column (10 μm, 250×20 mm ID) using a gradient of MeCN in H₂O/MeCN/FA (95/5/0.2) as mobile phase.

PrepMethod B: The compound was purified by preparative HPLC on a Waters™ Sunfire™ C18 OBD column (5 m, 150×30 mm ID) using a gradient of MeCN in H₂O/FA (0.1 M) as mobile phase.

PrepMethod C: The compound was purified by preparative HPLC on a XSelect CSH OBD column (5 μm, 150×30 mm ID) using a gradient of MeCN in H₂O/TFA (0.05%) as mobile phase.

PrepMethod D: The compound was purified by preparative SFC on a Waters™ BEH, (5 m, 250×30 mm ID) using MeOH/H₂O/NH₃ (20 mM) in CO₂ as mobile phase.

PrepMethod E: The compound was purified by preparative HPLC on a Waters™ Sunfire™ column (5 μm, 100×19 mm ID) using a gradient of MeCN in H₂O as mobile phase.

PrepMethod F: The compound was purified by preparative HPLC on a Kromasil C8 column (10 μm, 250×50 mm ID) using a gradient of MeCN in H₂O/MeCN/FA (95/5/0.2) as mobile phase.

PrepMethod G: The compound was purified by preparative HPLC on a Waters™ Sunfire™ column (5 m, 100×19 mm ID) using a gradient of MeCN in H₂O as mobile phase.

PrepMethod H: The compound was purified by preparative HPLC on a Waters™ Sunfire™ C18 OBD column (5 m, 150×30 mm ID) using a gradient of MeCN in H₂O/FA (0.1%) as mobile phase.

PrepMethod I: The compound was purified by preparative HPLC on a XSelect CSH C18 OBD column (5 μm, 150×30 mm ID) using a gradient of MeCN in H₂O/TFA (0.1%) as mobile phase.

PrepMethod J: The compound was purified by preparative HPLC on an Xbridge Prep OBD C18 column (5 μm, 150×30 mm ID) using a gradient of MeCN in a H₂O/NH₄HCO₃ (10 mM)/NH₃ (0.1%, aq) buffer system as mobile phase.

PrepMethod K: The compound was purified by preparative HPLC on an Xbridge Shield RP18 OBD column (5 μm, 150×30 mm ID) using a gradient of MeCN in a H₂O/NH₄HCO₃ (10 mM)/NH₃ (0.1%, aq) buffer system as mobile phase.

PrepMethod L: The compound was purified by preparative HPLC on a YMC-Actus Triart C18 (5 m, 150×30 mm ID) using a gradient of MeCN in H₂O/NH₄HCO₃ (10 mM)/NH3 (0.1% aq) buffer system as mobile phase.

PrepMethod M: The compound was purified by preparative HPLC on a XBridge™ C18 column (10 m, 250×50 mm ID) using a gradient of MeCN in H₂O/MeCN/NH₃ (95/5/0.2) as mobile phase.

Relevant fractions were collected, combined and freeze-dried to give the purified compound or relevant fractions were collected, combined and concentrated at reduced pressure, extracted with DCM or EtOAc, and the organic phase was dried either over Na₂SO₄ or by using a phase-separator, and then concentrated at reduced pressure to give the purified compound.
(x) chiral preparative chromatography was carried out using HPLC or SFC on a standard HPLC or SFC instruments, respectively, and using either isocratic or gradient run with mobile phase as described in the experimental section;
(xi) yields, where present, are not necessarily the maximum attainable, and when necessary, reactions were repeated if a larger amount of the reaction product was required;
(xii) where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data;
(xiii) in general, the structures of the end-products of the Formula (I) were confirmed by nuclear magnetic resonance (NMR) and/or mass spectral techniques; proton NMR chemical shift values were measured on the delta scale using Bruker Avance III 300, 400, 500 and 600 spectrometers, operating at ¹H frequencies of 300, 400, 500 and 600 MHz, respectively. The experiments were typically recorded at 25° C. Chemical shifts are given in ppm with the solvent as internal standard. Protons on heteroatoms such as NH and OH protons are only reported when detected in NMR and can therefore be missing. In certain instances, protons can be masked or partially masked by solvent peaks and will therefore is either be missing and not reported or reported as multiplets overlapping with solvent. The following abbreviations have been used (and derivatives thereof, e.g. dd, doublet of doublets, ddd, doublet of doublet of doublets, dt, doublet of triplets, dq, doublet of quartet etc.): s, singlet; d, doublet; t, triplet; q, quartet; m, multiplet; br, broad; qn, quintet; p, pentet; h, heptet; brs, broad singlet. It is understood, where the NMR spectra contains residual impurities and/or residual solvent(s), this is not reported unless it coincides or partially coincides with peaks of Intermediates and/or structures of Formula (I), in which case they are reported as multiplets, partially overlapping with said solvent or impurity. In some cases, the structures of the end-products of the Formula (I) might appear as rotamers in the NMR-spectrum, in which instances only peaks of the major rotamer are reported. In some cases, the structures of the end-products of Formula (I) might appear as rotamers in more equal portions, in such instances the peaks of such rotamers are either reported as multiplets, if the signals of said rotamers are partially overlapping, or as individual peaks, if the signals of said rotamers are well separated. Electrospray mass spectral data were obtained using a Waters Acquity UPLC coupled to a Waters single quadrupole mass spectrometer or similar equipment, acquiring both positive and negative ion data, and generally, only ions relating to the parent structure are reported; high resolution electrospray mass spectral data were obtained using a Waters XEVO qToF mass spectrometer or similar equipment, coupled to a Waters Acquity UPLC, acquiring either positive and negative ion data, and generally, only ions relating to the parent structure are reported;
(xiv) intermediates were not necessarily fully purified but their structures and purity were assessed by TLC, analytical IPLC/UPLC, and/or NMR analysis and/or mass spectrometry;
(xv) unless stated otherwise compounds containing an asymmetric carbon and/or sulfur atom were not resolved;
(xv) in general Examples and Intermediate compounds are named using ChemDraw Professional version 20.0.2.51 or version 21.0.0 from PerkinElmer. ChemDraw Professional version 20.0.2.51 or version 21.0.0 generates the names of chemical structures using the Cahn-Ingold-Prelog (CIP) rules for stereochemistry and follows IUPAC rules as closely as possible when generating chemical names. Stereoisomers are differentiated from each other by stereodescriptors cited in names and assigned in accordance with the CIP rules.

ChemDraw is optionally using labels in the graphical representation of stereocenters is such as ‘&’ and ‘or’ to describe the configuration of the stereochemical centers present in the structure.

In general chemical structures of Examples and Intermediates containing the label ‘&’ at a stereocenter, means the configuration of such Example or Intermediate at that stereocenter is a mixture of both (R) and (S); and a label ‘or’ means the configuration of such Example or Intermediate at that stereocenter is either (S) or (R). Absolute, unspecified, ‘&’, and ‘or’ stereocenters can all be present in a single structure.

In general for structures of Examples and Intermediates where all of the stereocenters are designated as ‘&’, the structure is named with a “rac-” prefix. For structures of Examples and Intermediates where all of the stereocenters are designated as ‘or’, the structure is named with a “rel-” prefix.

In general Examples and Intermediate compounds are named using the descriptors (RS) and (SR) to denote general ‘&’ centers for chemical structures with multiple chiral centers where only some are designated as ‘&’. The descriptors (R*) and (S*) are used to denote the general ‘or’ centers for chemical structures with multiple chiral centers where only some are designated as ‘or’.

In general Examples and Intermediate compounds containing stereocenters having a relationship that is either cis or trans, are named using the descriptors (RS, SR) or (RS, RS), to denote chemical structures with multiple chiral centers where only some are designated as ‘&’.

In general, for structures of Examples and Intermediates where all stereocenters present are racemic, no flag is designated to the stereocenter(s) and the structure is drawn with straight bond(s) at each stereocenter.

In general, for structures of Examples and Intermediates where two or more stereocenters are present in a ring and fixed to each other and do not vary independently of each other, e.g. are cis or trans to each other, said stereocenters are drawn with stereobonds representing their internal relationship. Said stereocenters are labelled with an “&1” flag representing a mixture of cis-configuration or a mixture of trans-configuration, or an “orn” flag representing a single cis-isomer or a single trans-isomer with unknown absolute stereochemistry. In general, should the structure of said Example or Intermediate further contain one or more stereocenters that are racemic and not fixed in relation to the former stereocenters, said stereocenter(s) is drawn with straight bond(s) at said stereocenters.

In general the descriptors (r) and (s) are used to describe the absolute configuration of any pseudoasymmetric centers in the structures of Examples and Intermediates.

In general the label “Isomer 1” corresponds to the first eluted isomer, and “Isomer 2” corresponds to the second eluted isomer, on a given chiral TIPLC column and eluent, and are used to distinguish two isomers containing one or more stereocenters with absolute unknown configuration;

(xvii) Unless otherwise specified, all crystallographic measurements were performed at 175K on a Bruker Smart Apex II diffractometer operating in the ω scans mode. The intensity data were collected within the θₘₐₓ≤26.0° using Mo-K_{α} radiation (λ=0.71078 Å).

The structure was solved by direct methods and refined by the full-matrix least-squares technique in the anisotropic approximation for non-hydrogen atoms using the Bruker SHELXTL program package.

(xviii) in addition to the ones mentioned above, the following abbreviations and units have been used:

### ABBREVIATIONS

aq aqueous
BF₃·OEt₂ boron trifluoride diethyl etherate
Boc tert-butyloxycarbonyl
calcd calculated
DCM dichloromethane
DEA diethyl amine
DIPEA N,N-diisopropylethylamine
DMAP 4-dimethylaminopyridine
DMF N,N-dimethylformamide
DMSO dimethyl sulfoxide
dppp 1,3-bis(diphenylphosphino)propane
EC50 half maximal effective concentration
ESI electrospray ionization
EtOAc ethyl acetate
EtOH ethanol
Et₂O diethyl ether
FA formic acid
is g gram
(g) gas
GC/MS gas chromatography/mass spectrometry
HPLC high performance liquid chromatography
HRMS high resolution mass spectrometry
IC50 half maximal inhibitory concentration
ID inner diameter
IPA 2-methylpropan-ol
(1) liquid
LCMS liquid chromatography mass spectrometry
MeCN acetonitrile
MeOH methanol
MS mass spectrometry
MTBE tert-butyl methyl ether
NMR nuclear magnetic resonance
Palladacycle Gen 4 Methanesulfonato(2-bis(3,5-di(trifluoromethyl)phenylphosphino)-3,6-dimethoxy-2′,6′-bis(dimethylamino)-1,1′-biphenyl)(2′-methylamino-1,1′-biphenyl-2-yl)palladium(II), Cas Reg No 1810068-35-9
Pd₂(dba)₃ Tris(dibenzylideneacetone)dipalladium(O)
Pd(dppf)Cl₂·DCM [1,1′-Bis(diphenylphosphino)ferrocene]dichloropalladium(II) (1:1), Pd(dppf)Cl₂·DCM
Pd-PEPPSI-1HeptC^{l} Cas Reg No 1814936-54-3
pTsOH para-toluenesulfonic acid
Rochelle salt potassium sodium tartrate
rt room temperature
RuPhos dicyclohexyl(2′,6′-diisopropoxy-[1,1′-biphenyl]-2-yl)phosphane
sat saturated
SFC supercritical fluid chromatography
t-Bu tert-butyl
TFA trifluoroacetic acid
THE tetrahydrofuran
TLC thin layer chromatography
is UPLC ultra performance liquid chromatography
UV ultraviolet
VCD vibrational circular dichroism

### Units

atm atmosphere
C Celcius
g gram
h hour(s)
L liter
M mole per liter
mg milligram
MHz megaherz
min minute(s)
mL milliliter
mm millimeter
mM millimole per liter
mmol millimole(s)
mol mole
μCi microcurie
μL microliter
μm micrometer
μM micromole per liter
nL nanoliter
N equivalents per liter
nm nanometer
nM nanomole per liter
μM picomole per liter
ppm parts per million
v/v volume by volume
W/v weight by volume

### INTERMEDIATES

### Intermediate 1

### Di-tert-butyl 5,8-dioxo-4,7-diazaspiro[2.5]octane-4,7-dicarboxylate

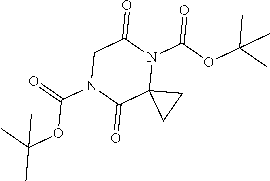 [SMILES: CC(C)(C)OC(=O)N1CC(=O)N(C(=O)OC(C)(C)C)C2(CC2)C1=O]

DMAP (16 g, 130.97 mmol) was added to a solution of 4,7-diazaspiro[2.5]octane-5,8-dione (185 g, 1.32 mol) in DCM (2.5 L). Di-tert-butyl dicarbonate (576 g, 2.64 mol) was added in several batches, and the resulting solution was stirred at rt for 2 h. The reaction was quenched by the addition of sat NH₄Cl (2.0 mL). The organic layer was combined and washed with brine (1.0 mL). The organic layer was dried over anhydrous Na₂SO₄, and concentrated under vacuum. The residue was purified by silica gel column chromatography (EtOAc:petroleum ether, 1:6) to give the title compound (380 g, 85%) as a white solid.

### Intermediate 2

Di-tert-butyl 5.8-dihydroxy-4.7-diazaspiro[2.5]octane-4.7-dicarboxylate

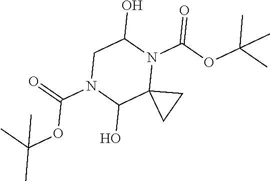 [SMILES: CC(C)(C)OC(=O)N1CC(O)N(C(=O)OC(C)(C)C)C2(CC2)C1O]

A solution of diisobutylaluminium hydride in toluene (1 N, 1.47 L, 103.36 mmol) was added dropwise with stirring at −78° C. to a solution of 4,7-di-tert-butyl 5,8-dioxo-4,7-diazaspiro[2.5]octane-4,7-dicarboxylate Intermediate 1 (125 g, 367.25 mmol) in THE (1.5 L), and the reaction mixture was stirred at −78° C. for 2 h. The reaction was quenched by the addition of Rochelle salt (2 N, 1.5 L). The mixture was diluted with of DCM (2 L), and the solids were filtered out. The filtrate was dried over anhydrous Na₂SO₄ and concentrated under vacuum. The residue was purified by silica gel column chromatography (DCM:MeOH, 50:1) to give the title compound (340 g, 90%) as a light yellow semi-solid; MS (ESI) m/z [M+H]⁺ 345.

### Intermediate 3

### Di-tert-butyl (1R,6S)-2,5-diazabicyclo[4.2.0]octane-2,5-dicarboxylate

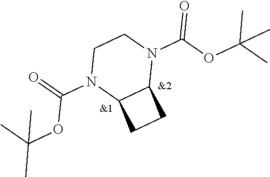 [SMILES: CC(C)(C)OC(=O)N1CCN(C(=O)OC(C)(C)C)[C@H]2CC[C@H]21]

is BF₃·OEt₂ (124 g, 873.68 mmol) and triethylsilane (101 g, 868.62 mmol) was added in several batches, at −78° C., to a solution of 4,7-di-tert-butyl 5,8-dihydroxy-4,7-diazaspiro[2.5]octane-4,7-dicarboxylate Intermediate 2 (75 g, 217.77 mmol) in DCM (750 mL). The resulting solution was stirred at −78° C. for 2 h. Another batch was prepared as described above. The reaction was then quenched by the addition of water/ice (500 mL). The resulting solution was extracted with DCM (2×500 mL) and the combined organic layer was dried over anhydrous Na₂SO₄ and concentrated under vacuum. The residue was purified by silica gel column chromatography (EtOAc/hexane, 1:20-1:10), to give the title compound (75 g, 55%) as an off-white solid; ¹H NMR (CDCl₃) δ 4.3(s, 1H), 3.4 (m, 2H), 2.1 (s, 2H), 1.47 (s, 9H).

### Intermediate 4

### rac-tert-Butyl (1R,6S)-2,5-diazabicyclo[4.2.0]octane-2-carboxylate

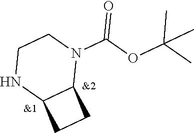

pTsOH (22 g, 127.76 mmol) was added to a solution of di-tert-butyl (1R,6S)-2,5-diazabicyclo[4.2.0]octane-2,5-dicarboxylate Intermediate 3 (40 g, 128.04 mmol) in THE (400 mL). The resulting solution was stirred at 60° C. for 3 h. Three additional batches were prepared as described above. The reaction mixture was cooled to rt, and the solids were filtered out. K₂CO₃ (320 g) was added with stirring to the resulting solution and stirred at rt for 1 h. The solids were filtered out. The resulting mixture was concentrated under vacuum. The residue was purified by silica gel column chromatography (DCM:MeOH, 1:0-10:1) to give the title compound (45 g, 41%) as yellow oil; MS (ESI) m/z [M+H]⁺ 213.

### Intermediate 5

### Methyl (S)-3-fluoro-4-nitro-5-((oxetan-2-ylmethyl)amino)benzoate

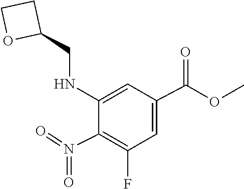 [SMILES: COC(=O)c1cc(F)c([N+](=O)[O-])c(NC[C@@H]2CCO2)c1]

is DIPEA (8.45 mL, 48.36 mmol) was added to a solution of methyl 3,5-difluoro-4-nitrobenzoate (3.50 g, 16.12 mmol) and (S)-oxetan-2-ylmethanamine (1.40 g, 16.12 mmol) in THF/DMF (125 mL, 5:2), and the reaction mixture was stirred at 20° C. for 4 h. The solvent was removed under reduced pressure, and the residue was suspended in water (300 mL). The aqueous layer was extracted with EtOAc (3×500 mL), and the combined organic layer was dried over Na₂SO₄, filtered and evaporated. The crude product was purified by straight phase flash chromatography on silica (gradient: 10-20% EtOAc in petroleum ether) to give the title compound (4.50 g, 98%) as a yellow solid; MS (ESI) m/z [M+H]⁺ 285.0.

### Intermediate 6

### Methyl (S)-4-amino-3-fluoro-5-((oxetan-2-ylmethyl)amino)benzoate

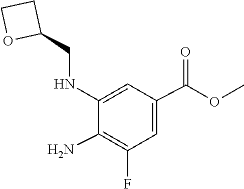 [SMILES: COC(=O)c1cc(F)c(N)c(NC[C@@H]2CCO2)c1]

A suspension of methyl (S)-3-fluoro-4-nitro-5-((oxetan-2-ylmethyl)amino)benzoate Intermediate 5 (4.2 g, 14.78 mmol) and 10% Pd-C(1.57 g, 1.48 mmol) in THE (150 mL) was stirred under an atmosphere of H₂ (g) at 3 atm and 25° C. for 2 h. The reaction mixture was filtered through celite and the filter cake was washed with MeOH (3×100 mL). The filtrate was concentrated under reduced pressure. The crude product was purified by straight phase flash chromatography on silica (gradient: 70-80% EtOAc in petroleum ether), to give the title compound (3.20 g, 85%) as a light red solid; MS (ESI) m/z [M+H]⁺ 254.95.

### Intermediate 7

### Methyl (S)-2-(chloromethyl)-4-fluoro-1-(oxetan-2-ylmethyl)-1H-benzo[d]imidazole-6-carboxylate

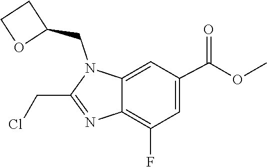 [SMILES: COC(=O)c1cc(F)c2nc(CCl)n(C[C@@H]3CCO3)c2c1]

pTsOH (0.108 g, 0.57 mmol) was added to a solution of methyl (S)-4-amino-3-fluoro-5-((oxetan-2-ylmethyl)amino)benzoate Intermediate 6 (1.45 g, 5.70 mmol) and 2-chloro-1,1,1-trimethoxyethane (1.06 g, 6.84 mmol) in MeCN (10 mL) and the reaction mixture was stirred at rt for 18 h. The solvent was evaporated at reduced pressure and the crude compound was purified by straight phase flash chromatography on silica (gradient: 50-100% EtOAc in heptane) to give the title compound (1.54 g, 86%); MS (ESI) m/z [M+H]⁺ 313.26.

### Intermediate 8

### Methyl (S)-3-methoxy-4-nitro-5-((oxetan-2-ylmethyl)amino)benzoate

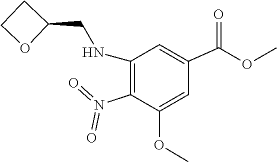 [SMILES: COC(=O)c1cc(NC[C@@H]2CCO2)c([N+](=O)[O-])c(OC)c1]

K₂CO₃ (5.43 g, 39.27 mmol) was added to a solution of methyl 3-fluoro-5-methoxy-4-nitrobenzoate (3 g, 13.09 mmol) and (S)-oxetan-2-ylmethanamine (1.14 g, 13.09 mmol) in THF/DMF (5:2, 110 mL) and the reaction mixture was stirred at 90° C. for 16 h. The solvent was removed under reduced pressure, and the residue was suspended in water (250 mL). The aqueous layer was extracted with EtOAc (3×250 mL), and the combined organic layer was dried over Na₂SO₄, filtered and evaporated at reduced pressure. The crude product was purified by straight phase flash column chromatography on silica (10-20% EtOAc in petroleum ether) to give the title compound (1.8 g, 46%) as a yellow solid; MS (ESI) m z [M+H]⁺ 297.1.

### Intermediate 9

### Methyl (S)-4-amino-3-methoxy-5-((oxetan-2-ylmethyl)amino)benzoate

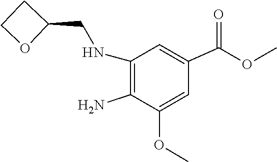 [SMILES: COC(=O)c1cc(NC[C@@H]2CCO2)c(N)c(OC)c1]

A suspension of Pd-C (0.144 g, 1.35 mmol) and methyl (S)-3-methoxy-4-nitro-5-((oxetan-2-ylmethyl)amino)benzoate Intermediate 8 (4 g, 13.50 mmol) in THE (100 mL) was stirred under an atmosphere of H₂(g) at 2 atm and 15° C. for 3 h. The reaction mixture was filtered through celite and the filtercake was washed with MeOH (3×300 mL). The filtrate was concentrated under reduced pressure. The crude product was purified by straight phase flash column chromatography on silica (50-70% EtOAc in petroleum ether) to give the title compound (3.00 g, 83%) as a light yellow solid; MS (ESI) m/z [M+H]⁺ 267.3.

### Intermediate 10

### Methyl (S)-2-(chloromethyl)-4-methoxy-1-(oxetan-2-ylmethyl)-1H-benzo[d]imidazole-6-carboxylate

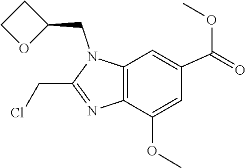 [SMILES: COC(=O)c1cc(OC)c2nc(CCl)n(C[C@@H]3CCO3)c2c1]

pTsOH (0.119 g, 0.63 mmol) was added to a solution of methyl (S)-4-amino-3-methoxy-5-((oxetan-2-ylmethyl)amino)benzoate Intermediate 9 (0.333 g, 1.25 mmol) and 2-chloro-1,1,1-trimethoxyethane (0.387 g, 2.50 mmol) in MeCN (10 mL) and the reaction mixture was stirred at 45° C. for 30 min. The reaction mixture was concentrated under reduced pressure and the residue was purified by straight phase flash column chromatography on silica (50-100% EtOAc in heptane) to give the title compound (0.155 g, 38%); MS (ESI) m/z [M+H]⁺ 325.0.

### Intermediate 11

### rac-tert-Butyl (1R,6S)-5-(2-(5-chloropyridin-2-yl)-2-methylbenzo[d][1,3]dioxol-4-yl)-2,5-diazabicyclo[4.2.0]octane-2-carboxylate

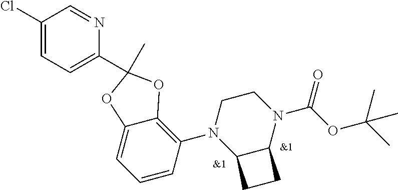 [SMILES: CC(C)(C)OC(=O)N1CCN(c2cccc3c2OC(C)(c2ccc(Cl)cn2)O3)[C@@H]2CC[C@@H]21]

Pd₂(dba)₃ (0.366 g, 0.40 mmol) was added to a solution of 2-(4-bromo-2-methylbenzo[d][1,3]dioxol-2-yl)-5-chloropyridine (WO2020234726) (1.306 g, 4 mmol), rac-is tert-butyl (1R,6S)-2,5-diazabicyclo[4.2.0]octane-2-carboxylate Intermediate 4 (0.849 g, 4.00 mmol), RuPhos (0.747 g, 1.60 mmol) and sodium 2-methylpropan-2-olate (1.538 g, 16.00 mmol) in degassed toluene (10 mL). The reaction mixture was evacuated and backfilled with N₂ (g) (×3) and then stirred at 100° C. for 10 min. The reaction mixture was cooled to rt and filtered through a pad of celite, and the solid cake was rinsed with toluene. The filtrate was concentrated under reduced pressure and the crude compound was purified by straight phase flash column chromatography on silica (gradient: 0-20% EtOAc:heptane) to give the title compound (0.640 g, 35%); MS (ESI) m/z [M+H]⁺ 458.1.

### Intermediate 12

### Methyl 2-(((1RS,6SR)-5-((SR)-2-(5-chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate

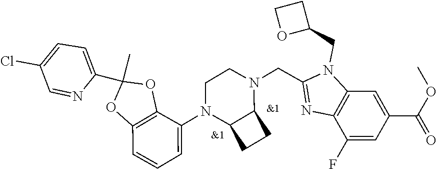 [SMILES: COC(=O)c1cc(F)c2nc(CN3CCN(c4cccc5c4OC(C)(c4ccc(Cl)cn4)O5)[C@@H]4CC[C@@H]43)n(C[C@@H]3CCO3)c2c1]

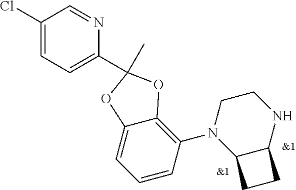 [SMILES: CC1(c2ccc(Cl)cn2)Oc2cccc(N3CCN[C@H]4CC[C@H]43)c2O1]

TFA (547 μl, 7.10 mmol) was added to a solution of rac-tert-butyl (1R,6S)-5-(2-(5-chloropyridin-2-yl)-2-methylbenzo[d][1,3]dioxol-4-yl)-2,5-diazabicyclo[4.2.0]octane-2-carboxylate Intermediate 11 (650 mg, 1.42 mmol) in MeCN (30 mL) and the reaction mixture was stirred at rt for 1 h. The reaction mixture was concentrated in vacuo, then co-evaporated with toluene (×1) and MeCN (×2) to give the crude step a) product; MS (ESI) m z [M+H]⁺ 359.9.

The crude product from Step a) was dissolved in MeCN (30 mL), and K₂CO₃ (588 mg, 4.26 mmol) and methyl (S)-2-(chloromethyl)-4-fluoro-1-(oxetan-2-ylmethyl)-1H-benzo[d]imidazole-6-carboxylate Intermediate 7 (533 mg, 1.70 mmol) was added and the reaction mixture was heated at 30° C. for 18 h. The reaction mixture was cooled to rt and filtered through celite. The filtrate was concentrated in vacuo and the residue was purified by straight phase flash column chromatography on silica (gradient: 0-100% EtOAc in heptane). The product containing fractions was pooled and the solvent was removed by evaporation to give a crude residue. Another batch was prepared as described above (130 mg), and the crude products were combined and purified by preparative HPLC, PrepMethod A, (gradient 40-70%), to give the title compound (0.730 g, 81%); MS (ESI) m/z [M+H]⁺ 634.47.

### Intermediate 13

### Methyl 2-(((1R*,6S*)-5-((S*)-2-(5-chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 2

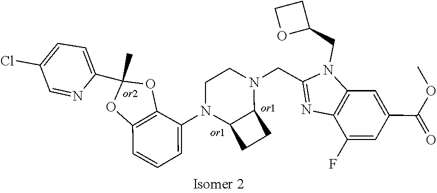 [SMILES: COC(=O)c1cc(F)c2nc(CN3CCN(c4cccc5c4O[C@@](C)(c4ccc(Cl)cn4)O5)[C@@H]4CC[C@@H]43)n(C[C@@H]3CCO3)c2c1]

### Intermediate 14

### Methyl 2-(((1R*,6S*)-5-((S*)-2-(5-chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 3

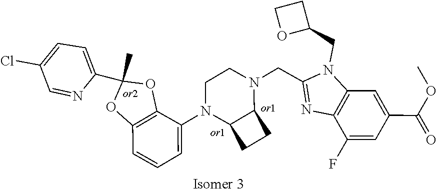 [SMILES: COC(=O)c1cc(F)c2nc(CN3CCN(c4cccc5c4O[C@@](C)(c4ccc(Cl)cn4)O5)[C@@H]4CC[C@@H]43)n(C[C@@H]3CCO3)c2c1]

### Intermediate 15

### Methyl 2-((1R*,6S*)-5-((S*)-2-(5-chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 4

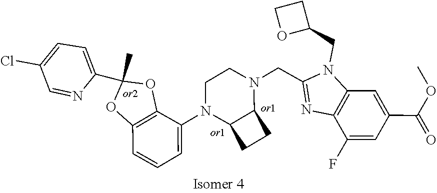 [SMILES: COC(=O)c1cc(F)c2nc(CN3CCN(c4cccc5c4O[C@@](C)(c4ccc(Cl)cn4)O5)[C@@H]4CC[C@@H]43)n(C[C@@H]3CCO3)c2c1]

The diastereomers of methyl 2-(((1RS,6SR)-5-((SR)-2-(5-chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate Intermediate 12 (730 mg, 1.13 mmol) were separated by chiral chromatography on a LUX C4 column (250×30 mm, 5 μm), eluted with 35% EtOH/DEA (100/20 mM) in CO₂, 130 bar, at a flow rate of 130 mL/min and detected at 220 nm;
the first eluted compound mixture was collected and evaporated to yield a mixture of isomers (417 mg); and
the second eluted compound was collected and evaporated to yield the title compound Isomer 4, Intermediate 15 (125 mg); MS (ESI) m/z [M+H]⁺ 634.3.

The stereoisomers of the first eluted compound mixture (417 mg) were separated by chiral chromatography on a LUX C3 (OJ) column (150×4.6 mm, 5 μm), eluted with 8% MeOH/DEA (100/20 mM) in CO₂, 130 bar, at a flow rate of 130 mL/min and detected at 220 nm;
is the second eluted compound was collected and evaporated to give the title compound Isomer 2, Intermediate 13 (106 mg): MS (ESI) m/z [M+H]⁺ 634.4.
the third eluted compound was collected and evaporated to give the title compound Isomer 3, Intermediate 14 (138 mg, 99.6 area %); MS (ESI) m/z [M+H]⁺ 634.3.

### Intermediate 16

### Methyl 2-(((1RS,6SR)-5-(2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate

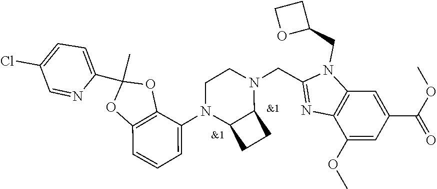 [SMILES: COC(=O)c1cc(OC)c2nc(CN3CCN(c4cccc5c4OC(C)(c4ccc(Cl)cn4)O5)[C@@H]4CC[C@@H]43)n(C[C@@H]3CCO3)c2c1]

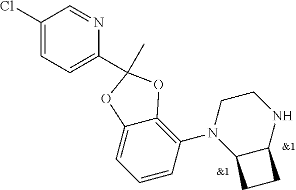 [SMILES: CC1(c2ccc(Cl)cn2)Oc2cccc(N3CCN[C@H]4CC[C@H]43)c2O1]

TFA (169 μl, 2.20 mmol) was added to a solution of rac-tert-butyl (1R,6S)-5-((S)-2-(5-chloropyridin-2-yl)-2-methylbenzo[d][1,3]dioxol-4-yl)-2,5-diazabicyclo[4.2.0]octane-2-carboxylate Intermediate 11 (202 mg, 0.44 mmol) in DCM (3.0 mL) and the reaction mixture was stirred at rt for 1 h. The mixture was evaporated at reduced pressure to give the sub-title compound; MS (ESI) m/z [M+H]⁺ 359.9. The crude product from Step a) was dissolved in MeCN (3 mL) and K₂CO₃ (182 mg, 1.32 mmol) and methyl (S)-2-(chloromethyl)-4-methoxy-1-(oxetan-2-ylmethyl)-1H-benzo[d]imidazole-6-carboxylate Intermediate 10 (144 mg, 0.44 mmol) was added, and the reaction mixture was heated at 60° C. for 18 h. The reaction mixture was cooled to rt and filtered through delite. The filtrate was concentrated at reduced pressure and the crude product is was purified by straight phase flash column chromatography on silica (30-100% EtOAc in hexane) to give the title compound (235 mg, 83%); MS (ESI) m/z [M]⁺/[M+2H]⁺646.5/648.3.

### Intermediate 17

### Methyl 2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 1

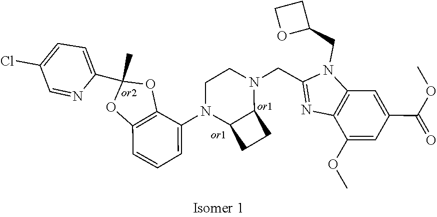 [SMILES: COC(=O)c1cc(OC)c2nc(CN3CCN(c4cccc5c4O[C@@](C)(c4ccc(Cl)cn4)O5)[C@@H]4CC[C@@H]43)n(C[C@@H]3CCO3)c2c1]

### Intermediate 18

### Methyl 2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 2

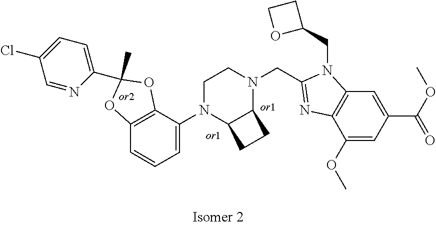 [SMILES: COC(=O)c1cc(OC)c2nc(CN3CCN(c4cccc5c4O[C@@](C)(c4ccc(Cl)cn4)O5)[C@@H]4CC[C@@H]43)n(C[C@@H]3CCO3)c2c1]

### Intermediate 19

### Methyl 2-((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 3

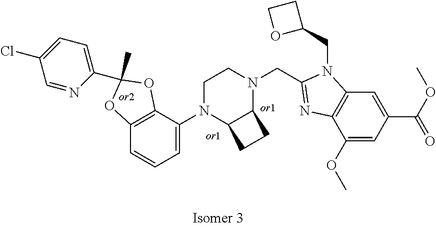 [SMILES: COC(=O)c1cc(OC)c2nc(CN3CCN(c4cccc5c4O[C@@](C)(c4ccc(Cl)cn4)O5)[C@@H]4CC[C@@H]43)n(C[C@@H]3CCO3)c2c1]

### Intermediate 20

### Methyl 2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 4

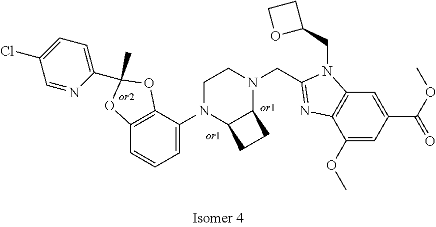 [SMILES: COC(=O)c1cc(OC)c2nc(CN3CCN(c4cccc5c4O[C@@](C)(c4ccc(Cl)cn4)O5)[C@@H]4CC[C@@H]43)n(C[C@@H]3CCO3)c2c1]

The diastereomers of methyl 2-(((1RS,6SR)-5-(2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate Intermediate 16 (235 mg, 0.36 mmol) were separated by chiral chromatography on a CHIRALPAK® IH column (5 μm, 250×30 mm ID), eluted with 25% EtOH/DEA (100/20 mM) in CO₂, 120 bar, at a flow rate of 130 mL/min and detected at 230 nm;
the first eluted compound mixture was collected and evaporated to yield a mixture of isomers (74 mg) and
the second eluted compound mixture was collected and evaporated to give a mixture of isomers (88 mg).

The stereoisomers of the first eluted compound mixture (74 mg) were separated by chiral chromatography on a YMC SA (IA) column (5 μm, 250×30 mm ID), eluted with 15% EtOH/DEA (100/20 mM) in CO₂, 120 bar, at a flow rate of 130 mL/min and detected at 220 nm;
the first eluted compound was collected and evaporated to give the title compound Isomer 1 Intermediate 17 (33 mg); MS (ESI) m/z [M+H]⁺ 646.3.
the second eluted compound was collected and evaporated to give the title compound Isomer 2 Intermediate 18 (29 mg); MS (ESI) m/z [M+H]⁺ 646.4.

The stereoisomers of the second eluted compound mixture (88 mg) were separated by chiral chromatography on a LUX C4 column (5 μm, 250×30 mm ID), eluted with 30% EtOH/DEA (100/20 mM) in CO₂, 120 bar, at a flow rate of 130 mL/min and detected at 230 nm; The first eluted compound was collected and evaporated to give the title compound Isomer 3 Intermediate 19 (58 mg); MS (ESI) m/z [M+H]⁺ 646.54.

The second eluted compound was collected and evaporated to give the title compound Isomer 4 Intermediate 20 (47 mg); MS (ESI) m/z [M+H]⁺ 646.2.

### Intermediate 21

### (S)-5-Bromo-3-chloro-2-nitro-N-(oxetan-2-ylmethyl)aniline

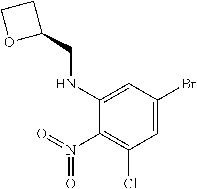 [SMILES: O=[N+]([O-])c1c(Cl)cc(Br)cc1NC[C@@H]1CCO1]

DIPEA (15.44 mL, 88.43 mmol) and 5-bromo-1-chloro-3-fluoro-2-nitrobenzene (7.5 g, 29.48 mmol) were added dropwise to a solution of (S)-oxetan-2-ylmethanamine (2.57 g, 29.48 mmol) in THE (200 mL), and the reaction mixture was stirred at 60° C. for 3 h. The reaction mixture was diluted with EtOAc (300 mL) and washed with sat brine (4×300 mL). The organic layer was dried over Na₂SO₄, filtered and evaporated. The crude product was purified by flash chromatography on silica (0-50% EtOAc in petroleum ether) to give the title compound (9.00 g, 95%) as a yellow oil; MS (ESI) m/z [M+H]⁺ 321/323.

### Intermediate 22

### (S)-5-Bromo-3-chloro-N¹-(oxetan-2-ylmethyl)benzene-1,2-diamine

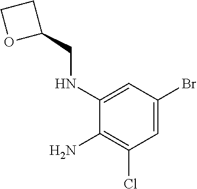 [SMILES: Nc1c(Cl)cc(Br)cc1NC[C@@H]1CCO1]

Fe(s) (24.66 g, 441.60 mmol) was added to a mixture of (S)-5-bromo-3-chloro-2-nitro-N-(oxetan-2-ylmethyl)aniline Intermediate 21 (14.2 g, 44.16 mmol) and NH₄Cl (23.62 g, 441.60 mmol) in MeOH (400 mL) and water (100 mL) at 20° C., and the reaction mixture was stirred at 60° C. for 6 h. The reaction mixture was filtered and the precipitates were washed with MeOH (4×100 mL). The filtrate was concentrated under reduced pressure and the crude product was diluted with EtOAc (500 mL). The organic layer was washed sequentially with water (500 mL) and sat brine (500 mL), dried over Na₂SO₄, filtered and evaporated. The is crude product was purified by flash chromatography on silica (30-50% EtOAc in petroleum ether) to give the title compound (12.00 g, 93%) as a white solid; MS (ESI) m/z [M+H]⁺ 292/291.

### Intermediate 23

### Methyl (S)-4-amino-3-chloro-5-((oxetan-2-ylmethyl)amino)benzoate

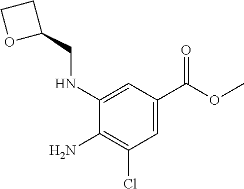 [SMILES: COC(=O)c1cc(Cl)c(N)c(NC[C@@H]2CCO2)c1]

A mixture of (S)-5-bromo-3-chloro-N¹-(oxetan-2-ylmethyl)benzene-1,2-diamine Intermediate 22 (1.5 g, 5.14 mmol), Pd(dppf)Cl₂·DCM (0.38 g, 0.51 mmol) and DIPEA (8.99 mL, 51.45 mmol) in MeOH (300 mL) was stirred under an atmosphere of CO(g) at 60 atm and 120° C. for 30 h. The solvent was removed under reduced pressure and the crude product was purified by flash chromatography on silica (20-25% EtOAc in petroleum ether) to give the title compound (1.0 g, 72%) as a white solid; MS (ESI) m/z [M+H]⁺ 271.

### Intermediate 24

### Methyl (S)-4-chloro-2-(chloromethyl)-1-(oxetan-2-ylmethyl)-1H-benzo[d]imidazole-6-carboxylate

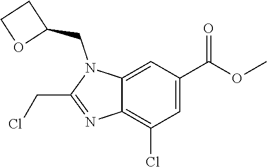 [SMILES: COC(=O)c1cc(Cl)c2nc(CCl)n(C[C@@H]3CCO3)c2c1]

pTsOH (0.357 g, 1.88 mmol) was added to a solution of methyl (S)-4-amino-3-chloro-5-((oxetan-2-ylmethyl)amino)benzoate Intermediate 23 (5.08 g, 18.77 mmol) and 2-chloro-1,1,1-trimethoxyethane (3.77 g, 24.40 mmol) in MeCN (20 mL) and the reaction mixture was stirred at 50° C. for 30 min. 2-Chloro-1,1,1-trimethoxyethane (1.16 g, 7.51 mmol) was added and the reaction mixture was stirred at 50° C. for 20 min. The reaction mixture was diluted with EtOAc (10 mL) and extracted with NaHCO₃ (aq, 2×3 mL). The organic layer was dried over MgSO₄, filtered and concentrated at reduced pressure. The crude compound was purified is by flash chromatography on silica (50-100% EtOAc in heptane) to give the title compound (5.30 g, 86%); MS (ESI) m/z [M+H]⁺ 329.1.

### Intermediate 25

### rac-tert-Butyl (1R,6R)-5-(2-(5-chloropyridin-2-yl)-2-methylbenzo[d][1,3]dioxol-4-yl)-2,5-diazabicyclo[4.2.0]octane-2-carboxylate

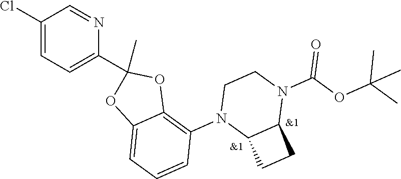 [SMILES: CC(C)(C)OC(=O)N1CCN(c2cccc3c2OC(C)(c2ccc(Cl)cn2)O3)[C@H]2CC[C@@H]21]

A mixture of 2-(4-bromo-2-methylbenzo[d][1,3]dioxol-2-yl)-5-chloropyridine (WO2020234726) (2.85 g, 8.73 mmol), rac-tert-butyl (1R,6R)-2,5-diazabicyclo[4.2.0]octane-2-carboxylate (2.78 g, 13.1 mmol), RuPhos (815 mg, 1.75 mmol), Pd₂(dba)₃ (800 mg, 873 mol) and sodium tert-butoxide (2.52 g, 26.2 mmol) in toluene (50 mL) was stirred at 100° C. under an atmosphere of N₂(g) overnight, and then cooled to rt. The reaction mixture was poured onto water (300 mL) and extracted with EtOAc (2×300 mL). The combined organic layer was washed with brine, dried over Na₂SO₄, and the solvent was removed under vacuum.

The crude product was purified by flash chromatography on silica (0-100% MTBE in hexane) to give the title compound (1.2 g 30%) as a yellow sticky oil; MS (ESI) m/z [M+H]⁺ 458.2.

### Intermediate 26 rac-(1R,6R)-2-(2-(5-Chloropyridin-2-yl)-2-methylbenzo[d][1,3]dioxol-4-yl)-2,5-diazabicyclo[4.2.0]octane

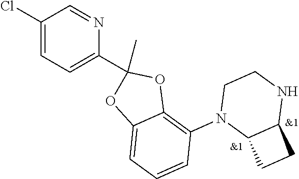 [SMILES: CC1(c2ccc(Cl)cn2)Oc2cccc(N3CCN[C@H]4CC[C@@H]43)c2O1]

A solution of HCl in Et₂O (2M, 439 mg, 0.6 ml) was added to a solution of rac-tert-butyl (1R,6R)-5-(2-(5-chloropyridin-2-yl)-2-methylbenzo[d][1,3]dioxol-4-yl)-2,5-diazabicyclo[4.2.0]octane-2-carboxylate Intermediate 25 (1.2 g, 2.2 mmol) in DCM (20 mL). The solution was stirred at rt for 8 h, and then concentrated under reduced pressure, and is the solids were dried in vacuo to give the hydrochloride of the title compound (1.03 g, 98%) as a beige solid; MS (ESI) m/z [M+H]⁺ 358.1.

### Intermediate 27

### Methyl 4-chloro-2-(((1RS,6RS)-5-(2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate

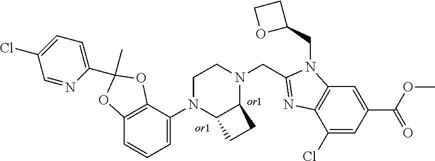 [SMILES: COC(=O)c1cc(Cl)c2nc(CN3CCN(c4cccc5c4OC(C)(c4ccc(Cl)cn4)O5)[C@H]4CC[C@@H]43)n(C[C@@H]3CCO3)c2c1]

K₂CO₃ (579 mg, 4.19 mmol) and methyl (S)-4-chloro-2-(chloromethyl)-1-(oxetan-2-ylmethyl)-1H-benzo[d]imidazole-6-carboxylate_Intermediate 24 (598 mg, 1.82 mmol) were added to a solution of rac-(1R,6R)-2-(2-(5-chloropyridin-2-yl)-2-methylbenzo[d][1,3]dioxol-4-yl)-2,5-diazabicyclo[4.2.0]octane Intermediate 26 (500 mg, 1.40 mmol) in MeCN (5 mL) and the reaction mixture was heated at 50° C. for 18 h. The reaction mixture was cooled to rt and filtered. The filtrate was purified by flash chromatography on silica (20-100% EtOAc in heptane) to give the title compound (0.555 g, 61%); MS (ESI) m/z [M+H]⁺ 652.1.

### Intermediate 28

### Methyl 4-chloro-2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 1

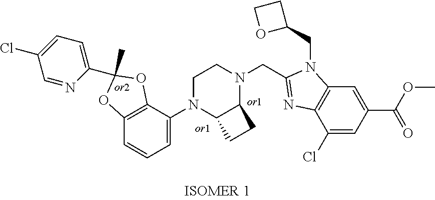 [SMILES: COC(=O)c1cc(Cl)c2nc(CN3CCN(c4cccc5c4O[C@@](C)(c4ccc(Cl)cn4)O5)[C@H]4CC[C@@H]43)n(C[C@@H]3CCO3)c2c1]

### Intermediate 29

### Methyl 4-chloro-2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 2

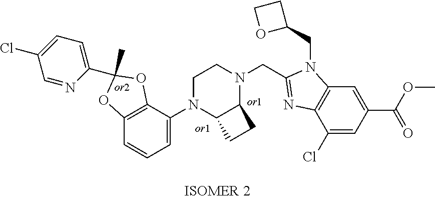 [SMILES: COC(=O)c1cc(Cl)c2nc(CN3CCN(c4cccc5c4O[C@@](C)(c4ccc(Cl)cn4)O5)[C@H]4CC[C@@H]43)n(C[C@@H]3CCO3)c2c1]

### Intermediate 30

### Methyl 4-chloro-2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate. Isomer 4

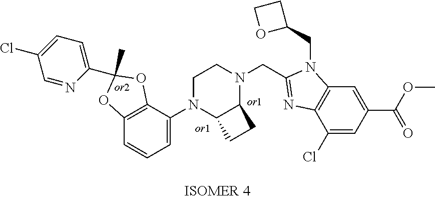 [SMILES: COC(=O)c1cc(Cl)c2nc(CN3CCN(c4cccc5c4O[C@@](C)(c4ccc(Cl)cn4)O5)[C@H]4CC[C@@H]43)n(C[C@@H]3CCO3)c2c1]

The diastereoisomers of methyl 4-chloro-2-(((1RS,6RS)-5-(2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate Intermediate 27 (555 mg) were separated by chiral chromatography on a LUX C4 column (250×30 mm, 5 μm), eluted with 30% MeCN/EtOH/DEA (70/30/20 mM) in CO₂, 125 bar, at a flow rate of 115 mL/min, and detected at 225 nm;
the first eluted compound was collected and evaporated to give the title compound Isomer 1 Intermediate 28 (89 mg); MS (ESI) m/z [M+H]⁺ 652.2;
the second eluted compound was collected and evaporated to give the title compound Isomer 2 Intermediate 29 (49 mg); MS (ESI) m/z [M+H]⁺ 652.45;
the fourth eluted compound was collected and evaporated to give the title compound Isomer 4 Intermediate 30 (150 mg); MS (ESI) m/z [M+H]⁺ 652.44.

### Intermediate 31

### rac-tert-Butyl (1R,6R)-5-(2-(4-chloro-2-fluorophenyl)-2-methylbenzo[d][1,3]dioxol-4-yl)-2,5-diazabicyclo[4.2.0]octane-2-carboxylate

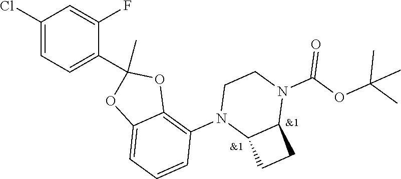 [SMILES: CC(C)(C)OC(=O)N1CCN(c2cccc3c2OC(C)(c2ccc(Cl)cc2F)O3)[C@H]2CC[C@@H]21]

A mixture of 4-bromo-2-(4-chloro-2-fluorophenyl)-2-methylbenzo[d][1,3]dioxole (WO2020234726) (2.89 g, 8.42 mmol), rac-tert-butyl (1R,6R)-2,5-diazabicyclo[4.2.0]octane-2-carboxylate (2.68 g, 12.63 mmol), RuPhos (786 mg, 1.68 mmol), Pd₂(dba)₃ (771 mg, 842 μmol) and sodium tert-butoxide (2.43 g, 25.27 mmol) in toluene (40 mL) was stirred under an atmosphere of N₂(g) at 100° C. overnight and then cooled to rt. The reaction mixture was poured into water (100 mL) and extracted with EtOAc (3×100 mL). The combined organic layer was washed with brine, dried over Na₂SO₄, and the solvent was removed under vacuum.

The crude product was purified by flash chromatography on silica (0-100% MTBE in hexane) to give the title compound (1.1 g, 35%) as a yellow oil; MS (ESI) m/z [M+H]⁺ 475.0.

### Intermediate 32 rac-(1R,6R)-2-(2-(4-Chloro-2-fluorophenyl)-2-methylbenzo[d][1,3]dioxol-4-yl)-2,5-diazabicyclo[4.2.0]octane

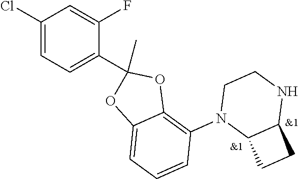 [SMILES: CC1(c2ccc(Cl)cc2F)Oc2cccc(N3CCN[C@H]4CC[C@@H]43)c2O1]

A solution of HCl in Et₂O (2M, 439 mg, 0.6 mL) was added to a solution of rac-tert-butyl (1R,6R)-5-(2-(4-chloro-2-fluorophenyl)-2-methylbenzo[d][1,3]dioxol-4-yl)-2,5-diazabicyclo[4.2.0]octane-2-carboxylate Intermediate 31 (1.1 g, 2.3 mmol) in DCM (15 mL). The reaction mixture was stirred at ambient temperature for 8 h and then concentrated under reduced pressure to give the hydrochloride of the title compound (950 mg, 98%) as a beige solid; MS (ESI) m/z [M+H]⁺ 375.0.

### Intermediate 33

### Methyl 2-(((1RS,6RS)-5-(2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate

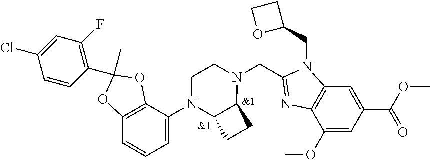 [SMILES: COC(=O)c1cc(OC)c2nc(CN3CCN(c4cccc5c4OC(C)(c4ccc(Cl)cc4F)O5)[C@H]4CC[C@@H]43)n(C[C@@H]3CCO3)c2c1]

DIPEA (824 mg, 6.38 mmol) was added to a suspension of rac-(1R,6R)-2-(2-(4-chloro-2-fluorophenyl)-2-methylbenzo[d][1,3]dioxol-4-yl)-2,5-diazabicyclo[4.2.0]octane hydrochloride Intermediate 32 (598 mg, 1.59 mmol), methyl (S)-2-(chloromethyl)-4-methoxy-1-(oxetan-2-ylmethyl)-1H-benzo[d]imidazole-6-carboxylate Intermediate 10 (518 25 mg, 1.59 mmol) and NaI (1.2 g, 7.97 mmol) in MeCN (5 mL) at 20° C., and the reaction mixture was heated at 60° C. overnight. The reaction mixture was concentrated under reduced pressure, and then diluted with EtOAc (10 mL). The organic layer was washed with sat NaHCO₃ (aq, 20 mL) and brine (20 mL), dried over Na₂SO₄, and concentrated under reduced pressure. The crude product was purified by preparative HPLC, PrepMethod E, (gradient: 40-65%) to give the title compound (102 mg, 35%); MS (ESI) m/z [M+H]⁺ 663.2.

### Intermediate 34

### Methyl 2-(((1R*,6R*)-5-((R*)-2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 1

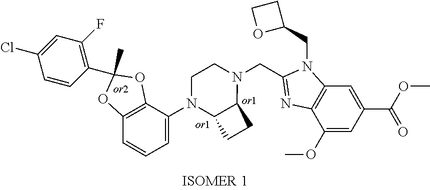 [SMILES: COC(=O)c1cc(OC)c2nc(CN3CCN(c4cccc5c4O[C@@](C)(c4ccc(Cl)cc4F)O5)[C@H]4CC[C@@H]43)n(C[C@@H]3CCO3)c2c1]

### Intermediate 35

### Methyl 2-(((1R*,6R*)-5-((R*)-2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 2

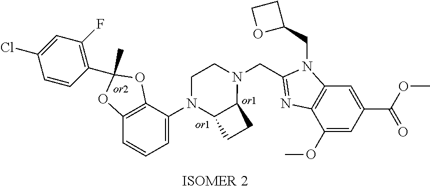 [SMILES: COC(=O)c1cc(OC)c2nc(CN3CCN(c4cccc5c4O[C@@](C)(c4ccc(Cl)cc4F)O5)[C@H]4CC[C@@H]43)n(C[C@@H]3CCO3)c2c1]

### Intermediate 36

### Methyl 2-(((1R*,6R*)-5-((R*)-2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate. Isomer 3

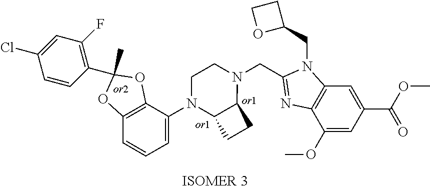 [SMILES: COC(=O)c1cc(OC)c2nc(CN3CCN(c4cccc5c4O[C@@](C)(c4ccc(Cl)cc4F)O5)[C@H]4CC[C@@H]43)n(C[C@@H]3CCO3)c2c1]

The diastereomers of Intermediate 33 (218 mg) were separated by chiral chromatography on a Whelk-01 column (250×50 mm, 5 μm), eluted with 35% MeCN/MeOH/DEA (50/50/20 mM) in CO₂, 120 bar, at a flow rate of 400 mL/min and detected at 220 nm; the first eluted compound mixture was collected and evaporated to yield a mixture of isomers (148 mg); and the second eluted compound was collected and evaporated to yield the title compound Isomer 3, Intermediate 36 (28 mg); MS (ESI) m/z [M+H]⁺ 663.4.

The stereoisomers of the first eluted compound mixture (148 mg) were separated by chiral chromatography on a Chiralpak IH column (250×30 mm, 5 μm), eluted with 10% MeCN/MeOH/DEA (50/50/20 mM) in CO₂, 120 bar, at a flow rate of 130 mL/min and detected at 230 nm;
the first eluted compound was collected and evaporated to give the title compound Isomer 1, Intermediate 34 (85 mg); MS (ESI) m/z [M+H]⁺ 663.3; and is the second eluted compound was collected and evaporated to give the title compound Isomer 2, Intermediate 35 (30 mg); MS (ESI) m/z [M+H]⁺ 663.3.

### Intermediate 37

### Methyl 2-(((1RS,6SR)-5-(2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6

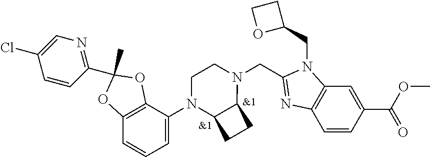 [SMILES: COC(=O)c1ccc2nc(CN3CCN(c4cccc5c4O[C@@](C)(c4ccc(Cl)cn4)O5)[C@@H]4CC[C@@H]43)n(C[C@@H]3CCO3)c2c1]

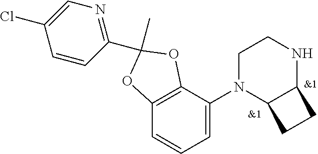 [SMILES: CC1(c2ccc(Cl)cn2)Oc2cccc(N3CCN[C@H]4CC[C@H]43)c2O1]

TFA (354 μl, 4.60 mmol) was added to a solution of rac-tert-butyl (1R,6S)-5-(2-(5-chloropyridin-2-yl)-2-methylbenzo[d][1,3]dioxol-4-yl)-2,5-diazabicyclo[4.2.0]octane-2-carboxylate Intermediate 11 (421 mg, 0.92 mmol) in DCM (3.0 mL) and the reaction mixture was stirred at rt for 20 min. The reaction mixture was concentrated at reduced pressure to give the crude Step a) subtitle compound; MS (ESI) m/z [M+H]⁺ 358.3.

The crude product from Step a) was dissolved in MeCN (3 mL) and K₂CO₃ (381 mg, 2.76 mmol) and methyl (S)-2-(chloromethyl)-1-(oxetan-2-ylmethyl)-1H-benzo[d]imidazole-6-carboxylate (270 mg, 0.92 mmol) were added, and the reaction mixture was heated at 75° C. for 1 h. The mixture was cooled to rt and filtered through celite. The filtrate was collected and purified by flash chromatography on silica (30-100% EtOAc in heptane, then 5% EtOH in is EtOAc) to give the title compound (0.450 g, 79%); MS (ESI) m/z [M+H]⁺ 616.3.

### Intermediate 38

### Methyl 2-(((1RS,6SR)-5-(2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 1 and Isomer 2

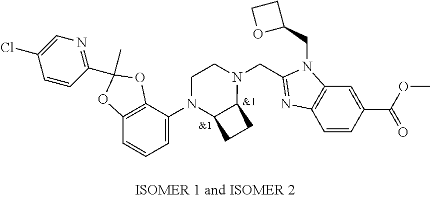 [SMILES: COC(=O)c1ccc2nc(CN3CCN(c4cccc5c4OC(C)(c4ccc(Cl)cn4)O5)[C@@H]4CC[C@@H]43)n(C[C@@H]3CCO3)c2c1]

### Intermediate 39

### Methyl 2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 3

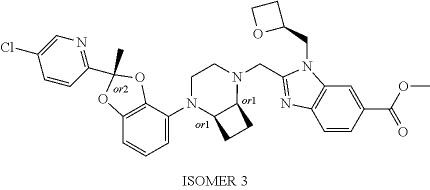 [SMILES: COC(=O)c1ccc2nc(CN3CCN(c4cccc5c4O[C@@](C)(c4ccc(Cl)cn4)O5)[C@@H]4CC[C@@H]43)n(C[C@@H]3CCO3)c2c1]

### Intermediate 40

### Methyl 2-((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 4

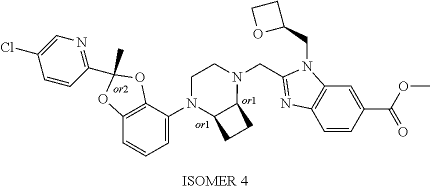 [SMILES: COC(=O)c1ccc2nc(CN3CCN(c4cccc5c4O[C@@](C)(c4ccc(Cl)cn4)O5)[C@@H]4CC[C@@H]43)n(C[C@@H]3CCO3)c2c1]

The diastereomers of methyl 2-(((1RS,6SR)-5-(2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate Intermediate 37 (480 mg) were separated by chiral chromatography on a Chiralpak IH column (250×30 mm, 5 μm), eluted with 25% EtOH/DEA (100/20 mM) in CO₂, 120 bar, at a flow rate of 140 mL/min and detected at 230 nm and;
the first eluted compound mixture was collected and evaporated to give a mixture of the title compounds Isomer 1 and Isomer 2, Intermediate 38 (158 mg);
the second eluted compound was collected and evaporated to give the title compound Isomer 3 Intermediate 39 (91 mg); MS (ESI) m/z [M+H]⁺ 616.50;
and the third eluted compound was collected and evaporated to give the title compound Isomer 4 Intermediate 40 (72 mg); MS (ESI) m/z [M+H]⁺ 616.40.

### Intermediate 41

### 2-(((1RS,6SR)-5-(2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid, Isomer 1 and Isomer 2

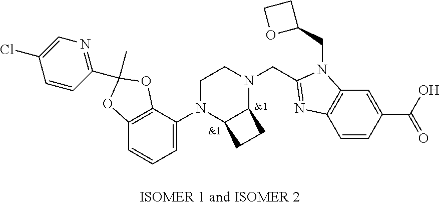 [SMILES: CC1(c2ccc(Cl)cn2)Oc2cccc(N3CCN(Cc4nc5ccc(C(=O)O)cc5n4C[C@@H]4CCO4)[C@H]4CC[C@H]43)c2O1]

A mixture of methyl 2-(((1RS,6SR)-5-(2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 1 and Isomer 2 Intermediate 38 (200 mg, 0.32 mmol) and 1,3,4,6,7,8-hexahydro-2H-pyrimido[1,2-a]pyrimidine (90 mg, 0.65 mmol) in a sealed flask was evacuated and backfilled with N₂(g) (×3). A mixture of MeCN (3 mL) and water (0.6 mL) was degassed by N₂(g) bubbling for 15 min and added. The reaction mixture was evacuated and backfilled with N₂(g) (×3) and then stirred at rt overnight. The reaction mixture was concentrated at reduced pressure, and the residue was dissolved in DMSO, filtered and purified by preparative HPLC, PrepMethod A, (gradient: 20-90%) to give the title is compound Isomer 1 and Isomer 2 (0.120 g, 61.4%); MS (ESI) m/z [M+H]⁺ 602.4.

### Intermediate 42

### Methyl 2-(((1RS,6RS)-5-(2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate

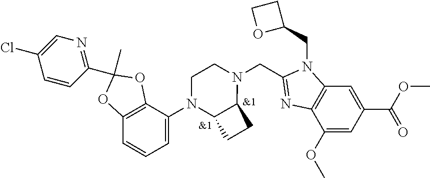 [SMILES: COC(=O)c1cc(OC)c2nc(CN3CCN(c4cccc5c4OC(C)(c4ccc(Cl)cn4)O5)[C@H]4CC[C@@H]43)n(C[C@@H]3CCO3)c2c1]

K₂CO₃ (463 mg, 3.35 mmol) and methyl (S)-2-(chloromethyl)-4-methoxy-1-(oxetan-2-ylmethyl)-1H-benzo[d]imidazole-6-carboxylate Intermediate 10 (399 mg, 1.23 mmol) was added to a solution of rac-(1R,6R)-2-(2-(5-chloropyridin-2-yl)-2-methylbenzo[d][1,3]dioxol-4-yl)-2,5-diazabicyclo[4.2.0]octane Intermediate 26 (400 mg, 1.12 mmol) in MeCN (3 mL) and the reaction mixture was heated at 60° C. for 40 h. The reaction mixture was cooled to rt and filtered over celite. The filtrate was collected and purified by flash chromatography on silica (0-100% EtOAc in heptane) to give the title compound (0.47 g, 65%); MS (ESI) m z [M+H]⁺ 646.4.

### Intermediate 43

### Methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 1

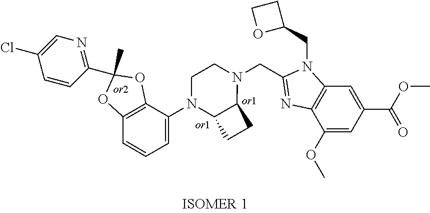 [SMILES: COC(=O)c1cc(OC)c2nc(CN3CCN(c4cccc5c4O[C@@](C)(c4ccc(Cl)cn4)O5)[C@H]4CC[C@@H]43)n(C[C@@H]3CCO3)c2c1]

### Intermediate 44

### Methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 2

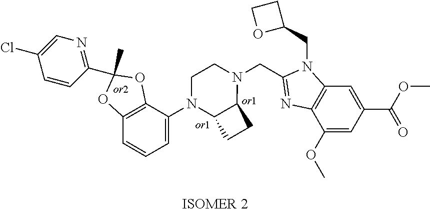 [SMILES: COC(=O)c1cc(OC)c2nc(CN3CCN(c4cccc5c4O[C@@](C)(c4ccc(Cl)cn4)O5)[C@H]4CC[C@@H]43)n(C[C@@H]3CCO3)c2c1]

### Intermediate 45 Methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate. Isomer 3

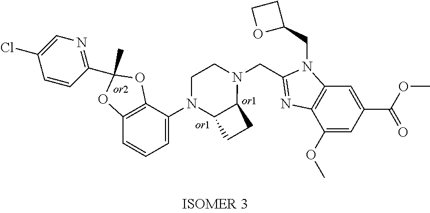 [SMILES: COC(=O)c1cc(OC)c2nc(CN3CCN(c4cccc5c4O[C@@](C)(c4ccc(Cl)cn4)O5)[C@H]4CC[C@@H]43)n(C[C@@H]3CCO3)c2c1]

### Intermediate 46

### Methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 4

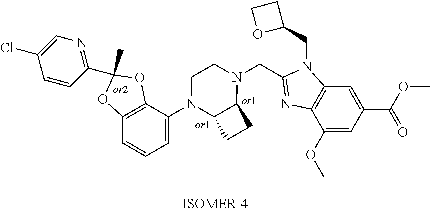 [SMILES: COC(=O)c1cc(OC)c2nc(CN3CCN(c4cccc5c4O[C@@](C)(c4ccc(Cl)cn4)O5)[C@H]4CC[C@@H]43)n(C[C@@H]3CCO3)c2c1]

The diastereoisomers of methyl 2-(((1RS,6RS)-5-(2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate Intermediate 42 (470 mg) were separated by chiral chromatography on a DCPakA column (250×20 mm, 5 μm), eluted with 18% MeOH/DEA (100/20 mM) in CO₂, 130 bar, at a flow rate of 70 mL/min and detected at 240 nm;
the first eluted compound mixture was collected and evaporated to give a mixture of all four stereoisomers.

The stereoisomers of the first eluted compound mixture were separated by chiral chromatography on a Whelk-01 column (250×50 mm, 5 μm), eluted with 35% MeOH/DEA (100/20 mM) in CO₂, 120 bar, at a flow rate of 400 mL/min and detected at 220 nm;
the first eluted compound mixture was collected and evaporated to yield a mixture of isomers (107 mg); and
the second eluted compound mixture was collected and evaporated to yield a mixture of isomers (120 mg);

The stereoisomers of the first eluted compound mixture (107 mg) were separated by chiral chromatography on a Lux C3 (OJ) column (250×30 mm, 5 um), eluted with 15%

MeOH/MeCN/DEA (85/15/20 mM) in CO₂, 130 bar, at a flow rate of 130 mL/min, and detected at 230 nm;
the first eluted compound was collected and evaporated to give the title compound Isomer 1, Intermediate 43 (53 mg); MS (ESI) m/z [M+H]⁺ 646.5;
and the second eluted compound was collected and evaporated to give the title compound Isomer 2, Intermediate 44 (43 mg); MS (ESI) m/z [M+H]⁺ 0.646.4.

The stereoisomers of the second eluted compound mixture (120 mg) were separated by chiral chromatography on a Kromasil XT column (250×20 mm, 5 μm), eluted with 10% MeCN/MeOH/NH₃ (50/50/20 mM) in CO₂, 130 bar, at a flow rate of 70 mL/min, and detected at 254 nm;

The first eluted compound was collected and evaporated to give the title compound Isomer 3, Intermediate 45, (30 mg); MS (ESI) m/z [M+H]⁺ 646.5; and the second eluted compound was collected and evaporated to give the title compound Isomer 4, Intermediate 46, (54 mg); MS (ESI) m/z [M+H]⁺ 646.7.

### Intermediate 47

### (S)-5-Bromo-3-methoxy-2-nitro-N-((tetrahydrofuran-2-yl)methyl)aniline

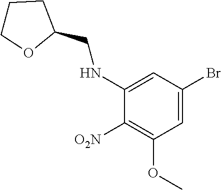 [SMILES: COc1cc(Br)cc(NC[C@@H]2CCCO2)c1[N+](=O)[O-]]

DIPEA (3.73 ml, 21.35 mmol) was added to a solution of (S)-(tetrahydrofuran-2-yl)methanamine (1.08 g, 10.68 mmol) and 5-bromo-1-fluoro-3-methoxy-2-nitrobenzene (2.67 g, 10.68 mmol) in MeCN (20 mL) and the reaction mixture was stirred at 57° C. for 3 h. The reaction mixture was concentrated at reduced pressure and the residue was purified by flash chromatography on silica (0-20% EtOAc in heptane) to give the title compound (2.39 g, 68%); MS (ESI) m/z [M+H]⁺ 333.1.

### Intermediate 48

### (S)-5-Bromo-3-methoxy-N1-((tetrahydrofuran-2-yl)methyl)benzene-1.2-diamine

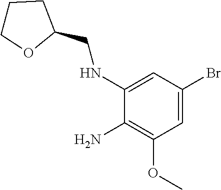 [SMILES: COc1cc(Br)cc(NC[C@@H]2CCCO2)c1N]

DIPEA (6.07 ml, 34.73 mmol) and HSiCl₃ (2.453 ml, 24.31 mmol) was added dropwise at 0° C. to a solution of (S)-5-bromo-3-methoxy-2-nitro-N-((tetrahydrofuran-2-yl)methyl)aniline Intermediate 47 (2.3 g, 6.95 mmol) in MeCN (20 mL). The reaction mixture was stirred at 0° C. for 2 min and then at rt for 30 min. NaHCO₃(aq, 10 mL) was added dropwise and the biphasic mixture was stirred at rt for 30 min and then extracted with EtOAc (2×5 mL). The combined organic layer was dried over MgSO₄, filtered and evaporated at reduced pressure to give the title compound (2.0 g, 96%); MS (ESI) m/z [M+H]⁺ 303.1.

### Intermediate 49

### Methyl (S)-4-amino-3-methoxy-5-(((tetrahydrofuran-2-yl)methyl)amino)benzoate

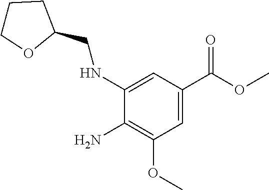 [SMILES: COC(=O)c1cc(NC[C@@H]2CCCO2)c(N)c(OC)c1]

DIPEA (8.70 ml, 49.80 mmol) was added to a suspension of (S)-5-bromo-3-methoxy-N¹-((tetrahydrofuran-2-yl)methyl)benzene-1,2-diamine Intermediate 48 (1.5 g, 4.98 mmol) and Pd(dppf)Cl₂ (292 mg, 0.40 mmol) in MeOH (20 mL) and the reaction mixture was stirred under an atmosphere of CO(g) at 9 atm and 85° C. for 16 h. The reaction mixture was filtered through a pad of celite, and the filter cake was rinsed with MeOH (10 mL). The filtrate was concentrated at reduced pressure and the residue was purified by flash chromatography on silica (20-60% EtOAc in heptane) to give the title compound (444 mg, 32%); MS (ESI) m z [M+H]⁺ 281.2.

### Intermediate 50

### Methyl (S)-2-(chloromethyl)-4-methoxy-1-((tetrahydrofuran-2-yl)methyl)-1H-benzo[d]imidazole-6-carboxylate

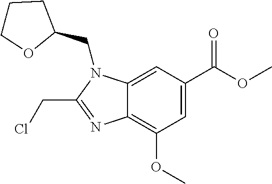 [SMILES: COC(=O)c1cc(OC)c2nc(CCl)n(C[C@@H]3CCCO3)c2c1]

pTsOH (149 mg, 0.78 mmol) was added to a solution of methyl (S)-4-amino-3-methoxy-5-(((tetrahydrofuran-2-yl)methyl)amino)benzoate Intermediate 49 (440 mg, 1.57 mmol) and 2-chloro-1,1,1-trimethoxyethane (485 mg, 3.14 mmol) in MeCN (10 mL) and the reaction mixture was stirred at 50° C. for 30 min. The reaction mixture was diluted with EtOAc (10 mL) and washed with NaHCO₃ (aq, 2×3 mL). The organic layer was dried over MgSO₄, filtered and evaporated at reduced pressure to give the title compound (450 mg, 85%); MS (ESI) m/z [M+H]⁺ 399.0.

### Intermediate 51

### Methyl 2-(((1RS,6RS)-5-(2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate

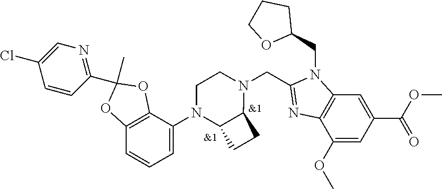 [SMILES: COC(=O)c1cc(OC)c2nc(CN3CCN(c4cccc5c4OC(C)(c4ccc(Cl)cn4)O5)[C@H]4CC[C@@H]43)n(C[C@@H]3CCCO3)c2c1]

is K₂CO₃ (406 mg, 2.93 mmol) and methyl (S)-2-(chloromethyl)-4-methoxy-1-((tetrahydrofuran-2-yl)methyl)-1H-benzo[d]imidazole-6-carboxylate Intermediate 50 (340 mg, 1.00 mmol) were added to a solution of rac-(1R,6R)-2-(2-(5-chloropyridin-2-yl)-2-methylbenzo[d][1,3]dioxol-4-yl)-2,5-diazabicyclo[4.2.0]octane Intermediate 26 (350 mg, 0.98 mmol) in MeCN (5 mL) and the reaction mixture was heated at 50° C. for 18 h. The reaction mixture was cooled to rt and EtOAc (10 mL) was added. The organic layer was washed with NaHCO₃ (aq, 10 mL), dried over MgSO₄ and the solvent was evaporated at reduced pressure. The crude product was purified by flash chromatography on silica (20-100% EtOAc in heptane, then 3% EtOH in EtOAc) to give the title compound (0.377 g, 58%); MS (ESI) m/z [M+H]⁺ 660.64.

### Intermediate 52

### Methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate, Isomer 1

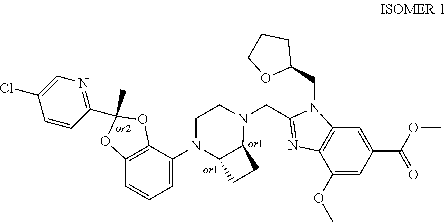 [SMILES: COC(=O)c1cc(OC)c2nc(CN3CCN(c4cccc5c4O[C@@](C)(c4ccc(Cl)cn4)O5)[C@H]4CC[C@@H]43)n(C[C@@H]3CCCO3)c2c1]

### Intermediate 53

### Methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate, Isomer 2

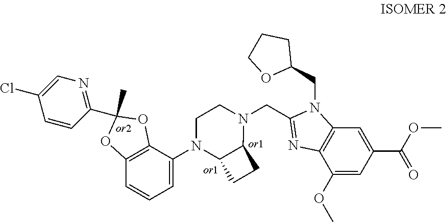 [SMILES: COC(=O)c1cc(OC)c2nc(CN3CCN(c4cccc5c4O[C@@](C)(c4ccc(Cl)cn4)O5)[C@H]4CC[C@@H]43)n(C[C@@H]3CCCO3)c2c1]

### Intermediate 54

### Methyl 2-(((1R*,6R*)-5-((R*)-2-(5-chloropyridin-2-yl)-2-methylbenzo[d][1,3]dioxol-4-yl)-is 2,5-diazabicyclo[4.2.0]octan-2-yl)methyl)-4-methoxy-1-(((S)-tetrahydrofuran-2-yl)methyl)-1H-benzo[d]imidazole-6-carboxylate, Isomer 3

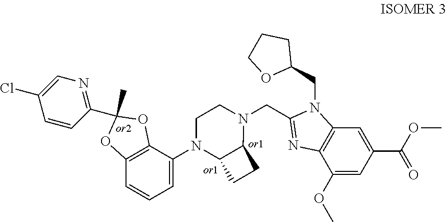 [SMILES: COC(=O)c1cc(OC)c2nc(CN3CCN(c4cccc5c4O[C@@](C)(c4ccc(Cl)cn4)O5)[C@H]4CC[C@@H]43)n(C[C@@H]3CCCO3)c2c1]

The diastereoisomers of methyl 2-(((1RS,6RS)-5-(2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate Intermediate 51 (377 mg) were separated by chiral chromatography on a Whelk-01 column (250×50, 10 μm), eluted with 40% MeOH/DEA (100/20 mM) in CO₂, 120 bar, at a flow rate of 400 mL/min, and detected at 220 nm;
the first eluted compound mixture was collected and evaporated to give a mixture of isomers (191 mg); and
the second eluted compound mixture was collected and evaporated to give a mixture of isomers (200 mg).

The stereoisomers of the first eluted compound mixture (191 mg) were separated by chiral chromatography on a Kromasil XT column (250×20, 5 μm), eluted with 10% MeOH/NH₃ (100/20 mM) in CO₂, 120 bar, at a flow rate of 100 mL/min, and detected at 254 nm; the first eluted compound was collected and evaporated to give the title compound Isomer 1, Intermediate 52 (29 mg); MS (ESI) m/z [M+H]⁺ 660.4; and the second eluted compound was collected and evaporated to give the title compound Isomer is 2, Intermediate 53 (52 mg); MS (ESI) m/z [M+H]⁺ 660.5.

The stereoisomers of the second eluted compound mixture (200 mg) were separated by chiral chromatography on a Kromasil XT (150×4.6, 5 μm), eluted with 6% MeOH/NH₃ (100/20 mM) in CO₂, 140 bar, at a flow rate of 70 mL/min, and detected at 240 nm; the first eluted compound was collected and evaporated to give the title compound Isomer 3, Intermediate 54 (31 mg); MS (ESI) m/z [M+H]⁺ 660.7.

### Intermediate 55

### Methyl 2-(((1RS,6SR)-5-(2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate

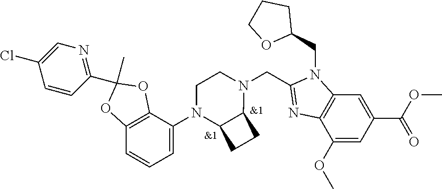 [SMILES: COC(=O)c1cc(OC)c2nc(CN3CCN(c4cccc5c4OC(C)(c4ccc(Cl)cn4)O5)[C@@H]4CC[C@@H]43)n(C[C@@H]3CCCO3)c2c1]

K₂CO₃ (406 mg, 2.93 mmol) and methyl (S)-2-(chloromethyl)-4-methoxy-1-((tetrahydrofuran-2-yl)methyl)-1H-benzo[d]imidazole-6-carboxylate Intermediate 50 (340 mg, 1.00 mmol) was added to a solution of rac-(1R,6S)-2-(2-(5-chloropyridin-2-yl)-2-methylbenzo[d][1,3]dioxol-4-yl)-2,5-diazabicyclo[4.2.0]octane Intermediate 12 Step a) (350 mg, 0.98 mmol) in MeCN (5 mL) and the reaction mixture was heated at 50° C. for 18 h. The reaction mixture was cooled to rt and filtered through celite, the filtrate was collected and purified by flash chromatography on silica (20-100% EtOAc in heptane) to give the title compound (0.382 g, 59%); MS (ESI) m/z [M+H]⁺ 660.4.

### Intermediate 56

### Methyl 2-((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate, Isomer 1

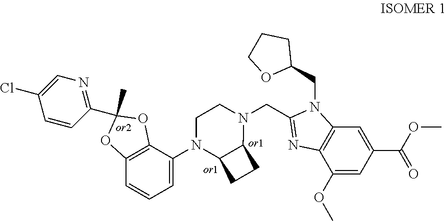 [SMILES: COC(=O)c1cc(OC)c2nc(CN3CCN(c4cccc5c4O[C@@](C)(c4ccc(Cl)cn4)O5)[C@@H]4CC[C@@H]43)n(C[C@@H]3CCCO3)c2c1]

### Intermediate 57

### Methyl 2-(((1R*,6S*)-5-((R*)-2-(5-chloropyridin-2-yl)-2-methylbenzo[d][1,3]dioxol-4-yl)-is 2,5-diazabicyclo[4.2.0]octan-2-yl)methyl)-4-methoxy-1-(((S)-tetrahydrofuran-2-yl)methyl)-1H-benzo[d]imidazole-6-carboxylate, Isomer 2

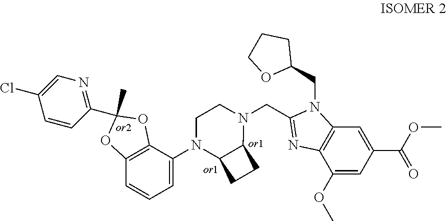 [SMILES: COC(=O)c1cc(OC)c2nc(CN3CCN(c4cccc5c4O[C@@](C)(c4ccc(Cl)cn4)O5)[C@@H]4CC[C@@H]43)n(C[C@@H]3CCCO3)c2c1]

### Intermediate 58

### Methyl 2-((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate. Isomer 3

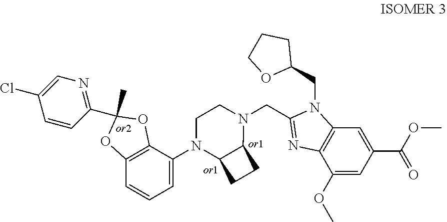 [SMILES: COC(=O)c1cc(OC)c2nc(CN3CCN(c4cccc5c4O[C@@](C)(c4ccc(Cl)cn4)O5)[C@@H]4CC[C@@H]43)n(C[C@@H]3CCCO3)c2c1]

### Intermediate 59

### Methyl 2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate, Isomer 4

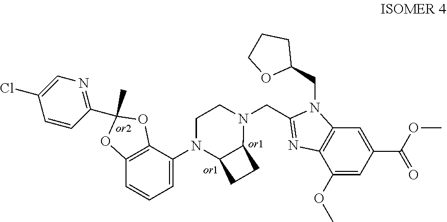 [SMILES: COC(=O)c1cc(OC)c2nc(CN3CCN(c4cccc5c4O[C@@](C)(c4ccc(Cl)cn4)O5)[C@@H]4CC[C@@H]43)n(C[C@@H]3CCCO3)c2c1]

The diastereoisomers of methyl 2-(((1RS,6SR)-5-(2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate Intermediate 55 (382 mg) were separated by chiral chromatography on a Whelk-01 column (250×50, 10 m), eluted with 45% MeOH/DEA (100/20 mM) in CO₂, 120 bar, at a flow rate of 400 mL/min, and detected at 220 nm;
the first eluted compound mixture was collected and evaporated to give a mixture of isomers is (224 mg); and
the second eluted compound mixture was collected and evaporated to give a mixture of isomers (162 mg).

The stereoisomers of the first eluted compound mixture were separated by chiral chromatography on a LUX C3 (OJ) column (250×30, 5 μm), eluted with 17% MeCN/MeOH/DEA (85/15/20 mM) in CO₂, 130 bar, at a flow rate of 120 mL/min and detected at 240 nm;
the first eluted compound was collected and evaporated to give the title compound Isomer 1, Intermediate 56 (74 mg); MS (ESI) m/z [M+H]⁺ 660.7; and
the second elute compound was collected and evaporated to give the title compound Isomer 2, Intermediate 57 (109 mg); MS (ESI) m/z [M+H]⁺ 660.5.

The stereoisomers of the second eluted compound mixture (162 mg) were separated by chiral chromatography on a LUX C3 (OJ) column (250×30, 5 μm), eluted with 18% MeCN/MeOH/DEA (85/15/20 mM) in CO₂, 130 bar, at a flow rate of 120 mL/min and detected at 240 nm;
the first eluted compound was collected and evaporated to give the title compound Isomer 3, Intermediate 58 (67 mg); MS (ESI) m/z [M+H]⁺ 660.4; and
the second eluted compound was collected and evaporated to give the title compound Isomer 4, Intermediate 59 (67 mg); MS (ESI) m/z [M+H]⁺ 660.7.

### Intermediate 60

### rac-tert-Butyl (1R,6S)-5-(2-(4-Chloro-2-fluorophenyl)-2-methylbenzo[d][1,3]dioxol-4-yl)-2,5-diazabicyclo[4.2.0]octane-2-carboxylate

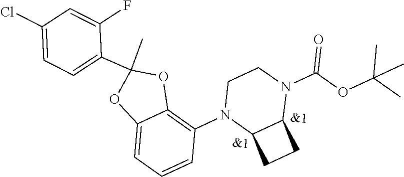 [SMILES: CC(C)(C)OC(=O)N1CCN(c2cccc3c2OC(C)(c2ccc(Cl)cc2F)O3)[C@@H]2CC[C@@H]21]

Pd₂(dba)₃ (366 mg, 0.40 mmol) was added to a mixture of 4-bromo-2-(4-chloro-2-fluorophenyl)-2-methylbenzo[d][1,3]dioxole (WO2020234726) (1.37 g, 4.0 mmol), rac-tert-butyl (1R,6S)-2,5-diazabicyclo[4.2.0]octane-2-carboxylate Intermediate 4 (849 mg, 4.00 mmol), RuPhos (747 mg, 1.60 mmol) and sodium 2-methylpropan-2-olate (1.54 g, 16.00 mmol) in degassed toluene (10 mL). The reaction mixture was evacuated and backfilled with N₂(g) (×3) then stirred at 100° C. for 10 min. The mixture was cooled to rt and filtered through a pad of celite, and the pad was rinsed with toluene. The filtrate was collected and the solvent was evaporated. The crude compound was purified by flash chromatography on silica (0-20% EtOAc in heptane) to give the title compound (950 mg, 50%); MS (ESI) m/z [M+H]⁺ 475.3.

### Intermediate 61

### Methyl 2-(((1RS,6SR)-5-(2-(4-chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate

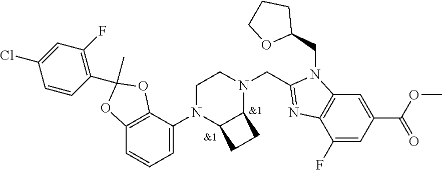 [SMILES: COC(=O)c1cc(F)c2nc(CN3CCN(c4cccc5c4OC(C)(c4ccc(Cl)cc4F)O5)[C@@H]4CC[C@@H]43)n(C[C@@H]3CCCO3)c2c1]

### Step a) rac-(1R,6S)-2-(2-(4-Chloro-2-fluorophenyl)-2-methylbenzo[d][1,3]dioxol-4-yl)-2,5-diazabicyclo[4,2,0]oxtane

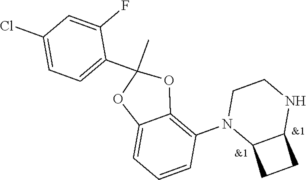 [SMILES: CC1(c2ccc(Cl)cc2F)Oc2cccc(N3CCN[C@H]4CC[C@H]43)c2O1]

TFA (770 μl, 10.0 mmol) was added to a solution of rac-tert-butyl (1R,6S)-5-(2-(4-Chloro-2-fluorophenyl)-2-methylbenzo[d][1,3]dioxol-4-yl)-2,5-diazabicyclo[4.2.0]octane-2-carboxylate Intermediate 60 (950 mg, 2.00 mmol) in MeCN (30 mL) and the reaction mixture was stirred at rt for 1 h. The reaction mixture was concentrated under reduced pressure and co-evaporated with toluene (×1) and then MeCN (×2) to give the crude sub-title compound; MS (ESI) m/z [M+H]⁺ 375.2.

K₂CO₃ (829 mg, 6.00 mmol) and methyl (S)-2-(chloromethyl)-4-fluoro-1-(oxetan-2-ylmethyl)-1H-benzo[d]imidazole-6-carboxylate Intermediate 7 (751 mg, 2.40 mmol) were added to a solution of the Step a) product in MeCN (30 mL) and the reaction mixture was heated at 30° C. for 18 h. The reaction mixture was cooled to rt and filtered. The filtrate was collected and the crude product was purified by flash chromatography on silica (0-100% EtOAc in heptane). The product containing fractions were collected and concentrated, and the crude product was purified by preparative HPLC, PrepMethod F, (gradient: 40-70%) to give the title compound (810 mg, 62%); MS (ESI) m/z [M+H]⁺ 651.2.

### Intermediate 62

### Methyl 2-(((1R*,6S*)-5-(R*)-2-(4-chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 1

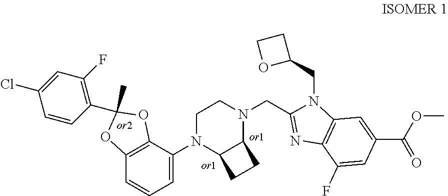 [SMILES: COC(=O)c1cc(F)c2nc(CN3CCN(c4cccc5c4O[C@@](C)(c4ccc(Cl)cc4F)O5)[C@@H]4CC[C@@H]43)n(C[C@@H]3CCO3)c2c1]

### Intermediate 63

### Methyl 2-(((1R*,6S*)-5-(R*)-2-(4-chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 2

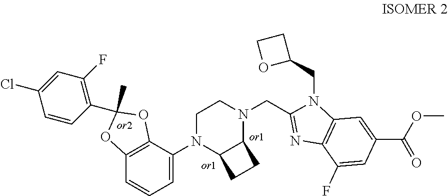 [SMILES: COC(=O)c1cc(F)c2nc(CN3CCN(c4cccc5c4O[C@@](C)(c4ccc(Cl)cc4F)O5)[C@@H]4CC[C@@H]43)n(C[C@@H]3CCO3)c2c1]

### Intermediate 64

### Methyl 2-(1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 3

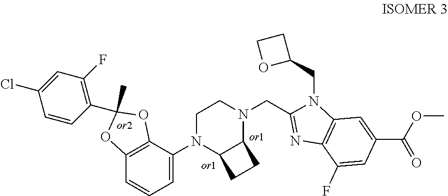 [SMILES: COC(=O)c1cc(F)c2nc(CN3CCN(c4cccc5c4O[C@@](C)(c4ccc(Cl)cc4F)O5)[C@@H]4CC[C@@H]43)n(C[C@@H]3CCO3)c2c1]

### Intermediate 65

### Methyl 2-(((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 4

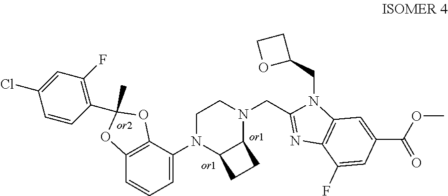 [SMILES: COC(=O)c1cc(F)c2nc(CN3CCN(c4cccc5c4O[C@@](C)(c4ccc(Cl)cc4F)O5)[C@@H]4CC[C@@H]43)n(C[C@@H]3CCO3)c2c1]

The stereoisomers of methyl 2-(((1RS,6SR)-5-(2-(4-chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate Intermediate 61 (810 mg) were separated by chiral chromatography on a Lux C4 column (250×50 mm, 5 μm), eluted with 18% EtOH/DEA (100/20 mM) in CO₂, 135 bar, at a flow rate of 350 mL/min, and detected at 220 nm;
the first eluted compound mixture was collected and evaporated to give a mixture of stereoisomers (318 mg);
the second eluted compound was collected and evaporated to give the title compound Isomer 3, Intermediate 64 (177 mg, 22%); MS (ESI) m/z [M+H]⁺ 651.3;
is the third eluted compound was collected and evaporated to give the title compound Isomer 4, Intermediate 65 (159 mg, 20%); MS (ESI) m/z [M+H]⁺ 651.4.

The stereoisomers of the first eluted compound mixture (318 mg) were separated by chiral chromatography on a Lux C3 (OJ) column (250×30 mm, 5 μm), eluted with 8% MeOH/DEA (100/20 mM) in CO₂, 130 bar, at a flow rate of 130 mL/min, and detected at 220 nm; the first eluted compound was collected and evaporated to give the title compound Isomer 1, Intermediate 62 (114 mg, 14%); MS (ESI) m/z [M+H]⁺ 651.3; and
the second eluted compound was collected and evaporated to give the title compound Isomer 2, Intermediate 63 (129 mg, 16%); MS (ESI) m/z [M+H]⁺ 651.3.

### Intermediate 66

### Methyl 3-chloro-5-(((1-ethyl-1H-imidazol-5-yl)methyl)amino)-4-nitrobenzoate

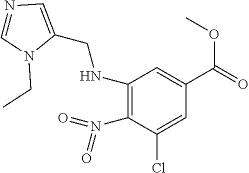 [SMILES: CCn1cncc1CNc1cc(C(=O)OC)cc(Cl)c1[N+](=O)[O-]]

Methyl 3-chloro-5-fluoro-4-nitrobenzoate (7.93 g, 33.95 mmol), (1-ethyl-1H-imidazol-5-yl)methanamine (4.25 g, 33.95 mmol) and DIPEA (10.97 g, 84.87 mmol) were mixed in DMF (80 mL), and the reaction mixture was heated at 50° C. for 16 h. The mixture were poured into water (100 mL) and filtered. The precipitate was collected and dried to give the title compound (11.5 g) as dark yellow solid; MS (ESI) m/z [M+H]⁺ 325.6.

### Intermediate 67

### Methyl 4-amino-3-chloro-5-(((1-ethyl-1H-imidazol-5-yl)methyl)amino)benzoate

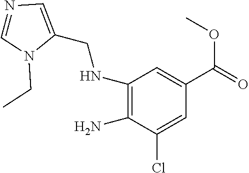 [SMILES: CCn1cncc1CNc1cc(C(=O)OC)cc(Cl)c1N]

Wet Pt/C (1%, 1.99 g, 10.2 mmol) was added to a suspension of methyl 3-chloro-5-(((1-ethyl-1H-imidazol-5-yl)methyl)amino)-4-nitrobenzoate Intermediate 66 (11.52 g, 34.01 mmol) in MeOH (100 mL) and the reaction mixture was stirred under an atmosphere of H₂ (g) at 1 atm and at 20° C. for 16 h. The reaction mixture was filtered, and the solids were washed with is MeOH (50 mL). The filtrate was concentrated under reduced pressure. The crude product was purified by flash chromatography on silica (0-40% MeOH in CHCl₃) to give the title compound (4 g, 38%); ¹H NMR (400 MHz, CDCl₃): δ 7.587 (s, 1H), 7.583 (s, 1H), 7.28 (s, 1H), 6.68 (s, 1H), 4.5 (brs, 1H), 4.24 (s, 2H), 3.99-3.97 (q, 2H), 3.85 (s, 3H), 1.43 (t, 3H)

### Intermediate 68

### Methyl 4-chloro-2-(chloromethyl)-1-((1-ethyl-1H-imidazol-5-yl)methyl)-1H-benzo[d]imidazole-6-carboxylate

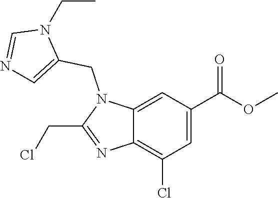 [SMILES: CCn1cncc1Cn1c(CCl)nc2c(Cl)cc(C(=O)OC)cc21]

2,2,2-Triethoxyethan-1-ol (3.11 g, 17.46 mmol) and pTsOH (100 mg, 582 μmol) were added to a stirred solution of methyl 4-amino-3-chloro-5-(((1-ethyl-1H-imidazol-5-yl)methyl)amino)benzoate Intermediate 67 (1.8 g, 5.82 mmol) in MeCN (100 mL) and the reaction mixture was heated at 60° C. 18 h. The reaction mixture concentrated under reduced pressure, and the crude residue was diluted with EtOAc. The organic layer was washed with water and NaHCO₃ (aq), dried and evaporated at reduced pressure to give the crude subtitle compound (2.04 g, 60% purity); MS (ESI) m/z [M+H]⁺ 349.2.

Methyl 4-chloro-1-((1-ethyl-1H-imidazol-5-yl)methyl)-2-(hydroxymethyl)-1H-benzo[d]imidazole-6-carboxylate Step a) (2.04 g, 5.85 mmol) was added in portions to a is vigorously stirred mixture of SOCl₂ (6.97 g, 58.55 mmol) and DMF (one drop). After complete addition the reaction mixture was stirred at rt for 1 h, and then concentrated at reduced pressure to give the hydrochloride of the title compound (1.8 g, 71%) as white solid; MS (ESI) m/z [M+H]⁺ 367.2.

### Intermediate 69

### rac-Methyl 4-chloro-2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylate

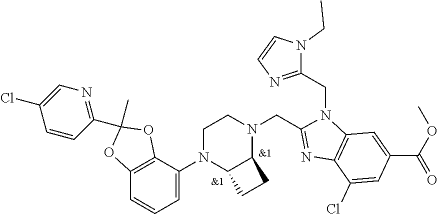 [SMILES: CCn1ccnc1Cn1c(CN2CCN(c3cccc4c3OC(C)(c3ccc(Cl)cn3)O4)[C@H]3CC[C@@H]32)nc2c(Cl)cc(C(=O)OC)cc21]

Methyl 4-chloro-2-(chloromethyl)-1-((1-ethyl-1H-imidazol-5-yl)methyl)-1H-benzo[d]imidazole-6-carboxylate Intermediate 68 (281 mg, 766 μmol), rac-(1R,6R)-2-(2-(5-chloropyridin-2-yl)-2-methylbenzo[d][1,3]dioxol-4-yl)-2,5-diazabicyclo[4.2.0]octane dihydrochloride Intermediate 26 (330 mg, 766 μmol), DIPEA (594 mg, 4.6 mmol) and NaI (459 mg, 3.06 mmol) were mixed in DMF (20 mL), and the reaction mixture was heated at 50° C. for 16 h. The mixture was cooled, diluted with water and extracted several times with EtOAc. The combined organic layer was washed with brine, dried over Na₂SO₄, and filtered.

The filtrate was concentrated under reduced pressure and the crude product was purified by preparative HPLC, PrepMethod E, (gradient: 40-65%) to give the title compound (152 mg, 29%); MS (ESI) m/z [M+H]⁺ 690.2.

### Intermediate 70

### rel-Methyl 4-chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylate, Isomer 1

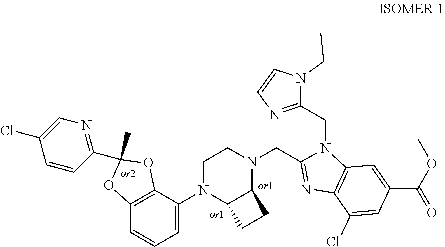 [SMILES: CCn1ccnc1Cn1c(CN2CCN(c3cccc4c3O[C@@](C)(c3ccc(Cl)cn3)O4)[C@H]3CC[C@@H]32)nc2c(Cl)cc(C(=O)OC)cc21]

### Intermediate 71

### rel-Methyl 4-chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylate, Isomer 3

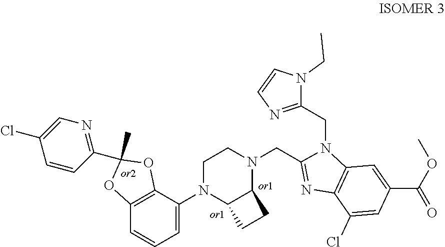 [SMILES: CCn1ccnc1Cn1c(CN2CCN(c3cccc4c3O[C@@](C)(c3ccc(Cl)cn3)O4)[C@H]3CC[C@@H]32)nc2c(Cl)cc(C(=O)OC)cc21]

### Intermediate 72

### rel-Methyl 4-chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylate, Isomer 4

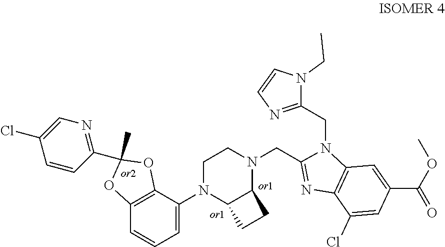 [SMILES: CCn1ccnc1Cn1c(CN2CCN(c3cccc4c3O[C@@](C)(c3ccc(Cl)cn3)O4)[C@H]3CC[C@@H]32)nc2c(Cl)cc(C(=O)OC)cc21]

The stereoisomers of rac-methyl 4-chloro-2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylate Intermediate 69 were separated by chiral chromatography on o Chiralpak IC III column (250×20 mm, 5 μm), eluted with hexane-IPA-MeOH (50:25:25), at a flow rate of 12 mL/min;
the first eluted compound was collected and evaporated to give the title compound Isomer 1 Intermediate 70 (39 mg); MS (ESI) m/z [M+H]⁺ 691.2;
the third eluted compound mixture was collected and evaporate to give a mixture of isomers, which were separated by chiral chromatography on a Chiralcel OD-H column (200×20 mm, Sum), eluted with hexane-IPA-MeOH (80:10:10), at a flow rate of 12 mL/min;
the first eluted compound was collected and evaporated to give the title compound Isomer 3 Intermediate 71 (16 mg); MS (ESI) m/z [M+H]⁺ 689.0; and
the second eluted compound was collected and evaporated to give the title compound Isomer 4 Intermediate 72 (35 mg); MS (ESI) m/z [M+H]⁺ 689.0.

### Intermediate 73

### 5-Fluoro-2-methoxy-4-nitrobenzoic acid

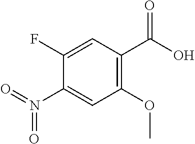

H₂θ₂(35% v/v) (148 mL, 3.51 mol) was added to a solution of 4-amino-5-fluoro-2-methoxybenzoic acid (25.0 g, 135.02 mmol) in TFA (375 mL, 4.86 mol). The reaction mixture was carefully heated at 50° C. for 16 h and the solution went from a dark orange clear solution to a pale yellow clear solution. The reaction mixture was concentrated in vacuo to give the title compound (30.0 g, 62%); MS (ESI) m/z [M−H]⁻ 214.0. Intermediate 74

### Methyl 5-fluoro-2-methoxy-4-nitrobenzoate

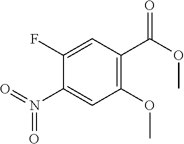 [SMILES: COC(=O)c1cc(F)c([N+](=O)[O-])cc1OC]

SOCl₂ (20.74 g, 174.31 mmol) was added dropwise to a solution of 5-fluoro-2-methoxy-4-nitrobenzoic acid Intermediate 73 (25.0 g, 116.21 mmol) in dry MeOH (150 mL), and the reaction mixture was heated to reflux for 16 h. The reaction mixture was concentrated under reduced pressure, and the crude product was dissolved in hot hexane and filtered. The filtrate was concentrated in vacuo to give the title compound (7.0 g, 18%); GC/MS [229.0] retention time 8.439 min.

### Intermediate 75

### Methyl (S)-2-methoxy-4-nitro-5-((oxetan-2-ylmethyl)amino)benzoate

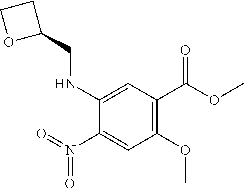 [SMILES: COC(=O)c1cc(NC[C@@H]2CCO2)c([N+](=O)[O-])cc1OC]

Methyl 5-fluoro-2-methoxy-4-nitrobenzoate Intermediate 74 (2.0 g, 8.73 mmol), 1-[(2S)-oxetan-2-yl]methanamine (836 mg, 9.6 mmol) and DIPEA (4.56 mL, 26.19 mmol) were mixed in DMSO (10 mL), and the reaction mixture was stirred at 90° C. for 16 h. The reaction mixture was cooled to rt, diluted with water and extracted with MTBE. The combined organic layer was washed with brine, dried over anhydrous Na₂SO₄, and filtered. The filtrate was concentrated to give the title compound (2.1 g, 65%) as a yellow oil; MS (ESI) m/z [M+H]⁺ 297.0 Intermediate 76

### Methyl (S)-4-amino-2-methoxy-5-((oxetan-2-ylmethyl)amino)benzoate

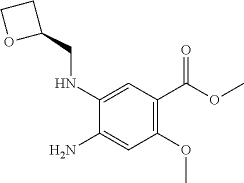 [SMILES: COC(=O)c1cc(NC[C@@H]2CCO2)c(N)cc1OC]

Pd/C (10%, 50 mg) was added to a solution of methyl (S)-2-methoxy-4-nitro-5-((oxetan-2-ylmethyl)amino)benzoate Intermediate 75 (2.1 g, 5.67 mmol) in THE (25 mL) and the is reaction mixture was stirred under an atmosphere of H₂(g) at ambient pressure and temperature until the reaction was complete. The reaction mixture was filtered and the filtrate was concentrated. The residue was purified by chromatography to give the title compound (260 mg, 13%); MS (ESI) m/z [M+H]⁺ 267.2.

### Intermediate 77

### Methyl (S)-2-(chloromethyl)-5-methoxy-1-(oxetan-2-ylmethyl)-1H-benzo[d]imidazole-6-carboxylate

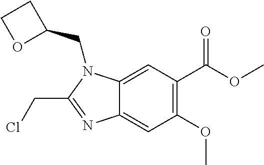 [SMILES: COC(=O)c1cc2c(cc1OC)nc(CCl)n2C[C@@H]1CCO1]

2-Chloro-1,1,1-trimethoxyethane (184 mg, 1.19 mmol) and pTsOH·H₂O (19 mg, 99 μmol) was added to a solution of methyl (S)-4-amino-2-methoxy-5-((oxetan-2-ylmethyl)amino)benzoate Intermediate 76 (265 mg, 995 μmol) in THE (50 mL), and the reaction mixture was stirred at 50° C. overnight. The mixture was poured into water and extracted with EtOAc. The combined organic layer was washed with brine and concentrated under vacuum to give the title compound (270 mg, 75%); MS (ESI) m/z [M+H]⁺ 325.2.

### Intermediate 78

### Methyl 3-((2-(2,2-difluorocyclopropoxy)ethyl)amino)-5-methoxy-4-nitrobenzoate

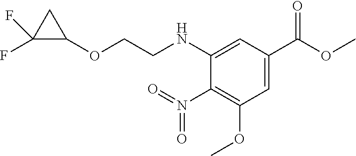 [SMILES: COC(=O)c1cc(NCCOC2CC2(F)F)c([N+](=O)[O-])c(OC)c1]

Methyl 3-fluoro-5-methoxy-4-nitrobenzoate (1.5 g, 6.55 mmol), 2-(2,2-difluorocyclopropoxy)ethanamine (1.36 g, 7.85 mmol) and DIPEA (3.42 mL, 19.63 mmol) were mixed in DMSO (5 mL), and the reaction mixture was stirred at 80° C. for 16 h. The is reaction mixture was cooled to rt, diluted with water and extracted with EtOAc. The combined organic layer was washed with brine, dried over anhydrous Na₂SO₄, and filtered. The filtrate was concentrated to give the title compound (2.2 g, 75%); MS (ESI) m/z [M+H]⁺ 347.1.

### Intermediate 79

### Methyl 4-amino-3-((2-(2,2-difluorocyclopropoxy)ethyl)amino)-5-methoxybenzoate

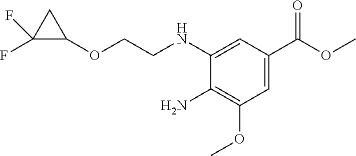 [SMILES: COC(=O)c1cc(NCCOC2CC2(F)F)c(N)c(OC)c1]

Pt/C (10%, 25 mg) was added to a solution of methyl 3-((2-(2,2-difluorocyclopropoxy)ethyl)amino)-5-methoxy-4-nitrobenzoate Intermediate 78 (2.2 g, 6.35 mmol) in MeOH (15 mL), and the reaction mixture was stirred under an atmosphere of H₂(g) at ambient pressure and temperature until the reaction was complete. The reaction mixture was filtered and the filtrate was concentrated. The residue was purified by flash chromatography on silica (hexane:EtOAc, 1:1) to give the title compound (1.15 g, 66%); MS (ESI) m/z [M+H]⁺ 317.2.

### Intermediate 80

### Methyl 2-(chloromethyl)-1-(2-(2,2-difluorocyclopropoxy)ethyl)-4-methoxy-1H-benzo[d]imidazole-6-carboxylate

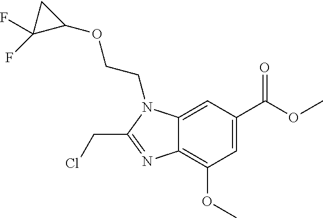 [SMILES: COC(=O)c1cc(OC)c2nc(CCl)n(CCOC3CC3(F)F)c2c1]

2-Chloro-1,1,1-trimethoxyethane (293 mg, 1.8 mmol) and pTsOH·H₂O (30 mg) were added to a solution of methyl 4-amino-3-((2-(2,2-difluorocyclopropoxy)ethyl)amino)-5-methoxybenzoate Intermediate 79 (0.5 g, 1.5 mmol) in THE (50 mL), and the reaction mixture was stirred at 50° C. overnight. The mixture was poured into water and extracted with EtOAc. The combined organic layer was washed with brine and concentrated under vacuum to give the title compound (0.50 g, 87%); MS (ESI) m/z [M+H]⁺ 375.0.

### Intermediate 81

### Methyl 3-((2-(2,2-difluorocyclopropoxy)ethyl)amino)-5-fluoro-4-nitrobenzoate

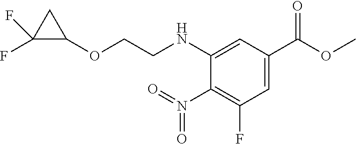 [SMILES: COC(=O)c1cc(F)c([N+](=O)[O-])c(NCCOC2CC2(F)F)c1]

Methyl 3,5-difluoro-4-nitrobenzoate (4.0 g, 18.42 mmol), 2-(2,2-difluorocyclopropoxy)ethan-1-amine (2.53 g, 18.42 mmol) and DIPEA (6.42 mL, 36.85 mmol) were mixed in THE (15 mL), and the reaction mixture was stirred at 45° C. for 16 h. The reaction mixture was cooled to rt, diluted with water and extracted with EtOAc. The combined organic layer was washed with brine, dried over anhydrous Na₂SO₄, and filtered. The filtrate was concentrated to give the title compound (6.0 g, 97%); MS (ESI) m/z [M+H]⁺ 335.2.

### Intermediate 82

### Methyl 4-amino-3-((2-(2,2-difluorocyclopropoxy)ethyl)amino)-5-fluorobenzoate

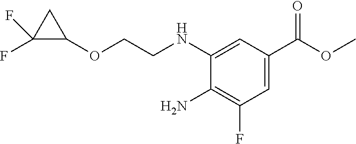 [SMILES: COC(=O)c1cc(F)c(N)c(NCCOC2CC2(F)F)c1]

Pt/C (1%, 40.8 mg) was added to a solution of methyl 3-((2-(2,2-difluorocyclopropoxy)ethyl)amino)-5-fluoro-4-nitrobenzoate Intermediate 81 (6.0 g, 17.9 mol) in MeOH (30 mL) and the reaction mixture was stirred under an atmosphere of H₂(g) at ambient pressure and temperature until the reaction was complete. The reaction mixture was filtered and the filtrate was concentrated. The residue was purified by flash chromatography on silica (hexane/EtOAc, 1/1) to give the title compound (4.5 g, 71%); MS (ESI) m/z [M+H]⁺ 305.0.

### Intermediate 83

### Methyl 2-(chloromethyl)-1-(2-(2,2-difluorocyclopropoxy)ethyl)-4-fluoro-1H-benzo[d]imidazole-6-carboxylate

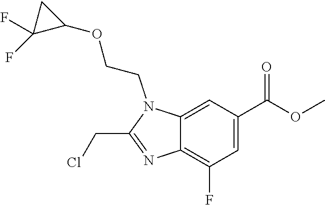 [SMILES: COC(=O)c1cc(F)c2nc(CCl)n(CCOC3CC3(F)F)c2c1]

2-Chloro-1,1,1-trimethoxyethane (2.74 g, 17.75 mmol) and pTsOH·H₂O (281 mg, 1.48 mmol) were added to a solution of methyl 4-amino-3-((2-(2,2-difluorocyclopropoxy)ethyl)amino)-5-fluorobenzoate Intermediate 82 (4.5 g, 14.79 mmol) in THE (50 mL), and the reaction mixture was stirred at 50° C. overnight. The reaction mixture was poured into water and extracted with EtOAc, and the combined organic layer was washed with brine and concentrated under vacuum to give the title compound (3.0 g, 56%); MS (ESI) m/z [M+H]⁺ 335.2.

### Intermediate 84

### Methyl 2-(((1RS,6RS)-5-(2-(5-chloropyridin-2-yl)-2-methylbenzo[d][1,3]dioxol-4-yl)-2,5-diazabicyclo[4.2.0]octan-2-yl)methyl)-5-methoxy-1-(((S)-oxetan-2-yl)methyl)-1H-benzo[d]imidazole-6-carboxylate

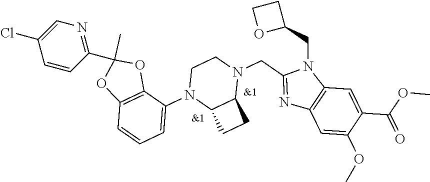 [SMILES: COC(=O)c1cc2c(cc1OC)nc(CN1CCN(c3cccc4c3OC(C)(c3ccc(Cl)cn3)O4)[C@H]3CC[C@@H]31)n2C[C@@H]1CCO1]

The title compound was prepared from rac-(1R,6R)-2-(2-(5-chloropyridin-2-yl)-2-methylbenzo[d][1,3]dioxol-4-yl)-2,5-diazabicyclo[4.2.0]octane hydrochloride Intermediate 26 (222 mg, 0.66 mmol) and methyl (S)-2-(chloromethyl)-5-methoxy-1-(oxetan-2-ylmethyl)-1H-benzo[d]imidazole-6-carboxylate Intermediate 77 (201 mg, 0.62 mmol) in analogy with the description for Intermediate 33 to give the title compound (26 mg, 6%); MS (ESI) m z [M+H]⁺ 646.2.

### Intermediate 85 rac-Methyl 2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-methoxy-1H-benzo[d]imidazole-6-carboxylate

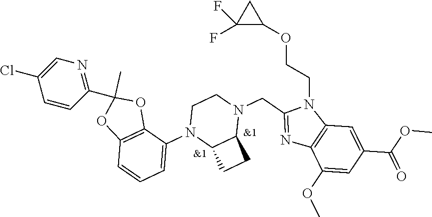 [SMILES: COC(=O)c1cc(OC)c2nc(CN3CCN(c4cccc5c4OC(C)(c4ccc(Cl)cn4)O5)[C@H]4CC[C@@H]43)n(CCOC3CC3(F)F)c2c1]

Methyl 2-(chloromethyl)-1-(2-(2,2-difluorocyclopropoxy)ethyl)-4-methoxy-1H-benzo[d]imidazole-6-carboxylate Intermediate 80 (440 mg, 1.1 mmol) and DIPEA (455 mg, 4.2 mmol)) were added to a solution of rac-(1R,6R)-2-(2-(5-chloropyridin-2-yl)-2-methylbenzo[d][1,3]dioxol-4-yl)-2,5-diazabicyclo[4.2.0]octane hydrochloride Intermediate 26 (504 mg, 1.4 mmol) in MeCN (5 mL) and the reaction mixture was stirred at 45° C. overnight. The reaction mixture was diluted with water and extracted with EtOAc. The combined organic layer was washed with sat NaCl (aq), dried over anhydrous Na₂SO₄, filtered and concentrated, and the crude product was purified by preparative HPLC, PrepMethod G, (gradient: 0-60%) to give the title compound (197 mg, 25%) as a yellow oil; MS (ESI) m/z [M+H]⁺ 696.2.

### Intermediate 86

### rac-Methyl 2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-fluoro-1H-benzo[d]imidazole-6-carboxylate

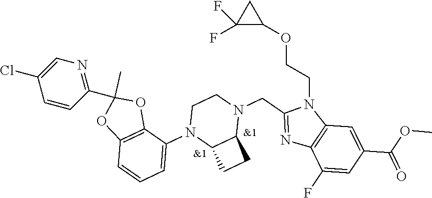 [SMILES: COC(=O)c1cc(F)c2nc(CN3CCN(c4cccc5c4OC(C)(c4ccc(Cl)cn4)O5)[C@H]4CC[C@@H]43)n(CCOC3CC3(F)F)c2c1]

The title compound was prepared from rac-(1R,6R)-2-(2-(5-chloropyridin-2-yl)-2-methylbenzo[d][1,3]dioxol-4-yl)-2,5-diazabicyclo[4.2.0]octane hydrochloride Intermediate 26 (403 mg, 1.12 mmol) and methyl 2-(chloromethyl)-1-(2-(2,2-difluorocyclopropoxy)ethyl)-4-fluoro-1H-benzo[d]imidazole-6-carboxylate Intermediate 83 (371 mg, 1.0 mmol) as described for Intermediate 85 to give the title compound (74 mg, 11%); MS (ESI) m z [M+H]⁺ 684.2.

### Intermediate 87

### rel-(R)-4-(4-Bromo-2-methylbenzo[d][1,3]dioxol-2-yl)-3-fluorobenzonitrile, Isomer 1

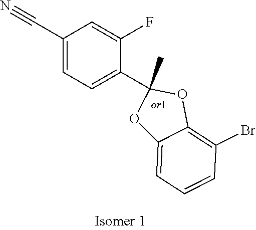 [SMILES: C[C@]1(c2ccc(C#N)cc2F)Oc2cccc(Br)c2O1]

### Intermediate 88

### rel-(R)-4-(4-Bromo-2-methylbenzo[d][1,3]dioxol-2-yl)-3-fluorobenzonitrile, Isomer 2

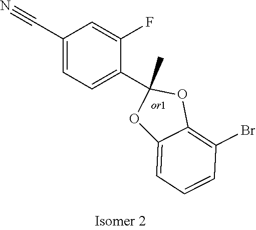 [SMILES: C[C@]1(c2ccc(C#N)cc2F)Oc2cccc(Br)c2O1]

The enantiomers of 4-(4-bromo-2-methylbenzo[d][1,3]dioxol-2-yl)-3-fluorobenzonitrile WO2020207474 (4.5 g, 13.47 mmol) were separated by chiral chromatography on a Chiral Art Amylose-C NEO column (250×50 mm, 10 μm), eluted with 25% IPA in hexane in CO₂, 100 bar, at a flow rate of 200 mL/min, and detected at 220 nm;
the first eluted compound was collected and evaporated to give the title compound Isomer 1, Intermediate 87 (1.0 g, 22%); ¹H NMR (400 MHz, DMSO-d₆) δ 2.11 (s, 3H), 6.84 (t, 1H), 7.04 (dd, 2H), 7.72-7.84 (m, 2H), 8.02 (d, 1H); and
the second eluted compound was collected and evaporated to give the title compound Isomer 2, Intermediate 88, (1.2 g, 27%); ¹H NMR (400 MHz, DMSO-d₆) δ 2.11 (s, 3H), 6.84 (t, 1H), 7.01 (d, 1H), 7.67-7.81 (m, 2H), 8.01 (dd, 1H).

### Intermediate 89

### 4-((R*)-4-((1SR, 6RS)-2,5-Diazabicyclo[4.2.0]octan-2-yl)-2-methylbenzo[d][1,3]dioxol-2-yl)-3-fluorobenzonitrile, Isomer mixture 1

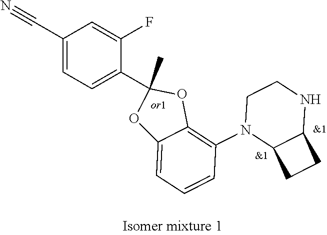 [SMILES: C[C@]1(c2ccc(C#N)cc2F)Oc2cccc(N3CCN[C@H]4CC[C@H]43)c2O1]

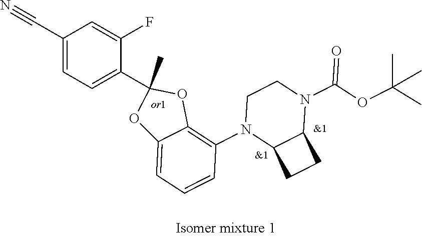 [SMILES: CC(C)(C)OC(=O)N1CCN(c2cccc3c2O[C@@](C)(c2ccc(C#N)cc2F)O3)[C@@H]2CC[C@@H]21]

Cs₂CO₃ (921 mg, 2.83 mmol) was added to a mixture of rel-(R)-4-(4-bromo-2-methylbenzo[d][1,3]dioxol-2-yl)-3-fluorobenzonitrile, Isomer 1 Intermediate 87 (472 mg, 1.41 mmol), rac-tert-butyl (1R,6S)-2,5-diazabicyclo[4.2.0]octane-2-carboxylate Intermediate 4 (300 mg, 1.41 mmol) and Pd-PEPPSI-IHeptCl (119 mg, 0.14 mmol) in 1,4-dioxane (20 mL) and the reaction mixture was stirred at 80° C. for 16 h. The reaction mixture was filtered through silica and then concentrated. The residue was diluted with EtOAc (100 mL), and the organic layer was washed with sat brine (2×50 mL), dried over Na₂SO₄, filtered and evaporated. The crude product was purified by flash chromatography on silica (gradient: 20-25% EtOAc in petroleum ether) to give the title compound Isomer mixture 1 (480 mg, 73%) as a yellow solid; MS (ESI) m/z [M+H]⁺ 466.0. pTsOH (533 mg, 3.09 mmol) was added to a solution of the product from Step a) (480 mg) in is DCM (20 mL) and the reaction mixture was stirred at 35° C. for 12 h. The solvent was removed under reduced pressure to give the title compound Isomer mixture 1 as the para-toluenesulfonic acid salt (900 mg, 100%) as a blue solid; MS (ESI) m/z [M+H]⁺ 365.9.

### Intermediate 90

### 4-((R*)-4-((1SR,6RS)-2,5-Diazabicyclo[4.2.0]octan-2-yl)-2-methylbenzo[d][1,3]dioxol-2-yl)-3-fluorobenzonitrile, Isomer mixture 2

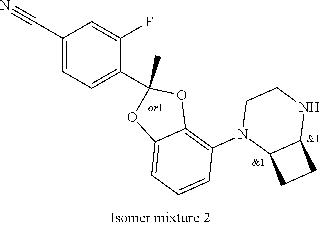 [SMILES: C[C@]1(c2ccc(C#N)cc2F)Oc2cccc(N3CCN[C@H]4CC[C@H]43)c2O1]

The title compound was prepared in two steps, as described for Intermediate 89, from rel-(R)-4-(4-bromo-2-methylbenzo[d][1,3]dioxol-2-yl)-3-fluorobenzonitrile, Isomer 2 Intermediate 88 (472 mg, 1.41 mmol) and rac-tert-butyl (1R,6S)-2,5-diazabicyclo[4.2.0]octane-2-carboxylate Intermediate 4 (300 mg, 1.41 mmol) to give the title compound Isomer mixture 2 as the para-toluenesulfonic acid salt (522 mg, 79%); MS (ESI) m/z [M+H]⁺ 366.0.

### Intermediate 91

### rac-(1R,6S)-2-(2-(4-Chloro-2-fluorophenyl)-2-methylbenzo[d][1,3]dioxol-4-yl)-2,5-diazabicyclo[4.2.0]octane

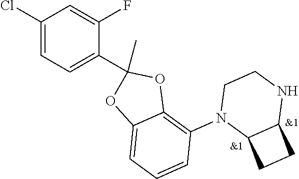 [SMILES: CC1(c2ccc(Cl)cc2F)Oc2cccc(N3CCN[C@H]4CC[C@H]43)c2O1]

pTsOH (1.396 g, 8.11 mmol) was added to a solution of rac-tert-butyl (1R,6S)-5-(2-(4-chloro-2-fluorophenyl)-2-methylbenzo[d][1,3]dioxol-4-yl)-2,5-diazabicyclo[4.2.0]octane-2-carboxylate Intermediate 60 (0.77 g, 1.62 mmol) in DCM (15 mL) at 20° C. under an atmosphere of N₂(g), and the reaction mixture was stirred at 35° C. for 3 h. The solvent was removed under reduced pressure to give the title compound as the para-toluensulfonic acid salt (1.8 g) as a crude yellow solid; MS (ESI) m/z [M+H]⁺ 374.9.

### Intermediate 92

Methyl 2-(((1RS,6SR)-5-((R*)-2-(4-cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer mixture 1

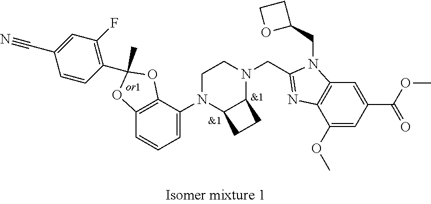 [SMILES: COC(=O)c1cc(OC)c2nc(CN3CCN(c4cccc5c4O[C@@](C)(c4ccc(C#N)cc4F)O5)[C@@H]4CC[C@@H]43)n(C[C@@H]3CCO3)c2c1]

K₂CO₃ (1.254 g, 9.07 mmol) was added to a solution of 4-((R*)-4-((1SR,6RS)-2,5-diazabicyclo[4.2.0]octan-2-yl)-2-methylbenzo[d][1,3]dioxol-2-yl)-3-fluorobenzonitrile para toluenesulfonate, Isomer mixture 1, Intermediate 89 (0.8 g, 0.91 mmol) and methyl (S)-2-(chloromethyl)-4-methoxy-1-(oxetan-2-ylmethyl)-1H-benzo[d]imidazole-6-carboxylate Intermediate 10 (0.295 g, 0.91 mmol) in MeCN (15 mL) and the reaction mixture was stirred at 60° C. for 4 h. The reaction mixture was filtered through celite, and then concentrated. The residue was diluted with EtOAc (125 mL), and washed with sat brine (50 mL). The organic layer was dried over Na₂SO₄, filtered and evaporated. The residue was purified by preparative TLC (EtOAc:petroleum ether, 1:5), to give the title compound Isomer mixture 1 (0.540 g, 91%) as a yellow solid; MS (ESI) m/z [M+H]⁺ 654.3.

### Intermediate 93

### Methyl 2-((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 1

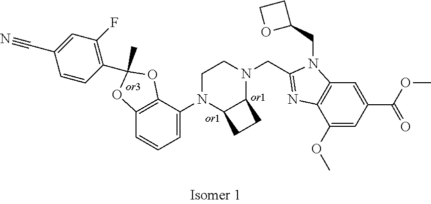 [SMILES: COC(=O)c1cc(OC)c2nc(CN3CCN(c4cccc5c4O[C@@](C)(c4ccc(C#N)cc4F)O5)[C@@H]4CC[C@@H]43)n(C[C@@H]3CCO3)c2c1]

### Intermediate 94

### Methyl 2-((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 2

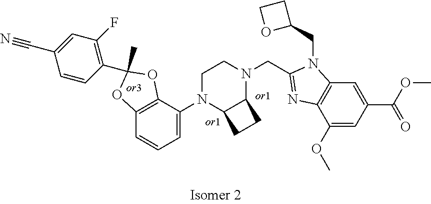 [SMILES: COC(=O)c1cc(OC)c2nc(CN3CCN(c4cccc5c4O[C@@](C)(c4ccc(C#N)cc4F)O5)[C@@H]4CC[C@@H]43)n(C[C@@H]3CCO3)c2c1]

The stereoisomers of methyl 2-(((1RS,6SR)-5-((R*)-2-(4-cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer mixture 1

Intermediate 92 (0.5 g) were separated by chiral chromatography on a Chiralpak IF column (250×50 mm, 5 μm), eluted with 50% EtOH in hexane (0.1% 2M NH₃ in MeOH), at a flow rate of 15 mL/min, and detected at 220 nm and 254 nm;
the first eluted compound was collected and evaporated to give the title compound, Isomer 1, Intermediate 93 (221 mg, 44%); MS (ESI) m/z [M+H]⁺ 654.2; and
the second eluted compound was collected and evaporated to give the title compound, Isomer 2, Intermediate 94 (230 mg, 46%); MS (ESI) m/z [M+H]⁺ 654.3.

### Intermediate 95

### Methyl 4-chloro-2-(((1RS,6SR)-5-((R*)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer mixture 1

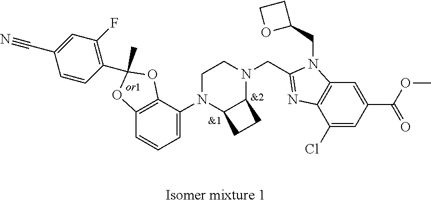 [SMILES: COC(=O)c1cc(Cl)c2nc(CN3CCN(c4cccc5c4O[C@@](C)(c4ccc(C#N)cc4F)O5)[C@@H]4CC[C@@H]43)n(C[C@@H]3CCO3)c2c1]

The title compound was prepared as described for Intermediate 92 from 4-((R*)-4-((1SR,6RS)-2,5-diazabicyclo[4.2.0]octan-2-yl)-2-methylbenzo[d][1,3]dioxol-2-yl)-3-fluorobenzonitrile para toluenesulfonate Isomer mixture 1, Intermediate 89 (800 mg, 0.91 mmol) and methyl (S)-4-chloro-2-(chloromethyl)-1-(oxetan-2-ylmethyl)-1H-benzo[d]imidazole-6-carboxylate Intermediate 24 (299 mg, 0.91 mmol). The crude product was purified by reversed phase flash chromatography on a C18 column (0-90% MeCN in water) to give the title compound Isomer mixture 1 (370 mg, 62%) as a white solid; MS (ESI) m/z [M+H]⁺ 658.2.

### Intermediate 96

### Methyl 4-chloro-2-(((1RS,6SR)-5-((R*)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate. Isomer mixture 2

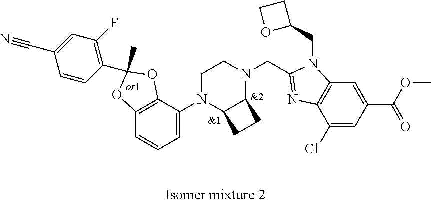 [SMILES: COC(=O)c1cc(Cl)c2nc(CN3CCN(c4cccc5c4O[C@@](C)(c4ccc(C#N)cc4F)O5)[C@@H]4CC[C@@H]43)n(C[C@@H]3CCO3)c2c1]

The title compound was prepared as described for Intermediate 92 from 4-((R*)-4-((1SR,6RS)-2,5-diazabicyclo[4.2.0]octan-2-yl)-2-methylbenzo[d][1,3]dioxol-2-yl)-3-fluorobenzonitrile para toluenesulfonate Isomer mixture 2, Intermediate 90 (680 mg, 0.77 mmol) and methyl (S)-4-chloro-2-(chloromethyl)-1-(oxetan-2-ylmethyl)-1H-benzo[d]imidazole-6-carboxylate Intermediate 24 (254 mg, 0.77 mmol). The crude product was purified by reversed phase flash chromatography on a C18 column (0-90% MeCN in water) to give the title compound Isomer mixture 2 (400 mg, 79%) as a white solid; MS (ESI) m/z [M+H]⁺ 658.1.

### Intermediate 97

### Methyl 4-chloro-2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 1

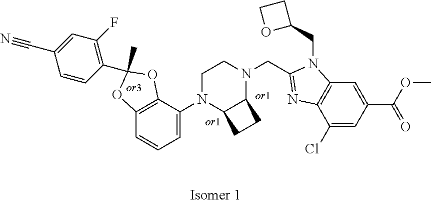 [SMILES: COC(=O)c1cc(Cl)c2nc(CN3CCN(c4cccc5c4O[C@@](C)(c4ccc(C#N)cc4F)O5)[C@@H]4CC[C@@H]43)n(C[C@@H]3CCO3)c2c1]

### Intermediate 98

### Methyl 4-chloro-2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate. Isomer 2

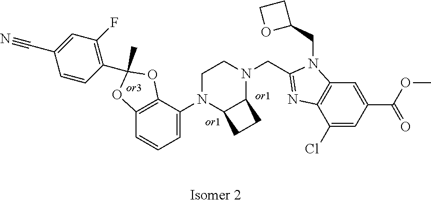 [SMILES: COC(=O)c1cc(Cl)c2nc(CN3CCN(c4cccc5c4O[C@@](C)(c4ccc(C#N)cc4F)O5)[C@@H]4CC[C@@H]43)n(C[C@@H]3CCO3)c2c1]

The stereoisomers of methyl 4-chloro-2-(((1RS,6SR)-5-((R*)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer mixture 1 Intermediate 95 (0.37 g) were separated by chiral chromatography on a Chiralpak IF column (250×50 mm, 5 μm), eluted with 50% EtOH in hexane (0.5% 2M NH₃ in MeOH), at a flow rate of 15 mL/min, and detected at 220 nm and 254 nm;
the first eluted compound was collected and evaporated to give the title compound, Isomer 1, Intermediate 97 (130 mg, 35%); MS (ESI) m/z [M+H]⁺ 658.2; and
the second eluted compound was collected and evaporated to give the title compound, Isomer 2, Intermediate 98 (160 mg, 43%); MS (ESI) m/z [M+H]⁺ 658.2.

### Intermediate 99

### Methyl 4-chloro-2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 3

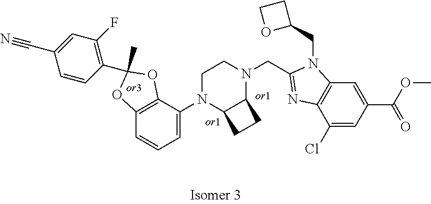 [SMILES: COC(=O)c1cc(Cl)c2nc(CN3CCN(c4cccc5c4O[C@@](C)(c4ccc(C#N)cc4F)O5)[C@@H]4CC[C@@H]43)n(C[C@@H]3CCO3)c2c1]

The stereoisomers of methyl 4-chloro-2-(((1RS,6SR)-5-((R*)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer mixture 2 Intermediate 96 (0.40 g) were separated by chiral chromatography on a Chiralpak IF column (250×50 mm, 5 μm), eluted with 50% EtOH in hexane (0.5% 2M NH₃ in MeOH), at a flow rate of 15 mL/min, and detected at 220 nm and 254 nm;
the first eluted compound was collected and evaporated to give the title compound, Isomer 3, Intermediate 99 (160 mg, 40%); MS (ESI) m/z [M+H]⁺ 658.1.

### Intermediate 100

### Methyl 2-(((1RS,6SR)-5-((R*)-2-(4-cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer mixture 1

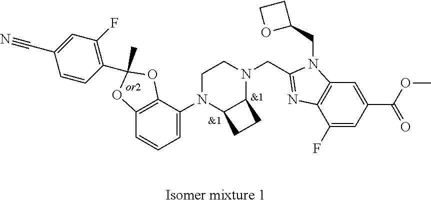 [SMILES: COC(=O)c1cc(F)c2nc(CN3CCN(c4cccc5c4O[C@@](C)(c4ccc(C#N)cc4F)O5)[C@@H]4CC[C@@H]43)n(C[C@@H]3CCO3)c2c1]

The title compound was prepared as described for Intermediate 92 from 4-((R*)-4-((1SR,6RS)-2,5-diazabicyclo[4.2.0]octan-2-yl)-2-methylbenzo[d][1,3]dioxol-2-yl)-3-fluorobenzonitrile para toluenesulfonate Isomer mixture 1, Intermediate 89 (800 mg, 0.91 mmol) and methyl (S)-2-(chloromethyl)-4-fluoro-1-(oxetan-2-ylmethyl)-1H-benzo[d]imidazole-6-carboxylate Intermediate 7 (284 mg, 0.91 mmol). The crude product was purified by preparative TLC (MeOH:DCM, 1:20), to give the title compound Isomer is mixture 1 (450 mg, 77%) as a yellow solid; MS (ESI) m/z [M+H]⁺ 642.2.

### Intermediate 101

### Methyl 2-(((1RS,6SR)-5-((R*)-2-(4-cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer mixture 2

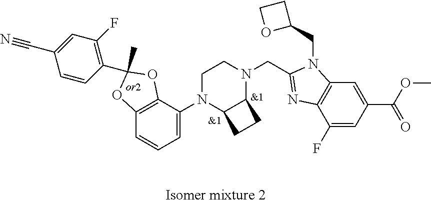 [SMILES: COC(=O)c1cc(F)c2nc(CN3CCN(c4cccc5c4O[C@@](C)(c4ccc(C#N)cc4F)O5)[C@@H]4CC[C@@H]43)n(C[C@@H]3CCO3)c2c1]

The title compound was prepared as described for Intermediate 92 from 4-((R*)-4-((1SR,6RS)-2,5-diazabicyclo[4.2.0]octan-2-yl)-2-methylbenzo[d][1,3]dioxol-2-yl)-3-fluorobenzonitrile para toluenesulfonate Isomer mixture 2, Intermediate 90 (700 mg, 0.79 mmol) and methyl (S)-2-(chloromethyl)-4-fluoro-1-(oxetan-2-ylmethyl)-1H-benzo[d]imidazole-6-carboxylate Intermediate 7 (248 mg, 0.79 mmol). The crude product was purified by preparative TLC (MeOH:DCM, 1:20), to give the title compound Isomer mixture 2 (500 mg, 98%) as a yellow solid; MS (ESI) m/z [M+H]⁺ 642.3.

### Intermediate 102

### Methyl 2-((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 1

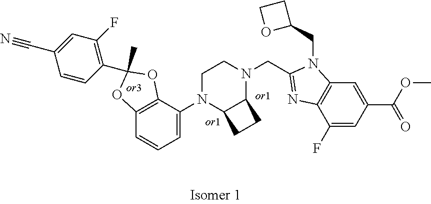 [SMILES: COC(=O)c1cc(F)c2nc(CN3CCN(c4cccc5c4O[C@@](C)(c4ccc(C#N)cc4F)O5)[C@@H]4CC[C@@H]43)n(C[C@@H]3CCO3)c2c1]

### Intermediate 103

### Methyl 2-((1R*,6S*)-5-((R*)-2-(4-cyano-2-fluorophenyl)-2-methylbenzo[d][1,3]dioxol-4-is yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 2

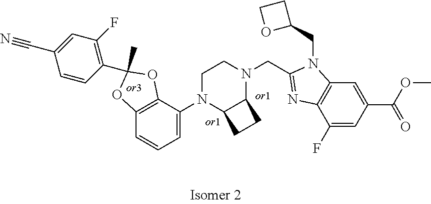 [SMILES: COC(=O)c1cc(F)c2nc(CN3CCN(c4cccc5c4O[C@@](C)(c4ccc(C#N)cc4F)O5)[C@@H]4CC[C@@H]43)n(C[C@@H]3CCO3)c2c1]

The stereoisomers of methyl 2-(((1RS,6SR)-5-((R*)-2-(4-cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer mixture 1 Intermediate 100 (0.45 g) were separated by chiral chromatography on a Chiralpak ID column (250×50 mm, 5 μm), eluted with 30% EtOH in hexane (0.5% 2M NH₃ in MeOH), at a flow rate of 20 mL/min, and detected at 220 nm and 254 nm;
the first eluted compound was collected and evaporated to give the title compound, Isomer 1, Intermediate 102 (200 mg, 44%); MS (ESI) m/z [M+H]⁺ 642.2; and
the second eluted compound was collected and evaporated to give the title compound, Isomer 2, Intermediate 103 (191 mg, 42%); MS (ESI) m/z [M+H]⁺ 642.0.

### Intermediate 104

### Methyl 2-((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 3

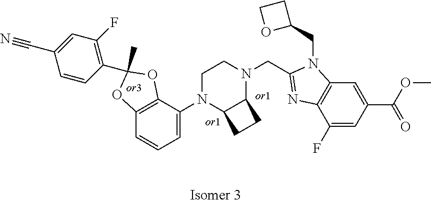 [SMILES: COC(=O)c1cc(F)c2nc(CN3CCN(c4cccc5c4O[C@@](C)(c4ccc(C#N)cc4F)O5)[C@@H]4CC[C@@H]43)n(C[C@@H]3CCO3)c2c1]

The stereoisomers of methyl 2-(((1RS,6SR)-5-((R*)-2-(4-cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer mixture 2 Intermediate 101 (0.495 g) were separated by chiral chromatography on a Chiralpak ID column (250×50 is mm, 5 μm), eluted with 20% EtOH in hexane (0.5% 2M NH₃ in MeOH), at a flow rate of 20 mL/min, and detected at 220 nm and 254 nm;
the first eluted compound was collected and evaporated to give the title compound, Isomer 3, Intermediate 104 (220 mg, 49%); MS (ESI) m/z [M+H]⁺ 642.3.

### Intermediate 105

### Methyl 2-(((1RS,6SR)-5-(2-(4-chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate

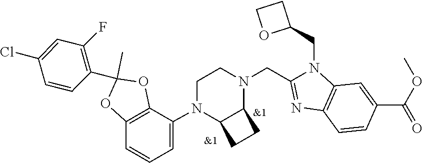 [SMILES: COC(=O)c1ccc2nc(CN3CCN(c4cccc5c4OC(C)(c4ccc(Cl)cc4F)O5)[C@@H]4CC[C@@H]43)n(C[C@@H]3CCO3)c2c1]

Methyl (S)-2-(chloromethyl)-1-(oxetan-2-ylmethyl)-1H-benzo[d]imidazole-6-carboxylate (132 mg, 0.45 mmol) was added to a mixture of rac-(1R,6S)-2-(2-(4-chloro-2-fluorophenyl)-2-methylbenzo[d][1,3]dioxol-4-yl)-2,5-diazabicyclo[4.2.0]octane para toluensulfonate Intermediate 91 (500 mg, 0.45 mmol) and K₂CO₃ (558 mg, 4.03 mmol) in MeCN (15 mL) and the reaction mixture was stirred at 60° C. for 3 h. The reaction mixture was concentrated and then diluted with EtOAc (100 mL). The organic layer was washed with sat brine (3×50 mL), dried over Na₂SO₄, filtered and evaporated. The residue was purified by preparative TLC (petroleum ether: EtOAc, 5: 1), to give the title compound (200 mg, 70%) as a yellow solid; MS (ESI) m/z [M+H]⁺ 633.2.

### Intermediate 106

### Methyl 2-((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 1

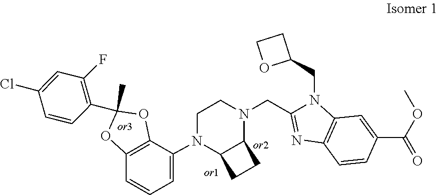 [SMILES: COC(=O)c1ccc2nc(CN3CCN(c4cccc5c4O[C@@](C)(c4ccc(Cl)cc4F)O5)[C@@H]4CC[C@@H]43)n(C[C@@H]3CCO3)c2c1]

### Intermediate 107

### Methyl 2-((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 2

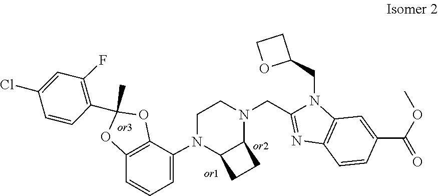 [SMILES: COC(=O)c1ccc2nc(CN3CCN(c4cccc5c4O[C@@](C)(c4ccc(Cl)cc4F)O5)[C@@H]4CC[C@@H]43)n(C[C@@H]3CCO3)c2c1]

### Intermediate 108

### Methyl 2-(((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 4

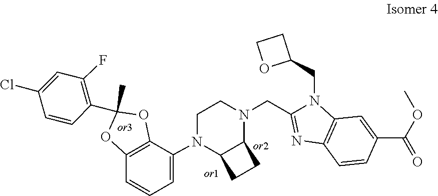 [SMILES: COC(=O)c1ccc2nc(CN3CCN(c4cccc5c4O[C@@](C)(c4ccc(Cl)cc4F)O5)[C@@H]4CC[C@@H]43)n(C[C@@H]3CCO3)c2c1]

The diastereomers of methyl 2-(((1RS,6SR)-5-(2-(4-chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate Intermediate 105 (657 mg, 1.04 mmol) were separated by chiral chromatography on a Chiralpak IE column (250×50 mm ID, 5 μm), eluted with 5% IPA in hexane/DCM (3:1, 0.5% 2M NH₃ in MeOH), at a flow rate of 20 mL/min and detected at 220 nm and 254 nm;
the first eluted compound mixture was collected and evaporated to yield a mixture of isomers (258 mg), and
the second eluted compound mixture was collected and evaporate to yield a mixture of isomers (256 mg).

The stereoisomers of the first eluted compound mixture (258 mg) were separated by chiral chromatography on a Lux Sum Amylose-1 column (250×50 mm ID, 10 μm), eluted with 20% IPA in hexane (0.5% 2M NH₃ in MeOH), at a flow rate of 20 mL/min and detected at 220 and 254 nm;
the first eluted compound was collected and evaporated to give the title compound Isomer 1 Intermediate 106 (122 mg); MS (ESI) m/z [M+H]⁺ 633.1.
the second eluted compound was collected and evaporated to give the title compound Isomer 2 Intermediate 107 (85 mg); MS (ESI) m/z [M+H]⁺ 633.1.

The stereoisomers of the second eluted compound mixture (256 mg) were separated by chiral chromatography on a CHIRALPAK IE column (250×20 mm ID, 5 μm), eluted with 10% IPA in hexane/DCM (3:1, 0.5% NH₃ in MeOH), at a flow rate of 20 mL/min and detected at 220 and 254 nm;
the second eluted compound was collected and evaporated to give the title compound Isomer 4 Intermediate 108 (75 mg); MS (ESI) m/z [M+H]⁺ 633.3.

### Intermediate 109

### Methyl 2-(((1RS,6SR)-5-(2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate

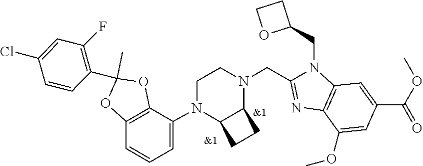 [SMILES: COC(=O)c1cc(OC)c2nc(CN3CCN(c4cccc5c4OC(C)(c4ccc(Cl)cc4F)O5)[C@@H]4CC[C@@H]43)n(C[C@@H]3CCO3)c2c1]

### Methyl (S)-2-(chloromethyl)-4-methoxy-1-(oxetan-2-ylmethyl)-1H-benzo[d]imidazole-6-carboxylate Intermediate 10 (641 mg, 1.97 mmol) was added to a mixture of rac-(1R,6S)-2-(2-(4-chloro-2-fluorophenyl)-2-methylbenzo[d][1,3]dioxol-4-yl)-2,5-diazabicyclo[4.2.0]octane para toluensulfonate Intermediate 91 (2.2 g, 1.97 mmol) and K₂CO₃ (1.36 g, 9.86 mmol) in MeCN (20 mL) and the reaction mixture was stirred at 60° C. for 8 h. The reaction mixture was filtered through Celite and the solids were washed with MeCN (3×25 mL). The filtrate was collected and evaporated and the residue was purified by reversed phase flash chromatography on a C18 column (gradient: 0-100% MeCN in water) to is give the title compound (850 mg, 65%) as a pale yellow solid; MS (ESI) m/z [M+H]⁺ 633

### Intermediate 110

### Methyl 2-(((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 1

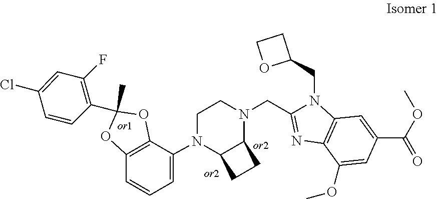 [SMILES: COC(=O)c1cc(OC)c2nc(CN3CCN(c4cccc5c4O[C@@](C)(c4ccc(Cl)cc4F)O5)[C@@H]4CC[C@@H]43)n(C[C@@H]3CCO3)c2c1]

### Intermediate 111

### Methyl 2-(((1R*,6S*)-5-(R*)-2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carbox late Isomer 2

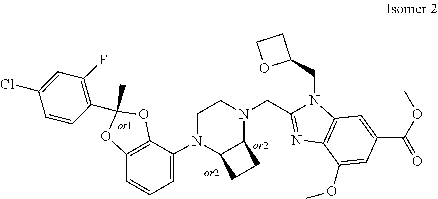 [SMILES: COC(=O)c1cc(OC)c2nc(CN3CCN(c4cccc5c4O[C@@](C)(c4ccc(Cl)cc4F)O5)[C@@H]4CC[C@@H]43)n(C[C@@H]3CCO3)c2c1]

### Methyl 2-(((1R*,6S*)-5-(R*)-2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 3

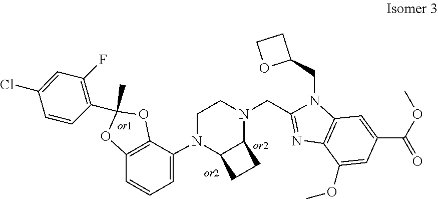 [SMILES: COC(=O)c1cc(OC)c2nc(CN3CCN(c4cccc5c4O[C@@](C)(c4ccc(Cl)cc4F)O5)[C@@H]4CC[C@@H]43)n(C[C@@H]3CCO3)c2c1]

### Intermediate 113

### Methyl 2-((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 4

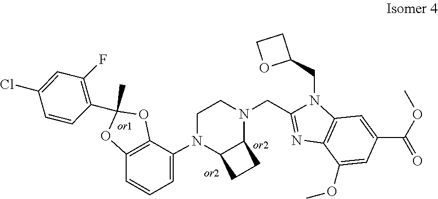 [SMILES: COC(=O)c1cc(OC)c2nc(CN3CCN(c4cccc5c4O[C@@](C)(c4ccc(Cl)cc4F)O5)[C@@H]4CC[C@@H]43)n(C[C@@H]3CCO3)c2c1]

The stereoisomers of methyl 2-(((1RS,6SR)-5-(2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate Intermediate 109 (800 mg, 1.21 mmol) were separated by chiral chromatography on a Chiralpak IH column (250×30 mm, 5 μm), eluted with 30% IPA/MeCN (1:1, 0.1% 2M NH₃ in MeOH) in CO₂, 100 bar, at a flow rate of 60 mL/min and detected at 220 nm;
the first eluted compound mixture was collected and evaporated to yield a mixture of isomers (400 mg); and
the second eluted compound mixture was collected and evaporated to yield a mixture of isomers (350 mg).

The stereoisomers of the first eluted compound mixture (400 mg) were separated by chiral chromatography on a (R, R)-Whelk-01 Kromasil column (250×21.1 mm, 5 μm), eluted with 10% IPA in MTBE (0.5% 2M NH₃ in MeOH), at a flow rate of 20 mL/min and detected at 220 nm and 254 nm;
the first eluted compound was collected and evaporated to give the title compound Isomer 1, Intermediate 110 (120 mg) as a white solid; MS (ESI) m/z [M+H]⁺ 663; and
the second eluted compound was collected and evaporated to give the title compound Isomer 2, Intermediate 111 (200 mg) as a white solid; MS (ESI) m/z [M+H]⁺ 663. The stereoisomers of the second eluted compound mixture (350 mg) were separated by chiral chromatography on a Chiralpak IH column (250×30 mm, 5 μm), eluted with 32% MeOH:MeCN (1:1, 1% 2M NH₃ in MeOH) in CO₂, 100 bar, at a flow rate of 70 mL/min and detected at 220 nm;
the first eluted compound was collected and evaporated to give the title compound Isomer 3, Intermediate 112 (160 mg) as a white solid; MS (ESI) m/z [M+H]⁺ 663; and
the second eluted compound was collected and evaporated to give the title compound Isomer 4 Intermediate 113 (90 mg) as a white solid; MS (ESI) m/z [M+H]⁺ 663.

### Intermediate 114

### rel-(R)-2-(4-Bromo-2-methylbenzo[d][1,3]dioxol-2-yl)-5-chloropyridine, Isomer 1

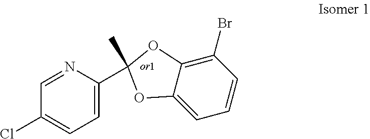 [SMILES: C[C@]1(c2ccc(Cl)cn2)Oc2cccc(Br)c2O1]

### Intermediate 115

### rel-(R)-2-(4-bromo-2-methylbenzo[d][1.3]dioxol-2-yl)-5-chloropyridine. Isomer 2

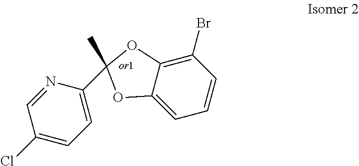 [SMILES: C[C@]1(c2ccc(Cl)cn2)Oc2cccc(Br)c2O1]

The stereoisomers of 2-(4-bromo-2-methylbenzo[d][1,3]dioxol-2-yl)-5-chloropyridine WO2020234726 (2 g, 6.12 mmol) were separated by chiral chromatography on an UniChiral OD-5H column (250×30 mm, 5 μm), eluted with 10% IPA/hexane (1:4, 0.5% 2M NH₃ in MeOH) in CO₂, 100 bar, at a flow rate of 100 mL/min and detected at 220 nm; The first eluted compound was collected and evaporated to give the title compound Isomer 1, Intermediate 114 (400 mg, 20%); MS (ESI) m/z [M+H]⁺ 326/328; and
the second eluted compound was collected and evaporated to give the title compound Isomer 2, Intermediate 115, (400 mg, 20%); MS (ESI) m/z [M+H]⁺ 326/328.

### Intermediate 116 (1RS,6SR)-2-((S*)-2-(5-Chloropyridin-2-yl)-2-methylbenzo[d][1,3]dioxol-4-yl)-2,5-diazabicyclo[4.2.0]octane, Isomer mixture 1

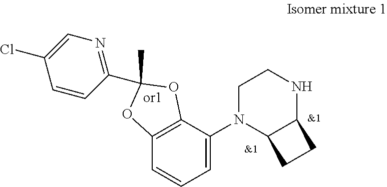 [SMILES: C[C@]1(c2ccc(Cl)cn2)Oc2cccc(N3CCN[C@H]4CC[C@H]43)c2O1]

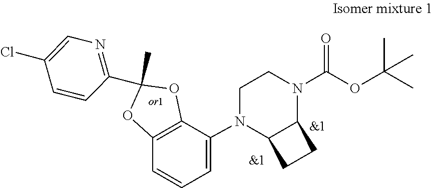 [SMILES: CC(C)(C)OC(=O)N1CCN(c2cccc3c2O[C@@](C)(c2ccc(Cl)cn2)O3)[C@@H]2CC[C@@H]21]

Cs₂CO₃ (1.247 g, 3.83 mmol) was added to a mixture of rel-(R)-2-(4-bromo-2-methylbenzo[d][1,3]dioxol-2-yl)-5-chloropyridine Isomer 1, Intermediate 114 (500 mg, 1.53 mmol), rac-tert-butyl (1R,6S)-2,5-diazabicyclo[4.2.0]octane-2-carboxylate Intermediate 4 (271 mg, 1.28 mmol), Palladacycle Gen 4 (73 mg, 0.060 mmol) and 2′-(bis(3,5-bis(trifluoromethyl)phenyl)phosphino)-3′,6′-dimethoxy-N².N².N⁶.N⁶-tetramethyl-[1,1′-biphenyl]-2,6-diamine (48 mg, 0.060 mmol) in 1,4-dioxane (10 mL) and the reaction mixture was stirred at 90° C. for 16 h. The reaction mixture was filtered through celite and the filter cake was washed with EtOAc (3×50 mL). The combined filtrate was collected and concentrated under reduced pressure and the residue was purified by preparative TLC (petroleum ether:EtOAc, 5:1), to give the sub-title compound Isomer mixture 1 (500 mg, 86%) as a white solid; MS (ESI) m/z [M+H]⁺ 458.2.

pTsOH (0.846 g, 4.91 mmol) was added to a solution of the product from Step a) (450 mg, 0.98 mmol) in DCM (10 mL) and the reaction mixture was stirred at 35° C. for 3 h. The reaction mixture was concentrated under reduced pressure to give the title compound Isomer mixture 1 as the para-toluensulfonic acid salt (1.0 g, 83%); MS (ESI) m/z [M+H]⁺ 358.0.

### Intermediate 117 (1RS,6SR)-2-((S*)-2-(5-Chloropyridin-2-yl)-2-methylbenzo[d][1,3]dioxol-4-yl)-2,5-diazabicyclo[4.2.0]octane, Isomer mixture 2

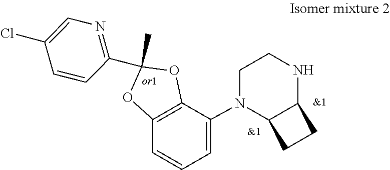 [SMILES: C[C@]1(c2ccc(Cl)cn2)Oc2cccc(N3CCN[C@H]4CC[C@H]43)c2O1]

The title compound was prepared in two steps in a similar way as as described for Intermediate 116 from Intermediate 115 (400 mg, 1.22 mmol) and Intermediate 4 (200 mg, 0.94 mmol) to give the title compound Isomer mixture 2 as the para-toluensulfonic acid salt (400 mg, 99%) as a yellow solid; MS (ESI) m/z [M+H]⁺ 358.

### Intermediate 118

### Methyl 4-chloro-2-(((1RS,6SR)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate_Isomer mixture 1

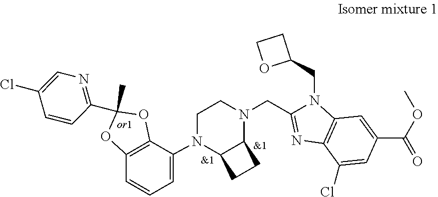 [SMILES: COC(=O)c1cc(Cl)c2nc(CN3CCN(c4cccc5c4O[C@@](C)(c4ccc(Cl)cn4)O5)[C@@H]4CC[C@@H]43)n(C[C@@H]3CCO3)c2c1]

K₂CO₃ (0.635 g, 4.59 mmol) was added to a solution of (1RS,6SR)-2-((S*)-2-(5-chloropyridin-2-yl)-2-methylbenzo[d][1,3]dioxol-4-yl)-2,5-diazabicyclo[4.2.0]octane para-toluensulfonate Isomer mixture 1 Intermediate 116 (0.8 g, 0.66 mmol) and methyl (S)-4-chloro-2-(chloromethyl)-1-(oxetan-2-ylmethyl)-1H-benzo[d]imidazole-6-carboxylate Intermediate 24 (0.216 g, 0.66 mmol) in MeCN (15 mL) and the reaction mixture was stirred at 60° C. for 8 h. The reaction mixture was quenched with water (100 mL), extracted with EtOAc (3×50 mL), and the organic layer was dried over Na₂SO₄, filtered and evaporated. The crude product was purified by reversed phase flash chromatography on a C18-column (0-80% MeCN in water) to give the title compound (0.400 g, 94%) as a white solid; MS (ESI) m/z [M+H]⁺ 650.2.

### Intermediate 119

### Methyl 4-chloro-2-(((1RS,6SR)-5-((R*)-2-(5-chloropyridin-2-yl)-2-is methylbenzo[d][1,3]dioxol-4-yl)-2,5-diazabicyclo[4.2.0]0ctan-2-yllmethyl)-1-(((S)-oxetan-2-yl)methyl)-1H-benzo[d]imidazol e-6-carboxylate, Isomer mixture 2

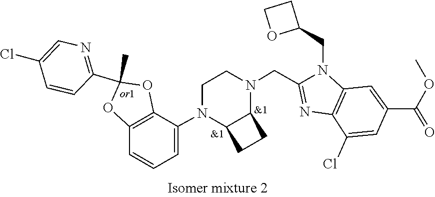 [SMILES: COC(=O)c1cc(Cl)c2nc(CN3CCN(c4cccc5c4O[C@@](C)(c4ccc(Cl)cn4)O5)[C@@H]4CC[C@@H]43)n(C[C@@H]3CCO3)c2c1]

Cs₂CO₃ (1.17 g, 3.58 mmol) was added to a solution of (1RS,6SR)-2-((S*)-2-(5-chloropyridin-2-yl)-2-methylbenzo[d][1,3]dioxol-4-yl)-2,5-diazabicyclo[4.2.0]octane para-toluenesulfonate, Isomer mixture 2 Intermediate 117 (380 mg, 0.72 mmol) and methyl (S)-4-chloro-2-(chloromethyl)-1-(oxetan-2-ylmethyl)-1H-benzo[d]imidazole-6-carboxylate Intermediate 24 (236 mg, 0.72 mmol) in MeCN (20 mL) and the reaction mixture was stirred at 60° C. for 8 h. The reaction mixture was filtered through celite and washed with MeCN (3×25 mL. The solvent was removed under reduced pressure and the crude product was purified by reversed phase flash chromatography on a C18 column (0-100% MeCN in water) to give the title compound Isomer mixture 2 (380 mg, 81%) as a yellow solid; MS (ESI) m/z [M+H]⁺ 650.

### Intermediate 120

### Methyl 4-chloro-2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 1

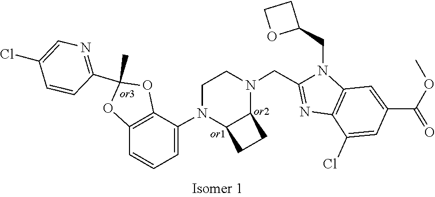 [SMILES: COC(=O)c1cc(Cl)c2nc(CN3CCN(c4cccc5c4O[C@@](C)(c4ccc(Cl)cn4)O5)[C@@H]4CC[C@@H]43)n(C[C@@H]3CCO3)c2c1]

### Intermediate 121

### Methyl 4-chloro-2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 2

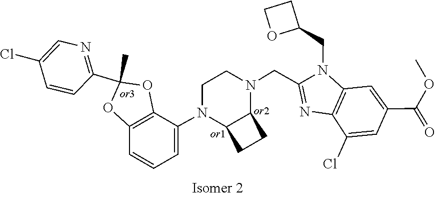 [SMILES: COC(=O)c1cc(Cl)c2nc(CN3CCN(c4cccc5c4O[C@@](C)(c4ccc(Cl)cn4)O5)[C@@H]4CC[C@@H]43)n(C[C@@H]3CCO3)c2c1]

The stereoisomers of methyl 4-chloro-2-(((1RS,6SR)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer mixture 1 Intermediate 118 (380 mg) were separated by chiral chromatography on a Chiralpak ID column (250×20 mm ID, μm), eluted with 25% EtOH in hexane (0.5% 2 M NH₃ in MeOH), at a flow rate of 20 mL/min and detected at 220 nm and 254 nm;

The first eluted compound was collected and evaporated to give the title compound Isomer 1, Intermediate 120 (150 mg, 39%) as a yellow solid; MS (ESI) m/z [M+H]⁺ 650.2; and The second eluted compound was collected and evaporated to give the title compound Isomer 2 Intermediate 121 (150 mg, 39%) as a white solid; MS (ESI) m/z [M+H]⁺ 650.1.

### Intermediate 122

### Methyl 4-chloro-2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 3

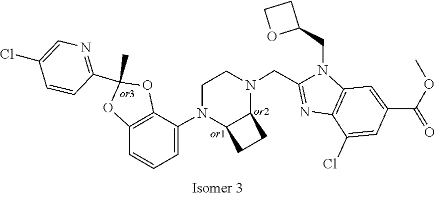 [SMILES: COC(=O)c1cc(Cl)c2nc(CN3CCN(c4cccc5c4O[C@@](C)(c4ccc(Cl)cn4)O5)[C@@H]4CC[C@@H]43)n(C[C@@H]3CCO3)c2c1]

### Intermediate 123

Methyl 4-chloro-2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 4

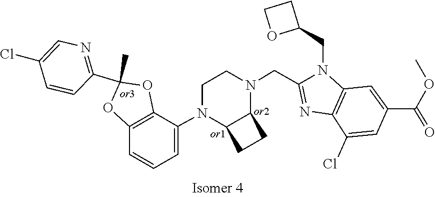 [SMILES: COC(=O)c1cc(Cl)c2nc(CN3CCN(c4cccc5c4O[C@@](C)(c4ccc(Cl)cn4)O5)[C@@H]4CC[C@@H]43)n(C[C@@H]3CCO3)c2c1]

The stereoisomers of methyl 4-chloro-2-(((1RS,6SR)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer mixture 2 Intermediate 119 (380 mg) were separated by chiral chromatography on a Chiralpak ID column (250×20 mm ID, 5 μm), eluted with 30% EtOH in hexane (0.5% 2 M NH₃ in MeOH), at a flow rate of 20 mL/min and detected at 220 nm and 254 nm;

The first eluted compound was collected and evaporated to give the title compound Isomer 3, Intermediate 122 (180 mg, 47%) as a yellow solid; MS (ESI) m/z [M+H]⁺ 650; and
the second eluted compound was collected and evaporated to give the title compound Isomer 4 Intermediate 123 (120 mg, 32%) as a pink solid; MS (ESI) m/z [M+H]⁺ 650.

### Intermediate 124

### Methyl 2-(((1RS,6RS)-5-(2-(4-chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate

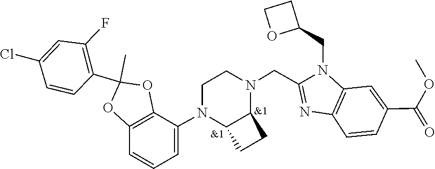 [SMILES: COC(=O)c1ccc2nc(CN3CCN(c4cccc5c4OC(C)(c4ccc(Cl)cc4F)O5)[C@H]4CC[C@@H]43)n(C[C@@H]3CCO3)c2c1]

Methyl (S)-2-(chloromethyl)-1-(oxetan-2-ylmethyl)-1H-benzo[d]imidazole-6-carboxylate (370 mg, 1.26 mmol) was added to a suspension of rac-(1R,6R)-2-(2-(4-Chloro-2-fluorophenyl)-2-methylbenzo[d][1,3]dioxol-4-yl)-2,5-diazabicyclo[4.2.0]octane hydrochloride Intermediate 32 (562 mg, 1.26 mmol), DIPEA (974 mg, 7.54 mmol) and NaI (753 mg, 5.0 mmol) in MeCN (150 mL) and the reaction mixture was stirred at 45° C. for 18 h. The reaction mixture was concentrated in vacuo, the residue was diluted with water and extracted with DCM (2×90 mL). The combined organic layer was dried over Na₂SO₄, filtered and concentrated in vacuo. The residue was purified by preparative HPLC, PrepMethod E, is (gradient: 50-70%) to give the title compound (246 mg, 33%); MS (ESI) m/z [M+H]⁺ 632.2.

### Intermediate 125

### Methyl 2-(((1R*,6R*)-5-((R*)-2-(4-chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 1

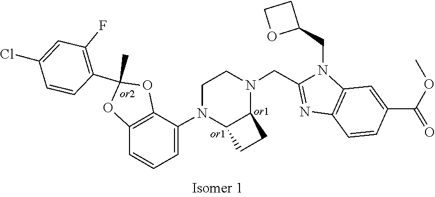 [SMILES: COC(=O)c1ccc2nc(CN3CCN(c4cccc5c4O[C@@](C)(c4ccc(Cl)cc4F)O5)[C@H]4CC[C@@H]43)n(C[C@@H]3CCO3)c2c1]

### Intermediate 126

### Methyl 2-(((1R*,6R*)-5-((R*)-2-(4-chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 2

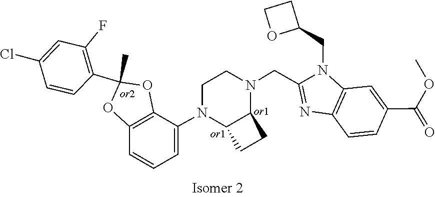 [SMILES: COC(=O)c1ccc2nc(CN3CCN(c4cccc5c4O[C@@](C)(c4ccc(Cl)cc4F)O5)[C@H]4CC[C@@H]43)n(C[C@@H]3CCO3)c2c1]

The stereoisomers of methyl 2-(((1RS,6RS)-5-(2-(4-chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate Intermediate 124 were separated by chiral chromatography on a Chiralcel OZ-H column (250×50 mm, 5p m), eluted with hexane:MeOH:IPA (3:1:1), at a flow rate of 12 mL/min; The first eluted compound was collected and evaporated to give the title compound Isomer 1, Intermediate 125 (78 mg); MS (ESI) m/z [M+H]⁺ 632.2; and
the second eluted compound mixture was collected and evaporated to give a mixture of isomers. The isomers were separated by chiral chromatography on a Chiralpak IA-III column (250×20 mm ID, 5 μm), eluted with hexane:MeOH:IPA (50:25:25), at a flow rate of 12 is mL/min;
the first eluted compound was collected and evaporated to give the title compound Isomer 2 Intermediate 126 (28 mg); MS (ESI) m/z [M+H]⁺ 632.2.

### Intermediate 127

### Methyl 2-(((1RS,6RS)-5-(2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate

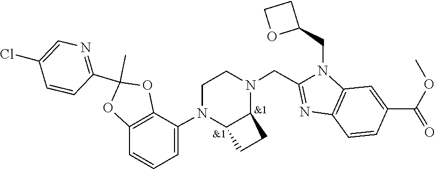 [SMILES: COC(=O)c1ccc2nc(CN3CCN(c4cccc5c4OC(C)(c4ccc(Cl)cn4)O5)[C@H]4CC[C@@H]43)n(C[C@@H]3CCO3)c2c1]

Methyl 2-(chloromethyl)-1-[(2S)-oxetan-2-yl]methyl-1H-1,3-benzodiazole-6-carboxylate (254 mg, 862 μmol) was added to a suspension of rac-(1R,6R)-2-(2-(5-chloropyridin-2-yl)-2-methylbenzo[d][1,3]dioxol-4-yl)-2,5-diazabicyclo[4.2.0]octane Intermediate 26 (370 mg, 1.03 mmol), DIPEA (557 mg, 4.31 mmol) and NaI (517 mg, 3.45 mmol) in MeCN (10 mL), and the reaction mixture was stirred at 60° C. for 12 h. The reaction mixture was concentrated in vacuo, then diluted with water (20 mL) and extracted with DCM (3×20 mL). The combined organic layer was dried over Na₂SO₄, filtered, and concentrated in vacuo. The residue was purified by preparative HPLC, PrepMethod E, (gradient: 30-70%) to give the title compound (250 mg, 49%); MS (ESI) m/z [M+H]⁺ 616.2.

### Intermediate 128

### Methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 3

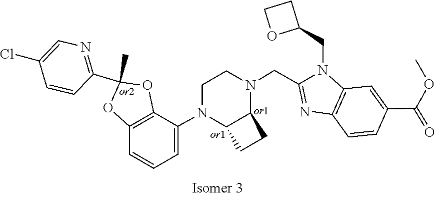 [SMILES: COC(=O)c1ccc2nc(CN3CCN(c4cccc5c4O[C@@](C)(c4ccc(Cl)cn4)O5)[C@H]4CC[C@@H]43)n(C[C@@H]3CCO3)c2c1]

### Intermediate 129

### Methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 4

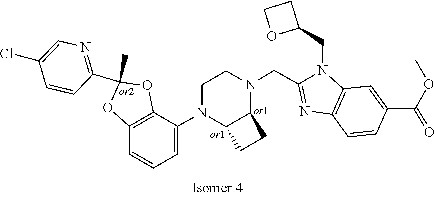 [SMILES: COC(=O)c1ccc2nc(CN3CCN(c4cccc5c4O[C@@](C)(c4ccc(Cl)cn4)O5)[C@H]4CC[C@@H]43)n(C[C@@H]3CCO3)c2c1]

The diastereoisomers of methyl 2-(((1RS,6RS)-5-(2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate Intermediate 127 were separated by chiral chromatography on a CHIRACEL OZ-H column (250×20 mm ID, 5 μm), eluted with hexane:IPA:MeOH (80:10:10), at a flow rate of 18 mL/min;
the second eluted compound mixture was collected and evaporated to yield a mixture of isomers. The stereoisomers of the second eluted compound mixture were separated by chiral chromatography on a Chiralpak AD-H column (250×21 mm ID, 5 μm) eluted with MeOH in CO₂ (70:30), at a flow rate of 50 mL/min;
the first eluted compound was collected and evaporated to yield the title compound Isomer 3, Intermediate 128 (41 mg); MS (ESI) m/z [M+H]⁺ 616.4; and
the second eluted compound was collected and evaporated to yield the title compound Isomer 4 Intermediate 129 (42 mg); MS (ESI) m/z [M+H]⁺ 616.4.

### Intermediate 130

### Methyl 3-(((1-(cyanomethyl)cyclopropyl)methyl)amino)-5-fluoro-4-nitrobenzoate

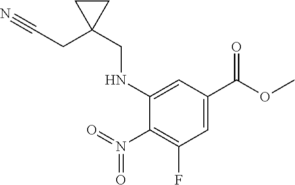 [SMILES: COC(=O)c1cc(F)c([N+](=O)[O-])c(NCC2(CC#N)CC2)c1]

Methyl 3,5-difluoro-4-nitrobenzoate (5.0 g, 23.03 mmol), 2-(1-(aminomethyl)cyclopropyl)acetonitrile hydrochloride (3.38 g, 23.03 mmol) and DIPEA (12 mL, 69.08 mmol) were mixed in THE (20 mL) and the reaction mixture was stirred at 60° C. for 16 h. The reaction mixture was cooled to rt and diluted with water. The aqueous phase was extracted with MTBE (3×50 mL), and the combined organic layer was washed with brine, dried over anhydrous Na₂SO₄, and filtered. The filtrate was concentrated in vacuo to give the title compound (5.1 g, 54%); MS (ESI) m/z [M+H]⁺ 308.0.

### Intermediate 131

### Methyl 4-amino-3-(((1-(cyanomethyl)cyclopropyl)methyl)amino)-5-fluorobenzoate

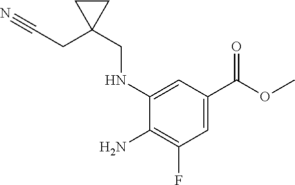 [SMILES: COC(=O)c1cc(F)c(N)c(NCC2(CC#N)CC2)c1]

Pd/C (10%, 0.5 g) was added to a solution of methyl 3-(((1-(cyanomethyl)cyclopropyl)methyl)amino)-5-fluoro-4-nitrobenzoate Intermediate 130 (5.1 g, 12.45 mmol) in dry MeOH (20 mL) and the reaction mixture was stirred under an atmosphere of H₂(g) (1 atm) at ambient temperature until the reaction was complete. The reaction mixture was filtered and concentrated in vacuo. The residue was purified by flash chromatography on silica (0-99% MTBE in hexane) to give the title compound (2.8 g, 77%); MS (ESI) m z [M+H]⁺ 278.2.

### Intermediate 132

### Methyl 2-(chloromethyl)-1-((1-(cyanomethyl)cyclopropyl)methyl)-4-fluoro-1H-benzo[d]imidazole-6-carboxylate

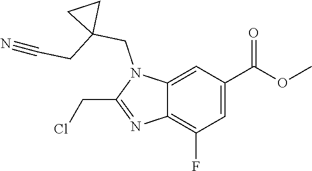 [SMILES: COC(=O)c1cc(F)c2nc(CCl)n(CC3(CC#N)CC3)c2c1]

2-Chloro-1,1,1-trimethoxyethane (1.34 mL, 9.92 mmol) and pTsOH (155 mg, 902 μmol) were added to a solution of methyl 4-amino-3-(((1-(cyanomethyl)cyclopropyl)methyl)amino)-5-fluorobenzoate Intermediate 131 (2.5 g, 9.02 mmol) in THE (50 mL) and the reaction mixture was stirred at 50° C. overnight. The reaction mixture was poured into water and extracted with EtOAc (3×10 mL). The combined organic layer was washed with brine and concentrated under vacuum to give the title compound (2.25 g, 67%); MS (ESI) m/z [M+H]⁺ 336.2.

### Intermediate 133

### rac-Methyl 2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylate

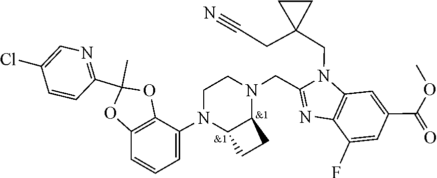 [SMILES: COC(=O)c1cc(F)c2nc(CN3CCN(c4cccc5c4OC(C)(c4ccc(Cl)cn4)O5)[C@H]4CC[C@@H]43)n(CC3(CC#N)CC3)c2c1]

rac-(1R,6R)-2-(2-(5-Chloropyridin-2-yl)-2-methylbenzo[d][1,3]dioxol-4-yl)-2,5-diazabicyclo[4.2.0]octane dihydrochloride Intermediate 26 (500 mg, 1.4 mmol), methyl 2-(chloromethyl)-1-((1-(cyanomethyl)cyclopropyl)methyl)-4-fluoro-1H-benzo[d]imidazole-6-carboxylate Intermediate 132 (389 mg, 1.16 mmol), DIPEA (749 mg, 5.8 mmol, 1.01 mL) and NaI (17 mg, 116 μmol) were mixed in MeCN (3 mL) and the reaction mixture was stirred at 40° C. for 16 h. The reaction mixture was diluted with water (5 mL) and extracted with EtOAc (3×5 mL). The combined organic layer was dried over anhydrous Na₂SO₄, filtered and evaporated. The residue was purified by preparative HPLC, PrepMethod E, (gradient: 30-50%) to give the title compound (330 mg, 43%); MS (ESI) m/z [M+H]⁺ 657.2.

### Intermediate 134

### rel-Methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylate, Isomer 1

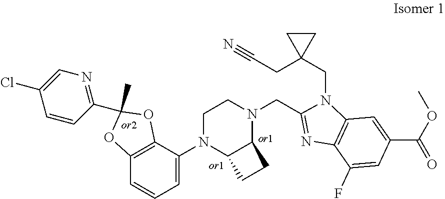 [SMILES: COC(=O)c1cc(F)c2nc(CN3CCN(c4cccc5c4O[C@@](C)(c4ccc(Cl)cn4)O5)[C@H]4CC[C@@H]43)n(CC3(CC#N)CC3)c2c1]

### Intermediate 135

### rel-Methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylate, Isomer 2

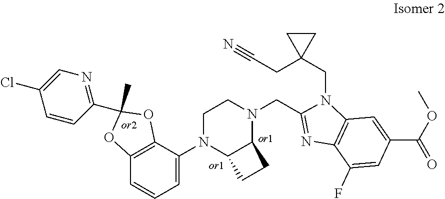 [SMILES: COC(=O)c1cc(F)c2nc(CN3CCN(c4cccc5c4O[C@@](C)(c4ccc(Cl)cn4)O5)[C@H]4CC[C@@H]43)n(CC3(CC#N)CC3)c2c1]

### Intermediate 136

### rel-Methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylate. Isomer 3

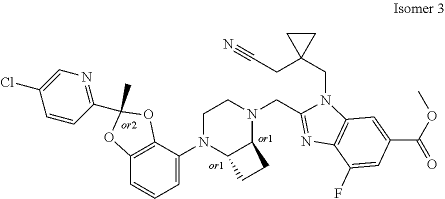 [SMILES: COC(=O)c1cc(F)c2nc(CN3CCN(c4cccc5c4O[C@@](C)(c4ccc(Cl)cn4)O5)[C@H]4CC[C@@H]43)n(CC3(CC#N)CC3)c2c1]

### Intermediate 137

### rel-Methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylate, Isomer 4

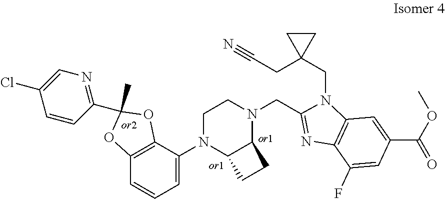 [SMILES: COC(=O)c1cc(F)c2nc(CN3CCN(c4cccc5c4O[C@@](C)(c4ccc(Cl)cn4)O5)[C@H]4CC[C@@H]43)n(CC3(CC#N)CC3)c2c1]

The stereoisomers of rac-methyl 2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylate Intermediate 133 were separated by chiral chromatography on a CHIRALPAK IC column (250×21 mm, 5 μm), eluted with IPA:MeOH:CHCl₃ (47.5:47.5:5), at a flow rate of 12 mL/min; the first eluted compound mixture was collected and evaporated to yield a mixture of isomers and the second eluted compound mixture was collected and evaporated to yield a mixture of isomers. The stereoisomers of the first eluted compound mixture were separated by chiral chromatography on a CHIRALPAK IF column (250×21 mm, 5 μm) eluted with hexane:IPA:MeOH (80:10:10) at a flow rate of 14 mL/min;
the first eluted compound was collected and evaporated to yield the title compound Isomer 1, Intermediate 134 (68 mg); MS (ESI) m/z [M+H]⁺ 657.2; and
the second eluted compound was collected and evaporated to yield the title compound Isomer 2 Intermediate 135 (62 mg); MS (ESI) m/z [M+H]⁺ 657.2.

The stereoisomers of the second eluted compound mixture were separated by chiral chromatography on a Chiralpak IA column (250×21 mm, 5 μm) eluted with 20% MeOH in CO₂ at a flow rate of 50 mL/min;
the first eluted compound was collected and evaporated to yield the title compound Isomer 3, Intermediate 136 (86 mg); MS (ESI) m/z [M+H]⁺ 656.4; and
the second eluted compound was collected and evaporated to yield the title compound Isomer 4 Intermediate 137 (59 mg); MS (ESI) m/z [M+H]⁺ 656.5.

### Intermediate 138

### Methyl 2-(chloromethyl)-1-((1-cyanocyclopropyl)methyl)-4-fluoro-1H-benzo[d]imidazole-6-carboxylate

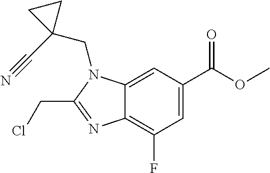 [SMILES: COC(=O)c1cc(F)c2nc(CCl)n(CC3(C#N)CC3)c2c1]

The title compound was prepared in three steps as described for Intermediate 132 from methyl 3,5-difluoro-4-nitrobenzoate (3.05 g, 23.03 mmol) and 1-(aminomethyl)cyclopropane-1-carbonitrile hydrochloride (5.0 g, 23.0 mmol) to give the title compound (2.35 g, 69%); MS (ESI) m/z [M+H]⁺ 322.0.

### Intermediate 139

### rac-Methyl 2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylate

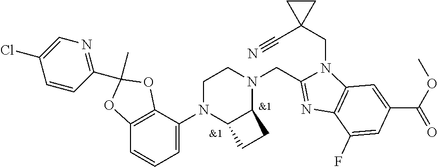 [SMILES: COC(=O)c1cc(F)c2nc(CN3CCN(c4cccc5c4OC(C)(c4ccc(Cl)cn4)O5)[C@H]4CC[C@@H]43)n(CC3(C#N)CC3)c2c1]

rac-(1R,6R)-2-(2-(5-Chloropyridin-2-yl)-2-methylbenzo[d][1,3]dioxol-4-yl)-2,5-diazabicyclo[4.2.0]octane Intermediate 26 (500 mg, 1.4 mmol), methyl 2-(chloromethyl)-1-((1-cyanocyclopropyl)methyl)-4-fluoro-1H-benzo[d]imidazole-6-carboxylate Intermediate 138 (373 mg, 1.16 mmol), DIPEA (1.01 mL, 5.8 mmol) and NaI (17 mg, 116 μmol) were mixed in MeCN (5 mL) and the reaction mixture was stirred at 40° C. for 16 h. The reaction mixture was diluted with water (5 mL) and extracted with EtOAc (3×5 mL). The combined organic layer was dried over anhydrous Na₂SO₄, filtered and evaporated. The residue was purified by preparative HPLC, PrepMethod E (gradient: 30-50%) to give the title compound (370 mg, 49%); MS (ESI) m/z [M+H]⁺ 643.2.

### Intermediate 140

### rel-Methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylate Isomer 2

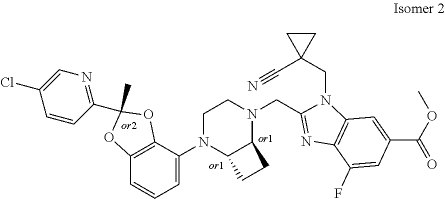 [SMILES: COC(=O)c1cc(F)c2nc(CN3CCN(c4cccc5c4O[C@@](C)(c4ccc(Cl)cn4)O5)[C@H]4CC[C@@H]43)n(CC3(C#N)CC3)c2c1]

### Intermediate 141

### rel-Methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylate Isomer 4

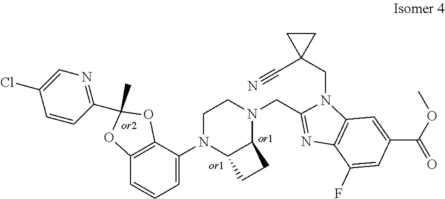 [SMILES: COC(=O)c1cc(F)c2nc(CN3CCN(c4cccc5c4O[C@@](C)(c4ccc(Cl)cn4)O5)[C@H]4CC[C@@H]43)n(CC3(C#N)CC3)c2c1]

The stereoisomers of rac-methyl 2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylate Intermediate 139 were separated by chiral chromatography on a CHIRALCEL OZ-H colum (250×21 mm, 5 μm) eluted with hexane:IPA:MeOH (60:20:20) at a flow rate of 14 mL/min; the second eluted compound mixture was collected and evaporated to give a mixture of stereoisomers; and
the fourth eluted compound was collected and evaporated to yield the title compound Isomer 4 Intermediate 141 (99 mg); MS (ESI) m/z [M+H]⁺ 643.2.

The stereoisomers of the second eluted compound mixture were separated by chiral chromatography on a Chiralpak IA column (250×30 mm, 5 μm) eluted with hexane:MeOH:IPA (60:20:20) at a flow rate of 12 mL/min;
the first eluted compound was collected and evaporated to yield the title compound Isomer 2, Intermediate 140 (65 mg); MS (ESI) m/z [M+H]⁺ 643.2.

### Intermediate 142

### Methyl 3-(((1-cyanocyclopropyl)methyl)amino)-5-methoxy-4-nitrobenzoate

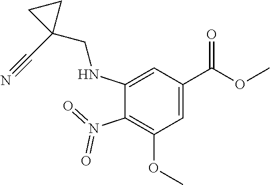 [SMILES: COC(=O)c1cc(NCC2(C#N)CC2)c([N+](=O)[O-])c(OC)c1]

Methyl 3-fluoro-5-methoxy-4-nitrobenzoate (901 g, 3.93 mmol) and DIPEA (1.5 g, 11.8 mmol) were slowly added to a solution of 1-(aminomethyl)cyclopropyl)-1-carbonitrile hydrochloride (522 mg, 3.93 mmol) in THF (20 mL) and the reaction mixture was stirred at 60° C. for 16 h. The reaction mixture was cooled to rt and diluted with water. The aqueous phase was extracted with DCM (3×30 mL), and the combined organic layer was dried over is anhydrous Na₂SO₄, filtered and concentrated. The residue was purified by flash chromatography on silica (DCM:MeOH, 4:1). The product containing fractions were collected and evaporated, and the obtained yellow solid was triturated with petroleum ether:EtOAc (30:1) to give the title compound (1.1 g, 94%) as a yellow solid; MS (ESI) m z [M+H]⁺ 306.1.

### Intermediate 143

### Methyl 4-amino-3-(((1-cyanocyclopropyl)methyl)amino)-5-methoxybenzoate

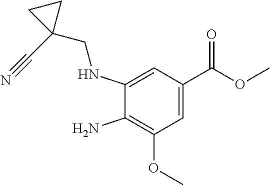 [SMILES: COC(=O)c1cc(NCC2(C#N)CC2)c(N)c(OC)c1]

Wet Pt/C (10%, 0.3 g) was added to a suspension of methyl 3-(((1-cyanocyclopropyl)methyl)amino)-5-methoxy-4-nitrobenzoate Intermediate 142 (1.104 g, 3.50 mmol) in MeOH (100 mL) and the reaction mixture was stirred under an atmosphere of H₂(g) (1 atm) at 20° C. for 36 h. The reaction mixture was filtered and the filter cake was washed with MeOH (50 mL). The filtrate was concentrated under reduced pressure and the crude product was purified by flash chromatography on silica (0-99% EtOAc in hexane) to give the title compound (515 mg, 50%); ¹H NMR (500 MHz, CDCl₃) δ 7.22 (d, 1H), 7.10 (d, 1H), 3.93-3.82 (m, 6H), 3.24 (s, 2H), 1.36-1.29 (m, 2H), 1.02-0.92 (m, 2H).

### Intermediate 144

### Methyl 2-(chloromethyl)-1-((1-cyanocyclopropyl)methyl)-4-methoxy-1H-benzo[d]imidazole-6-carboxylate

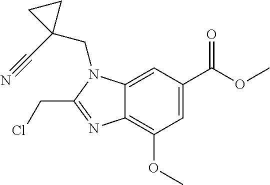 [SMILES: COC(=O)c1cc(OC)c2nc(CCl)n(CC3(C#N)CC3)c2c1]

2-Chloro-1,1,1-trimethoxyethane (318 mg, 2.06 mmol) and pTsOH (32 mg, 0.19 mmol) were added to a solution of methyl 4-amino-3-(((1-cyanocyclopropyl)methyl)amino)-5-methoxybenzoate Intermediate 143 (515 mg, 1.87 mmol) in MeCN (50 mL) and the reaction is mixture was stirred at 80° C. for 18 h. The reaction mixture was evaporated at reduced pressure and the residue was diluted with EtOAc. The organic layer was washed with NaHCO₃ (aq) and water, dried over Na₂SO₄, filtered and evaporated. The crude product was purified by preparative HPLC, PrepMethod E, (gradient: 0-55%) to give the title compound (211 mg, 47%); MS (ESI) m/z [M+H]⁺ 334.0.

### Intermediate 145

### rac-Methyl 2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylate

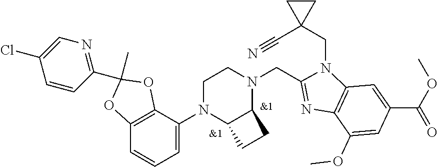 [SMILES: COC(=O)c1cc(OC)c2nc(CN3CCN(c4cccc5c4OC(C)(c4ccc(Cl)cn4)O5)[C@H]4CC[C@@H]43)n(CC3(C#N)CC3)c2c1]

Methyl 2-(chloromethyl)-1-((1-cyanocyclopropyl)methyl)-4-methoxy-1H-benzo[d]imidazole-6-carboxylate Intermediate 144 (212 mg, 634 μmol) was added to a suspension of rac-(1R,6R)-2-(2-(5-chloropyridin-2-yl)-2-methylbenzo[d][1,3]dioxol-4-yl)-2,5-diazabicyclo[4.2.0]octane dihydrochloride Intermediate 26 (273 mg, 634 μmol), DIPEA (491 mg, 3.8 mmol) and NaI (380 mg, 2.54 mmol) in MeCN (100 mL) and the reaction mixture was stirred at 60° C. for 18 h. The reaction mixture was concentrated in vacuo, diluted with water (40 mL) and extracted with DCM (2×70 mL). The combined organic layer was dried over Na₂SO₄, filtered, and concentrated in vacuo. The residue was purified by preparative HPLC, PrepMethod E, (gradient: 30-70%) to give the title compound (144 mg, 35%); MS (ESI) m/z [M+H]⁺ 655.0.

### Intermediate 146

### rel-Methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylate Isomer 1

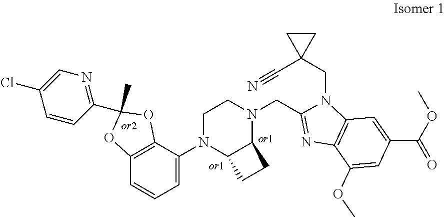 [SMILES: COC(=O)c1cc(OC)c2nc(CN3CCN(c4cccc5c4O[C@@](C)(c4ccc(Cl)cn4)O5)[C@H]4CC[C@@H]43)n(CC3(C#N)CC3)c2c1]

### Intermediate 147

### rel-Methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylate Isomer 2

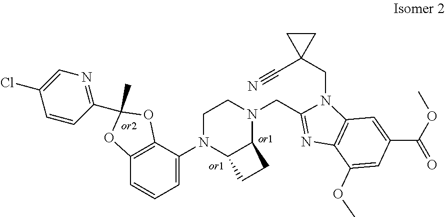 [SMILES: COC(=O)c1cc(OC)c2nc(CN3CCN(c4cccc5c4O[C@@](C)(c4ccc(Cl)cn4)O5)[C@H]4CC[C@@H]43)n(CC3(C#N)CC3)c2c1]

### Intermediate 148

### rel-Methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylate Isomer 3

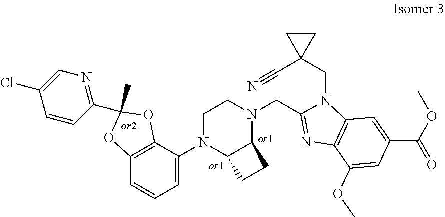 [SMILES: COC(=O)c1cc(OC)c2nc(CN3CCN(c4cccc5c4O[C@@](C)(c4ccc(Cl)cn4)O5)[C@H]4CC[C@@H]43)n(CC3(C#N)CC3)c2c1]

### Intermediate 149

### rel-Methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylate Isomer 4

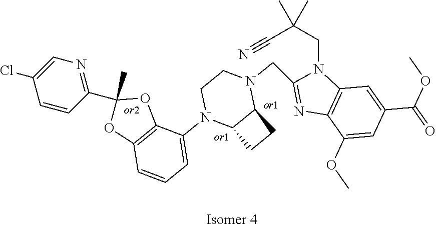 [SMILES: COC(=O)c1cc(OC)c2nc(CN3CCN(c4cccc5c4O[C@@](C)(c4ccc(Cl)cn4)O5)[C@H]4CC[C@@H]43)n(CC(C)(C)C#N)c2c1]

The stereoisomers of rac-methyl 2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylate Intermediate 145 were separated by chiral chromatography on a CHIRALPAK IA column (250×20 mm, μm), eluted with hexane:IPA:MeOH (70:15:15), at a flow rate of 12 mL/min; the first eluted is compound mixture was collected and evaporated to yield a mixture of isomers; and the second eluted compound mixture was collected and evaporated to yield a mixture of isomers.

The stereoisomers of the first eluted compound mixture were separated by chiral chromatography on a CHIRALPAK IB column (250×20 mm, 5 μm) eluted with hexane:IPA:MeOH (85:7.5:7.5) at a flow rate of 18 mL/min;
the first eluted compound was collected and evaporated to yield the title compound Isomer 1, Intermediate 146 (16 mg); MS (ESI) m/z [M+H+655.2; and
the second eluted compound was collected and evaporated to yield the title compound Isomer 2 Intermediate 147 (21 mg); MS (ESI) m/z [M+H]⁺ 655.2.

The stereoisomers of the second eluted compound mixture were separated by chiral chromatography on a Chiralpak IB column (250×20 mm, 5 μm) eluted with hexane:IPA:MeOH (85:7.5:7.5) at a flow rate of 18 mL/min;
the first eluted compound was collected and evaporated to yield the title compound Isomer 3, Intermediate 148 (19 mg); MS (ESI) m/z [M+H]⁺ 655.2; and
the second eluted compound was collected and evaporated to yield the title compound Isomer 4 Intermediate 149 (20 mg); MS (ESI) m/z [M+H]⁺ 655.2.

### Intermediate 150

### Methyl 3-chloro-5-(((1-(cyanomethyl)cyclopropyl)methyl)amino)-4-nitrobenzoate

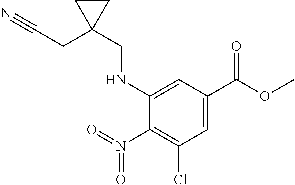 [SMILES: COC(=O)c1cc(Cl)c([N+](=O)[O-])c(NCC2(CC#N)CC2)c1]

Methyl 3-chloro-5-fluoro-4-nitrobenzoate (5.05 g, 21.62 mmol), 2-(1-(aminomethyl)cyclopropyl)acetonitrile hydrochloride (3.17 g, 21.62 mmol) and DIPEA (8.38 g, 64.87 mmol) were mixed in THE (200 mL), and the reaction mixture was heated at 50° C. for 14 h. The reaction mixture was evaporated under reduced pressure and the residue was diluted with EtOAc (150 mL). The organic layer was washed with water (2×50 mL), dried over Na₂SO₄, filtered, and concentrated under reduced pressure to give the crude title compound (5.73 g); MS (ESI) m/z [M+H]⁺ 324.0.

### Intermediate 151

### Methyl 4-amino-3-chloro-5-(((1-(cyanomethyl)cyclopropyl)methyl)amino)benzoate

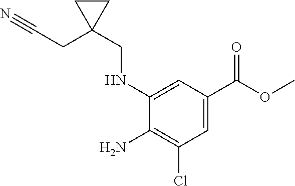 [SMILES: COC(=O)c1cc(Cl)c(N)c(NCC2(CC#N)CC2)c1]

Wet Pt/C (10%, 0.2 g) was added to a suspension of methyl 3-chloro-5-(((1-(cyanomethyl)cyclopropyl)methyl)amino)-4-nitrobenzoate Intermediate 150 (5.73 g, 17.7 mmol) in MeOH (200 mL) and the reaction mixture was stirred under an atmosphere of H₂(g) (1 atm) at 20° C. for 64 h. The reaction mixture was filtered, and the catalyst carefully washed with MeOH (100 mL). The filtrate was concentrated under reduced pressure. The crude compound was purified by flash chromatography on silica (0-99% MTBE in hexane) to give the title compound (3.7 g, 71%); ¹H NMR (500 MHz, CDCl₃) δ 7.66 (s, 1H), 7.34 (s, 1H), 3.87 (s, 3H), 3.18 (s, 2H), 2.61 (s, 2H), 1.58 (s, 3H), 0.76 (s, 4H).

### Intermediate 152

### Methyl 4-chloro-2-(chloromethyl)-1-((1-(cyanomethyl)cyclopropyl)methyl)-1H-benzo[d]imidazole-6-carboxylate

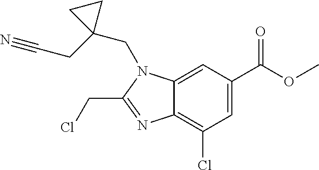 [SMILES: COC(=O)c1cc(Cl)c2nc(CCl)n(CC3(CC#N)CC3)c2c1]

2-Chloro-1,1,1-trimethoxyethane (435 mg, 2.81 mmol) and pTsOH (44 mg, 256 μmol) were is added to a stirred solution of methyl 4-amino-3-chloro-5-(((1-(cyanomethyl)cyclopropyl)methyl)amino)benzoate Intermediate 151 (751 mg, 2.56 mmol) in MeCN (100 mL) and the reaction mixture was heated at 60° C. for 2 h. The reaction mixture was cooled to rt and then evaporated under reduced pressure. The residue was diluted with EtOAc (70 mL) and the mixture was washed with NaHCO₃ (30 mL) and water (30 mL). The organic layer was dried over Na₂SO₄ and evaporated to give the crude title compound (0.72 g); MS (ESI) m/z [M+H]⁺ 352.0.

### Intermediate 153

### rac-Methyl 4-chloro-2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylate

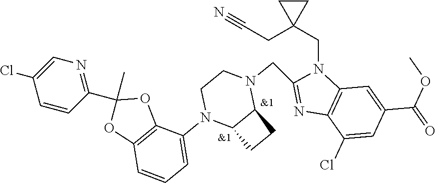 [SMILES: COC(=O)c1cc(Cl)c2nc(CN3CCN(c4cccc5c4OC(C)(c4ccc(Cl)cn4)O5)[C@H]4CC[C@@H]43)n(CC3(CC#N)CC3)c2c1]

Methyl 4-chloro-2-(chloromethyl)-1-((1-(cyanomethyl)cyclopropyl)methyl)-1H-benzo[d]imidazole-6-carboxylate Intermediate 152 (1.08 g, 3.08 mmol) was added to a suspension of rac-(1R,6R)-2-(2-(5-chloropyridin-2-yl)-2-methylbenzo[d][1,3]dioxol-4-yl)-2,5-diazabicyclo[4.2.0]octane dihydrochloride Intermediate 26 (1.33 g, 3.08 mmol), DIPEA (2.39 g, 18.47 mmol) and NaI (1.85 g, 12.32 mmol) in MeCN (150 mL) and the reaction mixture was stirred at 60° C. for 18 h. The reaction mixture was concentrated in vacuo, and the residue was diluted with water (60 mL) and extracted with DCM (2×90 mL). The combined organic layer was dried over anhydrous Na₂SO₄, filtered and evaporated. The residue was purified by preparative HPLC, PrepMethod E, (gradient: 30-50%) to give the title compound (150 mg, 49%); MS (ESI) m/z [M+H]⁺ 673.

### Intermediate 154

### rel-Methyl 4-chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylate Isomer 1

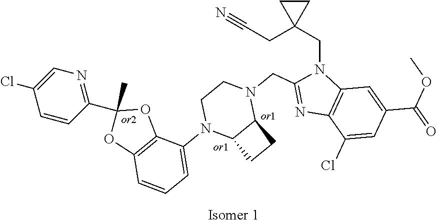 [SMILES: COC(=O)c1cc(Cl)c2nc(CN3CCN(c4cccc5c4O[C@@](C)(c4ccc(Cl)cn4)O5)[C@H]4CC[C@@H]43)n(CC3(CC#N)CC3)c2c1]

### Intermediate 155

### rel-Methyl 4-chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylate Isomer 2

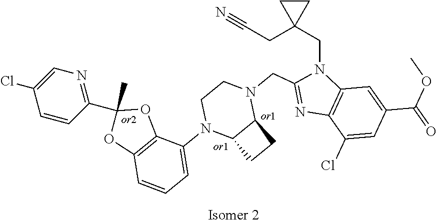 [SMILES: COC(=O)c1cc(Cl)c2nc(CN3CCN(c4cccc5c4O[C@@](C)(c4ccc(Cl)cn4)O5)[C@H]4CC[C@@H]43)n(CC3(CC#N)CC3)c2c1]

### Intermediate 156

### rel-Methyl 4-chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylate Isomer 3

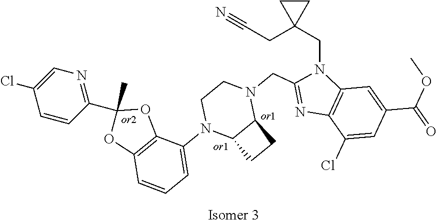 [SMILES: COC(=O)c1cc(Cl)c2nc(CN3CCN(c4cccc5c4O[C@@](C)(c4ccc(Cl)cn4)O5)[C@H]4CC[C@@H]43)n(CC3(CC#N)CC3)c2c1]

### Intermediate 157

### rel-Methyl 4-chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylate Isomer 4

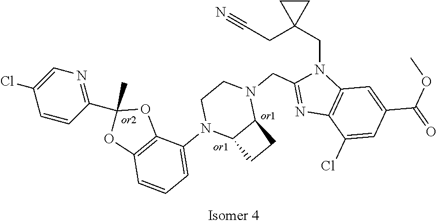 [SMILES: COC(=O)c1cc(Cl)c2nc(CN3CCN(c4cccc5c4O[C@@](C)(c4ccc(Cl)cn4)O5)[C@H]4CC[C@@H]43)n(CC3(CC#N)CC3)c2c1]

The stereoisomers of rac-methyl 4-chloro-2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylate Intermediate 153 is were separated by chiral chromatography on a CHIRALPAK IC column (250×20 mm, 5 μm) eluted with hexane:IPA:MeOH (50:25:25) at a flow rate of 13 mL/min;
the first eluted compound mixture was collected and evaporated to yield a mixture of isomers; and the second eluted compound mixture was collected and evaporated to yield a mixture of isomers.

The stereoisomers of the first eluted compound mixture were separated by chiral chromatography on a Chiralcel OZ-H column (250×20 mm, 5 μm), eluted with hexane:IPA:MeOH (40:30:30) at a flow rate of 12 mL/min;
the first eluted compound was collected and evaporated to give the title compound Isomer 1 Intermediate 154 (29 mg); MS (ESI) m/z [M+H]⁺ 673.2; and
the second eluted compound was collected and evaporated to yield the title compound Isomer 2 Intermediate 155 (31 mg); MS (ESI) m/z [M+H]⁺ 673.2.

The stereoisomers of the second eluted compound mixture were separated by chiral chromatography on a Chiralpak AS-H column (250×20 mm, 5 μm), eluted with hexane:IPA:MeOH (90:5:5) at a flow rate of 12 mL/min;
the first eluted compound was collected and evaporated to yield the title compound Isomer 3 Intermediate 156 (27 mg); MS (ESI) m/z [M+H]⁺ 673.2; and is the second eluted compound was collected and evaporated to yield the title compound Isomer 4 Intermediate 157 (29 mg); MS (ESI) m/z [M+H]⁺ 673.2.

### Intermediate 158

### Methyl 3-((2-cyclopropoxyethyl)amino)-5-methoxy-4-nitrobenzoate

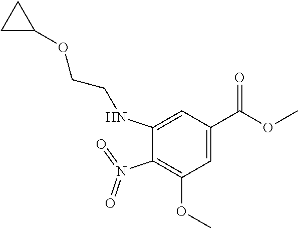 [SMILES: COC(=O)c1cc(NCCOC2CC2)c([N+](=O)[O-])c(OC)c1]

Methyl 3-fluoro-5-methoxy-4-nitrobensoat (3.0 g, 13.09 mmol), 2-cyclopropoxyethan-1-amine hydrochloride (1.8 g, 13.09 mmol) and DIPEA (5.02 mL, 28.8 mmol) were mixed in DMSO (15 mL) and the reaction mixture was stirred at 80° C. for 16 h. The reaction mixture was cooled to rt, diluted with water (20 mL), and the mixture was extracted with MTBE (3×20 mL). The combined organic layer was washed with brine (20 mL), dried over anhydrous Na₂SO₄, filtered and concentrated to give the title compound (3.5 g, 69%); MS (ESI) m/z [M+H]⁺ 311.0.

### Intermediate 159

### Methyl 4-amino-3-((2-cyclopropoxyethyl)amino)-5-methoxybenzoate

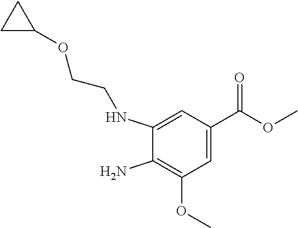 [SMILES: COC(=O)c1cc(NCCOC2CC2)c(N)c(OC)c1]

Pd/C (10%, 0.35 g) was added to a solution of methyl 3-((2-cyclopropoxyethyl)amino)-5-methoxy-4-nitrobenzoate Intermediate 158 (3.5 g, 9.02 mmol) in dry MeOH (15 mL) and the reaction mixture was stirred under an atmosphere of H₂(g) (1 atm) at ambient temperature until the reaction was complete. The reaction mixture was concentrated in vacuo, and the residue was purified by flash chromatography on silica (0-99% MTBE in hexane) to give the title compound (1.1 g, 41%); MS (ESI) m/z [M+H]⁺ 281.2.

### Intermediate 160

### Methyl 2-(chloromethyl)-1-(2-cyclopropoxyethyl)-4-methoxy-1H-benzo[d]imidazole-6-carboxylate

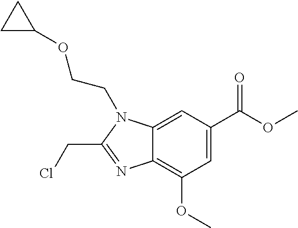 [SMILES: COC(=O)c1cc(OC)c2nc(CCl)n(CCOC3CC3)c2c1]

2-Chloro-1,1,1-trimethoxyethane (667 mg, 4.32 mmol) and pTsOH (68 mg, 0.39 mmol) were added to a solution of methyl 4-amino-3-((2-cyclopropoxyethyl)amino)-5-methoxybenzoate Intermediate 159 (1.1 g, 3.92 mmol) in THE (50 mL) and the reaction mixture was stirred at 50° C. overnight. The mixture was poured into water (15 mL) and extracted with EtOAc (3×15 mL). The combined organic layer was washed with brine (10 mL), dried over anhydrous Na₂SO₄, filtered and evaporated to give the title compound (1.2 g, 68%); MS (ESI) m z [M+H]⁺ 339.0.

### Intermediate 161

### rac-Methyl 2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-(2-cyclopropoxyethyl)-4-methoxy-1H-benzo[d]imidazole-6-carboxylate

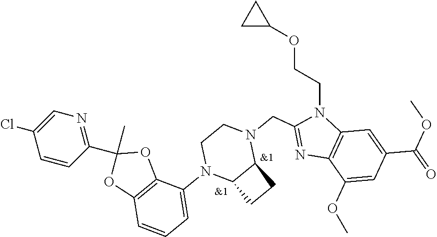 [SMILES: COC(=O)c1cc(OC)c2nc(CN3CCN(c4cccc5c4OC(C)(c4ccc(Cl)cn4)O5)[C@H]4CC[C@@H]43)n(CCOC3CC3)c2c1]

rac-(1R,6R)-2-(2-(5-Chloropyridin-2-yl)-2-methylbenzo[d][1,3]dioxol-4-yl)-2,5-diazabicyclo[4.2.0]octane Intermediate 26 (500 mg, 1.4 mmol), methyl 2-(chloromethyl)-1-(2-cyclopropoxyethyl)-4-methoxy-1H-benzo[d]imidazole-6-carboxylate Intermediate 160 (393 mg, 1.16 mmol), DIPEA (749 mg, 5.8 mmol) and NaI (17 mg, 116 μmol) were mixed in dry MeCN (7 mL), and the reaction mixture was stirred at 40° C. 16 h. The reaction mixture was diluted with water (10 mL) and extracted with EtOAc (3×10 mL). The combined organic layer was separated, dried over Na₂SO₄ and evaporated. The crude residue was purified by preparative HPLC PrepMethod E (gradient: 30-50%) to give the title compound (180 mg, 24%); MS (ESI) m/z [M+H]⁺ 660.2.

### Intermediate 162

### rel-Methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-(2-cyclopropoxyethyl)-4-methoxy-1H-benzo[d]imidazole-6-carboxylate Isomer 3

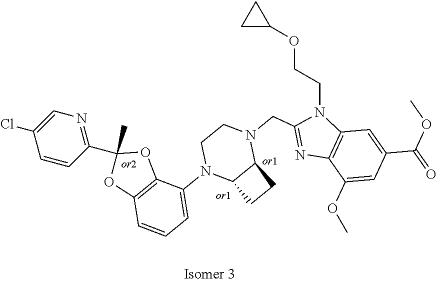 [SMILES: COC(=O)c1cc(OC)c2nc(CN3CCN(c4cccc5c4O[C@@](C)(c4ccc(Cl)cn4)O5)[C@H]4CC[C@@H]43)n(CCOC3CC3)c2c1]

The stereoisomers of rac-methyl 2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-(2-cyclopropoxyethyl)-4-methoxy-1H-benzo[d]imidazole-6-carboxylate Intermediate 161 were separated by chiral chromatography on a CHIRALPAK AD-H column (250×20 mm, 5 μm) eluted with 20% MeOH in CO₂, at a flow rate of 50 mL/min;
the second eluted compound mixture was collected and evaporated to yield a mixture of isomers.

The stereoisomers of the second eluted compound mixture were separated by chiral chromatography on a Chiralcel OZ-H column (250×20 mm, 5 μm) eluted with hexane:IPA:MeOH (80:10:10) at a flow rate of 12 mL/min;
the second eluted compound was collected and evaporated to give the title compound Isomer 3 Intermediate 162 (46 mg); MS (ESI) m/z [M+H]⁺ 660.02.

### Intermediate 163

### (S)-2-(4-Bromo-2-methylbenzo[d][1,3]dioxol-2-yl)-5-chloropyridine

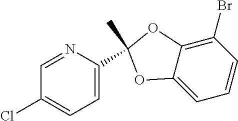 [SMILES: C[C@]1(c2ccc(Cl)cn2)Oc2cccc(Br)c2O1]

The stereoisomers of 2-(4-bromo-2-methylbenzo[d][1,3]dioxol-2-yl)-5-chloropyridine WO2020234726 (6 g, 18.37 mmol) were separated by chiral chromatography on an LUX Al (AD) column (250×30 mm, 5 μm), eluted with 3% (IPA, 20 mM DEA) in CO₂, 120 bar, at a flow rate of 150 mL/min and detected at 220 nm; The first eluted compound was collected and evaporated to give the title compound, Intermediate 163 (2 g, 33%); [α]_{D}²⁰+152 (c 1.00, MeCN); ¹H NMR (400 MHz, DMSO-d₆) δ 2.07 (3H, d), 6.83 (1H, td), 6.97 (1H, d), 7.06 (1H, d), 7.64 (1H, dd), 8.04 (1H, dd), 8.74 (1H, d).

The absolute configuration of Intermediate 163 was determined by vibrational circular dichroism (VCD) spectroscopy. The experimental spectrum recorded in CDCl₃ was compared to a simulated spectrum of the (S) enantiomer calculated using density functional theory at the B3PW91/cc-pVTZ level of theory. Based on the large number of points of agreement between the experimental and simulated spectra, the title compound was assigned as the (S) enantiomer.

### Intermediate 164

### rac-2-Benzyl 5-(tert-butyl) (1R,6R)-2,5-diazabicyclo[4.2.0]octane-2,5-dicarboxylate

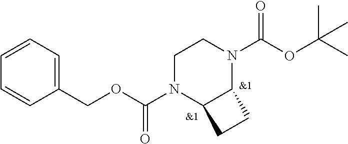 [SMILES: CC(C)(C)OC(=O)N1CCN(C(=O)OCc2ccccc2)[C@@H]2CC[C@H]21]

TEA (17.51 g, 0.1731 mol), followed by a solution of benzyl (2,5-dioxopyrrolidin-1-yl) carbonate (28.4 g, 0.1154 mol) in dry DCM (100 mL) was added to a solution of rac-tert-butyl (1R,6R)-2,5-diazabicyclo[4.2.0]octane-2-carboxylate (24.5 g, 0.1154 mol) in dry DCM (100 mL) and the reaction mixture was stirred at rt overnight. The reaction mixture was diluted with DCM (50 mL), washed with 10% citric acid (50 mL), sat NaHCO₃, (50 mL) and brine (100 mL). The organic layer was dried over anhydrous Na₂SO₄, filtered and concentrated under reduced pressure. The crude compound was purified by column chromatography (hexane:EtOAc, 1:1) to give the title compound (39 g, 98%); MS (ESI) m z [[(M −Boc)+H]⁺247.2; ¹H NMR (500 MHz, CDCl₃) δ 7.44-7.23 (m, 5H), 5.20-4.98 (m, 2H), 3.83-3.52 (m, 4H), 3.42 (dtt, 2H), 2.20 (d, 2H), 1.78 (s, 2H), 1.45 (d, 9H).

### Intermediate 165

### rel-2-Benzyl 5-(tert-butyl) (1R,6R)-2,5-diazabicyclo[4.2.0]octane-2,5-dicarboxylate Isomer 1

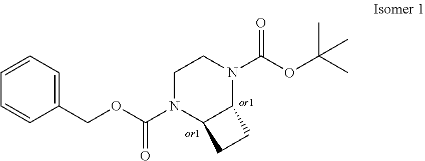 [SMILES: CC(C)(C)OC(=O)N1CCN(C(=O)OCc2ccccc2)[C@@H]2CC[C@H]21]

### Intermediate 166

### rel-2-Benzyl 5-(tert-butyl) (1R,6R)-2,5-diazabicyclo[4.2.0]octane-2,5-dicarboxylate Isomer 2

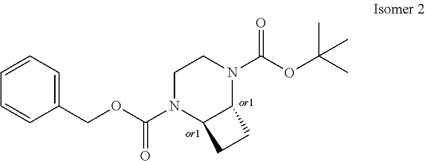 [SMILES: CC(C)(C)OC(=O)N1CCN(C(=O)OCc2ccccc2)[C@@H]2CC[C@H]21]

The stereoisomers of rac-2-benzyl 5-(tert-butyl) (1R,6R)-2,5-diazabicyclo[4.2.0]octane-2,5-dicarboxylate Intermediate 164 were separated by chiral chromatography on a CHIRALPAK IA column (250×30 mm, 5 μm), eluted with hexane:IPA:MeOH (80:10:10), at a flow rate of 40 mL/min;

The first eluted compound was collected and evaporated to give the title compound Isomer 1, Intermediate 165 (15.7 g); MS (ESI) m/z [[(M −Boc)+H]⁺247.4; and
the second eluted compound was collected and evaporated to give the title compound Isomer 2, Intermediate 166 (14.9 g); MS (ESI) m/z [[(M −Boc)+H] 247.2.

### Intermediate 167

### tert-Butyl (1R,6R)-2,5-diazabicyclo[4.2.0]octane-2-carboxylate

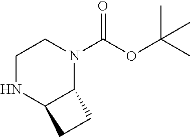

A solution of rel-2-benzyl 5-(tert-butyl) (1R,6R)-2,5-diazabicyclo[4.2.0]octane-2,5-dicarboxylate Isomer 2 Intermediate 166_(14.9 g 0.043 mol, retrospectively) and 10% Pd/C (1.4 g) in MeOH (100 mL) was evacuated and then purged with H₂(g) (×3). The reaction mixture was stirred at rt under pressure of H₂(g) (2 atm) until complete reaction (monitoring is by NMR). The catalyst was carefully removed by filtration and washed with MeOH (2×20 mL). The filtrate was concentrated in vacuo to give the title compound Intermediate 167 (8.8 g, 96%); [α]_{D}²⁰+6.18 (c 0.5, MeOH); ¹H NMR (400 MHz, CDCl₃) δ 3.66 (ddd, 1H), 3.08-2.93 (m, 1H), 2.87 (pt, 2H), 2.77-2.48 (m, 3H), 2.15 (q, 1H), 2.01-1.78 (m, 2H), 1.55 (ddd, 1H), 1.39-1.26 (m, 9H).

The absolute configuration of the title compound was determined by converting the title compound in two steps to the 4-chlorobenzoyl derivative Intermediate 169 as described below in the experimental descriptions for Intermediate 168 and Intermediate 169. Based on the X-ray data of Intermediate 169 the title compound Intermediate 167 was assigned as the (1R,6R) enantiomer.

### Intermediate 168

### rel-2-(tert-Butyl) 5-(4-chlorobenzyl) (1R,6R)-2,5-diazabicyclo[4.2.0]octane-2,5-dicarboxylate Isomer 2

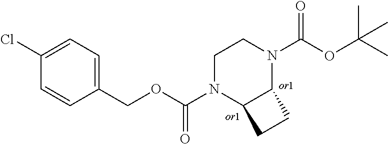 [SMILES: CC(C)(C)OC(=O)N1CCN(C(=O)OCc2ccc(Cl)cc2)[C@@H]2CC[C@H]21]

TEA (214 mg, 2.1 mmol), followed by a solution of 4-chlorobenzyl carbonochloridate (241 mg, 1.4 mmol) in dry DCM (15 mL), was added to a solution of tert-butyl (1R,6R)-2,5-diazabicyclo[4.2.0]octane-2-carboxylate Intermediate 167 (250 mg, 1.4 mmol) in dry DCM (5 mL) and the reaction mixture was stirred at rt overnight. The reaction mixture was diluted with DCM (5 mL) and washed with 10% citric acid (10 mL), sat NaHCO₃ (20 mL) and brine (10 mL). The organic layer was dried over anhydrous Na₂SO₄, filtered and concentrated at reduced pressure. The crude product was purified by column (hexane:MTBE, 1:1) to give the title compound (335 mg, 82%); MS (ESI) m/z [(M-Boc)+H]⁺281.2

### Intermediate 169

### 4-Chlorobenzyl (1R,6R)-2,5-diazabicyclo[4.2.0]octane-2-carboxylate

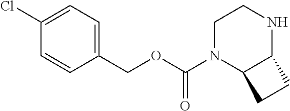 [SMILES: O=C(OCc1ccc(Cl)cc1)N1CCN[C@@H]2CC[C@H]21]

2 M HCl in Et₂O (2 mL) was added to a solution rel-2-(tert-butyl) 5-(4-chlorobenzyl) (1R,6R)-2,5-diazabicyclo[4.2.0]octane-2,5-dicarboxylate Isomer 2 Intermediate 168 (95 mg, 0.25 mmol) in DCM (10 mL) and the reaction mixture was stirred at ambient temperature for 8 h. The reaction mixture was concentrated under reduced pressure to give the hydrochloride of the title compound (80 mg, quantitative yield) as a white solid; MS (ESI) m/z 281.0.

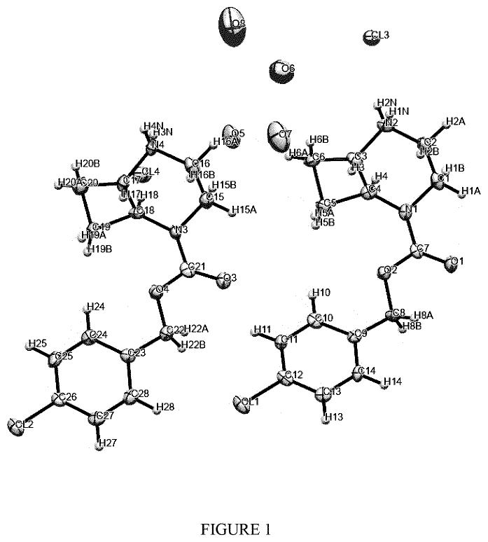

Crystals for X-ray diffraction studies was grown from acetonitrile. The molecular structure of Intermediate 169 is shown in FIG. 1.

Crystallographic data: C₁₄H₁₈ClN₂O₂, Cl, 2×(H₂O), M=380.87, monoclinic, space group P21, Cell Lengths: a=16.437(3), b=7.0509(10), c=14.574(2) A, cell angles: a 90 R 99.923(8) γ 90, cell volume: V=1663.8, crystal size ca. 0.11×0.25×0.49 mm, R-factor (%) 7.62.

### Intermediate 170

### tert-Butyl (1R,6R)-5-((S)-2-(5-chloropyridin-2-yl)-2-methylbenzo[d][1,3]dioxol-4-yl)-2,5-diazabicyclo[4.2.0]octane-2-carboxylate

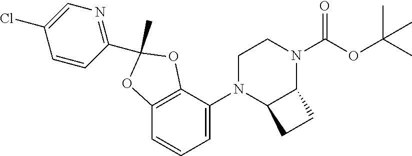 [SMILES: CC(C)(C)OC(=O)N1CCN(c2cccc3c2O[C@@](C)(c2ccc(Cl)cn2)O3)[C@@H]2CC[C@H]21]

A mixture of (S)-2-(4-bromo-2-methylbenzo[d][1,3]dioxol-2-yl)-5-chloropyridine Intermediate 163 (12.36 g, 37.86 mmol), tert-butyl (1R,6R)-2,5-diazabicyclo[4.2.0]octane-2-carboxylate Intermediate 167 (8.461 g, 37.86 mmol), Palladacycle Gen 4 (2.59 g, 2.27 mmol), and Cs₂CO₃ (24.67 g, 75.73 mmol) in a sealed flask was evacuated and backfilled with N₂(g) (×3). 1,4-Dioxane (60 mL) was degassed by N₂(g) bubbling for 15 min, and then added. The reaction mixture was evacuated and backfilled with N₂(g) (×3) and then stirred at 90° C. for 20 h. The reaction mixture was cooled to rt, diluted with EtOAc (100 mL) and washed with brine (2×50 mL). The organic layer was dried over MgSO₄, filtered, and concentrated at reduced pressure. The crude product was purified by flash chromatography on silica (0-12% EtOAc in heptane). The product was dissolved in EtOAc (100 mL), SiliaMetS Thiol (3 g, 40-63 μm) was added and the mixture was stirred at rt for 2 h, then filtered. The is filtrate was collected and concentrated at reduced pressure to give the title compound (10.80 g, 62.3%); [α]_{D}²⁰+79 (c 1.0, MeCN); MS (ESI) m/z [M+H]⁺ 458.3.

### Intermediate 171 (1R,6R)-2-((S)-2-(5-Chloropyridin-2-yl)-2-methylbenzo[d][1,3]dioxol-4-yl)-2,5-diazabicyclo[4.2.0]octane

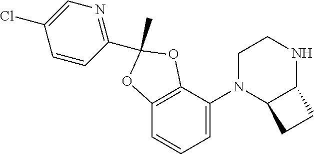 [SMILES: C[C@]1(c2ccc(Cl)cn2)Oc2cccc(N3CCN[C@@H]4CC[C@H]43)c2O1]

pTsOH hydrate (9.87 g, 51.88 mmol) was added to a solution of tert-butyl (1R,6R)-5-((S)-2-(5-chloropyridin-2-yl)-2-methylbenzo[d][1,3]dioxol-4-yl)-2,5-diazabicyclo[4.2.0]octane-2-carboxylate Intermediate 170 (10.8 g, 23.58 mmol) in EtOAc (80 mL) and the reaction mixture was stirred at 42° C. for 18 h. The reaction mixture was cooled to rt, diluted with EtOAc (50 mL) and sat K₂CO₃ (aq, 10 mL) was added drop wise. The organic layer was washed with sat K₂CO₃ (aq, 3×25 mL), dried over MgSO₄, filtered and concentrated at reduced pressure to give the title compound (8.30 g, 98%); MS (ESI) m/z [M+H]⁺ 358.2.

### Intermediate 172

### Methyl 4-chloro-2-(((1R,6R)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate

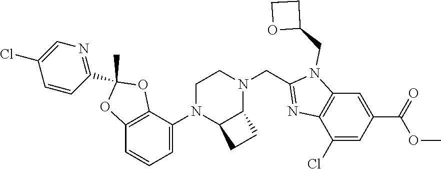 [SMILES: COC(=O)c1cc(Cl)c2nc(CN3CCN(c4cccc5c4O[C@@](C)(c4ccc(Cl)cn4)O5)[C@@H]4CC[C@H]43)n(C[C@@H]3CCO3)c2c1]

K₂CO₃ (9.27 g, 67.07 mmol) and methyl (S)-4-chloro-2-(chloromethyl)-1-(oxetan-2-ylmethyl)-1H-benzo[d]imidazole-6-carboxylate Intermediate 24 (7.73 g, 23.47 mmol) were added to a solution of (1R,6R)-2-((S)-2-(5-chloropyridin-2-yl)-2-methylbenzo[d][1,3]dioxol-4-yl)-2,5-diazabicyclo[4.2.0]octane Intermediate 171 (8 g, 22.36 mmol) in MeCN (50 mL) and the reaction mixture was stirred at 53° C. for 24 h. The reaction mixture was cooled to rt and EtOAc (100 mL) was added. The organic layer was washed with NaHCO₃ (aq, 2×50 mL), is dried over MgSO₄, filtered and concentrated at reduced pressure. The crude compound was purified by preparative HPLC, PrepMethod M, (gradient: 50-100%). Relevant fractions were pooled and most of the MeCN was evaporated and the residue was extracted with EtOAc (2×30 mL). The combined organic layer was dried over MgSO₄, filtered, and the filtrate was stirred with SiliaMetS Thiol (6 g, 40-63 μm) at rt for 2 h. The mixture was filtered and the filtrate was concentrated at reduced pressure. The residue was purified by flash chromatography (50-100% EtOAc in heptane) and the product containing fractions were pooled and concentrated at reduced pressure. MeOH (300 mL) was added to the residue and the mixture was stirred at rt for 30 min whereupon a solid formed. The mixture was cooled to 0° C., and the solid was isolated by filtration, rinsed with several portions of cooled MeOH (10 mL) and finally dried in vacuo to give the title compound (9.59 g, 65.9%) as a white solid; [α]_{D}²⁰+49 (c 1.0, MeCN); MS (ESI) m/z [M+H]⁺ 652.35.

### EXAMPLES

### Example 1a

### 2-((1R*,6S*)-5-(R*)-2-(5-Chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid, Isomer 2

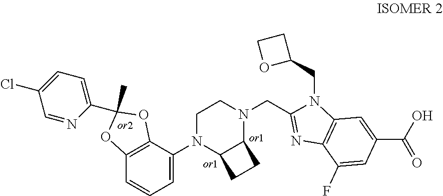 [SMILES: C[C@]1(c2ccc(Cl)cn2)Oc2cccc(N3CCN(Cc4nc5c(F)cc(C(=O)O)cc5n4C[C@@H]4CCO4)[C@H]4CC[C@H]43)c2O1]

A mixture of methyl 2-(((1R*,6S*)-5-((S*)-2-(5-chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 2 Intermediate 13 (106 mg, 0.17 mmol) and 1,3,4,6,7,8-hexahydro-2H-pyrimido[1,2-a]pyrimidine (61 mg, 0.44 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight.

The reaction mixture was concentrated in vacuo, and the residue was dissolved in DMSO, is filtered and purified by preparative HPLC, PrepMethod B, (gradient: 5-95%), to give the title compound (0.070 g, 67%); HRMS (ESI) m/z [M+H]⁺ calcd for C₃₂H₃₂ClFN₅O₅: 620.2070, found: 620.2098; ¹H NMR (600 MHz, DMSO-d₆) 1.51-1.57 (1H, m), 1.63-1.74 (1H, m), 1.85-1.91 (1H, m), 1.96 (3H, s), 2.14-2.2 (1H, m), 2.31-2.41 (2H, m), 2.61-2.73 (2H, m), 3.15-3.27 (3H, m), 3.28-3.34 (1H, m), 3.73 (1H, d), 4.15 (1H, d), 4.19-4.26 (2H, m), 4.42-4.48 (1H, m), 4.75 (1H, dd), 4.81-4.87 (1H, m), 5.13-5.2 (1H, m), 6.31 (1H, dd), 6.45 (1H, dd), 6.71 (1H, t), 7.49-7.55 (1H, m), 7.60 (1H, dd), 7.94-7.98 (1H, m), 8.14-8.17 (1H, m), 8.69 (1H, d).

### Example 1b

### 2-(((1R*,6S*)-5-((R*)-2-(5-Chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid. Isomer 3

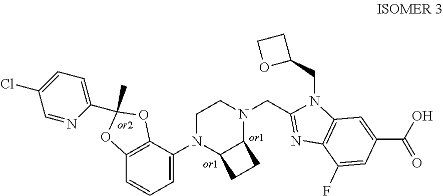 [SMILES: C[C@]1(c2ccc(Cl)cn2)Oc2cccc(N3CCN(Cc4nc5c(F)cc(C(=O)O)cc5n4C[C@@H]4CCO4)[C@H]4CC[C@H]43)c2O1]

A mixture of methyl 2-(((1R*,6S*)-5-((S*)-2-(5-chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 3 Intermediate 14 (138 mg, 0.22 mmol) and 1,3,4,6,7,8-hexahydro-2H-pyrimido[1,2-a]pyrimidine (61 mg, 0.44 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The reaction mixture was concentrated in vacuo, and the residue was dissolved in DMSO, filtered and purified by preparative HPLC, PrepMethod B, (gradient: 5-95%), to give the title compound (0.074 g, 54%); HRMS (ESI) m/z [M+H]⁺ calcd for C₃₂H₃₂ClFN₅O₅: 620.2070, found: 620.2086 ¹H NMR (600 MHz, DMSO-d₆) 1.52-1.64 (3H, m), 1.99 (4H, s), 2.3-2.41 (2H, m), 2.61-2.69 (1H, m), 2.7-2.76 (1H, m), 3.1-3.16 (1H, m), 3.17 (1H, s), 3.24-3.3 (2H, m), 3.77 (1H, d), 4.11 (1H, d), 4.21-4.28 (2H, m), 4.41-4.48 (1H, m), 4.71-4.84 (2H, m), is 5.11-5.18 (1H, m), 6.25-6.3 (1H, m), 6.44-6.48 (1H, m), 6.71 (1H, t), 7.5-7.55 (1H, m), 7.58 (1H, d), 7.95-8 (1H, m), 8.14-8.18 (1H, m), 8.67-8.71 (1H, m).

### Example 1c

2-((1R*,6S*)-5-(R*)-2-(5-Chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid, Isomer 4

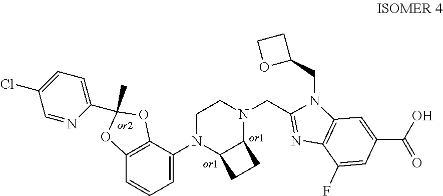 [SMILES: C[C@]1(c2ccc(Cl)cn2)Oc2cccc(N3CCN(Cc4nc5c(F)cc(C(=O)O)cc5n4C[C@@H]4CCO4)[C@H]4CC[C@H]43)c2O1]

A mixture of methyl 2-(((1R*,6S*)-5-((S*)-2-(5-chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 4 Intermediate 15 (125 mg, 0.20 mmol) and 1,3,4,6,7,8-hexahydro-2H-pyrimido[1,2-a]pyrimidine (55 mg, 0.39 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt for 90 min. The reaction mixture was concentrated in vacuo, and the residue was dissolved in DMSO. Acetic acid (0.5 mL) was added and the mixture was purified by preparative HPLC, PrepMethod A, (gradient: 30-60%), to give the title compound (0.059 g, 49%); HRMS (ESI) m/z [M+H]⁺ calcd for C₃₂H₃₂ClFN₅O₅: 620.2070, found: 620.2100; ¹H NMR (500 MHz, DMSO-d₆) 1.69-1.83 (2H, m), 1.93-2.04 (4H, m), 2.19-2.29 (1H, m), 2.32-2.41 (2H, m), 2.43-2.48 (1H, m), 2.6-2.74 (3H, m), 3.14-3.22 (1H, m), 3.61 (1H, d), 4.21 (1H, d), 4.24-4.31 (1H, m), 4.38-4.47 (1H, m), 4.48-4.57 (1H, m), 4.59-4.72 (1H, m), 4.89-5 (1H, m), 5.05-5.16 (1H, m), 6.32-6.37 (1H, m), 6.44-6.49 (1H, m), 6.73 (1H, t), 7.48-7.54 (1H, m), 7.62 (1H, d), 8.00 (1H, dd), 8.16 (1H, s), 8.71 (1H, d).

### Example 2a

### 2-((1R*,6S*)-5-(R*)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid, Isomer 1

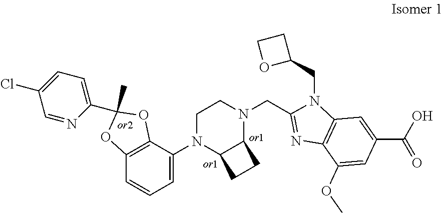 [SMILES: COc1cc(C(=O)O)cc2c1nc(CN1CCN(c3cccc4c3O[C@@](C)(c3ccc(Cl)cn3)O4)[C@@H]3CC[C@@H]31)n2C[C@@H]1CCO1]

A mixture of methyl 2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 1 Intermediate 17 (33 mg, 0.05 mmol) and 1,3,4,6,7,8-hexahydro-2H-pyrimido[1,2-a]pyrimidine (14 mg, 0.10 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The mixture was concentrated at reduced pressure, and the residue was purified by preparative HPLC, PrepMethod A, (gradient: 20-90%), to give the title compound (13 mg, 41%); HRMS (ESI) m/z [M+H]⁺ calcd for C₃₃H₃₅ClN₅O₆: 632.2270, found: 632.2292; ¹H NMR (500 MHz, CD₃OD) 1.6-1.68 (1H, m), 1.69-1.76 (2H, m), 2.02 (3H, s), 2.09 (1H, p), 2.39-2.49 (2H, m), 2.69-2.82 (2H, m), 3.13-3.19 (1H, m), 3.37-3.45 (1H, m), 3.82 (1H, d), 4.04 (3H, s), 4.20 (1H, d), 4.26-4.35 (2H, m), 4.55-4.63 (1H, m), 4.72 (1H, dd), 4.87-4.93 (1H, m), 5.21-5.33 (1H, m), 6.31 (1H, dd), 6.44 (1H, dd), 6.72 (1H, t), 7.45 (1H, d), 7.64 (1H, dd), 7.86 (1H, dd), 7.95 (1H, d), 8.11 (1H, s), 8.59 (1H, dd).

### Example 2b

### 2-(((R-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid, Isomer 2

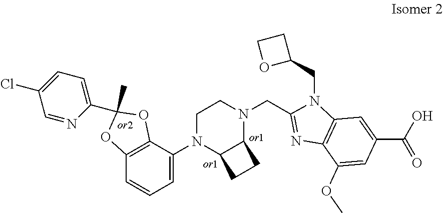 [SMILES: COc1cc(C(=O)O)cc2c1nc(CN1CCN(c3cccc4c3O[C@@](C)(c3ccc(Cl)cn3)O4)[C@@H]3CC[C@@H]31)n2C[C@@H]1CCO1]

A mixture of methyl 2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 2 Intermediate 18 (29 mg, 0.04 mmol) and 1,3,4,6,7,8-hexahydro-2H-pyrimido[1,2-a]pyrimidine (11 mg, 0.08 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight.

The mixture was concentrated at reduced pressure, and the residue was purified by preparative HPLC, PrepMethod A, (gradient: 20-90%) to give the title compound (13 mg, 21%); HRMS (ESI) m/z [M+H]⁺ calcd for C₃₃H₃₅ClN₅O₆: 632.2270, found: 632.2280; ¹H NMR (500 MHz, CD₃OD) 1.69-1.81 (2H, m), 1.87-1.95 (1H, m), 2.00 (3H, s), 2.16-2.27 (1H, m), 2.4-2.51 (2H, m), 2.7-2.81 (2H, m), 3.24-3.3 (2H, m), 3.34-3.42 (1H, m), 3.81 (1H, d), 4.05 (3H, s), 4.23 (1H, d), 4.27-4.37 (2H, m), 4.54-4.63 (1H, m), 4.73 (1H, dd), 4.93 (1H, dd), 5.24-5.33 (1H, m), 6.31-6.37 (1H, m), 6.42 (1H, dd), 6.72 (1H, t), 7.45 (1H, d), 7.65 (1H, dd), 7.87 (1H, dd), 7.96 (1H, d), 8.10 (1H, s), 8.57-8.61 (1H, m).

### Example 2c

### 2-(((R-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid, Isomer 3

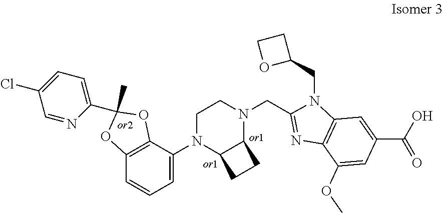 [SMILES: COc1cc(C(=O)O)cc2c1nc(CN1CCN(c3cccc4c3O[C@@](C)(c3ccc(Cl)cn3)O4)[C@@H]3CC[C@@H]31)n2C[C@@H]1CCO1]

A mixture of methyl 2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 3 Intermediate 19 (58 mg, 0.09 mmol) and 1,3,4,6,7,8-hexahydro-2H-pyrimido[1,2-a]pyrimidine (25 mg, 0.18 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The mixture was concentrated at reduced pressure, and the residue was purified by preparative HPLC, PrepMethod A, (gradient: 20-90%), to give the title compound (22 mg, is 39%); HRMS (ESI) m/z [M+H]⁺ calcd for C₃₃H₃₅ClN₅O₆: 632.2270, found: 632.2278; ¹H NMR (500 MHz, CD₃OD) 1.65-1.72 (1H, m), 1.76-1.91 (2H, m), 2.02 (3H, s), 2.1-2.2 (1H, m), 2.43-2.59 (2H, m), 2.72-2.83 (2H, m), 3.14-3.22 (1H, m), 3.25-3.3 (1H, m), 3.35-3.43 (1H, m), 3.75 (1H, d), 4.03 (3H, s), 4.21 (1H, d), 4.34 (1H, q), 4.45-4.53 (1H, m), 4.57-4.69 (2H, m), 5.00 (1H, dd), 5.2-5.29 (1H, m), 6.32 (1H, dd), 6.44 (1H, dd), 6.72 (1H, t), 7.44 (1H, d), 7.65 (1H, dd), 7.86 (1H, dd), 7.96 (1H, d), 8.09 (1H, s), 8.60 (1H, dd).

### Example 2d

### 2-((1R*,6S*)-5-(R*)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid. Isomer 4

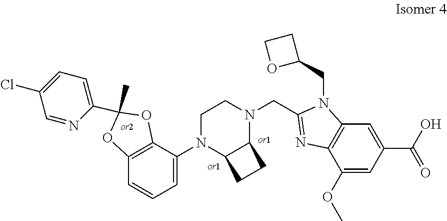 [SMILES: COc1cc(C(=O)O)cc2c1nc(CN1CCN(c3cccc4c3O[C@@](C)(c3ccc(Cl)cn3)O4)[C@@H]3CC[C@@H]31)n2C[C@@H]1CCO1]

A mixture of methyl 2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 4 Intermediate 20 (47 mg, 0.07 mmol) and 1,3,4,6,7,8-hexahydro-2H-pyrimido[1,2-a]pyrimidine (20 mg, 0.15 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The mixture was concentrated at reduced pressure, and the residue was purified by preparative HPLC, PrepMethod A, (gradient: 20-90%), to give the title compound (16 mg, 35%); HRMS (ESI) m/z [M+H]⁺ calcd for C₃₃H₃₅ClN₅O₆: 632.2270, found: 632.2280; ¹H NMR (500 MHz, CD₃OD) 1.81-1.89 (2H, m), 1.91-1.99 (1H, m), 2.00 (3H, s), 2.2-2.33 (1H, m), 2.41-2.5 (1H, m), 2.5-2.61 (1H, m), 2.7-2.83 (2H, m), 3.25-3.3 (2H, m), 3.32-3.4 (1H, m), 3.73 (1H, d), 4.03 (3H, s), 4.23 (1H, d), 4.35 (1H, q), 4.46-4.54 (1H, m), 4.57-4.7 (2H, is m), 5.02 (1H, dd), 5.2-5.3 (1H, m), 6.34 (1H, dd), 6.42 (1H, dd), 6.72 (1H, t), 7.44 (1H, d), 7.61-7.67 (1H, m), 7.84 (1H, dd), 7.96 (1H, d), 8.10 (1H, s), 8.58 (1H, dd).

### Example 3a

### 4-Chloro-2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid, Isomer 1

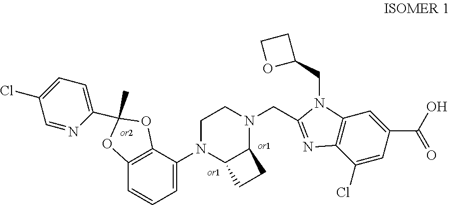 [SMILES: C[C@]1(c2ccc(Cl)cn2)Oc2cccc(N3CCN(Cc4nc5c(Cl)cc(C(=O)O)cc5n4C[C@@H]4CCO4)[C@H]4CC[C@@H]43)c2O1]

A mixture of methyl 4-chloro-2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 1 Intermediate 28 (49 mg, 0.08 mmol) and 1,3,4,6,7,8-hexahydro-2H-pyrimido[1,2-a]pyrimidine (21 mg, 0.15 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The reaction mixture was concentrated in vacuo, and the residue was dissolved in DMSO, filtered and purified by preparative HPLC, PrepMethod A, (gradient: 20-90%) to give the title compound Isomer 1 (0.023 g, 48%); IRMS (ESI) m/z [M+H]⁺ calcd for C₃₂H₃₂Cl₂N₅O₅: 636.1774, found: 636.1818; ¹H NMR (500 MHz, CD₃OD) 1.44-1.54 (1H, m), 1.74-1.87 (2H, m), 2.01 (3H, s), 2.17-2.25 (1H, m), 2.43-2.64 (3H, m), 2.7-2.9 (3H, m), 2.96-3.03 (1H, m), 3.84-4.08 (3H, m), 4.41-4.49 (1H, m), 4.6-4.68 (1H, m), 4.87-4.92 (overlapping with solvent peak, m), 5.21-5.3 (1H, m), 6.39 (1H, dd), 6.49-6.54 (1H, m), 6.74 (1H, t), 7.66-7.71 (1H, is m), 7.88 (1H, dd), 7.94 (1H, d), 8.27 (1H, d), 8.6-8.63 (1H, m).

### Example 3b

### 4-Chloro-2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid, Isomer 2

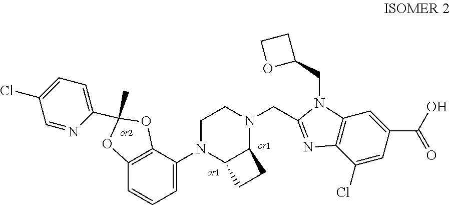 [SMILES: C[C@]1(c2ccc(Cl)cn2)Oc2cccc(N3CCN(Cc4nc5c(Cl)cc(C(=O)O)cc5n4C[C@@H]4CCO4)[C@H]4CC[C@@H]43)c2O1]

A mixture of methyl 4-chloro-2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 2 Intermediate 29 (150 mg, 0.23 mmol) and 1,3,4,6,7,8-hexahydro-2H-pyrimido[1,2-a]pyrimidine (64 mg, 0.46 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The reaction mixture was concentrated in vacuo, and the residue was dissolved in DMSO, filtered and purified by preparative HPLC, PrepMethod A, (gradient: 20-90%) to give the title compound (0.108 g, 74%); HRMS (ESI) m/z [M+H]⁺ calcd for C₃₂H₃₂Cl₂N₅O₅: 636.1774, found: 636.1788; ¹H NMR (500 MHz, CD₃OD) 1.44-1.54 (1H, m), 1.78-1.88 (2H, m), 1.99 (3H, s), 2.19-2.28 (1H, m), 2.42-2.54 (2H, m), 2.54-2.67 (2H, m), 2.72-2.85 (2H, m), 2.91-2.97 (1H, m), 3.81-3.88 (2H, m), 4.04 (1H, d), 4.43 (1H, dt), 4.56-4.64 (1H, m), 4.78 (1H, dd), 4.82-4.92 (overlapping with solvent peak, m), 5.17-5.25 (1H, m), 6.32-6.38 (1H, m), 6.47-6.53 (1H, m), 6.72 (1H, t), 7.60 (1H, d), 7.80 (1H, dd), 7.92 (1H, d), 8.24 (1H, d), 8.54 (1H, d).

### Example 3b, Alternative Preparation

### 4-Chloro-2-(((1R,6R)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6

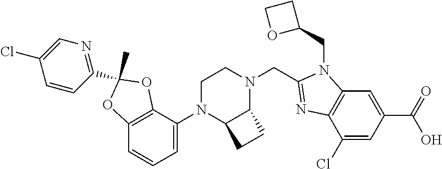 [SMILES: C[C@]1(c2ccc(Cl)cn2)Oc2cccc(N3CCN(Cc4nc5c(Cl)cc(C(=O)O)cc5n4C[C@@H]4CCO4)[C@@H]4CC[C@H]43)c2O1]

A solution of LiOH (0.953 g, 39.80 mmol) in water (40 mL) was added dropwise to a solution of methyl 4-chloro-2-(((1R,6R)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate Intermediate 172 (9.59 g, 14.74 mmol) in THE (80 mL) and the reaction mixture was stirred at rt for 4 h. The reaction mixture was concentrated at reduced pressure and the solid was suspended in water (200 mL). EtOAc (100 mL) was added and the pH was adjusted to pH 4.5 by addition of 1 M citric acid. The phases were separated and the aqueous layer was extracted with EtOAc (2×100 mL). The combined organic layer was washed with water (2×20 mL), dried over MgSO₄, filtered and concentrated under reduced pressure. The white solid was lyophilized for 72 h to give the title compound (8.80 g, 94%) as a white solid; HRMS (ESI) m/z [M+H]⁺ calcd for C₃₂H₃₂Cl₂N₅O₅: 636.1774, found: 636.1786; ¹H NMR (500 MHz, DMSO) δ 1.42-1.52 (1H, m), 1.7-1.86 (2H, m), 2.21-2.29 (1H, m), 2.34-2.49 (4H, m), 2.52-2.6 (1H, m), 2.68-2.78 (2H, m), 2.82-2.88 (1H, m), 3.26-3.33 (2H, m), 3.66 (1H, d), 3.77-3.83 (1H, m), 3.98-4.04 (1H, m), 4.35-4.43 (1H, m), 4.46-4.53 (1H, m), 4.7-4.77 (1H, m), 4.84-4.92 (1H, m), 5.04-5.13 (1H, m), 6.31-6.36 (1H, m), 6.5-6.56 (1H, m), 6.72 (1H, t), 7.59 (1H, d), 7.79 (1H, d), 7.92-7.98 (1H, m), 8.24-8.27 (1H, m), 8.69 (1H, d).

Single clear colorless blocked-shaped crystal of Example 3b was recrystallized from methyl tertiary-butyl ether. A suitable single crystal was selected and mounted on a mitigen sample holder (Mitegen, USA) in perflouroether oil. X-ray diffraction data was collected at 100 K using Cryostream 800 (Oxford Cryosystem, UK) in co-scan mode with XtaLab Synergy-S (Rigaku, Japan) equipped with Cu Kα micro focus source (50 kV, 0.01 mA) and Hypix-Arc 100 detector. The diffraction pattern was initially indexed and the total number of runs and images was based on the strategy calculation from the program CrysAlisPro 1.171.42.46a (Rigaku, Japan). Data reduction, scaling and absorption corrections were performed using CrysAlisPro 1.171.42.46a (Rigaku, Japan). The integrated and scaled data were corrected is using numerical absorption correction based on gaussian integration over a multifaceted crystal model and empirical absorption correction using spherical harmonics, implemented in SCALE3 ABSPACK scaling algorithm.

The structure was solved by the ShelXT (Acta Cryst. C71 (2015) 3-8) structure solution program using using dual methods and refined by full matrix least squares minimisation on F² using version 2018/3 of ShelXL 2018/3 (Acta Cryst. C71 (2015) 3-8) within Olex2 (J. Appl. Cryst. 42 (2009) 339-431). All non-hydrogen atoms were refined anisotropically. All hydrogen atoms attached to oxygen atoms were located from the differential Fourier map and were refined isotropically. All other hydrogen atoms were determined geometrically and refined isotropically.

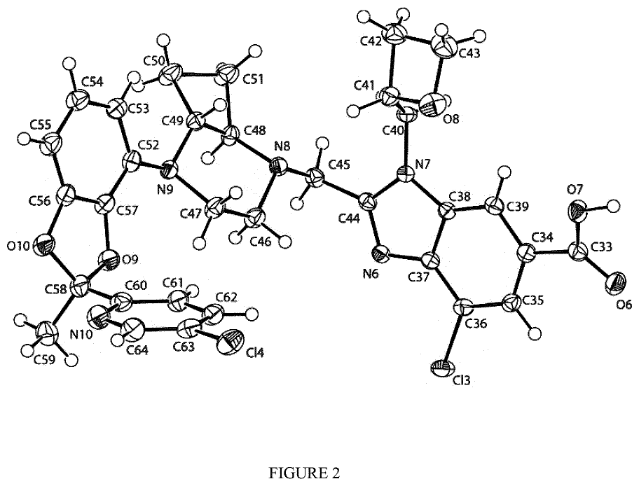

Crystallographic data is listed below. Asymmetric unit contains two molecules of Example 3b and four molecules of methyl tertiary-butyl ether, therefore this crystal structure is Example 3b methyl tertiary-butyl ether disolvate. Flack (Acta Cryst. B69 (2013) 249-259) and Hooft (J. Appl. Cryst. 43 (2010) 665-668) parameters were found to be 0.002(3) and −0.006(3), respectively. Thermal ellipsoid drawing of Example 3b is shown in FIG. 2.

### Crystallographic Data of Example 3b Methyl Tertiary-Butyl Ether Disolvate

| Formula | C₃₂H₃₁Cl₂N₅O₅, 2(C₅H₁₂O) |
|---|---|
| Dcalc | 1.250 |
| m/mm⁻¹ | 1.785 |
| Formula Weight | 812.81 |
| Colour | clear colourless |
| Shape | block-shaped |
| Size/mm³ | 0.25 × 0.06 × 0.05 |
| T/K | 100.01(10) |
| Crystal System | orthorhombic |
| Space Group | P2₁2₁2₁ |
| a/Å | 11.38770(10) |
| b/Å | 16.51010(10) |
| c/Å | 45.9583(4) |
| V/Å³ | 8640.71(12) |
| Z | 8 |
| Z′ | 2 |
| θₘᵢₙ/° | 3.296 |
| θₘₐₓ/° | 77.791 |
| Measured Reflections | 124989 |
| Independent reflections | 18214 |
| Reflections I ≥ 2s(I) | 17157 |
| Rint | 0.0541 |
| Parameters | 1030 |
| Restraints | 9 |
| Largest Peak | 0.684 |
| Deepest Hole | −0.696 |
| GooF | 1.030 |
| wR2 (all data) | 0.1089 |
| wR2 | 0.1073 |
| R₁ (all data) | 0.0464 |
| R₁ | 0.0437 |

By comparison of retention times on chiral HPLC and biological assay data, the title compound 4-chloro-2-(((1R,6R)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid was concluded to be identical to 4-Chloro-2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid, Isomer 2.

### Example 3c

### 4-Chloro-2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid, Isomer 4

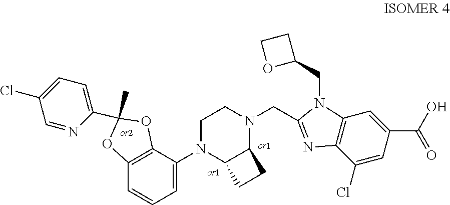 [SMILES: C[C@]1(c2ccc(Cl)cn2)Oc2cccc(N3CCN(Cc4nc5c(Cl)cc(C(=O)O)cc5n4C[C@@H]4CCO4)[C@H]4CC[C@@H]43)c2O1]

A mixture of methyl 4-chloro-2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 4 Intermediate 30 (68 mg, 0.10 mmol) and 1,3,4,6,7,8-hexahydro-2H-pyrimido[1,2-a]pyrimidine (29 mg, 0.21 mmol) in a sealed flask was evacuated and backfilled with N₂(g) (×3) three times. A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The reaction mixture was concentrated in vacuo, and the residue was dissolved in DMSO, filtered and purified by preparative HPLC, PrepMethod A, (gradient: 20-90%) to give the title compound (0.051 g, 77%); HRMS (ESI) m/z [M+H]⁺ calcd for C₃₂H₃₂Cl₂N₅O₅: 636.1774, found: 636.1816; ¹H NMR (500 MHz, CD₃OD) 1.24-1.35 (1H, m), 1.62-1.71 (1H, m), 1.7-1.82 (1H, m), 2.02 (3H, s), 2.12-2.2 (1H, m), 2.44-2.56 (2H, m), 2.63-2.72 (1H, m), 2.75-2.89 (3H, m), 3.11-3.18 (1H, m), 3.90 (1H, d), 3.93-4 (1H, m), 4.04 (1H, d), 4.46 (1H, dt), 4.6-4.67 (1H, m), 4.70 (1H, dd), 4.98 (1H, dd), 5.27-5.35 (1H, m), 6.35-6.41 (1H, m), 6.49-6.55 (1H, m), 6.74 (1H, t), 7.66-7.71 (1H, m), 7.88 (1H, dd), 7.94 (1H, d), 8.29 (1H, d), 8.58-8.63 (1H, m).

### Example 4a

### 2-(((1R*,6R*)-5-((R*)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid, Isomer 1

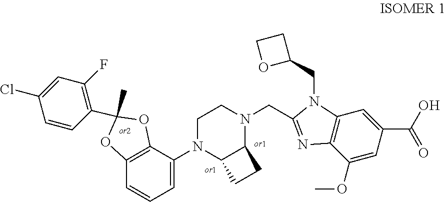 [SMILES: COc1cc(C(=O)O)cc2c1nc(CN1CCN(c3cccc4c3O[C@@](C)(c3ccc(Cl)cc3F)O4)[C@H]3CC[C@@H]31)n2C[C@@H]1CCO1]

A solution of methyl 2-(((1R*,6R*)-5-((R*)-2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 1 Intermediate 34 (85 mg, 0.13 mmol) and 1,3,4,6,7,8-hexahydro-2H-pyrimido[1,2-a]pyrimidine (43 mg, 0.31 mmol) in a mixture of MeCN (1 mL) and water (0.2 mL) was stirred at rt overnight. The reaction mixture was concentrated and the residue dissolved in DMSO, and purified by preparative HPLC, PrepMethod A, (gradient: 40-80%) to give the title compound (0.054 g, 65) as a white powder; HRMS (ESI) m/z [M+H]⁺ calcd for C₃₄H₃₅ClFN₄O₆: 649.2224, found: 649.2246; ¹H NMR (500 MHz, CD₃OD) 1.41-1.58 (1H, m), 1.81-1.93 (2H, m), 2.03 (3H, s), 2.27 (1H, q), 2.42-2.63 (3H, m), 2.63-2.73 (2H, m), 2.77-2.9 (2H, m), 2.96 (1H, dt), 3.83 (1H, d), 3.88 (1H, dt), 4.01 (1H, d), 4.05 (3H, s), 4.45 (1H, dt), 4.64 (1H, q), 4.77-4.86 (2H, m), 5.2-5.33 (1H, m), 6.39 (1H, d), 6.54 (1H, d), 6.74 (1H, t), 7.16-7.24 (1H, m), 7.24-7.32 (1H, m), 7.46 (1H, d), 7.57 (1H, td), 7.95 (1H, d).

### Example 4b

### 2-(((1R*,6R*)-5-((R*)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid, Isomer 2

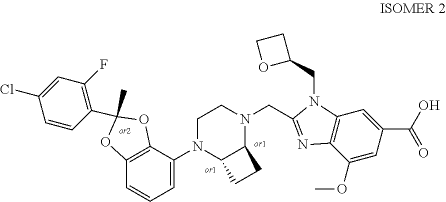 [SMILES: COc1cc(C(=O)O)cc2c1nc(CN1CCN(c3cccc4c3O[C@@](C)(c3ccc(Cl)cc3F)O4)[C@H]3CC[C@@H]31)n2C[C@@H]1CCO1]

A solution of methyl 2-(((1R*,6R*)-5-((R*)-2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 2 Intermediate 35 (30 mg, 0.05 mmol) and 1,3,4,6,7,8-hexahydro-2H-pyrimido[1,2-a]pyrimidine (19 mg, 0.14 mmol) in a mixture of MeCN (1 mL) and water (0.2 mL) was stirred at rt overnight. The reaction mixture was concentrated and the residue was dissolved in DMSO and purified by preparative HPLC, PrepMethod A, to give the title compound (0.023 g, 78%) as a white powder; HRMS (ESI) m/z [M+H]⁺ calcd for C₃₄H₃₅ClFN₄O₆: 649.2224, found: 649.2254; ¹H NMR (500 MHz, CD₃OD) 1.36-1.53 (1H, m), 1.73-1.89 (2H, m), 2.01 (3H, s), 2.13-2.25 (1H, m), 2.4-2.61 (3H, m), 2.65 (1H, s), 2.7-2.88 (3H, m), 2.98 (1H, dt), 3.83 (1H, d), 3.90 is (1H, dt), 3.98 (1H, d), 4.03 (3H, s), 4.43 (1H, dt), 4.63 (1H, td), 4.78-4.84 (2H, m), 5.18-5.33 (1H, m), 6.37 (1H, dd), 6.50 (1H, dd), 6.72 (1H, t), 7.20 (1H, dd), 7.27 (1H, dd), 7.44 (1H, d), 7.61 (1H, t), 7.94 (1H, d).

### Example 4c

### 2-(((1R*,6R*)-5-((R*)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid, Isomer 3

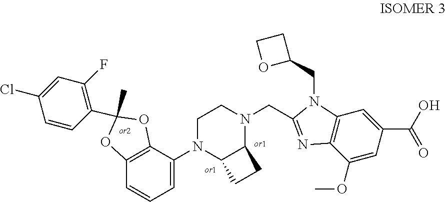 [SMILES: COc1cc(C(=O)O)cc2c1nc(CN1CCN(c3cccc4c3O[C@@](C)(c3ccc(Cl)cc3F)O4)[C@H]3CC[C@@H]31)n2C[C@@H]1CCO1]

A solution of methyl 2-(((1R*,6R*)-5-((R*)-2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 3 Intermediate 36 (28 mg, 0.04 mmol) and 1,3,4,6,7,8-hexahydro-2H-pyrimido[1,2-a]pyrimidine (18 mg, 0.13 mmol) in a mixture of MeCN (1 mL) and water (0.2 mL) was stirred at rt overnight. The reaction mixture was concentrated and the residue was dissolved in DMSO, and purified by preparative HPLC, PrepMethod A, to give the title compound (9.0 mg, 33%) as a white powder; HRMS (ESI) m/z [M+H]⁺ calcd for C₃₄H₃₅ClFN₄O₆: 649.2224, found: 649.2212; ¹H NMR (500 MHz, CD₃OD) 1.21-1.37 (1H, m), 1.67 (1H, dt), 1.79 (1H, qd), 2.02 (3H, s), 2.15 (1H, dt), 2.43 (1H, td), 2.47-2.56 (1H, m), 2.57-2.65 (1H, m), 2.66 (1H, s), 2.75-2.92 (3H, m), 3.09 (1H, dt), 3.83 (1H, d), 3.91-4.00 (2H, m), 4.03 (3H, s), 4.46 (1H, dt), 4.60-4.73 (2H, m), 4.95 (1H, dd), 5.30 (1H, qd), 6.37 (1H, dd), 6.50 (1H, dd), 6.72 (1H, t), 7.21 (1H, dd), 7.28 (1H, dd), 7.44 (1H, d), 7.62 (1H, t), 7.96 (1H, d).

### Example 5a

### 2-((1R*,6S*)-5-(R*)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid, Isomer 1

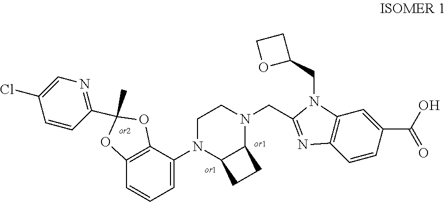 [SMILES: C[C@]1(c2ccc(Cl)cn2)Oc2cccc(N3CCN(Cc4nc5ccc(C(=O)O)cc5n4C[C@@H]4CCO4)[C@H]4CC[C@H]43)c2O1]

### Example 5b

### 2-((1R*,6S*)-5-(R*)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid. Isomer 2

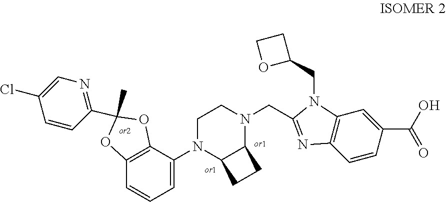 [SMILES: C[C@]1(c2ccc(Cl)cn2)Oc2cccc(N3CCN(Cc4nc5ccc(C(=O)O)cc5n4C[C@@H]4CCO4)[C@H]4CC[C@H]43)c2O1]

The stereoisomers of 2-(((1RS,6SR)-5-(2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid, Isomer 1 and Isomer 2 Intermediate 41 (115 mg) were separated by chiral chromatography on a Chiralcel OD column (250×20 mm, 5 μm), eluted with heptane/IPA (80/20), at a flow rate of 20 mL/min, and detected at 264 nm; the first eluted compound was collected and evaporated to give the title compound, Isomer 1, Example 5a (25 mg); HRMS (ESI) m/z [M+H]⁺ calcd for C₃₂H₃₃ClN₅O₅: 602.2164, found: 602.2180; ¹H NMR (500 MHz, CD₃OD) 1.61-1.68 (1H, m), 1.69-1.76 (2H, m), 2.02 (3H, s), 2.04-2.14 (1H, m), 2.41-2.52 (2H, m), 2.7-2.84 (2H, m), 3.13-3.19 (1H, m), 3.39-3.47 (1H, m), 3.85 (1H, d), 4.20 (1H, d), 4.26-4.38 (2H, m), 4.57-4.63 (1H, m), 4.77 (1H, dd), 4.94-4.87 (partly overlapping with solvent, m), 5.24-5.31 (1H, m), 6.32 (1H, dd), 6.44 (1H, dd), 6.72 (1H, t), 7.64 (1H, dd), 7.69 (1H, d), 7.86 (1H, dd), 7.98 (1H, dd), 8.32-8.35 (1H, m), is 8.59 (1H, dd).

the second eluted compound was collected and evaporated to give the title compound, Isomer 2, Example 5b (36 mg); MS (ESI) m/z [M+H]⁺ 602.4; ¹H NMR (500 MHz, CD₃OD) 1.68-1.81 (2H, m), 1.85-1.96 (1H, m), 1.99 (3H, s), 2.16-2.27 (1H, m), 2.41-2.51 (2H, m), 2.7-2.83 (2H, m), 3.23-3.3 (partly overlapping with solvent, m), 3.36-3.44 (1H, m), 3.84 (1H, d), 4.22 (1H, d), 4.26-4.38 (2H, m), 4.56-4.64 (1H, m), 4.77 (1H, dd), 4.93 (1H, dd), 5.25-5.33 (1H, m), 6.33 (1H, dd), 6.42 (1H, dd), 6.68-6.75 (1H, m), 7.6-7.67 (1H, m), 7.69 (1H, d), 7.85 (1H, dd), 7.99 (1H, dd), 8.32-8.36 (1H, m), 8.58 (1H, dd).

### Example 5c

### 2-((((R 2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid, Isomer 3

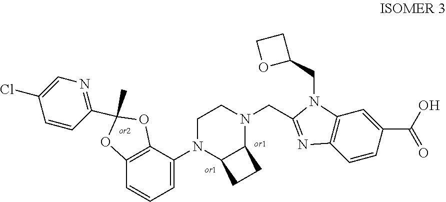 [SMILES: C[C@]1(c2ccc(Cl)cn2)Oc2cccc(N3CCN(Cc4nc5ccc(C(=O)O)cc5n4C[C@@H]4CCO4)[C@H]4CC[C@H]43)c2O1]

A mixture of methyl 2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 3 Intermediate 39 (70 mg, 0.11 mmol) and 1,3,4,6,7,8-hexahydro-2H-pyrimido[1,2-a]pyrimidine (32 mg, 0.23 mmol) in a sealed flask was evacuated and backfilled with N₂(g) (×3). A mixture of MeCN (3 mL) and water (0.6 mL) was degassed by N₂(g) bubbling for 15 min and added. The reaction mixture was evacuated and backfilled with N₂(g) (×3) and then stirred at rt overnight. The reaction mixture was concentrated at reduced pressure, and the residue was dissolved in DMSO, filtered and the crude compound was purified by preparative HPLC, PrepMethod A, (gradient: 20-90%) to give the title compound (0.050 g, 73%); HRMS (ESI) m/z [M+H]⁺ is calcd for C₃₂H₃₂ClN₅O₅: 602.2164, found: 602.2184; ¹H NMR (500 MHz, DMSO-d₆) 1.62-1.75 (3H, m), 2.01 (3H, s), 2.03-2.11 (1H, m), 2.31-2.4 (1H, m), 2.6-2.75 (2H, m), 3.13-3.2 (1H, m), 3.23 (2H, d), 3.29 (1H, s), 3.60 (1H, d), 4.17 (1H, d), 4.31 (1H, q), 4.38-4.46 (1H, m), 4.48-4.55 (1H, m), 4.57-4.64 (1H, m), 4.84-4.93 (1H, m), 5.05-5.13 (1H, m), 6.29-6.34 (1H, m), 6.45-6.5 (1H, m), 6.73 (1H, t), 7.58-7.67 (2H, m), 7.78-7.83 (1H, m), 8.02 (1H, dd), 8.24-8.28 (1H, m), 8.72 (1H, d).

### Example 5d

### 2-((1R*,6S*)-5-(R*)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid. Isomer 4

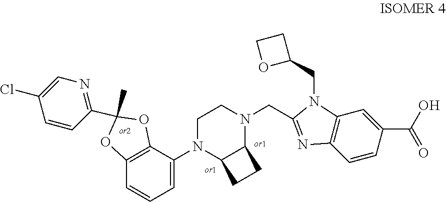 [SMILES: C[C@]1(c2ccc(Cl)cn2)Oc2cccc(N3CCN(Cc4nc5ccc(C(=O)O)cc5n4C[C@@H]4CCO4)[C@H]4CC[C@H]43)c2O1]

A mixture of methyl 2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 4 Intermediate 40 (70 mg, 0.11 mmol) and 1,3,4,6,7,8-hexahydro-2H-pyrimido[1,2-a]pyrimidine (32 mg, 0.23 mmol) in a sealed flask was evacuated and backfilled with N₂(g) (×3). A mixture of MeCN (3 mL) and water (0.6 mL) was degassed by N₂(g) bubbling for 15 min and added. The reaction mixture was evacuated and backfilled with N₂(g) (×3) and then stirred at rt overnight. The reaction mixture was concentrated at reduced pressure, and the residue was dissolved in DMSO, filtered and the crude compound was purified by preparative HPLC, PrepMethod A, (gradient: 20-90%) to give the title compound (0.042 g, 62%); HRMS (ESI) m/z [M+H]⁺ calcd for C₃₂H₃₂ClN₅O₅: 602.2164, found: 602.2166; ¹H NMR (500 MHz, CD₃OD) 1.83-1.9 (2H, m), 1.93-2 (1H, m), 2.01 (3H, s), 2.25-2.36 (1H, m), 2.42-2.51 (1H, m), 2.52-2.63 (1H, m), 2.71-2.77 (1H, m), 2.78-2.85 (1H, m), 3.27-3.33 (partly overlapping with solvent, m), is 3.34-3.42 (1H, m), 3.73 (1H, d), 4.26 (1H, d), 4.33-4.4 (1H, m), 4.53 (1H, dt), 4.63-4.72 (2H, m), 5.07 (1H, dd), 5.24-5.33 (1H, m), 6.36 (1H, dd), 6.4-6.45 (1H, m), 6.7-6.77 (1H, m), 7.67 (2H, td), 7.87 (1H, dd), 7.98 (1H, dd), 8.32-8.37 (1H, m), 8.60 (1H, dd).

### Example 6a

### 2-(((1R*,6R*)-5-((R*)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid, Isomer 1

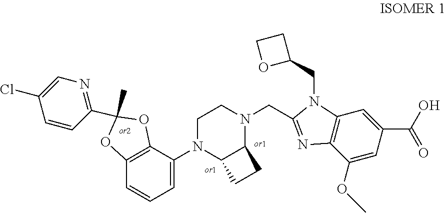 [SMILES: COc1cc(C(=O)O)cc2c1nc(CN1CCN(c3cccc4c3O[C@@](C)(c3ccc(Cl)cn3)O4)[C@H]3CC[C@@H]31)n2C[C@@H]1CCO1]

A mixture of methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 1 Intermediate 43 (53 mg, 0.08 mmol) and 1,3,4,6,7,8-hexahydro-2H-pyrimido[1,2-a]pyrimidine (23 mg, 0.16 mmol) in a sealed flask was evacuated and backfilled with N₂(g) (×3). A mixture of MeCN (3 mL) and water (0.6 mL) was degassed by N₂(g) bubbling for 15 min and added. The reaction mixture was evacuated and backfilled with N₂(g) (×3) and then stirred at rt overnight. The reaction mixture was concentrated at reduced pressure, and the residue was dissolved in DMSO, filtered and purified by preparative HPLC, PrepMethod A, (gradient: 20-90%) to give the title compound (15 mg, 29%); HRMS (ESI) m/z [M+H]⁺ calcd for C₃₃H₃₅ClN₅O₆: 632.2270, found: 632.2302; ¹H NMR (500 MHz, CD₃OD) 1.47 (1H, dd), 1.79-1.88 (2H, m), 2.01 (3H, s), 2.21-2.29 (1H, m), 2.39-2.59 (3H, m), 2.62 (1H, dd), 2.73-2.86 (2H, m), 2.91-2.97 (1H, m), 3.81 (1H, d), 3.83-3.88 (1H, m), 3.95-4.01 (1H, m), 4.03 (3H, s), 4.39-4.46 (1H, m), 4.58-4.65 (1H, m), 4.77-4.83 (2H, m), 5.19-5.28 (1H, m), 6.35-6.41 (1H, m), 6.52 is (1H, dd), 6.74 (1H, t), 7.43 (1H, d), 7.63 (1H, dd), 7.85 (1H, dd), 7.93 (1H, d), 8.57 (1H, dd).

### Example 6b

### 2-(((1R*,6R*)-5-((R*)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid, Isomer 2

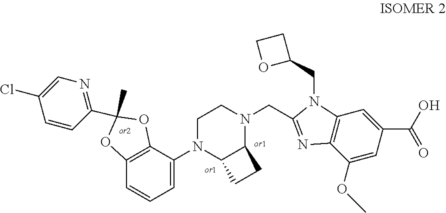 [SMILES: COc1cc(C(=O)O)cc2c1nc(CN1CCN(c3cccc4c3O[C@@](C)(c3ccc(Cl)cn3)O4)[C@H]3CC[C@@H]31)n2C[C@@H]1CCO1]

A mixture of methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 2 Intermediate 44 (43 mg, 0.07 mmol) and 1,3,4,6,7,8-hexahydro-2H-pyrimido[1,2-a]pyrimidine (19 mg, 0.14 mmol) in a sealed flask was evacuated and backfilled with N₂(g) (×3). A mixture of MeCN (3 mL) and water (0.6 mL) was degassed by N₂(g) bubbling for 15 min and added. The reaction mixture was evacuated and backfilled with N₂(g) (×3) and then stirred at rt overnight. The reaction mixture was concentrated at reduced pressure, and the residue was dissolved in DMSO, filtered and purified by preparative HPLC, PrepMethod A, (gradient: 20-90%) to give the title compound (22 mg, 51%); HRMS (ESI) m/z [M+H]⁺ calcd for C₃₃H₃₅ClN₅O₆: 632.2270, found: 632.2282; ¹H NMR (500 MHz, CD₃OD) 1.47-1.55 (1H, m), 1.74-1.88 (2H, m), 2.01 (3H, s), 2.21 (1H, dd), 2.47-2.58 (2H, m), 2.6-2.69 (1H, m), 2.71-2.84 (2H, m), 2.83-2.92 (1H, m), 3.05 (1H, d), 3.87-3.96 (2H, m), 4.03 (4H, s), 4.44 (1H, dt), 4.6-4.68 (1H, m), 4.82-4.84 (2H, m), 5.21-5.29 (1H, m), 6.39 (1H, dd), 6.52 (1H, dd), 6.75 (1H, t), 7.44 (1H, d), 7.69 (1H, dd), 7.87-7.91 (1H, m), 7.95 (1H, d), 8.61 (1H, dd).

### Example 6c

### 2-(((1R*,6R*)-5-((R*)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid, Isomer 3

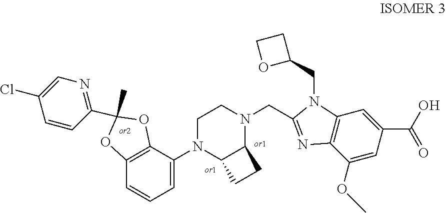 [SMILES: COc1cc(C(=O)O)cc2c1nc(CN1CCN(c3cccc4c3O[C@@](C)(c3ccc(Cl)cn3)O4)[C@H]3CC[C@@H]31)n2C[C@@H]1CCO1]

A mixture of methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 3 Intermediate 45 (30 mg, 0.05 mmol) and 1,3,4,6,7,8-hexahydro-2H-pyrimido[1,2-a]pyrimidine (13 mg, 0.09 mmol) in a sealed flask was evacuated and backfilled with N₂(g) (×3). A mixture of MeCN (3 mL) and water (0.6 mL) was degassed by N₂(g) bubbling for 15 min and added. The reaction mixture was evacuated and backfilled with N₂(g) (×3) and then stirred at rt overnight. The reaction mixture was concentrated at reduced pressure, and the residue was dissolved in DMSO, filtered and purified by preparative HPLC, PrepMethod A, (gradient: 20-90%) to give the title compound (23 mg, 80%); HRMS (ESI) m/z [M+H]⁺ calcd for C₃₃H₃₅ClN₅O₆: 632.2270, found: 632.2302; ¹H NMR (500 MHz, CD₃OD) 1.3-1.41 (1H, m), 1.67-1.81 (2H, m), 2.02 (3H, s), 2.13-2.22 (1H, m), 2.45-2.56 (2H, m), 2.66-2.74 (1H, m), 2.75-2.91 (3H, m), 3.14-3.2 (1H, m), 3.92 (1H, d), 3.94-3.99 (1H, m), 4.02 (4H, d), 4.41-4.49 (1H, m), 4.6-4.7 (2H, m), 4.93 (1H, dd), 5.25-5.34 (1H, m), 6.39 (1H, dd), 6.52 (1H, dd), 6.74 (1H, t), 7.44 (1H, d), 7.69 (1H, dd), 7.89 (1H, dd), 7.97 (1H, d), 8.59-8.63 (1H, m).

### Example 6d

### 2-(((1R*,6R*)-5-((R*)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid, Isomer 4

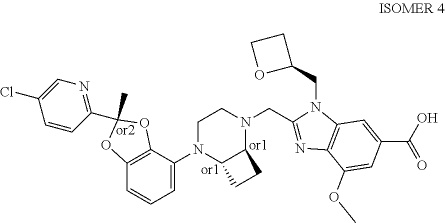 [SMILES: COc1cc(C(=O)O)cc2c1nc(CN1CCN(c3cccc4c3O[C@@](C)(c3ccc(Cl)cn3)O4)[C@H]3CC[C@@H]31)n2C[C@@H]1CCO1]

A mixture of methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 4 Intermediate 46 (54 mg, 0.08 mmol) and 1,3,4,6,7,8-hexahydro-2H-pyrimido[1,2-a]pyrimidine (23 mg, 0.17 mmol) in a sealed flask was evacuated and backfilled with N₂(g) (×3). A mixture of MeCN (3 is mL) and water (0.6 mL) was degassed by N₂(g) bubbling for 15 min and added. The reaction mixture was evacuated and backfilled with N₂(g) (×3) and then stirred at rt overnight. The reaction mixture was concentrated at reduced pressure, and the residue was dissolved in DMSO, filtered and purified by preparative HPLC, PrepMethod A, (gradient: 20-90%) to give the title compound (18 mg, 35%); HRMS (ESI) m/z [M+H]⁺ calcd for C₃₃H₃₅ClN₅O₆: 632.2270, found: 632.2306; ¹H NMR (500 MHz, CD₃OD) 1.26-1.37 (1H, m), 1.66-1.74 (1H, m), 1.75-1.85 (1H, m), 2.01 (3H, s), 2.16-2.24 (1H, m), 2.42-2.53 (2H, m), 2.58-2.72 (2H, m), 2.72-2.85 (2H, m), 3.05-3.11 (1H, m), 3.84-3.91 (2H, m), 3.96 (1H, d), 4.02 (3H, s), 4.39-4.47 (1H, m), 4.57-4.67 (2H, m), 4.91 (1H, dd), 5.23-5.32 (1H, m), 6.34-6.39 (1H, m), 6.52 (1H, dd), 6.73 (1H, t), 7.43 (1H, d), 7.61-7.66 (1H, m), 7.85 (1H, dd), 7.95 (1H, d), 8.56-8.59 (1H, m).

### Example 7a

### 2-(((1R*,6R*)-5-((R*)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid, Isomer 1

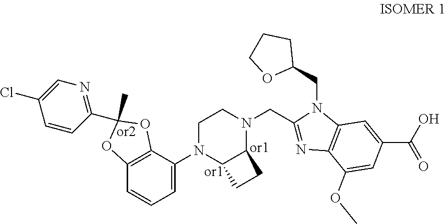 [SMILES: COc1cc(C(=O)O)cc2c1nc(CN1CCN(c3cccc4c3O[C@@](C)(c3ccc(Cl)cn3)O4)[C@H]3CC[C@@H]31)n2C[C@@H]1CCCO1]

A solution of methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate, Isomer 1 Intermediate 52 (29 mg, 0.04 mmol) and 1,3,4,6,7,8-hexahydro-2H-pyrimido[1,2-a]pyrimidine (21 mg, 0.15 mmol) in a mixture of MeCN (1 mL) and water (0.2 mL) was stirred at rt overnight. The reaction mixture was concentrated under reduced pressure and the residue was partitioned between DCM (2 mL) and 1M HCl (aq, 2 mL). The aqueous phase was extracted with another portion of DCM (2 mL), and the combined organic layer was concentrated and dried under reduced pressure to give the title compound (30 mg, 106%) as a white solid; HRMS (ESI) m/z [M+H]⁺ calcd for C₃₄H₃₇ClN₅O₆: 646.2426, found: 646.2480; is ¹H NMR (500 MHz, CD₃OD) 1.73 (1H, dq), 1.82-1.93 (2H, m), 1.93-2.01 (3H, m), 2.03 (3H, s), 2.23 (1H, h), 2.29-2.4 (1H, m), 3.14 (1H, td), 3.33-3.46 (2H, m), 3.60 (1H, td), 3.73-3.81 (1H, m), 3.92-4.01 (2H, m), 4.07 (3H, s), 4.11 (1H, ddd), 4.23 (1H, tdd), 4.51 (1H, dd), 4.68 (1H, dd), 4.74 (2H, s), 6.47 (1H, dd), 6.62 (1H, dd), 6.81 (1H, t), 7.51 (1H, d), 7.66 (1H, dd), 7.89 (1H, dd), 7.98 (1H, d), 8.61 (1H, d).

### Example 7b

### 2-(((1R*,6R*)-5-((R*)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid. Isomer 2

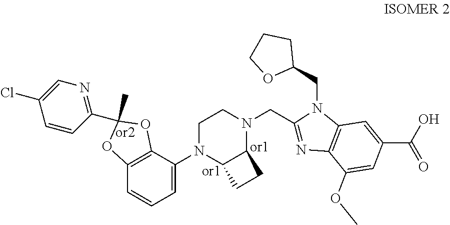 [SMILES: COc1cc(C(=O)O)cc2c1nc(CN1CCN(c3cccc4c3O[C@@](C)(c3ccc(Cl)cn3)O4)[C@H]3CC[C@@H]31)n2C[C@@H]1CCCO1]

A solution of methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate, Isomer 2 Intermediate 53 (52 mg, 0.08 mmol) and 1,3,4,6,7,8-hexahydro-2H-pyrimido[1,2-a]pyrimidine (25 mg, 0.18 mmol) in a mixture of MeCN (1 mL) and water (0.2 mL) was stirred at rt overnight. The reaction mixture was concentrated under reduced pressure and the residue was partitioned between DCM (2 mL) and 1M HCl (aq, 2 mL). The aqueous phase was extracted with another portion of DCM (2 mL), and the combined organic layer was concentrated and dried under reduced pressure to give the title compound (40 mg, 79%) as a white solid; HRMS (ESI) m/z [M+H]⁺ calcd for C₃₄H₃₇ClN₅O₆: 646.2426, found: 646.2462; ¹H NMR (500 MHz, CD₃OD) 1.72 (1H, dq), 1.85-2.06 (8H, m), 2.22 (1H, h), 2.35-2.43 (1H, m), 3.01 (1H, td), 3.31-3.38 (1H, m), 3.4-3.47 (1H, m), 3.58 (1H, td), 3.77 (1H, dt), 3.92-3.99 (2H, m), 4.01-4.11 (4H, m), 4.23 (1H, qd), 4.49 (1H, dd), 4.67 (1H, dd), 4.74 (2H, s), is 6.44 (1H, d), 6.62 (1H, dd), 6.80 (1H, t), 7.51 (1H, d), 7.66 (1H, d), 7.88 (1H, dd), 7.97 (1H, d), 8.59 (1H, d).

### Example 7c

### 2-(((1R*,6R*)-5-((R*)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid, Isomer 3

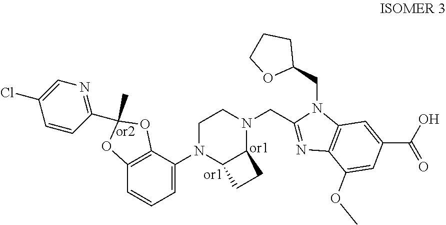 [SMILES: COc1cc(C(=O)O)cc2c1nc(CN1CCN(c3cccc4c3O[C@@](C)(c3ccc(Cl)cn3)O4)[C@H]3CC[C@@H]31)n2C[C@@H]1CCCO1]

A solution of methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate, Isomer 3 Intermediate 54 (31 mg, 0.05 mmol) and 1,3,4,6,7,8-hexahydro-2H-pyrimido[1,2-a]pyrimidine (17 mg, 0.12 mmol) in a mixture of MeCN (1 mL) and water (0.2 mL) was stirred at rt overnight. The reaction mixture was concentrated under reduced pressure and the residue was partitioned between DCM (2 mL) and 1M HCl (aq, 2 mL). The aqueous phase was extracted with another portion of DCM (2 mL), and the combined organic layer was concentrated and dried under reduced pressure to give the title compound (27 mg, 89%) as a white solid; HRMS (ESI) m/z [M+H]⁺ calcd for C₃₄H₃₇ClN₅O₆: 646.2426, found: 646.2456; ¹H NMR (500 MHz, CD₃OD) 1.67 (1H, dq), 1.82-1.97 (4H, m), 1.97-2.02 (1H, m), 2.04 (3H, s), 2.20 (1H, h), 2.35 (1H, dt), 3.16 (1H, td), 3.33-3.44 (2H, m), 3.54 (1H, td), 3.74 (1H, td), 3.91 (2H, ddd), 4.05-4.14 (4H, m), 4.24 (1H, qd), 4.48 (1H, dd), 4.6-4.75 (3H, m), 6.49 (1H, d), 6.64 (1H, d), 6.82 (1H, t), 7.54 (1H, s), 7.68 (1H, d), 7.90 (1H, dd), 8.00 (1H, d), 8.62 (1H, is d).

### Example 8a

### 2-(((R-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid, Isomer 1

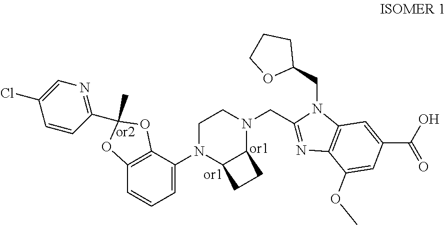 [SMILES: COc1cc(C(=O)O)cc2c1nc(CN1CCN(c3cccc4c3O[C@@](C)(c3ccc(Cl)cn3)O4)[C@@H]3CC[C@@H]31)n2C[C@@H]1CCCO1]

A solution of methyl 2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate, Isomer 1 Intermediate 56 (74 mg, 0.11 mmol) and 1,3,4,6,7,8-hexahydro-2H-pyrimido[1,2-a]pyrimidine (62 mg, 0.45 mmol) in a mixture of MeCN (1 mL) and water (0.2 mL) was stirred at rt overnight. The reaction mixture was concentrated under reduced pressure and the residue was partitioned between DCM (2 mL) and 1M HCl (aq, 2 mL). The aqueous phase was extracted with another portion of DCM (2 mL) and the combined organic layer was concentrated and dried under reduced pressure to give the title compound (60 mg, 83%) as a white solid; HRMS (ESI) m/z [M+H]⁺ calcd for C₃₄H₃₇ClN₅O₆: 646.2426, found: 646.2418; ¹H NMR (500 MHz, CD₃OD) 1.75 (1H, dq), 1.93-2.14 (8H, m), 2.21-2.31 (2H, m), 3.26-3.33 (2H, m), 3.43-3.54 (1H, m), 3.77 (1H, q), 3.8-3.84 (1H, m), 3.97 (1H, q), 4.04-4.1 (1H, m), 4.11 (3H, s), 4.26-4.38 (2H, m), 4.51 (1H, d), 4.58-4.67 (2H, m), 4.73 (1H, dd), 6.37 (1H, d), 6.55 (1H, d), 6.80 (1H, t), 7.60 (1H, s), 7.69 (1H, d), 7.90 (1H, dd), 8.06 (1H, s), 8.62 (1H, d).

### Example 8b

### 2-((1R*,6S*)-5-(R*)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid, Isomer 2

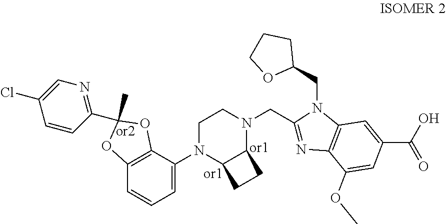 [SMILES: COc1cc(C(=O)O)cc2c1nc(CN1CCN(c3cccc4c3O[C@@](C)(c3ccc(Cl)cn3)O4)[C@@H]3CC[C@@H]31)n2C[C@@H]1CCCO1]

A solution of methyl 2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate, Isomer 2 Intermediate 57 (109 mg, 0.17 mmol) and 1,3,4,6,7,8-hexahydro-2H-pyrimido[1,2-a]pyrimidine (57 mg, 0.41 mmol) in a mixture of MeCN (1 mL) and water (0.2 mL) was stirred at rt overnight. The reaction mixture was concentrated under reduced pressure and the residue was partitioned between DCM (2 mL) and 1M HCl (aq, 2 mL). The aqueous phase was extracted with another portion of DCM (2 mL) and the combined organic layer was concentrated and dried under reduced pressure to give the title compound (93 mg, 87%) as a white solid; HRMS (ESI) m/z [M+H]⁺ calcd for C₃₄H₃₇ClN₅O₆: 646.2426, found: 646.2410; ¹H NMR (500 MHz, CD₃OD) 1.72 (1H, dq), 1.86-2.02 (4H, m), 2.03 (3H, s), 2.05-2.11 (1H, m), 2.17-2.35 (2H, m), 3.12-3.2 (1H, m), 3.19-3.27 (1H, m), 3.44 (1H, t), 3.73-3.85 (2H, m), 3.95 (1H, dt), 4.00-4.08 (1H, m), 4.10 (3H, s), 4.22-4.35 (2H, m), 4.48 (1H, d), 4.54-4.64 (2H, m), 4.71 (1H, dd), 6.33 (1H, d), 6.55 (1H, d), 6.78 (1H, t), 7.59 (1H, s), 7.67 (1H, d), 7.88 (1H, dd), 8.05 (1H, s), 8.60 (1H, d).

### Example 8c

### 2-(((R-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid, Isomer 3

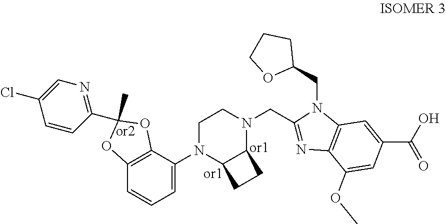 [SMILES: COc1cc(C(=O)O)cc2c1nc(CN1CCN(c3cccc4c3O[C@@](C)(c3ccc(Cl)cn3)O4)[C@@H]3CC[C@@H]31)n2C[C@@H]1CCCO1]

A solution of methyl 2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate, Isomer 3 Intermediate 58 (67 mg, 0.10 mmol) and 1,3,4,6,7,8-hexahydro-2H-pyrimido[1,2-a]pyrimidine (36 mg, 0.26 mmol) in a mixture of MeCN (1 mL) and water (0.2 mL) was is stirred at rt overnight. The reaction mixture was concentrated under reduced pressure and the residue was partitioned between DCM (2 mL) and 1M HCl (aq, 2 mL). The aqueous phase was extracted with another portion of DCM (2 mL) and the combined organic layer was concentrated and dried under reduced pressure to give the title compound (67 mg, 102%) as a white solid; HRMS (ESI) m/z [M+H]⁺ calcd for C₃₄H₃₇ClN₅O₆: 646.2426, found: 646.2416; ¹H NMR (500 MHz, CD₃OD) 1.70 (1H, dq), 1.86-1.98 (4H, m), 2.03 (3H, s), 2.08-2.17 (1H, m), 2.21 (1H, dq), 2.35-2.46 (1H, m), 3.08-3.2 (1H, m), 3.33-3.39 (1H, m), 3.46-3.54 (1H, m), 3.75 (1H, dt), 3.85-3.96 (2H, m), 4.07-4.12 (4H, m), 4.19-4.32 (2H, m), 4.47-4.58 (2H, m), 4.61 (1H, d), 4.76 (1H, dd), 6.33 (1H, dd), 6.56 (1H, dd), 6.78 (1H, t), 7.58 (1H, d), 7.67 (1H, dd), 7.88 (1H, dd), 8.04 (1H, d), 8.59 (1H, dd).

### Example 8d

### 2-(((R 2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid, Isomer 4

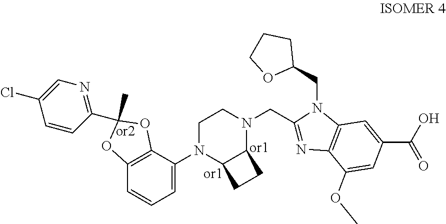 [SMILES: COc1cc(C(=O)O)cc2c1nc(CN1CCN(c3cccc4c3O[C@@](C)(c3ccc(Cl)cn3)O4)[C@@H]3CC[C@@H]31)n2C[C@@H]1CCCO1]

A solution of methyl 2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylate, Isomer 4 Intermediate 59 (67 mg, 0.10 mmol) and 1,3,4,6,7,8-hexahydro-2H-pyrimido[1,2-a]pyrimidine (73 mg, 0.52 mmol) in a mixture of MeCN (1 mL) and water (0.2 mL) was stirred at rt overnight. The reaction mixture was concentrated under reduced pressure and the residue was partitioned between DCM (2 mL) and 1M HCl (aq, 2 mL). The aqueous phase was extracted with another portion of DCM (2 mL) and the combined organic layer was concentrated and dried under reduced pressure to give the title compound (62 mg, 95%) as a white solid; HRMS (ESI) m/z [M+H]⁺ calcd for C₃₄H₃₇ClN₅O₆: 646.2426, found: 646.2414; is ¹H NMR (500 MHz, CD₃OD) 1.73 (1H, dq), 1.95 (2H, p), 1.99-2.08 (5H, m), 2.11-2.2 (1H, m), 2.23 (1H, h), 2.33-2.41 (1H, m), 3.25-3.32 (1H, m), 3.35-3.41 (1H, m), 3.52-3.6 (1H, m), 3.75 (1H, dt), 3.86-3.96 (2H, m), 4.08-4.17 (4H, m), 4.25-4.34 (2H, m), 4.54-4.6 (2H, m), 4.64 (1H, d), 4.78 (1H, dd), 6.37 (1H, d), 6.56 (1H, d), 6.80 (1H, t), 7.58 (1H, s), 7.69 (1H, d), 7.90 (1H, dd), 8.05 (1H, d), 8.62 (1H, d).

### Example 9a

2-2(((R-(4-Chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid. Isomer 1

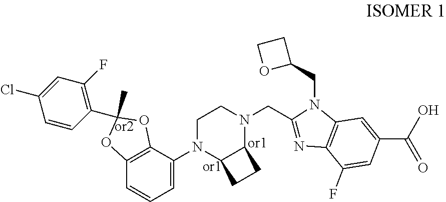 [SMILES: C[C@]1(c2ccc(Cl)cc2F)Oc2cccc(N3CCN(Cc4nc5c(F)cc(C(=O)O)cc5n4C[C@@H]4CCO4)[C@H]4CC[C@H]43)c2O1]

A mixture of methyl 2-(((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 1 Intermediate 62 (114 mg, 0.18 mmol) and 1,3,4,6,7,8-hexahydro-2H-pyrimido[1,2-a]pyrimidine (49 mg, 0.35 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The reaction mixture was concentrated in vacuo, and the residue was dissolved in DMSO, filtered and purified by preparative HPLC, PrepMethod B, (gradient: 5-95%) to give the title compound Isomer 1 (65 mg, 58%); HRMS (ESI) m/z [M+H]⁺ calcd for C₃₃H₃₂ClF₂N₄O₅: 637.2024, found: 637.2040; ¹H NMR (600 MHz, DMSO-d₆) 1.5-1.62 (1H, m), 1.63-1.75 (1H, m), 1.88-1.94 (1H, m), 1.97 (3H, s), 2.13-2.23 (1H, m), 2.29-2.42 (2H, m), 2.62-2.74 (2H, m), 3.12-3.22 (1H, m), 3.22-3.3 (1H, m), 3.33-3.45 (1H, m), 3.74 (1H, d), 4.12-4.26 is (3H, m), 4.45 (1H, td), 4.75 (1H, dd), 4.85 (1H, dd), 5.12-5.22 (1H, m), 6.24-6.33 (1H, m), 6.43-6.48 (1H, m), 6.70 (1H, t), 7.30 (1H, dd), 7.47-7.57 (3H, m), 8.15-8.19 (1H, m).

### Example 9b

2(((R-(4-Chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid, Isomer 2

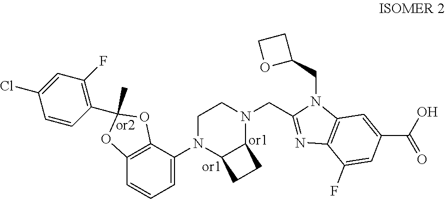 [SMILES: C[C@]1(c2ccc(Cl)cc2F)Oc2cccc(N3CCN(Cc4nc5c(F)cc(C(=O)O)cc5n4C[C@@H]4CCO4)[C@H]4CC[C@H]43)c2O1]

A mixture of methyl 2-(((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 2 Intermediate 63 (129 mg, 0.20 mmol) and 1,3,4,6,7,8-hexahydro-2H-pyrimido[1,2-a]pyrimidine (55 mg, 0.40 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight. The reaction mixture was concentrated in vacuo, and the residue was dissolved in DMSO, filtered and purified by preparative HPLC, PrepMethod B, (gradient: 5-95%) to give the title compound Isomer 2 (88 mg, 70%); HRMS (ESI) m/z [M+H]⁺ calcd for C₃₃H₃₂ClF₂N₄O₅: 637.2024, found: 637.2030; ¹H NMR (600 MHz, DMSO-d₆) 1.5-1.59 (2H, m), 1.59-1.65 (1H, m), 2.01 (4H, s), 2.29-2.43 (2H, m), 2.49-2.51 (1H, m), 2.6-2.69 (1H, m), 2.71-2.77 (1H, m), 3.09-3.15 (1H, m), 3.21-3.31 (2H, m), 3.78 (1H, d), 4.11 (1H, d), 4.21-4.26 (1H, m), 4.31 (1H, q), 4.41-4.48 (1H, m), 4.71-4.83 (2H, m), 5.11-5.18 (1H, m), 6.26 (1H, dd), is 6.46-6.5 (1H, m), 6.70 (1H, t), 7.26-7.31 (1H, m), 7.48-7.56 (3H, m), 8.15-8.19 (1H, m).

### Example 9c

### 2-(((1R*,6S*)-5-((R*)-2-(4-Chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid, Isomer 3

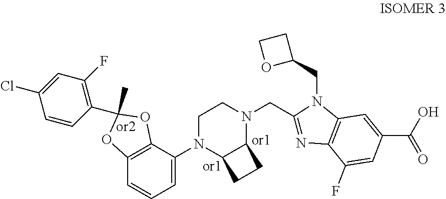 [SMILES: C[C@]1(c2ccc(Cl)cc2F)Oc2cccc(N3CCN(Cc4nc5c(F)cc(C(=O)O)cc5n4C[C@@H]4CCO4)[C@H]4CC[C@H]43)c2O1]

A mixture of methyl 2-(((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 3 Intermediate 64 (177 mg, 0.27 mmol) and 1,3,4,6,7,8-hexahydro-2H-pyrimido[1,2-a]pyrimidine (76 mg, 0.54 mmol) in a sealed flask was evacuated and backfilled with N₂(g) (×3). A mixture of MeCN (3 mL) and water (0.6 mL) was degassed by N₂(g) bubbling for 15 min, and then added. The reaction mixture was evacuated and backfilled with N₂(g) (×3) and then stirred at rt overnight.

The reaction mixture was concentrated in vacuo, and the residue was dissolved in DMSO, filtered and purified by preparative HPLC, PrepMethod B, (gradient: 5-95%) to give the title compound Isomer 3 (102 mg, 59%); HRMS (ESI) m/z [M+H]⁺ calcd for C₃₃H₃₂ClF₂N₄O₅: 637.2024, found: 637.2062; ¹H NMR (600 MHz, DMSO-d₆) 1.57-1.64 (1H, m), 1.68-1.74 (2H, m), 2.01 (3H, s), 2.02-2.09 (1H, m), 2.32-2.4 (1H, m), 2.41-2.47 (1H, m), 2.49-2.51 (1H, m), 2.65-2.72 (2H, m), 3.12-3.27 (3H, m), 3.63 (1H, d), 4.18 (1H, d), 4.33-4.44 (2H, m), 4.47-4.54 (1H, m), 4.62 (1H, dd), 4.91 (1H, dd), 5.06-5.13 (1H, m), 6.25-6.29 (1H, m), 6.48 (1H, dd), 6.70 (1H, t), 7.30 (1H, dd), 7.5-7.56 (3H, m), 8.15-8.18 (1H, m).

### Example 9d

### 2-((1R*,6S*)-5-(R*)-2-(4-Chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid, Isomer 4

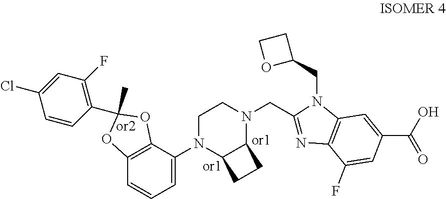 [SMILES: C[C@]1(c2ccc(Cl)cc2F)Oc2cccc(N3CCN(Cc4nc5c(F)cc(C(=O)O)cc5n4C[C@@H]4CCO4)[C@H]4CC[C@H]43)c2O1]

A mixture of methyl 2-(((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 4 Intermediate 65 (159 mg, 0.24 mmol) and 1,3,4,6,7,8-hexahydro-2H-pyrimido[1,2-a]pyrimidine (68 mg, 0.49 mmol) in a sealed flask was evacuated and backfilled with N₂(g) (×3). A mixture of MeCN (3 mL) and water (0.6 mL) was degassed by N₂ (g) bubbling for 15 min, and then added. The reaction mixture was evacuated and backfilled with N₂(g) (×3) and then stirred at rt overnight.

The reaction mixture was concentrated in vacuo, and the residue was dissolved in DMSO, filtered and purified by preparative HPLC, PrepMethod B, (gradient: 5-95%) to give the title compound Isomer 4 (90 mg, 58%); HRMS (ESI) m/z [M+H]⁺ calcd for C₃₃H₃₂ClF₂N40₅: 637.2024, found: 637.2052; ¹H NMR (600 MHz, DMSO-d₆) 1.69-1.79 (2H, m), 1.97 (3H, s), 2.22-2.28 (1H, m), 2.34-2.4 (1H, m), 2.44-2.5 (2H, m), 2.63-2.67 (1H, m), 2.68-2.74 (1H, m), 3.13-3.2 (1H, m), 3.2-3.25 (1H, m), 3.39-3.45 (1H, m), 3.61 (1H, d), 4.18-4.26 (2H, m), 4.39-4.46 (1H, m), 4.48-4.55 (1H, m), 4.6-4.66 (1H, m), 4.94 (1H, dd), 5.07-5.15 (1H, m), 6.29-6.34 (1H, m), 6.43-6.48 (1H, m), 6.71 (1H, t), 7.26-7.31 (1H, m), 7.47-7.5 (1H, m), 7.51-7.57 (2H, m), 8.15-8.2 (1H, m).

### Example 10a

### rel-4-Chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylic acid, Isomer 1

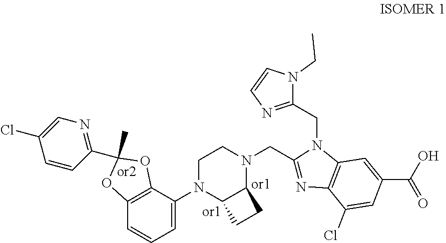 [SMILES: CCn1ccnc1Cn1c(CN2CCN(c3cccc4c3O[C@@](C)(c3ccc(Cl)cn3)O4)[C@H]3CC[C@@H]32)nc2c(Cl)cc(C(=O)O)cc21]

LiOH·H₂O (4 mg, 89 μmol) and rel-methyl 4-chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylate, Isomer 1 Intermediate 70 (39 mg, 0.057 mmol) were dissolved in a mixture of THE and water (1:1, 5 mL), and the reaction mixture was stirred at ambient temperature for 24 h. The reaction mixture was concentrated at reduced pressure to give the lithium salt of the title compound, which was is purified by preprative HPLC, PrepMethod E, (gradient 35-50%) to give the title compound as the lithium salt (28 mg, 72%); HRMS (ESI) m/z [M+H]⁺ calcd for C₃₄H₃₄Cl₂N₇O₄: 674.2044, found: 674.2080; ¹H NMR (600 MHz, DMSO-d₆) δ 8.68 (d, 1H), 7.97 (dd, 1H), 7.90 (s, 1H), 7.77 (s, 1H), 7.62 (s, 1H), 7.58 (d, 1H), 6.70 (t, 1H), 6.51 (d, 1H), 6.42 (s, 1H), 6.27 (d, 1H), 5.72 (d, 1H), 5.62 (d, 1H), 3.98 (dt, 2H), 3.73 (dd, 2H), 3.61 (d, 1H), 2.85 (d, 1H), 2.57 (t, 1H), 2.41 (d, 2H), 2.28 (q, 1H), 2.14 (q, 1H), 1.97 (s, 3H), 1.72-1.55 (m, 2H), 1.34-1.21 (m, 1H), 1.16 (t, 3H).

### Example 10b

rel-4-Chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylic acid. Isomer 3

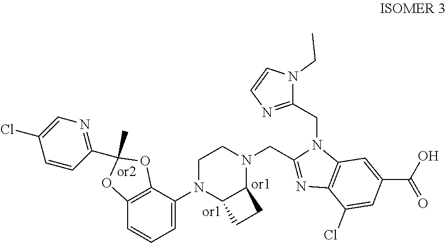 [SMILES: CCn1ccnc1Cn1c(CN2CCN(c3cccc4c3O[C@@](C)(c3ccc(Cl)cn3)O4)[C@H]3CC[C@@H]32)nc2c(Cl)cc(C(=O)O)cc21]

LiOH·H₂O (4 mg, 89 μmol) and rel-methyl 4-chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylate, Isomer 3 Intermediate 71 (16 mg, 0.023 mmol) were dissolved in a mixture of THE and water (1:1, 5 mL), and the reaction mixture was stirred at ambient temperature for 24 h. The reaction mixture was concentrated at reduced pressure to give the lithium salt of the title compound, which was purified by preprative HPLC, PrepMethod E, (gradient 35-50%) to give the title compound as the lithium salt (14 mg, 89%); HRMS (ESI) m/z [M+H]⁺ calcd for C₃₄H₃₄Cl₂N₇O₄: 674.2044, found: 674.2050; ¹H NMR (500 MHz, DMSO-d₆) δ 8.71 (d, 1H), 8.01 (dd, 1H), 7.90 (s, 1H), 7.76 (s, 1H), 7.67-7.51 (m, 2H), 6.71 (t, 1H), 6.54 (d, 1H), 6.41 (s, 1H), 6.28 (d, 1H), 5.84-5.50 (m, 2H), 3.99 (q, 2H), 3.83-3.56 (m, 3H), 2.88 (d, 1H), 2.54 (d, 2H), 2.32 (m, 1H), 2.25 (q, 1H), 2.06 (d, 1H), 1.97 (s, 3H), 1.60 (dq, 2H), 1.29-1.07 (m, 4H).

### Example 10c

### rel-4-Chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylic acid, Isomer 4

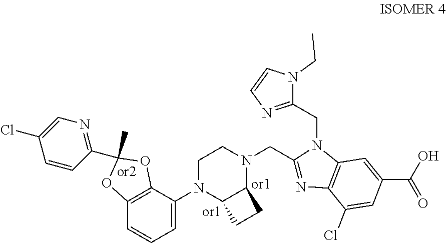 [SMILES: CCn1ccnc1Cn1c(CN2CCN(c3cccc4c3O[C@@](C)(c3ccc(Cl)cn3)O4)[C@H]3CC[C@@H]32)nc2c(Cl)cc(C(=O)O)cc21]

LiOH·H₂O (4 mg, 89 μmol) and rel-methyl 4-chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylate, Isomer 4 Intermediate 72 (36 mg, 0.052 mmol) were dissolved in a mixture of THE and water (1:1, 5 mL), and the reaction mixture was stirred at ambient temperature for 24 h. The reaction mixture was concentrated at reduced pressure to give the lithium salt of the title compound, which was purified by preprative HPLC, PrepMethod E, (gradient 35-50%) to give the title compound as the lithium salt (13 mg, 37%); HIRMS (ESI) m/z [M+H]⁺ calcd for C₃₄H₃₄Cl₂N₇O₄: 674.2044, found: 674.2046; ¹H NMR (600 MHz, DMSO-d₆) δ 8.68 (d, 1H), 7.96 (dd, 1H), 7.90 (d, 1H), 7.76 (d, 1H), 7.67-7.60 (m, 1H), 7.58 (d, 1H), 6.70 (t, 1H), 6.51 (d, 1H), 6.42 (s, 1H), 6.27 (d, 1H), 5.72 (d, 1H), 5.62 (d, 1H), 3.98 (qd, 2H), 3.73 (dd, 2H), 3.61 (d, 1H), 2.85 (d, 1H), 2.57 (dd, 1H), 2.46-2.37 (m, 2H), 2.28 (q, 1H), 2.14 (q, 1H), 1.97 (s, 3H), 1.73-1.54 (m, 2H), 1.27 (p, 1H), 1.16 (t, 3H).

### Example 11

### 2-(((1RS,6RS)-5-(2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid

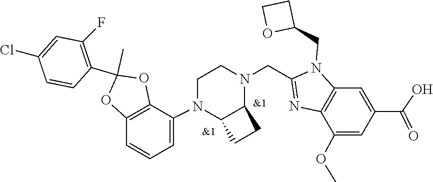 [SMILES: COc1cc(C(=O)O)cc2c1nc(CN1CCN(c3cccc4c3OC(C)(c3ccc(Cl)cc3F)O4)[C@H]3CC[C@@H]31)n2C[C@@H]1CCO1]

LiOH monohydrate (9.7 mg, 231 μmol) was added to a solution of methyl 2-(((1RS,6RS)-5-(2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate Intermediate 33 (102 mg, 154 μmol) in THF:H₂O (1:1, 5 mL) at 0° C. and the reaction mixture was stirred for 12 h. The reaction mixture was concentrated and the residue was acidified with 1 M citric acid (aq). The solids were collected by filtration to give the title compound (96 mg, 95%) as a white solid; IRMS (ESI) m z [M+H]⁺ calcd for C₃₄H₃₅ClFN₄O₆: 649.2224, found: 649.2258; ¹H NMR (400 MHz, DMSO-d₆) δ 12.54 (s, 1H), 7.88 (d, 1H), 7.62-7.44 (m, 2H), 7.42-7.16 (m, 2H), 6.73-6.68 (m, 1H), 6.54 (d, 1H), 6.32 (t, 1H), 5.20-4.98 (m, 1H), 4.81-4.74 (dt, 1H), 4.67-4.60 (m, 1H), 4.49-4.43 (m, 1H), 4.38-4.27 (m, 1H), 3.96 (s, 4H), 3.91-3.42 (m, 3H), 3.01-2.85 (m, 1H), 2.74-2.56 (m, 4H), 2.45-2.28 (m, 1H), 2.25-2.07 (m, 1H), 1.99 (s, 3H), 1.90-1.44 (m, 2H), 1.28-1.22 (m, 1H).

### Example 12

### 2-(((1RS,6RS)-5-(2-(5-Chloropyridin-2-yl)-2-methylbenzo[d][1,3]dioxol-4-yl)-2,5-diazabicyclo[4.2.0]octan-2-yl)methyl)-5-methoxy-1-(((S)-oxetan-2-yl)methyl)-1H-benzo[d]imidazole-6-carboxylic acid

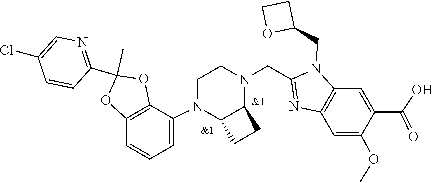 [SMILES: COc1cc2nc(CN3CCN(c4cccc5c4OC(C)(c4ccc(Cl)cn4)O5)[C@H]4CC[C@@H]43)n(C[C@@H]3CCO3)c2cc1C(=O)O]

LiOH monohydrate (5 mg, 0.114 mmol) was added to a solution of methyl 2-(((1RS,6RS)-5-(2-(5-chloropyridin-2-yl)-2-methylbenzo[d][1,3]dioxol-4-yl)-2,5-diazabicyclo[4.2.0]octan-2-yl)methyl)-5-methoxy-1-(((S)-oxetan-2-yl)methyl)-1H-benzo[d]imidazole-6-carboxylate Intermediate 84 (26 mg, 38 μmol) in THF:H₂O (2:1, 1 mL) and the reaction mixture was stirred at rt for 16 h. The reaction mixture was purified by preparative HPLC, Prep Method E (gradient: 0-60%) to give the lithium salt of the title compound (9.9 mg, 48%); HRMS (ESI) is m/z [M+H]⁺ calcd for C₃₃H₃₅ClN₅O₆: 632.2270, found: 632.2284; ¹H NMR (600 MHz, DMSO-d₆) δ 8.75-8.63 (m, 1H), 8.06-7.90 (m, 1H), 7.64-7.51 (m, 1H), 7.36 (d, 1H), 6.95 (s, 1H), 6.71-6.69 (m, 1H), 6.52 (dd, 1H), 6.32 (q, 1H), 5.10-5.00 (m, 1H), 4.72-4.50 (m, 1H), 4.48-4.44 (m, 1H), 4.33-4.31 (m, 1H), 3.86-3.70 (m, 2H), 3.69 (s, 3H), 3.68-3.52 (m, 1H), 2.91 (dt, 1H), 2.70-2.64 (m, 2H), 2.55 (s, 2H), 2.28-2.15 (m, 3H), 1.98 (s, 3H), 1.78-0.99 (m, 4H).

### Example 13

rac-2-(((1R,6R)-5-(2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-methoxy-1H-benzo[d]imidazole-6-carboxylic acid

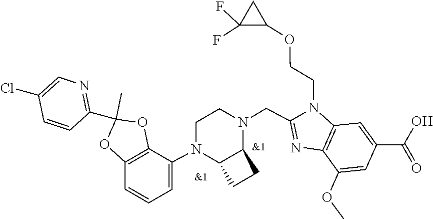 [SMILES: COc1cc(C(=O)O)cc2c1nc(CN1CCN(c3cccc4c3OC(C)(c3ccc(Cl)cn3)O4)[C@H]3CC[C@@H]31)n2CCOC1CC1(F)F]

LiOH monohydrate (19 mg, 0.43 mmol) was added to a solution of rac-methyl 2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-methoxy-1H-benzo[d]imidazole-6-carboxylate Intermediate 85 (197 mg, 0.28 mmol) in THF:H₂O (2:1, 5 mL) and the reaction mixture was stirred at rt for 16 h. The reaction mixture was acidified with NaH₂PO₄ (aq) solution and diluted with water. The formed precipitate was collected by filtration, washed with water and dried in vacuo to give the title compound (112 mg, 62%); HRMS (ESI) m z [M+H]⁺ calcd for C₃₄H₃₅ClF₂N₅O₆: 682.2238, found: 682.2252;_¹H NMR (500 MHz, DMSO-d₆) δ 12.40 (s, 1H), 8.83-8.50 (m, 1H), 8.04-7.89 (m, 1H), 7.81 (s, 1H), 7.58 (t, 1H), 7.24 (s, 1H), 6.70 (q, 1H), 6.52 (dd, 1H), 6.31 (t, 1H), 4.72-4.50 (m, 2H), 3.94 (s, 4H), 3.89-3.81 (m, 1H), 3.81-3.69 (m, 2H), 3.67-3.60 (m, 1H), 2.96-2.79 (m, 1H), 2.72-2.63 (m, 2H), 2.61-2.55 (m, 2H), 2.42-2.36 (m, 1H), 2.36-2.26 (m, 1H), 2.21-2.07 (m, 1H), 1.97 (s, 3H), 1.74-1.55 (m, 2H), 1.39-1.17 (m, 2H).

### Example 14

### rac-2-(((1R,6R)-5-(2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-fluoro-1H-benzo[d]imidazole-6-carboxylic acid

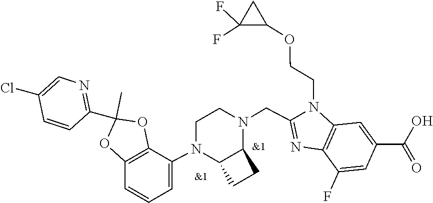 [SMILES: CC1(c2ccc(Cl)cn2)Oc2cccc(N3CCN(Cc4nc5c(F)cc(C(=O)O)cc5n4CCOC4CC4(F)F)[C@H]4CC[C@@H]43)c2O1]

LiOH monohydrate (7 mg, 0.16 mmol) was added to a solution of rac-methyl 2-(((1R,6R)-5-(2-(5-chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-fluoro-1H-benzo[d]imidazole-6-carboxylate Intermediate 86 (74 mg, 0.11 mmol) in THF:H₂O (2:1, 5 mL) and the reaction mixture was stirred at rt for 16 h. The reaction mixture was acidified with NaH₂PO₄ (aq) and diluted with water. The formed precipitate was collected by filtration, washed with water and dried in vacuo to give the title compound (53 mg, 73%); HRMS (ESI) m/z [M+H]⁺ calcd for C₃₃H₃₂ClF₃N₅O₅: 670.2038, found: 670.2042; ¹H NMR (500 MHz, DMSO-d₆) δ 12.42 (s, 1H), 8.80-8.55 (m, 1H), 8.15-7.86 (m, 2H), 7.67-7.54 (m, 1H), 7.48 (d, 1H), 6.71 (q, 1H), 6.53 (dd, 1H), 6.34-6.28 (m, 1H), 4.78-4.59 (m, 2H), 3.96 (s, 2H), 3.90-3.60 (m, 3H), 3.02-2.79 (m, 1H), 2.69 (d, 2H), 2.62-2.58 (m, 2H), 2.38-2.30 (m, 1H), 2.21-2.07 (m, 1H), 1.97 (s, 3H), 1.78-1.55 (m, 3H), 1.41-1.07 (m, 2H).

### Example 15a

### 2-((1R*,6S*)-5-(R*)-2-(4-Cano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid, Isomer 1

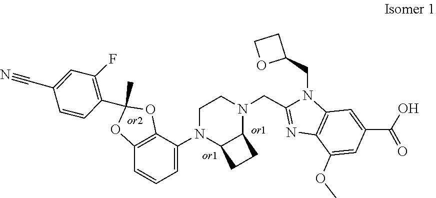 [SMILES: COc1cc(C(=O)O)cc2c1nc(CN1CCN(c3cccc4c3O[C@@](C)(c3ccc(C#N)cc3F)O4)[C@@H]3CC[C@@H]31)n2C[C@@H]1CCO1]

1,3,4,6,7,8-Hexahydro-2H-pyrimido[1,2-a]pyrimidine (38 mg, 0.28 mmol) was added to a solution of methyl 2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 1 Intermediate 93 (120 mg, 0.18 mmol) in MeCN (12 mL) and water (3 mL) and the reaction mixture was stirred at 20° C. for 4 h. The reaction mixture was concentrated and the residue was diluted with EtOAc (100 mL). The organic layer was washed with sat brine (25 mL), dried over Na₂SO₄, filtered and evaporated. The crude product was purified by preparative HPLC, PrepMethod H (gradient: 35-60%) to give the title compound Isomer 1 (64 mg, 55%) as a white solid; MS (ESI) m/z [M+H]⁺ 640.3; ¹H NMR (400 MHz, DMSO-d₆) δ 1.50-1.71 (m, 3H), 1.90-2.02 (m, 1H), 2.05 (s, 3H), 2.30-2.39 (m, 2H), 2.59-2.79 (m, 2H), 3.13 (d, 1H), 3.21-3.29 (m, 2H), 3.72 (d, 1H), 3.97 (s, 3H), 4.10 (d, 1H), 4.22 (dt, 1H), 4.31 (q, 1H), 4.44 (td, 1H), 4.64-4.78 (m, 2H), 5.12 (d, 1H), 6.29 (dd, 1H), 6.51 (dd, 1H), 6.73 (t, 1H), 7.27 (d, 1H), 7.66-7.80 (m, 2H), 7.89 (d, 1H), 7.98 (dd, 1H), 12.82 (s, 1H).

### Example 15b

### 2-(((1R*,6S*)-5-((R*)-2-(4-Cvano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid, Isomer 2

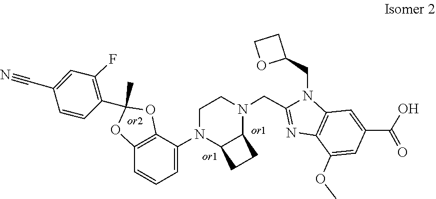 [SMILES: COc1cc(C(=O)O)cc2c1nc(CN1CCN(c3cccc4c3O[C@@](C)(c3ccc(C#N)cc3F)O4)[C@@H]3CC[C@@H]31)n2C[C@@H]1CCO1]

The title compound was prepared as described for Example 15a from methyl 2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 2 Intermediate 94 (130 mg, 0.20 mmol) to give the title compound Isomer 2 (87 mg, 68%) as a white solid; MS (ESI) m/z [M+H]⁺ 640.3; ¹H NMR (400 MHz, DMSO-d₆) δ 1.73-1.76 (m, 2H), 2.02 (s, 4H), 2.21-2.39 (m, 2H), 2.42-is 2.49 (m, 1H), 2.61-2.75 (m, 2H), 3.12-3.16 (m, 2H), 3.40-3.50 (m, 1H), 3.56 (d, 1H), 3.97 (s, 3H), 4.13-4.29 (m, 2H), 4.40-4.46 (m, 1H), 4.49-4.62 (m, 2H), 4.88 (dd, 1H), 5.06-5.15 (m, 1H), 6.30-6.40 (m, 1H), 6.48 (dd, 1H), 6.73 (t, 1H), 7.28 (d, 1H), 7.67-7.78 (m, 2H), 7.86-8.04 (m, 2H), 12.85 (s, 1H).

### Example 16a

### 4-Chloro-2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid Isomer 1

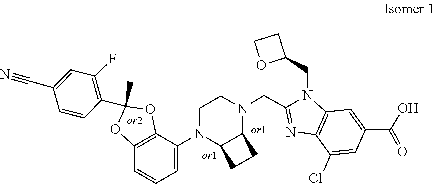 [SMILES: C[C@]1(c2ccc(C#N)cc2F)Oc2cccc(N3CCN(Cc4nc5c(Cl)cc(C(=O)O)cc5n4C[C@@H]4CCO4)[C@H]4CC[C@H]43)c2O1]

1,3,4,6,7,8-Hexahydro-2H-pyrimido[1,2-a]pyrimidine (35 mg, 0.25 mmol) was added to a solution of methyl 4-chloro-2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 1 Intermediate 97 (110 mg, 0.17 mmol) in MeCN (5 mL) and water (1.6 mL) and the reaction mixture was stirred at 20° C. for 2 h. The pH of the reaction mixture was adjusted to 7 by citric acid (0.5 M) and water (20 mL) was added. The aqueous layer was extracted with EtOAc (3×20 mL) and the combined organic layer was dried over Na₂SO₄, filtered and evaporated. The crude product was purified by preparative HPLC, PrepMethod H (gradient: 48-70%) to give the title compound Isomer 1 (66 mg, 61%) as a white solid; MS (ESI) m/z [M+H]⁺ 644.2; ¹H NMR (300 MHz, DMSO-d₆) δ 1.39-1.81 (m, 3H), 1.99 (t, 1H), 2.06 (s, 3H), 2.37 (dt, 2H), 2.58-2.85 (m, 2H), 3.15 (d, 1H), 3.31-4.10 (m, 2H), 3.80 (d, 1H), 4.15 (d, 1H), 4.21-4.42 (m, 2H), 4.46 (q, 1H), 4.67-4.93 (m, 2H), 5.10-5.21 (m, 1H), 6.29 (d, 1H), 6.52 (d, 1H), 6.74 (t, 1H), 7.73-7.80 (m, is 2H), 7.82 (d, 1H), 7.89-8.04 (m, 1H), 8.27 (d, 1H).

### Example 16b

### 4-Chloro-2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid, Isomer 2

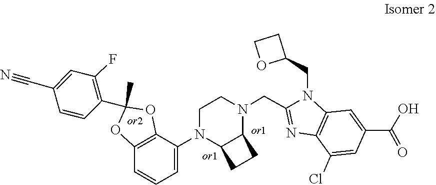 [SMILES: C[C@]1(c2ccc(C#N)cc2F)Oc2cccc(N3CCN(Cc4nc5c(Cl)cc(C(=O)O)cc5n4C[C@@H]4CCO4)[C@H]4CC[C@H]43)c2O1]

The title compound was prepared from methyl 4-chloro-2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 2 Intermediate 98 (140 mg, 0.21 mmol) as described for Example 16a to give the title compound Isomer 2 (74 mg, 54%) as a white solid; MS (ESI) m/z [M+H]⁺ 644.1; ¹H NMR (300 MHz, DMSO-d₆) δ 1.76 (d, 2H), 2.02 (s, 4H), 2.26 (q, 1H), 2.41 (q, 2H), 2.60-2.81 (m, 2H), 3.17 (t, 2H), 3.45 (d, 1H), 3.65 (d, 1H), 4.24 (d, 2H), 4.38-4.51 (m, 1H), 4.55 (dd, 1H), 4.66 (d, 1H), 4.97 (dd, 1H), 5.11 (q, 1H), 6.35 (d, 1H), 6.49 (dd, 1H), 6.74 (t, 1H), 7.71-7.77 (m, 2H), 7.82 (d, 1H), 7.91-8.05 (m, 1H), 8.29 (d, 1H), 13.21 (s, 1H).

### Example 16c

### 4-Chloro-2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid, Isomer 3

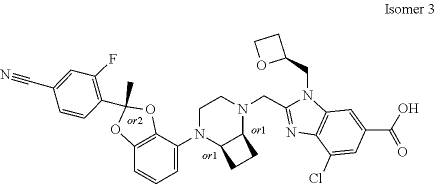 [SMILES: C[C@]1(c2ccc(C#N)cc2F)Oc2cccc(N3CCN(Cc4nc5c(Cl)cc(C(=O)O)cc5n4C[C@@H]4CCO4)[C@H]4CC[C@H]43)c2O1]

The title compound was prepared from methyl 4-chloro-2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 3 Intermediate 99 (140 mg, 0.21 mmol) as described for Example 16a to give the title compound Isomer 3 (86 mg, 63%) as a white solid; MS (ESI) m/z [M+H]⁺ 644.1; ¹H NMR (300 MHz, DMSO-d₆) δ 1.60 (d, 1H), 1.70 (s, 1H), 1.85-1.98 (m, 1H), 2.02 (s, 3H), 2.21 (t, 1H), 2.38 (dt, 2H), 2.60-2.85 (m, 2H), 3.20 (t, 1H), 3.39-3.45 (m, 2H), 3.76 (d, 1H), 4.07-4.36 (m, 3H), 4.47 (q, 1H), 4.77 (dd, 1H), 4.88 (dd, 1H), 5.15-5.25 (m, 1H), 6.17-6.40 (m, 1H), 6.48 (d, 1H), 6.74 (t, 1H), 7.72-7.78 (m, 2H), 7.83 (d, 1H), 7.89-8.05 (m, 1H), 8.28 (d, 1H), 13.26 (s, 1H).

### Example 17a

### 2-((1R*,6S*)-5-(R*)-2-(4-Cano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid Isomer 1

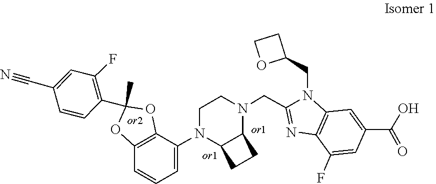 [SMILES: C[C@]1(c2ccc(C#N)cc2F)Oc2cccc(N3CCN(Cc4nc5c(F)cc(C(=O)O)cc5n4C[C@@H]4CCO4)[C@H]4CC[C@H]43)c2O1]

1,3,4,6,7,8-Hexahydro-2H-pyrimido[1,2-a]pyrimidine (59 mg, 0.42 mmol) was added to a solution of methyl 2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 1 Intermediate 102 (180 mg, 0.28 mmol) in MeCN (6 mL) and water (1.5 mL) and the reaction mixture was stirred at 20° C. for 2 h. The reaction mixture was concentrated and the residue was diluted with EtOAc (50 mL). The organic layer was washed with sat brine (25 mL), dried over Na₂SO₄, filtered and evaporated. The crude product was purified by preparative HPLC, PrepMethod I (gradient: 40-65%), to give the title compound Isomer 1 (102 mg, 58%) as a white solid; HRMS (ESI) m/z [M+H]⁺ calcd for C₃₄H₃₂F₂N₅O₅: 628.2366, found: 628.2400; ¹H NMR (400 MHz, DMSO-d₆) δ 1.50-1.61 (m, 2H), 1.61-1.69 (m, 1H), 1.93 (dd, 1H), 2.01 (s, 3H), 2.28-2.46 (m, 2H), 2.62-2.78 (m, 2H), 3.11-3.18 (m, 1H), 3.25-3.40 (m, overlapped with solvent), 3.75 (d, 1H), 4.12 (d, 1H), 4.15-4.28 (m, 2H), 4.44-4.52 (m, 1H), 4.70-4.86 (m, is 2H), 5.14-5.24 (m, 1H), 6.33 (dd, 1H), 6.48 (dd, 1H), 6.73 (t, 1H), 7.53 (dd, 1H), 7.69-7.77 (m, 2H), 7.94-8.02 (m, 1H), 8.18 (d, 1H), 13.04 (s, 1H).

### Example 17b

### 2-(((R-(4-Cano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid Isomer 2

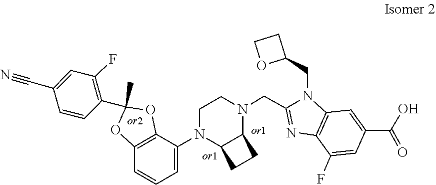 [SMILES: C[C@]1(c2ccc(C#N)cc2F)Oc2cccc(N3CCN(Cc4nc5c(F)cc(C(=O)O)cc5n4C[C@@H]4CCO4)[C@H]4CC[C@H]43)c2O1]

The title compound was prepared in analogy with the description for Example 17a from methyl 2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 2 Intermediate 103 (150 mg, 0.23 mmol). The crude product was purified by preparative HPLC, PrepMethod H (gradient: 40-70%), to give the title compound Isomer 2 (100 mg, 68%) as a white solid; HRMS (ESI) m/z [M+H]⁺ calcd for C₃₄H₃₂F₂N₅O₅: 628.2366, found: 628.2404; ¹H NMR (400 MHz, DMSO-d₆) δ 1.68-1.89 (m, 2H), 2.02 (s, 4H), 2.26 (p, 1H), 2.33-2.48 (m, 1H), 2.58-2.79 (m, 2H), 3.18 (t, 1H), 3.28-3.35 (m, 1H), 3.44 (dt, 1H), 3.62 (d, 1H), 4.23 (dd, 2H), 4.43-4.51 (m, 1H), 4.52-4.58 (m, 1H), 4.65 (dd, 1H), 4.95 (dd, 1H), 5.05-5.20 (m, 1H), 6.35 (dd, 1H), 6.48 (dd, 1H), 6.74 (t, 1H), 7.53 (dd, 1H), 7.67-7.78 (m, 2H), 7.92-8.02 (m, 1H), 8.21 (d, 1H), 13.01 (s, 1H).

### Example 17c

2-((1R*,6S*)-5-((R*)-2-(4-C₃ano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid Isomer 3

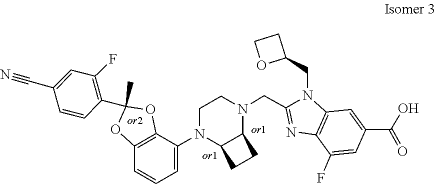 [SMILES: C[C@]1(c2ccc(C#N)cc2F)Oc2cccc(N3CCN(Cc4nc5c(F)cc(C(=O)O)cc5n4C[C@@H]4CCO4)[C@H]4CC[C@H]43)c2O1]

The title compound was prepared in analogy with the description for Example 17a from methyl 2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylate, Isomer 3 Intermediate 104 (180 mg, 0.28 mmol). The crude product was purified by preparative HPLC, PrepMethod H (gradient: 40-70%), to give the title compound Isomer 3 (97 mg, 55%) as a white solid; HRMS (ESI) m/z [M+H]⁺ calcd for C₃₄H₃₂F₂N₅O₅: 628.2366, found: 628.2394; ¹H NMR (400 MHz, DMSO-d₆) δ 1.55-1.62 (m, 1H), 1.65-1.76 (m, 1H), 1.90-2.00 (m, 1H), 2.01 (s, 3H), 2.17-2.22 (m, 1H), 2.31-2.45 (m, 2H), 2.60-2.77 (m, 2H), 3.20 (t, 1H), 3.21-3.27 (s, 1H), 3.39-3.43 (m, 1H), 3.75 (d, 1H), 4.15-4.28 (m, 3H), 4.46 (td, 1H), 4.73-4.93 (m, 2H), 5.12-5.28 (m, 1H), 6.33 (dd, 1H), 6.48 (dd, 1H), 6.73 (t, 1H), 7.53 (dd, 1H), 7.67-7.83 (m, 2H), 7.91-8.04 (m, 1H), 8.18 (d, 1H), 13.04 (s, 1H).

### Example 18a

### 2-(((1R*,6S*)-5-((R*)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid Isomer 1

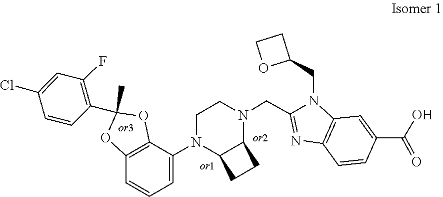 [SMILES: C[C@]1(c2ccc(Cl)cc2F)Oc2cccc(N3CCN(Cc4nc5ccc(C(=O)O)cc5n4C[C@@H]4CCO4)[C@H]4CC[C@H]43)c2O1]

NaOH (38 mg, 0.95 mmol) was added to a solution of methyl 2-(((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 1 Intermediate 106 (120 mg, 0.19 mmol) in MeOH (10 mL) and water (2 mL) and the reaction mixture was stirred at 25° C. for 12 h. The reaction mixture was concentrated and the pH was adjusted to 5-6 with 0.1 M HCl (1.5 mL). The aqueous layer was concentrated and the residue is was diluted with EtOAc (20 mL). The organic layer was washed with sat brine (2×50 mL), dried over Na₂SO₄, filtered and evaporated. The residue was purified by preparative HPLC, PrepMethod J (gradient: 30-60%) to give the title compound Isomer 1 (62 mg, 53%) as a white solid; HRMS (ESI) m/z [M+H]⁺ calcd for C₃₃H₃₃ClFN₄O₅: 619.2118, found: 619.2142; ¹H NMR (400 MHz, DMSO-d₆) δ 1.51-1.71 (m, 3H), 1.91-2.00 (m, 1H), 2.02 (s, 3H), 2.36 (q, 2H), 2.65 (dt, 1H), 2.71-2.78 (m, 1H), 3.06-3.18 (m, 1H), 3.15-3.30 (m, 2H), 3.75 (d, 1H), 4.10 (d, 1H), 4.19-4.37 (m, 2H), 4.45 (q, 1H), 4.74 (qd, 2H), 5.10-5.20 (m, 1H), 6.28 (d, 1H), 6.49 (d, 1H), 6.72 (t, 1H), 7.32 (dd, 1H), 7.54 (dd, 2H), 7.64 (d, 1H), 7.82 (d, 1H), 8.25 (s, 1H).

### Example 18b

### 2-((1R*,6S*)-5-(R*)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid Isomer 2

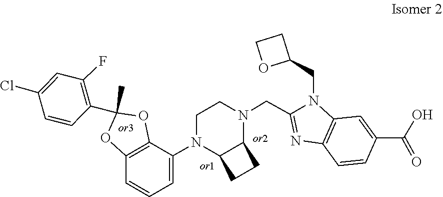 [SMILES: C[C@]1(c2ccc(Cl)cc2F)Oc2cccc(N3CCN(Cc4nc5ccc(C(=O)O)cc5n4C[C@@H]4CCO4)[C@H]4CC[C@H]43)c2O1]

The title compound was prepared as described for Example 18a from methyl 2-(((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 2 Intermediate 107 (80 mg, 0.13 mmol) to give the title compound Isomer 2 (47 mg, 60%) as a white solid; HRMS (ESI) m/z [M+H]⁺ calcd for C₃₃H₃₃ClFN₄O₅: 619.2118, found: 619.2156; ¹H NMR (400 MHz, DMSO-d6) δ 1.59 (d, 1H), 1.69 (s, 1H), 1.90-1.98 (m, 1H), 1.98 (s, 3H), 2.20 (p, 1H), 2.38 (t, 2H), 2.63-2.77 (m, 2H), 3.19 (t, 1H), 3.41 (m, overlapping with solvent), 3.71 (d, 1H), 4.11-4.28 (m, 3H), 4.46 (q, 1H), 4.72 (dd, 1H), 4.83 (d, 1H), 5.14-5.25 (m, 1H), 6.32 (d, 1H), 6.46 (d, 1H), 6.71 (t, 1H), 7.30-7.37 (m, 1H), 7.51-7.60 (m, 2H), 7.66 (d, 1H), 7.82 (d, 1H), 8.27 (s, 1H).

### Example 18c

2-2(((R-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid Isomer 4

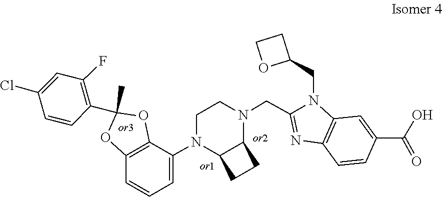 [SMILES: C[C@]1(c2ccc(Cl)cc2F)Oc2cccc(N3CCN(Cc4nc5ccc(C(=O)O)cc5n4C[C@@H]4CCO4)[C@H]4CC[C@H]43)c2O1]

The title compound was prepared as described for Example 18a from methyl 2-(((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 4 Intermediate 108 (80 mg, 0.13 mmol) to give the title compound Isomer 4 (45 mg, 57%) as a white solid; HRMS (ESI) m/z [M+H]⁺ calcd for C₃₃H₃₃ClFN₄O₅: 619.2118, found: 619.2132; ¹H NMR (400 MHz, DMSO-d₆) δ 1.76 (d, 2H), 1.99 (s, 4H), 2.21-2.40 (m, 2H), 2.62-2.72 (m, 2H), 3.16 (t, 1H), 3.25 (s, 2H), 3.44 (d, 1H), 3.58 (d, 1H), 4.16-4.30 (m, 2H), 4.44 (q, 1H), 4.49-4.53 (m, 1H), 4.60 (d, 1H), 4.91 (dd, 1H), 5.05-5.16 (m, 1H), 6.34 (d, 1H), 6.47 (d, 1H), 6.72 (t, 1H), 7.33 (dd, 1H), 7.51-7.59 (m, 2H), 7.63 (d, 1H), 7.83 (d, 1H), 8.26 (s, 1H).

### Example 19a

### 2-((1R*,6S*)-5-(R*)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid Isomer 1

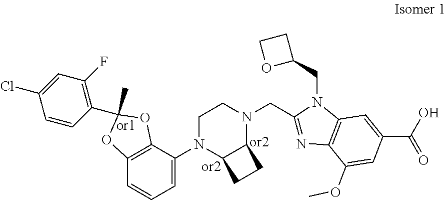 [SMILES: COc1cc(C(=O)O)cc2c1nc(CN1CCN(c3cccc4c3O[C@@](C)(c3ccc(Cl)cc3F)O4)[C@@H]3CC[C@@H]31)n2C[C@@H]1CCO1]

NaOH (30 mg, 0.75 mmol) was added to a solution of methyl 2-(((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 1 Intermediate 110 (100 mg, 0.15 mmol) in MeOH (10 mL) and water (2 mL) and the reaction mixture was stirred at 20° C. for 3 h. The reaction mixture was concentrated and acidified with 0.1 M HCl. The solvent was removed under reduced pressure and the residue was purified by preparative HPLC, PrepMethod K (gradient: 18-55%) to give the title compound Isomer 1 (14 mg, 14%) as a white solid; HRMS (ESI) m/z [M+H]⁺ calcd for C₃₄H₃₅ClFN₄O₆: 649.2224, found: 649.2238; ¹H NMR (400 MHz, DMSO-d₆) δ 1.58 (s, 1H), 1.69 (s, 1H), 1.90-1.97 (m, 1H), 1.99 (s, 3H), 2.21 (t, 1H), 2.27-2.42 (m, 2H), 2.67 (t, 2H), 3.15-3.15 (m, 2H), 3.38 (m, overlapped with solvent), 3.67 (d, 1H), 3.97 (s, 3H), 4.11-4.28 (m, 3H), 4.45 (q, 1H), 4.59-4.72 (m, 1H), 4.78-4.85 (m, 1H), 5.11-5.21 (m, 1H), 6.32 (d, 1H), 6.46 (d, 1H), 6.72 (t, 1H), 7.28 (s, 1H), 7.31-7.40 (m, 1H), 7.51-7.62 (m, 2H), 7.88 (s, 1H).

### Example 19b

### 2-(((1R*,6S*)-5-((R*)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid Isomer 2

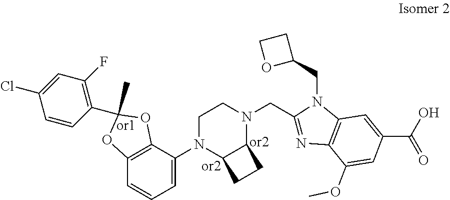 [SMILES: COc1cc(C(=O)O)cc2c1nc(CN1CCN(c3cccc4c3O[C@@](C)(c3ccc(Cl)cc3F)O4)[C@@H]3CC[C@@H]31)n2C[C@@H]1CCO1]

The title compound was prepared as described for Example 19a from methyl 2-(((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 2 Intermediate 111 (180 mg, 0.27 mmol). The crude product was purified by preparative HPLC, PrepMethod L (gradient: 24-54%) to give the title compound Isomer 2 (58 mg, 33%) as a yellow solid; HRMS (ESI) m/z [M+H]⁺ calcd for C₃₄H₃₅ClFN₄O₆: 649.2224, found: 649.2262; ¹H NMR (400 MHz, DMSO-d₆) δ 1.58 (s, 3H), 2.02 (s, 4H), 2.25-2.41 (m, 2H), 2.658-2.78 (m, 2H), 3.13 (s, 1H), 3.20-3.30 (m, 2H), 3.70 (d, 1H), 3.95 (s, 3H), 4.09 (d, 1H), 4.20 (s, 1H), 4.32 (q, 1H), 4.44 (q, 1H), 4.63 (d, 1H), 4.73 (d, 1H), 5.13 (s, 1H), 6.28 (d, 1H), 6.49 (d, 1H), 6.72 (t, 1H), 7.32 (d, 2H), 7.54 (t, is 2H), 7.85 (s, 1H).

### Example 19c

2-2(((R-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid Isomer 3

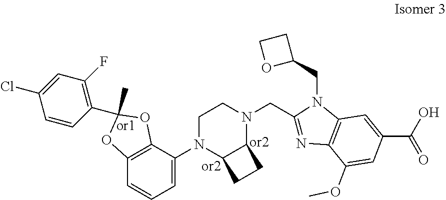 [SMILES: COc1cc(C(=O)O)cc2c1nc(CN1CCN(c3cccc4c3O[C@@](C)(c3ccc(Cl)cc3F)O4)[C@@H]3CC[C@@H]31)n2C[C@@H]1CCO1]

The title compound was prepared as described for Example 19a from methyl 2-(((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 3 Intermediate 112 (140 mg, 0.21 mmol). The crude product was purified by preparative HPLC, PrepMethod J (gradient: 30-60%) to give the title compound Isomer 3 (81 mg, 59%) as a white solid; HIRMS (ESI) m/z [M+H]⁺ calcd for C₃₄H₃₅ClFN₄O₆: 649.2224, found: 649.2256; ¹H NMR (400 MHz, DMSO-d₆) δ 1.61 (s, 1H), 1.71 (s, 2H), 2.03 (s, 4H), 2.34 (t, 1H), 2.40-2.48 (m, 1H), 2.63-2.73 (m, 2H), 3.11-3.29 (m, 3H), 3.58 (d, 1H), 3.95 (s, 3H), 4.14 (d, 1H), 4.38 (p, 2H), 4.44-4.61 (m, 2H), 4.84 (dd, 1H), 5.07 (q, 1H), 6.29 (d, 1H), 6.50 (d, 1H), 6.72 (t, 1H), 7.28 (s, 1H), 7.33 (dd, 1H), 7.50-7.61 (m, 2H), 7.87 (s, 1H).

### Example 19d

### 2-((1R*,6S*)-5-(R*)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid Isomer 4

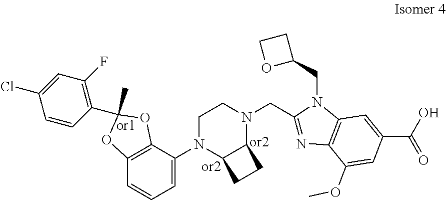 [SMILES: COc1cc(C(=O)O)cc2c1nc(CN1CCN(c3cccc4c3O[C@@](C)(c3ccc(Cl)cc3F)O4)[C@@H]3CC[C@@H]31)n2C[C@@H]1CCO1]

The title compound was prepared as described for Example 19a from methyl 2-(((1R*,6S*)-5-((R*)-2-(4-chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylate, Isomer 4 Intermediate 113 (70 mg, 0.11 mmol) to give the title compound Isomer 4 (35 mg, 52%) as a white solid; HIRMS (ESI) m/z [M+H]⁺ calcd for C₃₄H₃₅ClFN₄O₆: 649.2224, found: 649.2252; ¹H NMR (400 MHz, DMSO-d₆) δ 1.75 (d, 2H), 1.99 (s, 4H), 2.27 (t, 1H), 2.35 (t, 1H), 2.46 (d, 1H), 2.59-2.78 (m, 2H), 3.15 (t, 1H), 3.23 (s, 1H), 3.43 (d, 1H), 3.55 (d, 1H), 3.96 (s, 3H), 4.12-4.28 (m, 2H), 4.37-4.46 (m, 1H), 4.46-4.64 (m, 2H), 4.87 (dd, 1H), 5.01-5.14 (m, 1H), 6.34 (d, 1H), 6.47 (d, 1H), 6.72 (t, 1H), 7.28 (s, 1H), 7.33 (dd, 1H), 7.49-7.62 (m, 2H), 7.89 (d, 1H).

### Example 20a

### 4-Chloro-2-(((1R*,6S*)-5-((R*)-2-(5-chloropyridin-2-yl)-2-methylbenzo[d1[1,3]dioxol-4-yl)-2,5-diazabicyclo[4.2.0]]octan-2-yl)methyl)-1-(((S)-oxetan-2-yl)methyl)-1H-benzo[d]imidazole-6-carboxylic acid Isomer 1

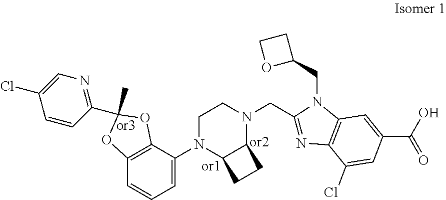 [SMILES: C[C@]1(c2ccc(Cl)cn2)Oc2cccc(N3CCN(Cc4nc5c(Cl)cc(C(=O)O)cc5n4C[C@@H]4CCO4)[C@H]4CC[C@H]43)c2O1]

NaOH (37 mg, 0.92 mmol) was added to a solution of methyl 4-chloro-2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 1 Intermediate 120 (120 mg, 0.18 mmol) in MeOH (10 mL) and water (2 mL) and the reaction mixture was stirred at 20° C. for 3 h. The reaction mixture was concentrated at reduced pressure and the residue was diluted with water (15 mL) and the pH was adjusted to 7 with 2 M HCl. The aqueous layer was extracted with EtOAc (3×15 mL) and the combined organic layer was dried over Na₂SO₄, filtered and concentrated at reduced pressure. The crude compound was purified by preparative HPLC, PrepMethod K (gradient: 17-51%) to give the title compound Isomer 1 (88 mg, 75%) as a white solid; HIRMS (ESI) m/z [M+H]⁺ calcd for C₃₂H₃₂Cl₂N₅O₅: 636.1774, found: 636.1810; ¹H NMR (300 MHz, CD₃OD) δ 1.77 (q, 2H), 1.87-2.01 (m, 1H), 2.02 (s, 3H), 2.25 (p, 1H), 2.39-2.59 (m, 2H), 2.70-2.81 (m, 2H), 3.25-3.45 (m, overlapped with solvent), 3.87 (d, 1H), 4.27 (d, 1H), 4.31-4.46 (m, 2H), 4.49-4.68 (m, 1H), 4.78 (dd, 1H), 5.00 (dd, 1H), 5.25-5.36 (m, 1H), 6.36 (dd, 1H), 6.44 (dd, 1H), 6.74 (t, 1H), 7.68 (dd, 1H), 7.89 (dd, 1H), 7.99 (d, 1H), 8.26 (d, 1H), 8.61 (dd, 1H).

### Example 20b

### 4-Chloro-2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid Isomer 2

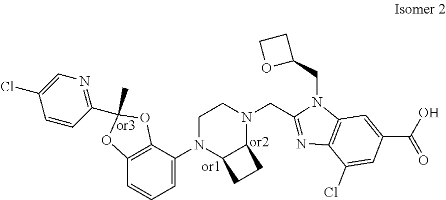 [SMILES: C[C@]1(c2ccc(Cl)cn2)Oc2cccc(N3CCN(Cc4nc5c(Cl)cc(C(=O)O)cc5n4C[C@@H]4CCO4)[C@H]4CC[C@H]43)c2O1]

The title compound was prepared as described for Example 20a from methyl 4-chloro-2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 2 Intermediate 121 (120 mg, 0.18 mmol) to give the title compound Isomer 2 (82 mg, 70%) as a white solid; HIRMS (ESI) m/z [M+H]⁺ calcd for C₃₂H₃₂Cl₂N₅O₅: 636.1774, found: 636.1820; ¹H NMR (300 MHz, DMSO-d₆) δ 1.72 (s, 3H), 2.01 (s, 3H), 2.05-2.19 (m, 1H), 2.30-2.50 (m, 2H), 2.60-2.78 (m, 2H), 3.12-3.30 (m, 3H), 3.64 (d, 1H), 4.19 (d, 1H), 4.30-4.55 (m, 3H), 4.65 (d, 1H), 4.83-4.99 (m, 1H), 5.05-5.19 (m, 1H), 6.32 (d, 1H), 6.48 (d, 1H), 6.74 (t, 1H), 7.61 (d, 1H), 7.83 (s, 1H), 8.02 (dd, 1H), 8.23 (d, 1H), 8.72 (d, 1H).

### Example 20c

### 4-Chloro-2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid Isomer 3

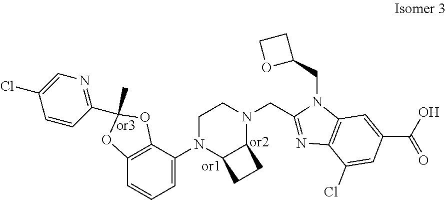 [SMILES: C[C@]1(c2ccc(Cl)cn2)Oc2cccc(N3CCN(Cc4nc5c(Cl)cc(C(=O)O)cc5n4C[C@@H]4CCO4)[C@H]4CC[C@H]43)c2O1]

The title compound was prepared as described for Example 20a from methyl 4-chloro-2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 3 Intermediate 122 (130 mg, 0.20 mmol) to give the title compound Isomer 3 (52 mg, 41%) as a white solid; HIRMS (ESI) m/z [M+H]⁺ calcd for C₃₂H₃₂Cl₂N₅O₅: 636.1774, found: 636.1810; 1H NMR (400 MHz, DMSO-d₆) δ 1.61 (s, 3H), 2.00 (s, 4H), 2.30-3.41 (m, 2H), 2.60-2.72 (m, 2H), 3.12-3.17 (m, 1H), 3.23-3.38 (m, overlapped with solvent), 3.77 (d, 1H), 4.14 (d, 1H), 4.22-4.30 (m, 2H), 4.45 (q, 1H), 4.68-4.87 (m, 2H), 5.15 (s, 1H), 6.30 (d, 1H), 6.47 (d, 1H), 6.72 (t, 1H), 7.60 (d, 1H), 7.81 (s, 1H), 8.00 (dd, 1H), 8.22 (s, 1H), 8.71 (d, 1H).

### Example 20d

### 4-Chloro-2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid Isomer 4

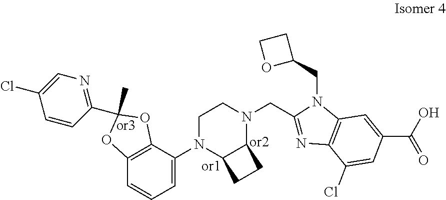 [SMILES: C[C@]1(c2ccc(Cl)cn2)Oc2cccc(N3CCN(Cc4nc5c(Cl)cc(C(=O)O)cc5n4C[C@@H]4CCO4)[C@H]4CC[C@H]43)c2O1]

The title compound was prepared as described for Example 20a from methyl 4-chloro-2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 4 Intermediate 123 (130 mg, 0.20 mmol) to give the title compound is Isomer 4 (38 mg, 30%) as a white solid; HRMS (ESI) m/z [M+H]⁺ calcd for C₃₂H₃₂Cl₂N₅O₅: 636.1774, found: 636.1818; ¹H NMR (400 MHz, DMSO-d₆) δ 1.74 (d, 2H), 1.98 (s, 4H), 2.26 (t, 1H), 2.34 (t, 1H), 2.43 (s, 1H), 2.59-2.79 (m, 2H), 3.15-3.21 (m, 1H), 3.19 (s, 1H), 3.24 (s, 1H), 3.61 (d, 1H), 4.25-4.34 (m, 2H), 4.38-4.48 (m, 1H), 4.53 (q, 1H), 4.63 (d, 1H), 4.94 (dd, 1H), 5.10-5.16 (m, 1H), 6.35 (d, 1H), 6.47 (d, 1H), 6.73 (t, 1H), 7.62 (d, 1H), 7.80 (s, 1H), 8.00 (dd, 1H), 8.22 (s, 1H), 8.71 (d, 1H).

### Example 21a

### 2-(((1R*,6R*)-5-((R*)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid Isomer 1

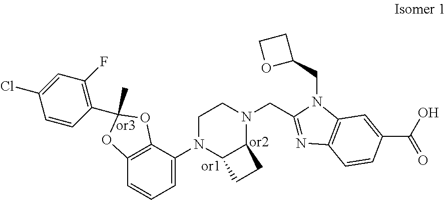 [SMILES: C[C@]1(c2ccc(Cl)cc2F)Oc2cccc(N3CCN(Cc4nc5ccc(C(=O)O)cc5n4C[C@@H]4CCO4)[C@H]4CC[C@@H]43)c2O1]

LiOH monohydrate (13 mg, 0.31 mmol) was added to a solution of methyl 2-(((1R*,6R*)-5-((R*)-2-(4-chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 1 Intermediate 125 (78 mg, 0.12 mmol) in THF:water (1:1, 2 mL) and the reaction mixture was stirred at rt overnight. The reaction mixture was concentrated in vacuo, the residue was diluted with water (3 mL) and acidified to pH 3 with 10% NaH₂PO₄ (aq). The precipitate was filtered off and dried in vacuo to give the title compound Isomer 1 (30 mg, 32%) as a solid; MS (ESI) m/z [M+H]⁺ 618.2; ¹H NMR (600 MHz, DMSO-d₆) δ 12.75 (s, 1H), 8.67 (d, 1H), 8.25 (s, 1H), 7.99 (dd, 1H), 7.80 (dd, 1H), 7.67-7.64 (d, 1H), 7.61-7.57 (d, 1H), 6.72 (t, 1H), 6.51 (d, 1H), 6.39 (d, 1H), 5.21-5.05 (m, 1H), 4.84-4.75 (m, 1H), 4.66 (dd, 1H), 4.51 (q, 1H), 4.43 (q, 1H), 4.38 (d, 1H), 4.16 (dt, 1H), 4.05 (d, 1H), 3.73 (dd, 1H), 3.62-3.56 (m, 2H), 3.37-3.32 (m, overlapped with solvent), 3.08 (d, 1H), 2.73 (d, 1H), 2.65-2.60 (m, overlapped with solvent), 2.43-2.37 (m, 2H), 2.31-2.21 (m, is 1H), 2.00 (s, 3H)

### Example 21b

### 2-(((1R*,6R*)-5-((R*)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid Isomer 2

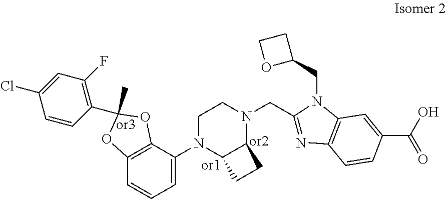 [SMILES: C[C@]1(c2ccc(Cl)cc2F)Oc2cccc(N3CCN(Cc4nc5ccc(C(=O)O)cc5n4C[C@@H]4CCO4)[C@H]4CC[C@@H]43)c2O1]

The title compound was prepared as described for Example 21a from methyl 2-(((1R*,6R*)-5-((R*1-2-(4-chloro-2-fluoronhenyl)-2-methylbenzo[d][1.31dioxol-4-yll-2.5-diazabicyclo[4.2.0]octan-2-yl)methyl)-1-(((S)-oxetan-2-yl)methyl)-1H-benzo[d]imidazole-6-carboxylate, Isomer 2 Intermediate 126 (28 mg, 44 μmol) to give the title compound Isomer 2 (21 mg, 93%) as a solid; MS (ESI) m/z [M+H]⁺ 618.2; ¹H NMR (600 MHz, DMSO-d₆) δ 12.76 (s, 1H), 8.70 (d, 1H), 8.25 (s, 1H), 7.99 (d, 1H), 7.80 (d, 1H), 7.67-7.64 (d, 1H), 7.62-7.59 (d, 1H), 6.72 (t, 1H), 6.50 (d, 1H), 6.41 (d, 1H), 5.15 (s, 1H), 4.83 (d, 1H), 4.66 (dd, 1H), 4.49-4.53 (m, 1H), 4.47-4.37 (m, 2H), 4.15 (d, 1H), 4.07 (d, 1H), 3.76 (dd, 1H), 3.72 (t, 1H), 3.66 (t, 1H), 3.57 (d, 1H), 3.21 (d, 2H), 3.12-3.00 (m, 2H), 2.76-2.70 (m, 1H), 2.60 (s, overlapped with solvent), 2.30-2.25 (m, 1H), 1.98 (s, 3H).

### Example 22a

### 2-(((1R*,6R*)-5-((R*)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid, Isomer 3

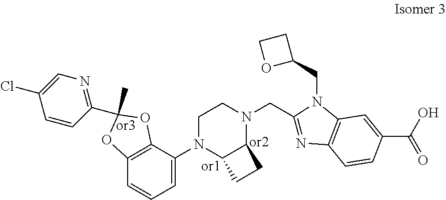 [SMILES: C[C@]1(c2ccc(Cl)cn2)Oc2cccc(N3CCN(Cc4nc5ccc(C(=O)O)cc5n4C[C@@H]4CCO4)[C@H]4CC[C@@H]43)c2O1]

The title compound was prepared as described for Example 21a from methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 3 Intermediate 128 (41 mg, 0.066 mmol) to give the title compound Isomer 3 (35 mg, 86%) as a solid; MS (ESI) m/z [M+H]⁺ 602.2; ¹H NMR (600 MHz, DMSO-d₆) δ 12.69 (s, 1H), 8.72 (d, 1H), 8.25 (s, 1H), 8.01 (dd, 1H), 7.78 (dd, 1H), 7.65-7.56 (m, 2H), 6.71 (t, 1H), 6.55 (d, 1H), 6.32 (d, 1H), 5.14-5.11 (m, 1H), 4.84 (dd, 1H), 4.63 (dd, 1H), 4.50-4.43 (m, 1H), 4.33 (dt, 1H), 3.86 (s, 1H), 3.79 (d, 1H), 3.72 (s, 1H), 3.03 (s, 1H), 2.81-2.60 (m, 4H), 2.41-2.35 (m, 1H), 2.31 (s, 1H), 2.09 (q, 1H), 1.97 (s, 3H), 1.64 (p, 1H), 1.55-1.49 (m, 1H), 1.16 (s, 1H).

### Example 22b

### 2-(((1R*,6R*)-5-((R*)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid, Isomer 4

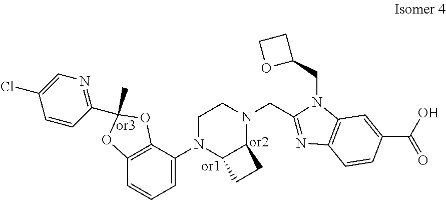 [SMILES: C[C@]1(c2ccc(Cl)cn2)Oc2cccc(N3CCN(Cc4nc5ccc(C(=O)O)cc5n4C[C@@H]4CCO4)[C@H]4CC[C@@H]43)c2O1]

The title compound was prepared as described for Example 21a from methyl 2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylate, Isomer 4 Intermediate 129 (42 mg, 0.68 mmol) to give the title compound Isomer 4 (35 mg, 84%) as a solid; MS (ESI) m/z [M+H]⁺ 602.2; ¹H NMR (600 MHz, DMSO-d₆) δ 12.72 (s, 1H), 8.67 (d, 1H), 8.24 (s, 1H), 7.93 (dd, 1H), 7.78 (dd, 1H), 7.68-7.47 (m, 2H), 6.71 (t, 1H), 6.51 (d, 1H), 6.32 (d, 1H), 5.09-5.05 (m, 1H), 4.81 (dd, 1H), 4.68 (dd, 1H), 4.48 (q, 1H), 4.37 (dt, 1H), 3.98 (d, 1H), 3.78 (d, 1H), 3.62 (s, 1H), 2.79-2.52 (m, 5H), 2.45-2.38 (m, overlaps with solvent), 2.23 (q, 1H), 1.97 (s, 3H), 1.86-1.68 (m, 2H), 1.54-1.38 (m, 1H).

### Example 23a

rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylic acid Isomer 1

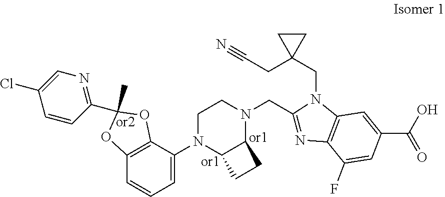 [SMILES: C[C@]1(c2ccc(Cl)cn2)Oc2cccc(N3CCN(Cc4nc5c(F)cc(C(=O)O)cc5n4CC4(CC#N)CC4)[C@H]4CC[C@@H]43)c2O1]

The title compound was prepared as described for Example 21a from rel-methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylate, Isomer 1 Intermediate 134 (68 mg, 0.10 mmol) to give the title compound Isomer 1 (65 mg, 94%) as a solid; HRMS (ESI) m/z [M+H]⁺ calcd for C₃₄H₃₃ClFN₆O₄: 643.2230, found: 643.2268; ¹H NMR (500 MHz, DMSO-d₆) δ 13.09 (s, 1H), 8.68 (d, 1H), 8.13 (s, 1H), 7.95 (dd, 1H), 7.58 (d, 1H), 7.50 (d, 1H), 6.70 (t, 1H), 6.51 (d, 1H), 6.30 (d, 1H), 4.74 (d, 1H), 4.55 (d, 1H), 3.86-3.72 (m, 3H), 2.97 (d, 1H), 2.74-2.66 (m, 1H), 2.66-2.54 (m, 3H), 2.39-2.29 (m, overlapped with solvent), 2.21-2.15 (m, 1H), 1.97 (s, 3H), 1.72-1.56 (m, 2H), 1.33-1.30 (m, 1H), 0.79-0.63 (m, 4H).

### Example 23b

### rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylic acid Isomer 2

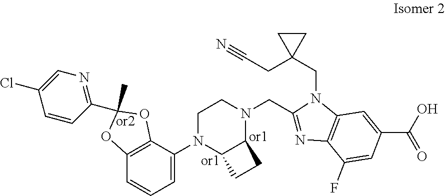 [SMILES: C[C@]1(c2ccc(Cl)cn2)Oc2cccc(N3CCN(Cc4nc5c(F)cc(C(=O)O)cc5n4CC4(CC#N)CC4)[C@H]4CC[C@@H]43)c2O1]

The title compound was prepared as described for Example 21a from rel-methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylate, Isomer 2 Intermediate 135 (62 mg, 94 μmol) to give the title compound Isomer 2 (50 mg, 93%) as a solid; HRMS (ESI) m/z [M+H]⁺ calcd for C₃₄H₃₃ClFN₆O₄: 643.2230, found: 643.2246; ¹H NMR (500 MHz, DMSO-d₆) δ 13.08 (s, 1H), 8.72 (d, 1H), 8.13 (s, 1H), 8.01 (dd, 1H), 7.59 (d, 1H), 7.50 (d, 1H), 6.71 (t, 2H), 6.55 (d, 1H), 6.32 (d, 1H), 4.78 (d, 1H), 4.54 (d, 1H), 3.84-3.77 (m, 3H), 3.07-2.90 (m, 1H), 2.74-2.52 (m, 4H), 2.43-2.38 (m, overlapped with solvent), 2.35-2.27 (m, 1H), 2.11-2.08 (m, lfH), 1.97 (s, 3H), 1.64 (dt, 1H), 1.57 (d, 1H), 1.26 (t, 1H), 0.78-0.65 (m, 4H).

### Example 23c

### rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylic acid Isomer 3

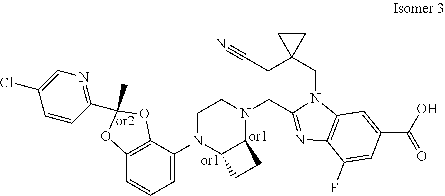 [SMILES: C[C@]1(c2ccc(Cl)cn2)Oc2cccc(N3CCN(Cc4nc5c(F)cc(C(=O)O)cc5n4CC4(CC#N)CC4)[C@H]4CC[C@@H]43)c2O1]

The title compound was prepared as described for Example 21a from rel-methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylate, Isomer 3 Intermediate 136 (84 mg, 0.13 mmol) to give the title compound Isomer 3 (52 mg, 73%) as a solid; HRMS (ESI) m/z [M+H]⁺ calcd for C₃₄H₃₃ClFN₆O₄: 643.2230, found: 643.2244; ¹H NMR (500 MHz, DMSO-d₆) δ 13.08 (s, 1H), 8.68 (d, 1H), 8.13 (s, 1H), 7.95 (dd, 1H), 7.58 (d, 1H), 7.50 (d, 1H), 6.70 (t, 1H), 6.51 (d, 1H), 6.30 (d, 1H), 4.74 (d, 1H), 4.55 (d, 1H), 3.85-3.71 (m, 3H), 2.97 (d, 1H), 2.75-2.66 (m, 1H), 2.66-2.53 (m, 3H), 2.42-2.29 (m, overlapped with solvent), 2.25-2.12 (m, 1H), 1.97 (s, 3H), 1.72-1.65 (m, 1H), 1.62 (d, 1H), 1.32 (t, 1H), 0.79-0.65 (m, 4H).

### Example 23d

### rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylic acid Isomer 4

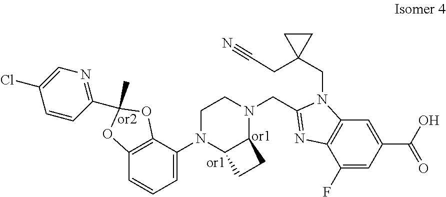 [SMILES: C[C@]1(c2ccc(Cl)cn2)Oc2cccc(N3CCN(Cc4nc5c(F)cc(C(=O)O)cc5n4CC4(CC#N)CC4)[C@H]4CC[C@@H]43)c2O1]

The title compound was prepared as described for Example 21a from rel-methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylate, Isomer 4 Intermediate 137 (59 mg, 90 μmol) to give the title compound Isomer 4 (18 mg, 31%) as a solid; HRMS (ESI) m/z [M+H]⁺ calcd for C₃₄H₃₃ClFN₆O₄: 643.2230, found: 643.2280; ¹H NMR (500 MHz, DMSO-d₆) δ 13.07 (s, 1H), 8.72 (d, 1H), 8.12 (d, 1H), 8.01 (dd, 1H), 7.59 (d, 1H), 7.50 (d, 1H), 6.71 (t, 1H), 6.54 (d, 1H), 6.31 (d, 1H), 4.78 (d, 1H), 4.54 (d, 1H), 3.86-3.71 (m, 3H), 3.01 (d, 1H), 2.72-2.57 (m, 4H), 2.44-2.39 (m, overlapped with solvent), 2.35-2.29 (m, 1H), 2.12-2.05 (m, 1H), 1.97 (s, 3H), 1.69-1.62 (m, 1H), 1.61-1.54 (m, 1H), 1.31-1.19 (m, 1H), 0.79-0.67 (m, 4H).

### Example 24a

### rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylic acid Isomer 2

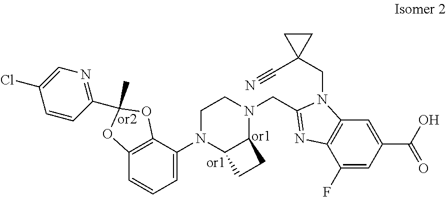 [SMILES: C[C@]1(c2ccc(Cl)cn2)Oc2cccc(N3CCN(Cc4nc5c(F)cc(C(=O)O)cc5n4CC4(C#N)CC4)[C@H]4CC[C@@H]43)c2O1]

The title compound was prepared as described for Example 21a from rel-methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylate Isomer 2 Intermediate 140 (65 mg, 0.10 mmol) to give the title compound Isomer 2 (56 mg, 87%) as a solid; HIRMS (ESI) m/z [M+H]⁺ calcd for C₃₃H₃₁ClFN₆O₄: 629.2074, found: 629.2102; ¹H NMR (500 MHz, DMSO-d₆) δ 13.02 (s, 1H, exchange), 8.72 (d, 1H), 8.23 (s, 1H), 8.01 (dd, 1H), 7.59 (d, 1H), 7.53 (d, 1H), 6.71 (t, 1H), 6.55 (d, 1H), 6.32 (d, 1H), 4.90 (d, 1H), 4.72 (d, 1H), 3.92-3.81 (m, 2H), 3.80-3.72 (m, 1H), 2.99 (dd, 1H), 2.75-2.62 (m, 3H), 2.38-2.27 (m, 1H), 2.13-2.05 (m, 1H), 1.97 (s, 3H), 1.70-1.63 (m, 1H), 1.58 (dd, 1H), 1.47-1.22 (m, 5H).

### Example 24b

### rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylic acid Isomer 4

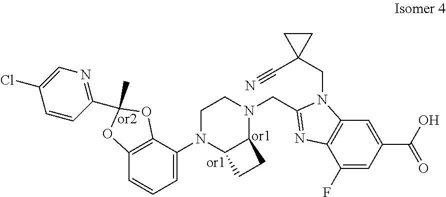 [SMILES: C[C@]1(c2ccc(Cl)cn2)Oc2cccc(N3CCN(Cc4nc5c(F)cc(C(=O)O)cc5n4CC4(C#N)CC4)[C@H]4CC[C@@H]43)c2O1]

The title compound was prepared as described for Example 21a from rel-methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylate Isomer 4 Intermediate 141 (99 mg, 0.15 mmol) to give the title compound Isomer 4 (73 mg, 87%) as a solid; HRMS (ESI) m/z [M+H]⁺ calcd for C₃₃H₃₁ClFN₆O₄: 629.2074, found: 629.2110; ¹H NMR (500 MHz, DMSO-d₆) δ 13.1 (s, 1H, exchange), 8.68 (d, 1H), 8.23 (d, 1H), 7.95 (dd, 1H), 7.67-7.41 (m, 2H), 6.70 (t, 1H), 6.52 (d, 1H), 6.30 (d, 1H), 4.87 (d, 1H), 4.74 (d, 1H), 3.86 (q, 2H), 3.77 (d, 1H), 2.96 (dd, 1H), 2.76-2.66 (m, 1H), 2.63-2.58 (m, 1H), 2.38-2.32 (m, 1H), 2.22-2.12 (m, 1H), 1.97 (s, 3H), 1.76-1.63 (m, 2H), 1.46-1.28 (m, 6H).

### Example 25a

### rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylic acid, Isomer 1

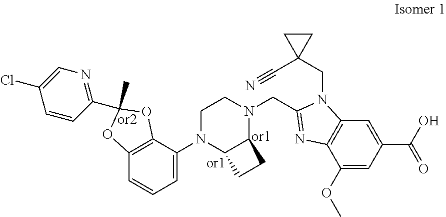 [SMILES: COc1cc(C(=O)O)cc2c1nc(CN1CCN(c3cccc4c3O[C@@](C)(c3ccc(Cl)cn3)O4)[C@H]3CC[C@@H]31)n2CC1(C#N)CC1]

The title compound was prepared as described for Example 21a from rel-methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylate Isomer 1 Intermediate 146 (16 mg, 24 μmol) to give the title compound Isomer 1 (15 mg, 94%) as a solid; HRMS (ESI) m/z [M+H]⁺ calcd for C₃₄H₃₄ClN₆05: 642.2274, found: 642.2324; ¹H NMR (500 MHz, DMSO-d₆) δ 12.85 (s, 1H), 8.67 (d, 1H), 7.97-7.93 (m, 2H), 7.57 (d, 1H), 7.28 (s, 1H), 6.70 (t, 1H), 6.51 (d, 1H), 6.30 (d, 1H), 4.80 (d, 1H), 4.67 (d, 1H), 3.95 (s, 3H), 3.87-3.73 (m, 3H), 2.93 (d, 1H), 2.71 (q, 1H), 2.45-2.39 (m, overlapped with solvent), 2.33 (q, 1H), 2.17 (q, 1H), 1.97 (s, 3H), 1.73-1.60 (m, 2H), 1.44-1.18 (m, 5H).

### Example 25b

### rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylic acid, Isomer 2

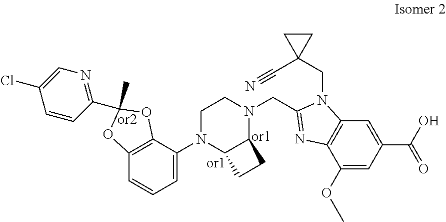 [SMILES: COc1cc(C(=O)O)cc2c1nc(CN1CCN(c3cccc4c3O[C@@](C)(c3ccc(Cl)cn3)O4)[C@H]3CC[C@@H]31)n2CC1(C#N)CC1]

The title compound was prepared as described for Example 21a from rel-methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylate Isomer 2 Intermediate 147 (20 mg, 31 μmol) to give the title compound Isomer 2 (20 mg, 93%) as a solid; HIRMS (ESI) m/z [M+H]⁺ calcd for C₃₄H₃₄ClN₆05: 642.2274, found: 642.2310; ¹H NMR (500 MHz, DMSO-d₆) δ 12.85 (s, 1H), 8.72 (d, 1H), 8.01 (dd, 1H), 7.96 (d, 1H), 7.59 (d, 1H), 7.27 (d, 1H), 6.71 (t, 1H), 6.60-6.52 (m, 1H), 6.32 (d, 1H), 4.83 (d, 1H), 4.64 (d, 1H), 3.95 (s, 3H), 3.87-3.72 (m, 3H), 2.97 (d, 1H), 2.78-2.62 (m, 2H), 2.45-2.38 (m, 1H), 2.30 (q, 1H), 2.15-2.05 (m, 1H), 1.97 (s, 3H), 1.65 (p, 1H), 1.56 (q, 1H), 1.44-1.20 (m, 5H).

### Example 25c

### rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylic acid. Isomer 3

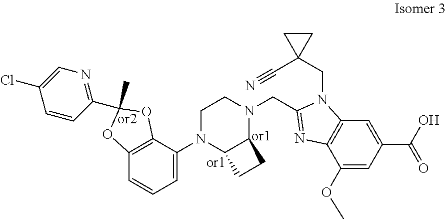 [SMILES: COc1cc(C(=O)O)cc2c1nc(CN1CCN(c3cccc4c3O[C@@](C)(c3ccc(Cl)cn3)O4)[C@H]3CC[C@@H]31)n2CC1(C#N)CC1]

The title compound was prepared as described for Example 21a from rel-methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylate Isomer 3 Intermediate 148 (19 mg, 29 μmol) to give the title compound Isomer 3 (18 mg, 96%) as a solid; HRMS (ESI) m/z [M+H]⁺ calcd for C₃₄H₃₄ClN₆05: 642.2274, found: 642.2284; ¹H NMR (500 MHz, DMSO-d₆) δ 12.85 (s, 1H), 8.67 (d, 1H), 7.97-7.93 (m, 2H), 7.57 (dd, 1H), 7.28 (d, 1H), 6.70 (t, 1H), 6.51 (d, 1H), 6.30 (d, 1H), 4.80 (d, 1H), 4.67 (d, 1H), 3.95 (s, 3H), 3.89-3.73 (m, 3H), 2.93 (d, 1H), 2.71 (q, 1H), 2.59-2.39 (m, overlapped with solvent), 2.37-2.29 (m, 1H), 2.18 (q, 1H), 1.97 (s, 3H), 1.70 (p, 1H), 1.63 (q, 1H), 1.43-1.18 (m, 5H).

### Example 25d

### rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylic acid, Isomer 4

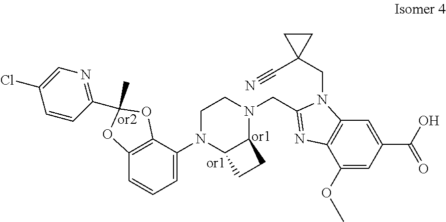 [SMILES: COc1cc(C(=O)O)cc2c1nc(CN1CCN(c3cccc4c3O[C@@](C)(c3ccc(Cl)cn3)O4)[C@H]3CC[C@@H]31)n2CC1(C#N)CC1]

The title compound was prepared as described for Example 21a from rel-methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylate Isomer 4 Intermediate 149 (20 mg, 31 μmol) to give the title compound Isomer 4 (18 mg, 92%) as a solid; HRMS (ESI) m/z [M+H]⁺ calcd for C₃₄H₃₄ClN₆05: 642.2274, found: 642.2278; ¹H NMR (500 MHz, DMSO-d₆) δ 12.86 (s, 1H), 8.72 (d, 1H), 8.01 (dd, 1H), 7.95 (d, 1H), 7.59 (dd, 1H), 7.27 (d, 1H), 6.71 (t, 1H), 6.54 (d, 1H), 6.32 (d, 1H), 4.83 (d, 1H), 4.64 (d, 1H), 3.95 (s, 3H), 3.87-3.72 (m, 3H), 2.97 (d, 1H), 2.77-2.63 (m, 2H), 2.45-2.38 (m, 1H), 2.30 (q, 1H), 2.09 (q, 1H), 1.97 (s, 3H), 1.65 (p, 1H), 1.56 (q, 1H), 1.43-1.20 (m, 5H).

### Example 26a

### rel-4-Chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylic acid Isomer 1

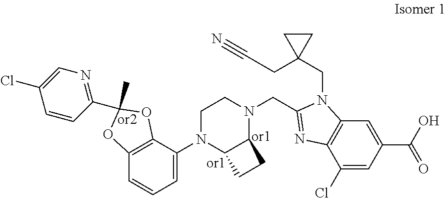 [SMILES: C[C@]1(c2ccc(Cl)cn2)Oc2cccc(N3CCN(Cc4nc5c(Cl)cc(C(=O)O)cc5n4CC4(CC#N)CC4)[C@H]4CC[C@@H]43)c2O1]

The title compound was prepared as described for Example 21a from rel-methyl 4-chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylate Isomer 1 Intermediate 154 (29 mg, 43 μmol) to give the title compound Isomer 1 (28 mg, 92%) as a solid; MS (ESI) m/z [M+H]⁺ 659.2; ¹H NMR (500 is MHz, DMSO-d₆) δ 13.12 (s, 1H), 8.72 (d, 1H), 8.23 (s, 1H), 8.01 (dd, 1H), 7.78 (s, 1H), 7.59 (d, 1H), 6.71 (t, 1H), 6.54 (d, 1H), 6.31 (d, 1H), 4.78 (d, 1H), 4.55 (d, 1H), 3.90-3.75 (m, 3H), 3.01 (d, 1H), 2.74-2.57 (m, 4H), 2.32 (q, 1H), 2.09 (q, 1H), 1.97 (s, 3H), 1.66 (p, 1H), 1.56 (q, 1H), 1.29-1.20 (m, 2H), 0.80-0.65 (m, 4H).

### Example 26b

### rel-4-Chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylic acid Isomer 2

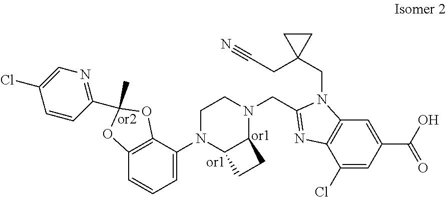 [SMILES: C[C@]1(c2ccc(Cl)cn2)Oc2cccc(N3CCN(Cc4nc5c(Cl)cc(C(=O)O)cc5n4CC4(CC#N)CC4)[C@H]4CC[C@@H]43)c2O1]

The title compound was prepared as described for Example 21a from rel-methyl 4-chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylate Isomer 2 Intermediate 155 (32 mg, 46 μmol) to give the title compound Isomer 2 (27 mg, 87%) as a solid; MS (ESI) m/z [M+H]⁺ 659.2; ¹H NMR (500 MHz, DMSO-d₆) δ 13.12 (s, 1H), 8.68 (d, 1H), 8.23 (d, 1H), 7.95 (dd, 1H), 7.79 (d, 1H), 7.58 (d, 1H), 6.70 (t, 1H), 6.51 (d, 1H), 6.29 (d, 1H), 4.75 (d, 1H), 4.56 (d, 1H), 3.92-3.73 (m, 3H), 2.96 (d, 1H), 2.71-2.59 (m, 4H), 2.47-2.31 (m, 2H), 2.20-2.13 (m, 1H), 1.97 (s, 3H), 1.74-1.65 (m, 1H), 1.65-1.58 (m, 1H), 1.31 (p, 1H), 0.80-0.62 (m, 4H).

### Example 26c

### rel-4-Chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylic acid Isomer 3

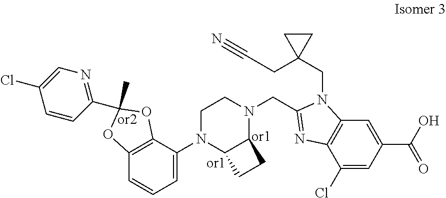 [SMILES: C[C@]1(c2ccc(Cl)cn2)Oc2cccc(N3CCN(Cc4nc5c(Cl)cc(C(=O)O)cc5n4CC4(CC#N)CC4)[C@H]4CC[C@@H]43)c2O1]

The title compound was prepared as described for Example 21a from rel-methyl 4-chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylate Isomer 3 Intermediate 156 (27 mg, 40 μmol) to give the title compound Isomer 3 (26 mg, 94%) as a solid; MS (ESI) m/z [M+H]⁺ 659.0; ¹H NMR (500 MHz, DMSO-d₆) δ 13.12 (s, 1H), 8.68 (d, 1H), 8.23 (d, 1H), 7.95 (dd, 1H), 7.79 (d, 1H), 7.58 (d, 1H), 6.70 (t, 1H), 6.51 (d, 1H), 6.29 (d, 1H), 4.75 (d, 1H), 4.56 (d, 1H), 3.93-3.68 (m, 3H), 2.96 (d, 1H), 2.74-2.66 (m, 1H), 2.63 (d, 2H), 2.61-2.54 (m, 2H), 2.39-2.32 (m, 1H), 2.17 (q, 1H), 1.97 (s, 3H), 1.76-1.55 (m, 2H), 1.35-1.26 (m, 1H), 0.79-0.65 (m, 4H

### Example 26d

### rel-4-Chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylic acid Isomer 4

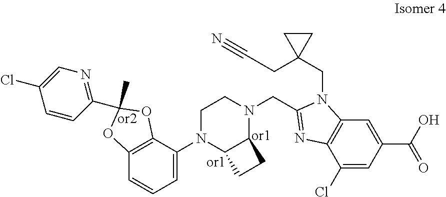 [SMILES: C[C@]1(c2ccc(Cl)cn2)Oc2cccc(N3CCN(Cc4nc5c(Cl)cc(C(=O)O)cc5n4CC4(CC#N)CC4)[C@H]4CC[C@@H]43)c2O1]

The title compound was prepared as described for Example 21a from rel-methyl 4-chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylate Isomer 4 Intermediate 157 (29 mg, 43 μmol) to give the title compound Isomer 4 (27 mg, 93%) as a solid; MS (ESI) m/z [M+H]⁺ 659.0; ¹H NMR (500 MHz, DMSO-d₆) δ 13.12 (s, 1H), 8.72 (d, 1H), 8.23 (d, 1H), 8.01 (dd, 1H), 7.78 (d, 1H), 7.59 (d, 1H), 6.71 (t, 1H), 6.54 (d, 1H), 6.31 (d, 1H), 4.78 (d, 1H), 4.55 (d, 1H), 3.87-3.76 (m, 3H), 3.01 (d, 1H), 2.72-2.58 (m, 4H), 2.46-2.40 (d, 1H, overlapped with solvent), 2.32 (q, 1H), 2.09 (q, 1H), 1.97 (s, 3H), 1.71-1.62 (m, 1H), 1.60-1.53 (m, 1H), 1.29-1.20 (m, 1H), 0.81-0.63 (m, 4H).

### Example 27

### rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-(2-cyclopropoxvethyl)-4-methoxy-1H-benzo[d]imidazole-6-carboxylic acid, Isomer 3

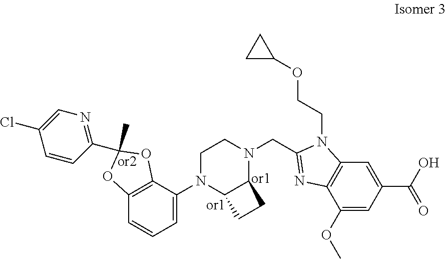 [SMILES: COc1cc(C(=O)O)cc2c1nc(CN1CCN(c3cccc4c3O[C@@](C)(c3ccc(Cl)cn3)O4)[C@H]3CC[C@@H]31)n2CCOC1CC1]

The title compound was prepared as described for Example 21a from rel-methyl 2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-(2-cyclopropoxyethyl)-4-methoxy-1H-benzo[d]imidazole-6-carboxylate Isomer 3 Intermediate 162 (46 mg, 70 μmol) to give the title compound Isomer 3 (32 mg, 96%) as a solid; MS (ESI) m/z [M+H]⁺ 646.4; ¹H NMR (500 MHz, DMSO-d₆) δ 12.75 (s, 1H), 8.72 (d, 1H), 8.02 (dd, 1H), 7.79 (s, 1H), 7.59 (d, 1H), 7.23 (s, 1H), 6.71 (t, 1H), 6.61-6.45 (m, 1H), 6.32 (d, 1H), 4.66-4.46 (m, 2H), 3.93 (s, 3H), 3.84-3.81 (m, 2H), 3.78-3.72 (m, 1H), 3.72-3.64 (m, 2H), 3.25-3.19 (m, 1H), 2.92 (d, 1H), 2.71-2.62 (m, 2H), 2.28 (q, 1H), 2.12-2.09 (m, 1H), 1.97 (s, 3H), 1.69-1.54 (m, 2H), 1.25-1.22 (m, 2H), 0.34-0.32 (m, 2H), 0.27-0.19 (m, 2H).

### PHARMACOLOGICAL ACTIVITY

CHOK1 GLP-1R cAMP Assay

A cell line stably expressing the human GLP-1R receptor (NM_002062.5, including the naturally-occurring variant Leu260Phe) in a CHO-K₁ (ATCC® CCL-61™) was used for assay.

GLP-1 receptor mediated agonist activity was determined in a cell-based assay measuring cyclic adenosine monophosphate (cAMP) levels in cells using Homogeneous Time-Resolved Fluorescence (HTRF) cAMP detection kit (CisBio catalog #62AM4PEC, cAMP Gs Dynamic range kit). The cAMP detection method is based on a competitive immunoassay, in which cAMP produced by the cells and cAMP labeled with the dye d2 compete for binding to a Europium-Cryptate-labeled anti-cAMP antibody. The specific HTRF signal is inversely proportional to the concentration of cAMP.

Compounds were added to individual well in 384 well-assay plates (Greiner #784076) using an Echo (LabCyte) dispenser from 10 mM stocks. Varying concentration of compounds were added to wells, and DMSO was used to normalize each well to a volume of 100 nL. A dose response curve of GLP-1(7-36)NH₂ (Bachem H-6795) was included in each run. 5 μL of cAMP concentration response standards are applied in specified wells in the assay plates. Cryo-preserved cells are thawed and resuspended in assay buffer pre-heated to 37° C. (20 mM 2-[4-(2-hydroxyethyl)piperazin-1-yl]ethanesulfonic acid (HEPES) pH 7.4, 1× Hank's Balanced Salt Solution (HBSS, Life Technologies #14065) supplemented with 0.1% (w/v) bovine serum albumin (Sigma, A-7030)). Cells were centrifuged at 250*g for 5 min at rt, and resuspended in room tempered assay buffer to a final density of 0.16*10⁶ cell/mL, to deliver 800 cells/well. 5 μL of assay buffer with 1 mM 3-isobutyl-1-methylxanthin (IBMX; Sigma cat I-7018) was dispensed per well in assay plates using a multidrop combi (Thermo Scientific) subsequently 5 μL of cell suspension was distributed to relevant wells in the assay using a multidrop dispenser. Assay plates were incubated 20 min at rt.

Detection reagents, Europium-Cryptate-labeled anti-cAMP antibody and cAMP labeled with the dye d2, are diluted in lysis buffer, provided by the manufacturer. 5 μL of each detection reagent is supplemented to each assay well using a multidrop dispenser. Assay plates are incubated in the dark for at least one h. The HTRF signal is measured using the HTRF module (excitation: 337 nm, emission A: 665 nm and emission B: 620 nm) in Pherastar FSX (BMG Labtech).

Raw data were converted to pM cAMP using the cAMP standard curve included in each run. Converted data were further analyzed in Genedata Screener (Genedata) and EC50 determinations were made from agonist dose-response curves analyzed with a curve fitting is program using a 4-parameter logistic dose response equation (Equation y=A+((B-A)/1+((C/x)\hat{ }D))) where A is no stimulation, B is full stimulation, C is the EC50 and D is the Hill slope). The percent effect was determined relative to a saturating concentration of a full GLP-1R agonist (GLP-1(7-36)NH₂ has 100% effect in this assay setup).

The GLP-1R EC₅₀ values for the Example compounds are set forth in Table 1 herein below.

TABLE 1

| Ex. No. | GLP-1R EC50 (nM) |
|---|---|
| 1a | 66 |
| 1b | 1.2 |
| 1c | 2.2 |
| 2a | 0.42 |
| 2b | 25 |
| 2c | 430 |
| 2d | 1.4 |
| 3a | 220 |
| 3b | 0.9 |
| 3c | 11 |
| 4a | 0.4 |
| 4b | 8.3 |
| 4c | 8.5 |
| 5a | 1.9 |
| 5b | 18 |
| 5c | 180 |
| 5d | 4.7 |
| 6a | 1.9 |
| 6b | 29 |
| 6c | 34 |
| 6d | 120 |
| 7a | 27 |
| 7b | 2.5 |
| 7c | 110 |
| 8a | 6.2 |
| 8b | 400 |
| 8c | 1.6 |
| 8d | 93 |
| 9a | 57 |
| 9b | 0.8 |
| 9c | 28 |
| 9d | 2.3 |
| 10a | 0.9 |
| 10b | 19 |
| 10c | 240 |
| 11 | 2.7 |
| 12 | 200 |
| 13 | 5.2 |
| 14 | 45 |
| 15a | 0.69 |
| 15b | 1.3 |
| 16a | 0.69 |
| 16b | 2.5 |
| 16c | 59 |
| 17a | 0.58 |
| 17b | 3.4 |
| 17c | 47 |
| 18a | 0.98 |
| 18b | 120 |
| 18c | 5.8 |
| 19a | 92 |
| 19b | 0.61 |
| 19c | 83 |
| 19d | 1.5 |
| 20a | 42 |
| 20b | 140 |
| 20c | 0.81 |
| 20d | 2.5 |
| 21a | 7 |
| 21b | 340 |
| 22a | 29 |
| 22b | 5.3 |
| 23a | 1.1 |
| 23b | 27 |
| 23c | 140 |
| 23d | 11 |
| 24a | 32 |
| 24b | 2.1 |
| 25a | 130 |
| 25b | 6.2 |
| 25c | 1.2 |
| 25d | 36 |
| 26a | 350 |
| 26b | 1.5 |
| 26c | 64 |
| 26d | 10 |
| 27 | 65 |

EndoC cAMP Accumulation Assay

A HTRF cAMP assay (cAMP Gs dynamic kit; CisoBio Cat #62AM4PEJ) was used to identify agonists of GLP-1R in a pancreatic insulinoma cell line (EndoC-PH1). The EndoC-PH1 cell line was sourced from Univercell Biosolutions and is a genetically engineered human pancreatic R cell line which exhibits glucose-inducible insulin secretion. EndoC-PHlcells have detectable GLP-1R messenger ribonucleic acid (mRNA) as detected by quantitative polymerase chain reaction (qPCR). The functionality of GLP-1R signalling in EndoC-PH1 has been demonstrated by Exendin-4 treatment leading to augmented insulin secretion; an effect which is blunted with short hairpin ribonucleic acid (shRNA)-mediated knockdown of GLP-1R. The EndoC-PH1 cell line is a valid model of human beta cells and applicable for screenings to identify novel drug target candidates (Mol. Metab., 2018, 8, 144-157). CisBio HTRF cAMP kits are based on a competitive immunoassay using cryptate-labelled anti-cAMP antibody and d2-labeled cAMP. The detection kit is intended for the direct is quantitative determination of cAMP. The specific signal (i.e. energy transfer) is inversely proportional to the concentration of cAMP in the standard or sample. Test compounds (10 mM in DMSO) were diluted into assay buffer (HBSS (Sigma #H8264) supplemented with 25 mM HEPES (Gibco #15630, pH 7.4), 0.1% BSA (Sigma #A3059) and 0.5 mM IBMX (Sigma #I7018) included fresh on the day of the assay) into 96 well U-bottom plates (Greiner #650201). Diluted compounds were transferred to ECHO source polypropelene plates (Labcyte #P-05525) and dose response curves were dispensed acoustically using ECHO 550 into black shallow-well u-bottom 384-well HTRF Assay Plates (Corning 4514). Cryovials of EndoC-H1 (supplied at 1×10e⁷ cells/vial) were used directly for screening. The cryovials and were removed from N₂(1) and thawed rapidly in a 37° C. water bath. The cells were resuspended in assay buffer and centrifuged at 300 g for 5 min. Cells were resuspended in assay buffer at the appropriate concentration, typically at 12e5 cells per mL (3000 cells per well, dependent on cell batch) and 2.5 μL diluted cells were added to all wells of destination plate by Multidrop combi reagent dispenser (Thermofisher). The plates were incubated at rt for 30 min. The assay was stopped by adding 2.5 μL anti-cAMP cryptate solution to all wells and 2.5 μL cAMP-d2 solution (both diluted 1:20 in lysis buffer) to columns 1-22 by Combi drop. A volume of 2.5 μL cAMP-d2 solution was added to wells E23 to P24 and 2.5 μL lysis buffer added to wells A23 to D24 by multichannel pipette. The plates were incubated at rt for 1 h and read on an Envision plate reader using excitation wavelength of 320 nm and emission of 590 nm and 660 nm.

Raw data from Envision is converted to % DeltaF according to the manufacturer's instructions. Dose response curves are analysed via 4-Parameter Logistical Analysis and assay plate Z’ values obtained. Samples are graphed as percentage (%) activation plots compared to GIP (1-42, Bachem H-5645) with assay window defined by negative control as basal cell is cAMP levels and positive control are defined by maximum GIP (82.5 nM) signal. GLP-1 (7-36 amide, Bachem H-6795) dose response curve was included on all plates.

The EndoC EC₅₀ values for the Example compounds are set forth in Table 2 herein below.

TABLE 2

| Ex. No. | EndoC EC50 (nM) |
|---|---|
| 1a | 280 |
| 1b | 2.9 |
| 1c | 3.2 |
| 2a | 1.7 |
| 2b | 120 |
| 2c | 640 |
| 2d | 1.9 |
| 3a | 4400 |
| 3b | 2.6 |
| 3c | 8.8 |
| 4a | 1.6 |
| 4b | 18 |
| 4c | 4.3 |
| 5a | 6.5 |
| 5b | 120 |
| 5c | 1800 |
| 5d | 8.6 |
| 6a | 3.7 |
| 6b | 64 |
| 6c | 10 |
| 6d | 330 |
| 7a | 72 |
| 7b | 6.1 |
| 7c | 32 |
| 8a | 6.6 |
| 8b | 590 |
| 8c | 2.6 |
| 8d | 370 |
| 9a | 340 |
| 9b | 3.3 |
| 9c | 74 |
| 9d | 4.3 |
| 10a | 3.5 |
| 10b | 76 |
| 10c | 1000 |
| 11 | 5.8 |
| 12 | 320 |
| 13 | |
| 14 | 38 |
| 15a | 0.9 |
| 15b | 1.4 |
| 16a | 1.3 |
| 16b | 2.3 |
| 16c | 65 |
| 17a | 1.6 |
| 17b | 4.6 |
| 17c | |
| 18a | 3.3 |
| 18b | 480 |
| 18c | 10 |
| 19a | |
| 19b | 1.9 |
| 19c | |
| 19d | 2 |
| 20a | |
| 20b | |
| 20c | 2.2 |
| 20d | 2.8 |
| 21a | 7.3 |
| 21b | 2200 |
| 22a | 49 |
| 22b | 18 |
| 23a | 2.8 |
| 23b | 85 |
| 23c | 360 |
| 23d | 7.6 |
| 24a | 16 |
| 24b | 8 |
| 25a | 260 |
| 25b | 7.5 |
| 25c | 3.1 |
| 25d | 74 |
| 26a | 450 |
| 26b | 2 |
| 26c | 160 |
| 26d | 7 |
| 27 | 200 |

Inhibition of phosphodiesterase-3 (PDE3) has been shown to result in an increase in cardiovascular mortality in clinical trials (Movsesian M.A., Kukreja R.C. (2011) Phosphodiesterase Inhibition in Heart Failure. In: Francis S., Conti M., Houslay M. (eds) Phosphodiesterases as Drug Targets. Handbook of Experimental Pharmacology, vol 204. Springer, Berlin, Heidelberg. https://doi.org/10.1007/978-3-642-17969-3 10). Chronic treatment with PDE3 inhibitors has been shown to result in increased mortality, primarily as a result of arrhythmias and sudden death (Expert Opinion on InvestigationalDrugs, 2002, 11, 1529-1536; J. of Cardiovasc. Trans. Res., 2010, 3, 507-515) and it may therefore be an advantage to as far as possible avoid PDE3 inhibitory activity.

### PDE3 Assay

Evaluation of the effects of compounds on the activity of the human phosphodiesterase-3A is quantified by measuring the formation of 5′AMP from cAMP using a human recombinant is enzyme expressed in a clonal isolate of Spodoptera frugiperda cells (Sf9) cells.

The test compound, reference compound or water (control) are added to a buffer containing 40 mM tris(hydroxymethyl)aminomethane (Tris)/HCl (pH 7.4) and 8 mM MgCl₂, 450 nMcAMP and 0.25 μCi [³H]cAMP.

Thereafter, the reaction is initiated by addition of the enzyme (about 1U) and the mixture is incubated for 20 min at 22° C.

For basal control measurements, the enzyme is omitted from the reaction mixture.

Following incubation SPA beads are added. After 30 min at 22° C. under shaking, the amount of [³H]5′AMP is quantified with a scintillation counter (Topcount, Packard).

The results are expressed as a percent inhibition of the control enzyme activity. The standard inhibitory reference compound is milrinone (CAS number 78415-72-2), which is tested in each experiment at several concentrations to obtain an inhibition curve from which its IC₅₀ value is calculated.

The PDE3 IC₅₀ values for Example compounds and reference compounds are set forth in Table 3 herein below.

TABLE 3

| Ex. No. | PDE3 IC50 (μM) |
|---|---|
| 1b | >87 |
| 1c | >100 |
| 2a | >100 |
| 2d | >100 |
| 3b | >100 |
| 3c | >100 |
| 4a | >74 |
| 5a | >100 |
| 6a | >100 |
| 7b | >100 |
| 8c | >100 |
| 9b | >100 |
| 10a | >100 |
| Ref Comp A* | 4 |
| Ref Comp B** | 11 |
| Ref Comp C*** | 2 |
| Ref Comp D**** | 5 |

*Ref Comp A may be prepared as disclosed in WO2020103815, Ex 19
**Ref Comp B may be prepared as disclosed in WO2018109607, Ex 4A-01
***Ref Comp C may be prepared as disclosed in WO2021112538, Ex 73 or as disclosed in WO2021081207, Ex 67, or as disclosed in WO2020263695, Ex 3.
****Ref Comp D may be prepared as disclosed in WO2020263695, Ex 2

1. A compound of Formula (I) 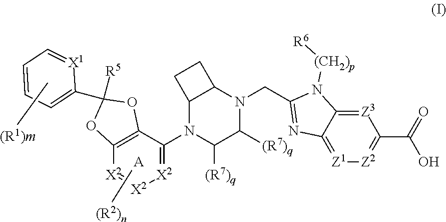
wherein
X¹ is N or C;
X² is independently N or C, provided that no more than two atoms in the aromatic ring A are N;
Z¹ is N or CR³;
Z² and Z³ are each independently N or CR⁴, provided that when Z¹ or Z³ is N, Z² is CR⁴;
R¹ is 0, 1, 2 or 3 substituents independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
R⁷ is independently selected from F, C₁₋₂alkyl and OC₁₋₂alkyl, wherein said C₁₋₂alkyl is substituted by 0, 1, 2 or 3 substituents independently selected from F;
m is 1, 2 or 3;
n is 0 or 1;
p is 1, 2 or 3;
q is 0, 1 or 2;
or a pharmaceutically acceptable salt thereof.

2. A compound according to claim 1 of Formula (Ta) 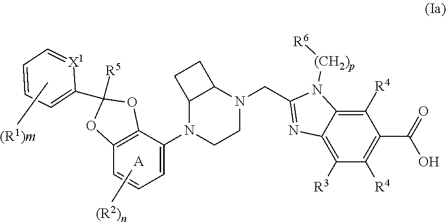
wherein
X¹ is Nor C;
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
m is 0, 1, 2 or 3;
n is 0 or 1;
p is 1, 2 or 3;
or a pharmaceutically acceptable salt thereof.

3. A compound according to claim 2,
wherein
X¹ is N;
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl and (5- to 6-membered)heteroaryl wherein said (4- to 6-membered)heterocycloalkyl and (5- to 6-membered)heteroaryl is substituted by 0 or 1 substituent selected from C₁₋₂alkyl and;
m is 0, 1 or 2;
n is 0 or 1;
p is 1;
or a pharmaceutically acceptable salt thereof.

4. A compound according to claim 1 of Formula (Id) 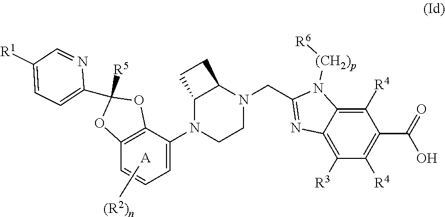
wherein
R¹ is independently selected from F, Cl, Br, CN, OCH₃, OCFH₂, OCF₂H, OCF₃, CH₃, CFH₂, CF₂H and CF₃;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃, CFH₂, CF₂H, CF₃, OCH₃, OCFH₂, OCF₂H and OCF₃;
R⁵ is selected from H, CH₃, CFH₂, CF₂H and CF₃;
R⁶ is selected from (4- to 6-membered)heterocycloalkyl, (5- to 6-membered)heteroaryl, CN, C₁₋₄alkyl, O(C₁₋₄alkyl), S(C₁₋₄alkyl), cyclopropyl, cyclobutyl, O(cyclopropyl) or S(cyclopropyl), wherein said (4- to 6-membered)heterocycloalkyl and (5- to 6-membered)heteroaryl is substituted by 0 or 1 substituent selected from C₁₋₂alkyl and wherein is said C₁₋₄alkyl is substituted by 0 or 1 substituent selected from CN or OCH₃, and 0, 1, 2 or 3 F and wherein said cyclopropyl and cyclobutyl is substituted by 0 or 1 substituent selected from CN, OCH₃, OCFH₂, OCF₂H, OCF₃ and CH₂CN and 0, 1, 2 or 3 F;
n is 0 or 1;
p is 1, 2 or 3;
or a pharmaceutically acceptable salt thereof.

5. A compound according to claim 4,
wherein
R¹ is independently selected from F, Cl and CN;
R² is selected from F, Cl or CN;
R³ is selected from H, F, Cl, N(CH₃)₂, C₁₋₂alkyl and OC₁₋₂alkyl, wherein said C₁₋₂alkyl is substituted by 0, 1, 2 or 3 F;
R⁴ is independently selected from H, F, Cl, OH, CH₃ and OCH₃;
R⁵ is selected from H, CH₃, CFH₂, CF₂H and CF₃;
R⁶ is selected from (4- to 6-membered)heterocycloalkyl wherein said (4- to 6-membered)heterocycloalkyl is substituted by 0 or 1 substituent selected from C₁₋₂alkyl and;
n is 0 or 1;
p is 1;
or a pharmaceutically acceptable salt thereof.

6. A compound according to claim 1 selected from:
2-(((1R*,6S*)-5-((R*)-2-(5-Chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-((((2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
4-Chloro-2-(((1R*,6R*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid, 2-(((1R*,6R*)-5-((R*)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-((((2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-((((2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1R*,6S*)-5-((R*)-2-(4-Chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
rel-4-Chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylic acid,
2-(((1RS,6RS)-5-(2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1RS,6RS)-5-(2-(5-Chloropyridin-2-yl)-2-methylbenzo[d][1,3]dioxol-4-yl)-2,5-diazabicyclo[4.2.0]octan-2-yl)methyl)-5-methoxy-1-(((S)-oxetan-2-yl)methyl)-1H-benzo[d]imidazole-6-carboxylic acid,
rac-2-(((1R,6R)-5-(2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-methoxy-1H-benzo[d]imidazole-6-carboxylic acid,
rac-2-(((1R,6R)-5-(2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-fluoro-1H-benzo[d]imidazole-6-carboxylic acid,
2-((((R 2-(4-Cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
4-Chloro-2-(((1R*,6S*)-5-((R*)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-((((R 2-(4-Cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-((((2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R*,6S*)-5-((R*)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
4-Chloro-2-(((1R*,6S*)-5-((R*)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-((((R 2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylic acid,
rei-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylic acid,
rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylic acid,
rel-4-Chloro-2-(((1R,6R)-5-((R)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylic acid,
rel-2-(((1R,6R)-5-((R)-2-(5-Chloropyridin-2-yl)-2-methylbenzo[d][1,3]dioxol-4-yl)-2,5-diazabicyclo[4.2.0]octan-2-yl)methyl)-1-(2-cyclopropoxyethyl)-4-methoxy-1H-benzo[d]imidazole-6-carboxylic acid,
or a pharmaceutically acceptable salt thereof.

7. A compound according to claim 1 selected from:
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
4-Chloro-2-(((1R,6S)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1S,6S)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1S,6R)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-4-methoxy-1-(((S)-tetrahydrofuran-2-yl)methyl)-1H-benzo[d]imidazole-6-carboxylic acid,
2-(((1R,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1R,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1S,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1S,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
4-Chloro-2-(((1R,6S)-5-((S)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylic acid,
4-Chloro-2-(((1R,6R)-5-((S)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylic acid,
4-Chloro-2-(((1S,6S)-5-((S)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylic acid,
4-Chloro-2-(((1S,6R)-5-((S)-2-(5-chloropyridin-2-yl)-2-methylbenzo[d][1,3]dioxol-4-yl)-2,5-diazabicyclo[4.2.0]octan-2-yl)methyl)-1-((1-ethyl-1H-imidazol-2-yl)methyl)-1H-benzo[d]imidazole-6-carboxylic acid,
2-(((1R,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-5-methoxy-1-(((S)-oxetan-2-yl)methyl)-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-5-methoxy-1-(((S)-oxetan-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-5-methoxy-1-(((S)-oxetan-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-5-methoxy-1-(((S)-oxetan-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-methoxy-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-methoxy-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-methoxy-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-methoxy-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-fluoro-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-fluoro-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-fluoro-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(2-(2,2-difluorocyclopropoxy)ethyl)-4-fluoro-1H-benzo[d]imidazole-6-carboxylic acid,
2-(((1R,6S)-5-((S)-2-(4-Cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6R)-5-((S)-2-(4-Cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6S)-5-((S)-2-(4-Cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6R)-5-((S)-2-(4-Cyano-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
4-Chloro-2-(((1R,6S)-5-((S)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1R,6R)-5-((S)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1S,6S)-5-((S)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1S,6R)-5-((S)-2-(4-cyano-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6S)-5-((S)-2-(4-Cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1R,6R)-5-((S)-2-(4-Cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1S,6S)-5-((S)-2-(4-Cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1S,6R)-5-((S)-2-(4-Cyano-2-fluorophenyl)-2-methylbenzo[d][1,3]dioxol-4-yl)-2,5-diazabicyclo[4.2.0]octan-2-yl)methyl)-4-fluoro-1-(((S)-oxetan-2-yl)methyl)-1H-benzo[d]imidazole-6-carboxylic acid,
2-(((1R,6S)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6R)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1S,6S)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1S,6R)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1R,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6S)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
2-(((1S,6R)-5-((S)-2-(4-Chloro-2-fluorophenyl)-2-methylbenzo[d][1,3]dioxol-4-yl)-2,5-diazabicyclo[4.2.0]octan-2-yl)methyl)-4-methoxy-1-(((S)-oxetan-2-yl)methyl)-1H-benzo[d]imidazole-6-carboxylic acid,
4-Chloro-2-(((1R,6S)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1R,6R)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1S,6S)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
4-Chloro-2-(((1S,6R)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6S)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6R)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1S,6S)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-is carboxylic acid,
2-(((1S,6R)-5-((S)-2-(4-Chloro-2-fluorophenyl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6r)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-4-fluoro-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-fluoro-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-((1-cyanocyclopropyl)methyl)-4-methoxy-1H-benzo[d]imidazole-6-carboxylic acid,
4-Chloro-2-(((1R,6S)-5-((S)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylic acid,
4-Chloro-2-(((1R,6R)-5-((S)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylic acid,
4-Chloro-2-(((1S,6S)-5-((S)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylic acid,
4-Chloro-2-(((1S,6R)-5-((S)-2-(5-chloropyridin-2-yl)-2-methylbenzo[d][1,3]dioxol-4-yl)-2,5-diazabicyclo[4.2.0]octan-2-yl)methyl)-1-((1-(cyanomethyl)cyclopropyl)methyl)-1H-benzo[d]imidazole-6-carboxylic acid,
2-(((1R,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(2-cyclopropoxyethyl)-4-methoxy-1H-benzo[d]imidazole-6-carboxylic acid,
2-(((1R,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(2-cyclopropoxyethyl)-4-methoxy-1H-benzo[d]imidazole-6-carboxylic acid,
2-(((1S,6S)-5-((S)-2-(5-Chloropyridin-2-yl)-2-methylbenzo[d][1,3]dioxol-4-yl)-2,5-diazabicyclo[4.2.0]octan-2-yl)methyl)-1-(2-cyclopropoxyethyl)-4-methoxy-1H-benzo[d]imidazole-6-carboxylic acid,
2-(((1S,6R)-5-((S)-2-(5-Chloropyridin-2-yl)-2-methylbenzo[d][1,3]dioxol-4-yl)-2,5-diazabicyclo[4.2.0]octan-2-yl)methyl)-1-(2-cyclopropoxyethyl)-4-methoxy-1H-benzo[d]imidazole-6-carboxylic acid,
or a pharmaceutically acceptable salt thereof.

8. A compound according to claim 1 selected from:
4-Chloro-2-(((1R,6R)-5-((S)-2-(5-chloropyridin-2-yl)-2-methylbenzo[d][1,3]dioxol-4-yl)-2,5-diazabicyclo[4.2.0]octan-2-yl)methyl)-1-(((S)-oxetan-2-yl)methyl)-1H-benzo[d]imidazole-6-carboxylic acid,
or a pharmaceutically acceptable salt thereof.

9. A compound according to any of claims 1-8, or a pharmaceutically acceptable salt thereof, for use as a medicament.

10. A pharmaceutical composition comprising a compound according to any one of claims 1-8, or a pharmaceutically acceptable salts thereof, optionally in admixture with a pharmaceutically acceptable adjuvant, diluents or carrier.

11. A method of treating, or reducing the risk of, cardiovascular disease or metabolic conditions which comprises administering to a person suffering from or at risk of, said disease or condition, a therapeutically effective amount of a compound according to any one of claims 1-8, or a pharmaceutically acceptable salt thereof.

12. The method according to claim 11, wherein said disease is type 2 diabetes.

13. A compound according to any one of claims 1-8, or a pharmaceutically acceptable salt thereof, for use in the treatment of type 2 diabetes.

14. The use of a compound according to any one of claims 1-8, or a pharmaceutically acceptable salt thereof, in the manufacture of a medicament, for the treatment or prophylaxis of cardiovascular disease or metabolic conditions.
